(19) United States
(12) Patent Application Publication
(10) Pub. No.: US 2025/0188106 A1
(43) Pub. Date: Jun. 12, 2025

(54) SUBSTITUTED IMIDAZO[1,5-A]PYRIDINE N-HETEROCYCLIC CARBENE (NHC) LIGANDS, CATALYST COMPLEXES THEREOF, AND METHODS USING SAME

(71) Applicant: Rutgers, The State University of New Jersey, New Brunswick, NJ (US)

(72) Inventors: Michal SZOSTAK, Newark, NJ (US); Tongliang ZHOU, Newark, NJ (US); Shiyi YANG, Newark, NJ (US)

(73) Assignee: Rutgers, The State University of New Jersey, New Brunswick, NJ (US)

(21) Appl. No.: 18/845,317

(22) PCT Filed: Mar. 9, 2023
(86) PCT No.: PCT/US2023/014878

§ 371 Date: Sep. 9, 2024

Related U.S. Application Data
(60) Provisional application No. 63/318,481, filed on Mar. 10, 2022.

(51) Int. Cl. C07F 15/00; B01J 31/22; C07D 471/04; C07F 1/00; C07F 1/08

(52) U.S. Cl. CPC C07F 15/006; B01J 31/2273; C07D 471/04; C07F 1/00; C07F 1/08; C07F 15/0046; B01J 2231/4283; B01J 2531/824

(57) ABSTRACT

The present disclosure provides substituted imidazo[1,5-a]pyridine N-heterocyclic carbene (NHC) ligands, catalyst complexes thereof, and methods using same. The present disclosure further provides synthetic methods of preparing N-heterocyclic carbene ligands and catalyst complexes thereof.

### CROSS-REFERENCE TO RELATED APPLICATIONS

The present application claims priority under 35 U.S.C. § 119(e) to U.S. Provisional Patent Application No. 63/318,481, filed Mar. 10, 2022, which is incorporated herein by reference in its entirety.

### STATEMENT REGARDING FEDERALLY SPONSORED RESEARCH

This invention was made with government support under grant number CHE-1650766 awarded by the National Science Foundation and grant number R35GM133326 awarded by the National Institutes of Health. The government has certain rights in this invention.

### BACKGROUND

The Buchwald dialkylbiarylphosphines are among the most widely used class of ligands in catalysis. The key application of these electron-rich and bulky phosphines is to promote a broad range of C—C and C—X bond forming processes in the classical Pd(0)/(II) cross-coupling chemistry. The products of these processes are fundamentally important in both industrial and academic settings in the areas of medicinal chemistry, drug discovery, biochemistry, agrochemistry, natural product synthesis, small molecule synthesis, and polymer synthesis, among other applications. The application of Buchwald dialkylbiarylphosphines is not limited to Pd(0)/(II) catalysis and these ligands are also among the most popular supporting catalysts in the optimization and development of new processes using Pd, Au, Cu, Ni, Rh, and Ir complexes beyond cross-coupling. There are currently a number of commercially available dialkylbiarylphosphine type ligands with the generic dialkyl-biaryl phosphine architecture.

The highly desirable properties of dialkylbiarylphosphines are a result of combining electron-rich dialkylphosphine moiety with the ortho-substituted biaryl motif, which may be modified to accommodate sterically-bulky, electron-rich, coordinating or sterically-differentiated substituents at the ortho, meta or para positions of the biaryl ring. There are two main effects of such modifications: (1) electronically, electron-rich dialkyphosphines facilitate oxidative addition; and (2) sterically, the pivotal biaryl arrangement of dialkylbiarylphosphines (i.e., perpendicular arrangement of the biaryl rings) stabilizes the reactive intermediates through the sterically-defined interaction between the phosphine-metal complexes and the ipso position of the ortho-aromatic ring in a rigid five-membered arrangement. This unique feature renders Buchwald dialkylbiarylphosphines highly valuable in Pd, Au, Cu, Ni, Rh, and Ir catalysis, where two effects are generally observed: (1) enhancement of reactivity compared with other ligands; and (2) unique reactivity not attained with other ligands.

Despite the overwhelming success of Buchwald dialkylbiarylphosphines, the development of novel N-heterocyclic carbene (NHC) analogues has remained elusive, due to a lack of suitable NHC scaffolds.

Thus, there is a need in the art for NHC ligands, and/or catalyst complexes thereof, and methods of using the same. The present disclosure addresses this need.

### BRIEF SUMMARY OF THE INVENTION

In one aspect, the present disclosure provides a compound of formula (I), wherein T¹, T², T³, R², R³, R⁴, and X are defined elsewhere herein:

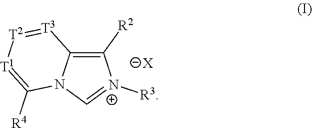

In another aspect, the present disclosure provides a compound of formula (II), wherein T¹, T², T³, R², R³, R⁴, M, L, X, m, and n are defined elsewhere herein:

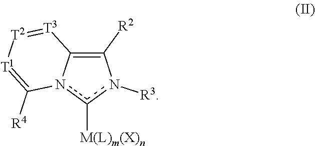

In another aspect, the present disclosure provides a method of promoting a reaction between a pair of compounds selected from the group consisting of:
(a) a boronic acid and a nitroarene;
(b) an alkyl sulfoxide and an aniline;
(c) a thiophenol and an aniline;
(d) an aryl halide and a hydrazine;
(e) an aryl mesylate or aryl tosylate and a boronic acid;
(f) an aryl mesylate or aryl tosylate and an aniline;
(g) an aryl sulfamate and a boronic acid;
(h) an aryl sulfamate and an aniline;
(i) an aryl halide and a hydroxide salt;
(j) an aryl methyl thioether and an aniline;
(k) an aryl sulfoxide and an aniline;
(l) an amine and an aryl chloride;
(m) an aryl chloride and a boronic acid;
(n) an aryl fluoride and an aniline; and
(o) an alkyne and a nitroarene;
   the method comprising contacting the pair of compounds and the compound of the present disclosure according to the conditions described elsewhere herein.

In another aspect, the present disclosure provides a method of preparing a compound of formula (IV), wherein T¹, T², T³, R², R^{3a}, R^{3b}, R^{3c}, R^{3d}, R^{3e}, R^{4a}, R^{4b}, R^{4c}, R^{4d}, R^{4e}, and X are defined elsewhere herein:

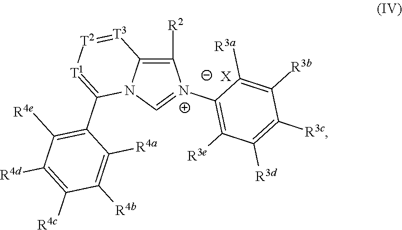

the method comprising contacting (Z):

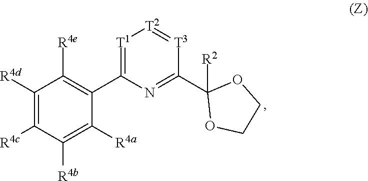

### and (Y):

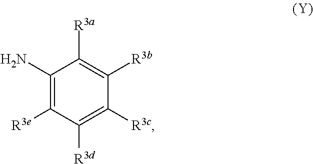

in the presence of paraformaldehyde and an acid.

### BRIEF DESCRIPTION OF THE FIGURES

The drawings illustrate generally, by way of example, but not by way of limitation, various embodiments of the present application.

FIG. 1 provides a comparison of the N-heterocyclic carbenes (NHCs) of the present disclosure and a generic representation of Buchwald dialkylbiarylphosphines.

FIGS. 2A-2B provide structures of imidazo[1,5-α]pyridine NHC—Pd(II) complexes with allyl-type throw-away ligands (FIG. 2A) and pyridine-type throw-away ligands (FIG. 2B).

FIGS. 3A-3C provide structures of NHC ligands of the present disclosure, wherein the 5-position of the imidazo[1,5-α]pyridine is substituted with a higher order 2,4,6-trialkyl-substituted aryl or peralkyl-substituted aryl (FIG. 3A), a 2,4,6-tricycloalkyl-substituted aryl (FIG. 3B), or a 2,6-dialkyl-substituted aryl (FIG. 3C).

FIGS. 4A-4B provides structures of NHC ligands of the present disclosure, wherein the 5-position of the imidazo[1,5-α]pyridine is substituted with an aryl substituent with at least 2,6-dialkoxy substitution (FIG. 4A) or 2-aminoalkyl substitution (FIG. 4B).

FIG. 5 provides structures of NHC ligands of the present disclosure, wherein the ligands combine the principle of L-shaped ligand design of imidazo[1,5-α]pyridine with bulky-yet-flexible properties of N-wingtip substitution.

FIGS. 6A-6C provide structures of NHC ligands of the present disclosure, wherein the 5-position of the imidazo[1,5-α]pyridine is substituted with a cycloalkyl substituent (FIG. 6A), a 3,5-dialkyl-substituted aryl (FIG. 6B), or an ortho-substituted aryl (FIG. 6C).

FIGS. 7A-7B provide x-ray structures of 52 with views from the front (FIG. 7A) and side (FIG. 7B).

FIGS. 8A-8B provide x-ray structures of 39 with views from the front (FIG. 8A) and side (FIG. 8B).

FIGS. 9A-9B provide x-ray structures of 56 with views from the front (FIG. 9A) and side (FIG. 9B).

FIGS. 10A-10F provide topographical steric maps of [Cu(33)Cl] (FIG. 10A), [Cu(34)Cl] (FIG. 10B), [Cu(17)Cl] (FIG. 10C), [Cu(35)Cl] (FIG. 10D), [Cu(36)Cl] (FIG. 10E), and [Cu(19)Cl] (FIG. 10F), determined at the B3LYP 6-311++g(d,p) level.

FIGS. 11A-11C provide representations of the HOMO (n-donating orbital), HOMO (π-donating orbital) and LUMO (π-accepting orbital), respectively, calculated at the B3LYP 6-311++g(d,p) level.

FIG. 12A: Substrate scope for Suzuki-Miyaura cross-coupling of nitroarenes using well-defined, air- and moisture-stable NHC-precatalysts; ^{a}Conditions: nitroarene (1.0 equiv), boronic acid (1.5 equiv), 39 (5 mol %), K₃PO₄ (3 equiv), 1,4-dioxane (0.5 M), TD (10 mol %), H₂O (3 equiv), 36 h, isolated yield; ^{b16} h; ° 2 equiv boronic acid; ^{d}10 mol % 39; TDA=(tris(3,6-dioxaheptylamine). FIG. 12B: Plot of conversion vs. time in the cross-coupling of 4-nitroanisole and phenylboronic acid catalyzed by NHC catalysts of the present disclosure.

FIG. 13A: Substrate scope for Suzuki-Miyaura cross-coupling of nitroarenes using well-defined, air- and moisture-stable [Pd-PEPPSI] NHC-precatalysts; ^{a}nitroarene (1.0 equiv), boronic acid (1.5 equiv), 47 (5 mol %), K₃PO₄ (3 equiv), 1,4-dioxane (0.5 M), TDA (10 mol %); H₂O (3 equiv), 36 h, isolated yield; ^{b}2 equiv boronic acid; TDA=(tris(3,6-dioxaheptyl)amine). FIG. 13B: Plot of conversion vs. time in the cross-coupling of 4-nitroanisole with phenylboronic acid catalyzed by NHC catalysts of the present disclosure.

FIG. 14A: Substrate scope of anilines in Buchwald-Hartwig amination of alkyl sulfoxides (Ar—S(═O)(alkyl)). FIG. 14B: Substrate scope of sulfoxides (i.e., aryl component varied) in Buchwald-Hartwig amination of alkyl sulfoxides. FIG. 14C: Substrate scope of sulfoxides (i.e., alkyl component varied) in Buchwald-Hartwig amination of alkyl sulfoxides. FIG. 14D: Examples of late-stage functionalizations utilizing a NHC—Pd catalyzed Buchwald-Hartwig amination of alkyl sulfoxides. FIG. 14E: provides optimization conditions for the Buchwald-Hartwig amination of alkyl sulfoxides.

FIG. 15A: Substrate scope of anilines in Buchwald-Hartwig amination of thiophenols. FIG. 15B: Substrate scope of thiophenols in Buchwald-Hartwig amination of thiophenols. FIG. 15C: provides optimization conditions for the Buchwald-Hartwig amination of thiophenols.

FIG. 16A: Substrate scope of aryl chlorides in reaction sequence comprising hydrazination and aromatization; ^{a}without THF. FIG. 16B: Substrate scope of aryl bromides in reaction sequence comprising hydrazination and aromatization; ^{a}without TFA. FIG. 16C: Examples of late stage functionalizations using a NHC—Pd catalyzed hydrazination with subsequent aromatization. FIG. 16D: provides optimization conditions for the hydrazination/aromatization sequence.

FIG. 17A: Substrate scope of Suzuki-Miyaura cross-coupling with aryl mesylates; Conditions: aryl mesylate (1.0 equiv), aryl boronic acid (4.5 equiv), K₃PO₄ (3.0 equiv), 43 (5 mol %), iPrOH (0.2 M), 130° C., 12 h. FIGS. 17B-17C: provide optimization conditions for the Suzuki-Miyaura cross-coupling with aryl mesylates; L₁ (9), L₂ (35), and L₃ (34).

FIG. 18A: Substrate scope of Buchwald-Hartwig cross-coupling with aryl mesylates; Conditions: aryl mesylate (1.0 equiv), aniline (1.2 equiv), K₃PO₄ (2.0 equiv), 42 (5 mol %), tAmOH (0.2 M), 120° C., 12 h. FIG. 18B: provides optimization conditions for the Buchwald-Hartwig cross-coupling with aryl mesylates; L₁ (9), L₂ (35), and L₃ (34).

FIG. 19A: Substrate scope of Suzuki-Miyaura cross-coupling with aryl sulfamates; Conditions: aryl sulfamate (1.0 equiv), aryl boronic acid (4.5 equiv), K₃PO₄ (3.0 equiv), 42 (5 mol %), iPrOH (0.2 M), 130° C., 12 h. FIG. 19B: provides optimization conditions for the Suzuki-Miyaura cross-coupling with aryl sulfamates; L₁ (9), L₂ (35), and L₃ (34).

FIG. 20A: Substrate scope of Buchwald-Hartwig cross-coupling with aryl sulfamates; Conditions: aryl sulfamate (1.0 equiv), aniline (1.2 equiv), K₃PO₄ (2.0 equiv), 42 (5 mol %), tAmOH (0.2 M), 120° C., 12 h. FIG. 20B: provides optimization conditions for the Buchwald-Hartwig cross-coupling with aryl sulfamates; L₁ (9), L₂ (35), and L₃ (34).

FIGS. 21A-21E provide non-limiting examples of additional reactions catalyzed using the NHC ligands and/or catalyst complexes thereof described herein, non-limiting examples including hydroxylation of aryl chlorides (FIG. 21A), Buchwald-Hartwig amination of alkyl thioethers (FIG. 21B), Buchwald-Hartwig amination of aryl sulfoxides (FIG. 21C), Buchwald-Hartwig amination of aryl chlorides (FIG. 21D), and Suzuki-Miyaura cross-coupling of aryl chlorides (FIG. 21E).

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Introduction

Despite the overwhelming success of Buchwald dialkylbiarylphosphines, the development of related N-heterocyclic carbene (NHC) ligands has been difficult due to the inefficiency of common NHC architectures to support the biaryl arrangement of dialkylbiarylphosphines that stabilizes the reactive ligand-metal intermediates through the sterically-defined rigid five-membered interaction.

NHC ligands are well recognized as extremely valuable ligands on its own due to (1) stronger σ-donation and π-acceptance (cf. phosphine ligands), (2) unique umbrella shape of the steric bulk around the donor center (cf. cone of phosphines), and (3) ease of peripheral N-wingtip and scaffold modification to further tune steric and electronic properties of the ligand. As a result, NHCs among the most popular ligands in transition-metal-catalysis, organocatalysis, organic synthesis, and materials science, inter alia, with many NHCs currently commercially available.

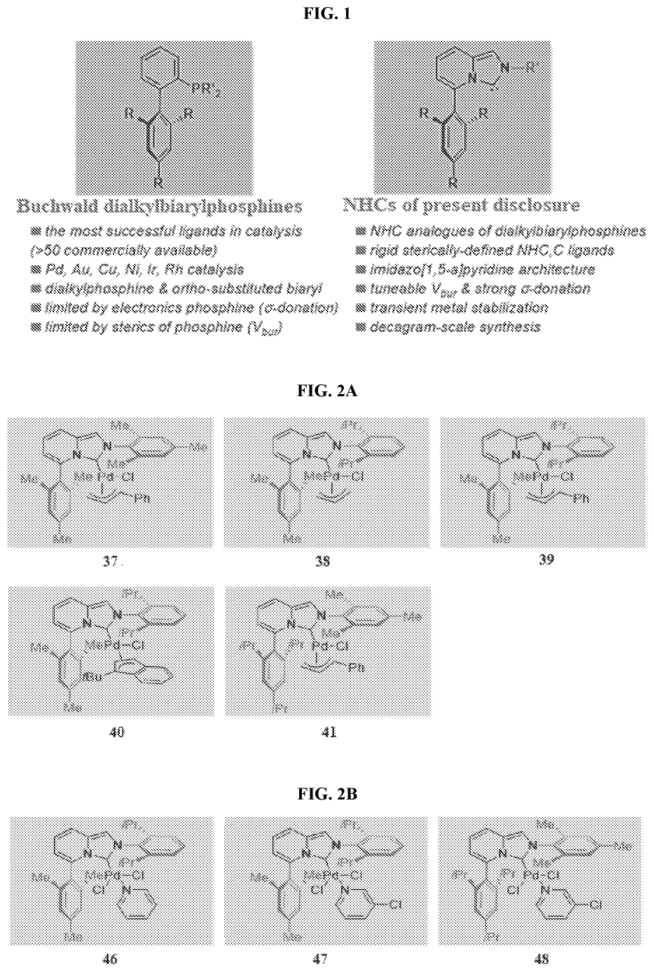

The present disclosure provides a sterically-defined L-shaped NHC technology platform for the development of new cross-coupling reactions, which have yet to be achieved using other NHC ligands, and improvement of efficiency of the existing cross-coupling methods using other ligands (FIG. 1). Further, the present disclosure describes: (1) novel sterically-defined L-shaped biaryl NHC ligands on imidazo[1,5-α]pyridine architecture; (2) well-defined, air- and moisture-stable complexes of NHCs with transition-metals; and (3) application of NHC-catalyst complexes in palladium-catalyzed cross-coupling reactions. Additionally, feasibility studies have demonstrated the synthesis of well-defined complexes with Au, Cu, Ag, and Rh.

As described herein, in certain embodiments, the aryl substituent is placed at the C5-position of the imidazo[1,5-α]pyridine scaffold, which, without wishing to be bound by theory, results in the well-defined rigid perpendicular arrangement of the biaryl moiety that enables a stabilizing interaction between the ipso position of the C5-aromatic ring and the metal center coordinated to the NHC carbon, thereby providing the geometric properties of Buchwald dialkylbiarylphosphines with NHC ligation.

In one aspect, the present disclosure describes the following classes, features, and/or aspects of the compounds disclosed herein: (1) well-defined complexes; (2) C5 differentiation; (3) RuPhos NHC analogues; (4) N2 differentiation; and (5) C5 alkyl Substitution, C5 meta-aryl substitution and C5-mono-ortho-aryl substitution.

Well-defined complexes. Development of well-defined sterically-defined Pd(II)—NHC complexes of imidazo[1,5-α]pyridine (FIGS. 2A-2B). Specifically, it has been demonstrated herein that the use of Pd(II)—NHC-cinnamyl complexes with L-shaped imidazo[1,5-α]pyridine ligands of the general formula [Pd(NHC)(cin)Cl]results in a significantly more active catalysts based on imidazo[1,5-α]pyridine scaffold for Suzuki cross-coupling of nitroarenes (R—NO₂). In certain embodiments, well-defined PEPPSI-type catalysts with L-shaped imidazo[1,5-α]pyridine ligands of the general formula of [Pd(NHC)(3-Cl-py)Cl₂] are preferred due to the ease of synthesis and stability.

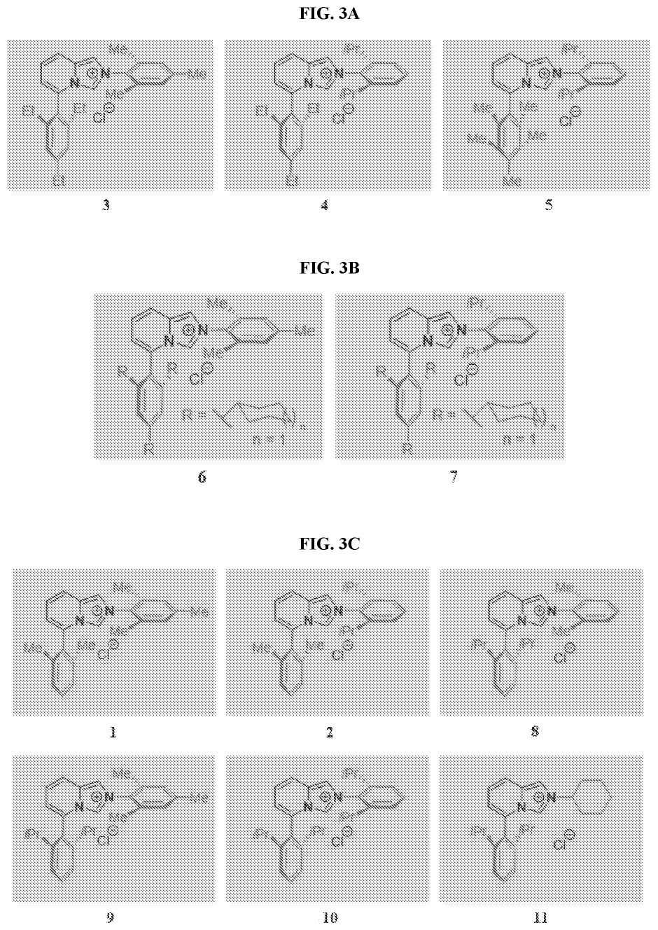

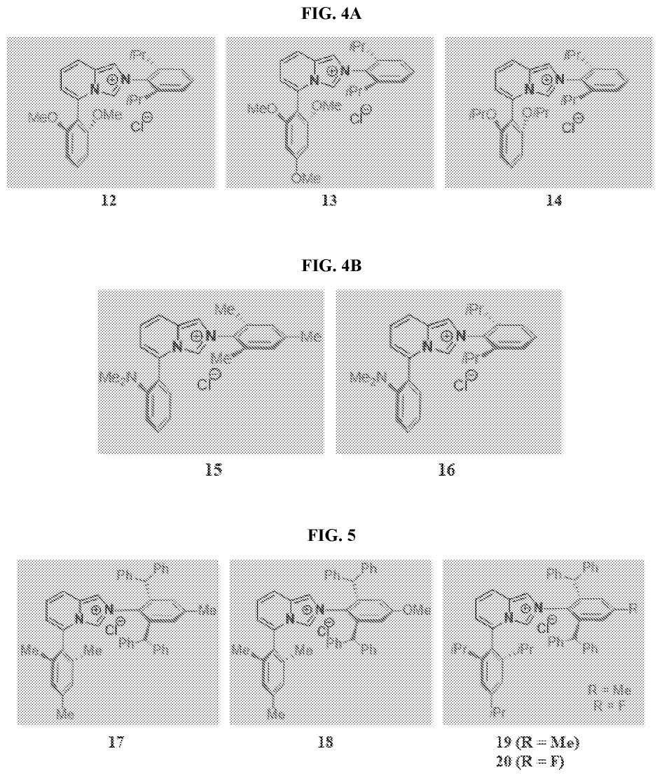

C5 Differentiation. Development of new catalysts with steric differentiation at the C5 aromatic ring (FIGS. 3A-3C). The ortho-substituted biaryl motif is critical for activity due to stabilizing interaction between the C5-aromatic ring and the metal center coordinated to the NHC carbon. Thus, rational modification of the C5 substitution has a major impact on elementary steps of catalytic cycle. Specifically, the synthesis and catalytic activity of L-shaped imidazo[1,5-α]pyridine ligands has been demonstrated herein, wherein the C5 aromatic ring is substituted with any of a number of substituents, including but not limited to: (a) higher order 2,4,6-trialkyl and per-alkyl hydrocarbon chains; (b) 2,4,6-trialkyl cyclic hydrocarbon chains; (c) sterically-flexible 2,6-dialkyl hydrocarbon chains, which, in certain embodiments, open-up steric space at the 4-position of the aromatic ring.

RuPhos NHC Analogues. The development of highly active sterically-defined L-shaped NHC ligands that are analogous to RuPhos (i.e., 2,6-alkoxyaryl at the 5-position of the imidazo[1,5-α]pyridine) (FIGS. 4A-4B). Specifically, structure-activity relationship studies of sterically-defined L-shaped NHC ligands with imidazo[1,5-α]pyridine are provided herein, which resulted in the establishment of steric reactivity maps demonstrating steric impact of the C5-aromatic ring and N2-aromatic ring. In one aspect, these findings resulted in the development of a highly active carbene analogue of RuPhos, which is characterized by the presence of electron-rich chelating 2,6-O-i-Pr₂-substitution of the C5 aromatic ring. Related ligands with variation of chelating heteroatoms and steric hindrance at the ortho-position of the C5 aromatic ring have been also developed (e.g., 2-NR₂-phenyl, 2,6-dimethoxyphenl, and 2,4,6-trimethoxyphenyl).

N2 Differentiation. Development of new catalysts with steric differentiation at the N2 position (FIG. 5). The unique umbrella shape of NHC ligands enables to use peripheral N-wingtip modification to tune steric and electronic properties of the ligand. A class of sterically-defined L-shaped NHC ligands with imidazo[1,5-α]pyridine with sterically-bulky N2-substitution has been developed. Specifically, the synthesis and catalytic activity of catalysts with N2-substitution of the general formula of Ar=2,6-(Ph₂CH)₂-4-R—C₆H₂, (R=Me, OMe, F), is demonstrated herein. Such catalysts combine the features of bulky-yet-flexible IPr* series of NHC ligands with L-shaped imidazo[1,5-α]pyridines.

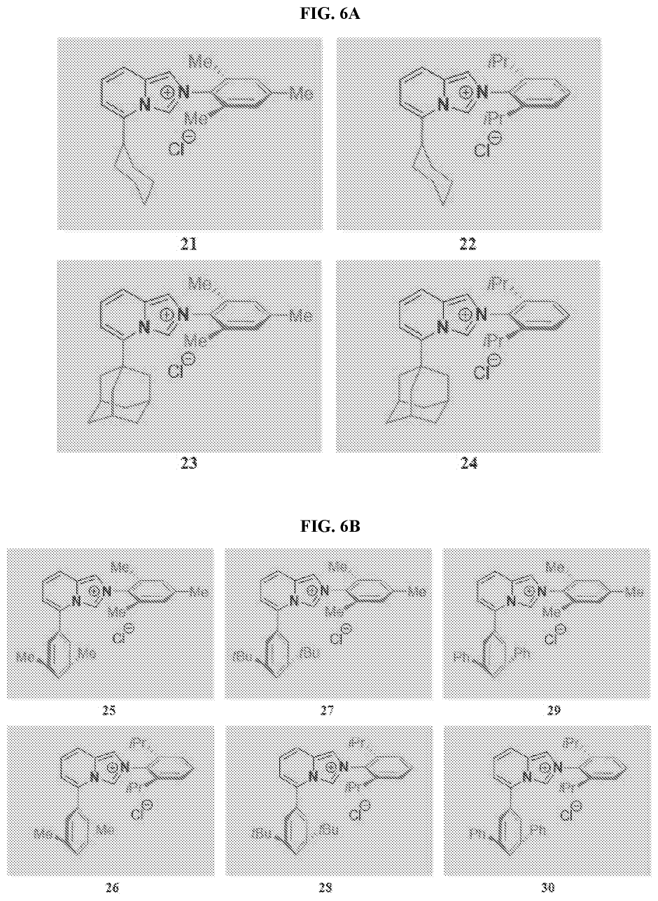

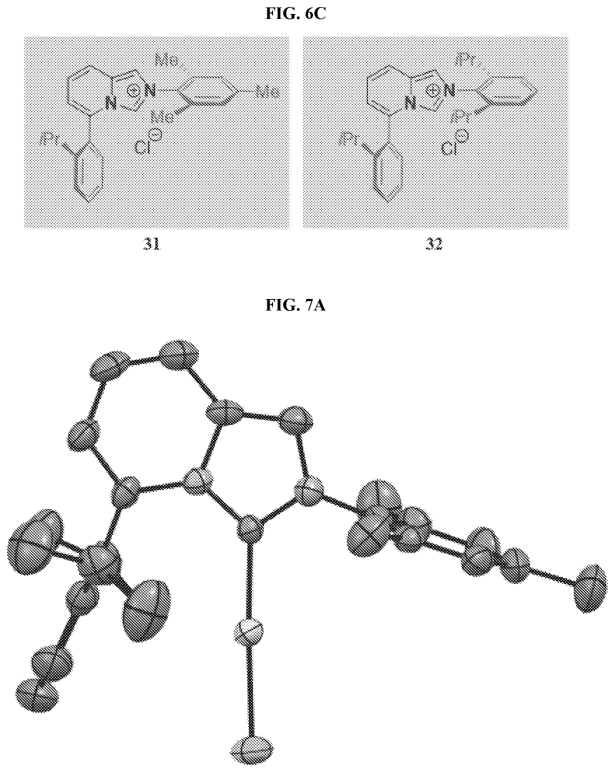

C5-Alkyl Substitution, C5-meta-Aryl Substitution and C5-mono-ortho-Aryl Substitution. Development of new catalysts with C5-alkyl, C5 meta-aryl and C5-mono-orho-aryl substitution (FIGS. 6A-6C). Feasibility studies have demonstrated the synthesis of sterically-defined NHC ligands with imidazo[1,5-α]pyridine that feature: C5-alkyl-substitution instead of the more common aromatic ring, C5-meta-aryl substitution, or C5-mono-ortho aryl substitution. The present disclosure demonstrates the use of the catalysts described herein in palladium-catalysis, however, these classes of ligands are expected to show unique reactivity with metals that do not require di-ortho-substitution for the reductive elimination step, including but not limited to Ru, Rh, Ir, Cu, Ag and Ni.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)=CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “amine” as used herein refers to primary, secondary, and tertiary amines having, e.g., the formula N (group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aniline” as used herein refers to an amine having at least one aryl or heteroaryl substituent.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aroyl” as used herein refers to an aryl group, as defined elsewhere herein, substituted at any one position with a carbonyl moiety (i.e., C(═O)). Use of the term “aroyl” in combination with another term (e.g., chloride), indicates that the carbonyl linked to the aryl group is substituted with the substituent defined by the term used in the combination. For example, PhC(═O)Cl (i.e., benzoyl chloride) is an aroyl chloride.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups.

Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof. Use of the term “aryl” in combination with another term (e.g., iodide, chloride, boronic acid, and magnesium halide, inter alia), indicates that the aryl group is substituted at one or more positions with the substituent defined by the term used in the combination. For example, an aryl chloride indicates that the aryl is substituted with at least one chloride.

The term “atm” as used herein refers to a pressure in atmospheres under standard conditions. Thus, 1 atm is a pressure of 101 kPa, 2 atm is a pressure of 202 kPa, and so on.

The term “counter anion” as used herein refers to a negatively charged ion that accompanies a cationic species (i.e. positively charged ion) in order to maintain electric neutrality. For example, the chloride ion (Cl⁻) is the counter anion to sodium (Na⁺) in NaCl. Non-limiting examples of counter anions include F⁻, Cl⁻, Br⁻, I⁻, H⁻, N₃⁻, PhCH═CHCH₂⁻, PhCH⁻ CH═CH₂, F₃CS(═O)₂O⁻ (OTf), (F₃CS(═O)₂)₂N—(NTf₂), F₃CC(═O)O⁻ (TFA), BF₄⁻, and PF₆⁻.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “electrophile” as used herein refers to a chemical species that forms a bond with a nucleophile by accepting an electron pair in a chemical reaction (e.g. S_{N}1, S_{N}2, and carbonyl [1,2]-addition). Non-limiting examples of electrophiles include alkyl halides (e.g. Mel), benzyl halides (e.g. BnBr), dihalides (e.g. Br₂), aldehydes (e.g. Ph-CHO), acyl halides (e.g. Ph(C═O)Cl), N-electrophiles (e.g. RC(═O)ONR₂), and O-electrophiles (e.g. RC(═O)O₂R).

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxy butyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaralkynyl” as used herein refers to alkynyl groups as defined herein in which a hydrogen or carbon bond of an alkynyl group is replaced with a bond to a heteroaryl group as defined herein. Representative aralkynyl groups include, but are not limited to, 2-ethynylpyridine and 2-ethynylthiophene.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “IMes” as used herein refers to 1,3-bis(2,4,6-trimethylphenyl)-1,3-dihydro-2H-imidazol-2-ylidene.

The term “ImPy” as used herein refers to imidazo[1,5-α]pyridine.

The term “IPr” as used herein refers to 1,3-bis(2,6-diisopropylphenyl)-1,3-dihydro-2H-imidazol-2-ylidene.

The term “IPr*” as used herein refers to 1,3-bis((2,6-(dibenzhydryl))-4-methylphenyl)-1,3-dihydro-2H-imidazol-2-ylidene.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “Lewis acid” as used herein refers to a chemical species that possesses an empty orbital which is capable of accepting a pair of non-bonding electrons from a species having a filled orbital containing an electron pair (i.e., a Lewis base).

The term “lithium amide” as used herein refers to a lithium salt of an anionic amine of the formula N(R)(R), wherein each R is independently selected from the group consisting of H, C₁-C₁₂ alkyl, and Si(C₁-C₁₂ alkyl)₃. Non-limiting examples of lithium amides include lithium diisopropylamide (LDA) and lithium hexadimethylsilazide (LiHMDS).

The terms “[M(#)X]” or “[M(NHC)X]” as used herein indicate a N-heterocyclic carbene (NHC) complex comprising a metal (M) (e.g., Au, Pd, Ag, Cu, and Rh), an anionic counter ion (X) (e.g., Cl), and an NHC derived from an imidazolium salt (i.e., NHC or compound #).

Additional terms may be included to indicate further ligands and/or counter ions. In such compounds, the compound number or name provided for the NHC may correspond to an imidazolium salt, however, the catalyst complex comprises the carbene derived from the indicated imidazolium salt complexed to the indicated metal.

The term “monovalent” as used herein refers to a substituent connecting via a single bond to a substituted molecule. When a substituent is monovalent, such as, for example, F or Cl, it is bonded to the atom it is substituting by a single bond.

The term “neutral ligand” or “ligand” as used herein, refers to a ligand having no net charge prior to association with, or after dissociation from, a metal center. Non-limiting examples of neutral ligands include alkene (e.g., cyclooctadiene), CO, amine (e.g., NMe₃), phosphine (e.g., PPh₃), and pyridyl ligands, wherein coordination occurs via the nitrogen lone pair of the pyridyl group.

The term “nitrile” as used herein refers to an organic compound comprising a cyano group (C≡N).

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “protic solvent” as used herein refers to a solvent that has a hydrogen atom bound to a heteroatom such as O, N, or S, such that the H⁺ is labile. Non-limiting examples of protic solvents include methanol, ethanol, isopropanol, acetic acid, water, n-butanol, and formic acid. Conversely, an “aprotic solvent” as used herein refers to a solvent lacking dissociable hydrogen ions (i.e. non-acidic) to an appreciable extent. Non-limiting examples of aprotic solvents include ethyl acetate (EtOAc), diethyl ether (Et₂O), tetrahydrofuran (THF), dimethylformamide (DMF), and 1,4-dioxane.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

### Preparation of Compounds

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

The compounds of the present disclosure can be prepared by the general schemes described herein, using synthetic methods known by those skilled in the art. The following examples illustrate non-limiting embodiments of the compound(s) described herein and their preparation.

In certain illustrative embodiments, N-heterocyclic carbene ligands of the present disclosure were prepared according to Schemes 1-2.

In certain embodiments, the N-heterocyclic carbene ligands are prepared as provided in Scheme 1 (Method A), utilizing Pd-catalyzed Suzuki cross-coupling of an aryl boronic acid (1-1) and 1-2, followed by condensation with an amine and cyclization to form the imidazolium under standard conditions (i.e., (CH₂O)₈₋₁₀₀ and HCl).

In certain embodiments, the N-heterocyclic carbene ligands are prepared as provided in Scheme 2 (Method B), utilizing Ni-catalyzed Kumada cross-coupling of 1-6 and arylmagnesium halide 1-8 to provide 1-9, followed by condensation with an amine and cyclization to form the imidazolium under standard conditions (i.e., (CH₂O)₈₋₁₀₀ and HCl). In certain embodiments, the acetal or ketal of 1-9 undergoes hydrolysis during under acidic reaction conditions, including but not limited to HCl in toluene at 100° C. In addition, Pd-catalyzed Negishi cross-coupling has been developed for the synthesis of sterically-demanding C5-alkyl-substituted ligands with imidazo[1,5-α]pyridine.

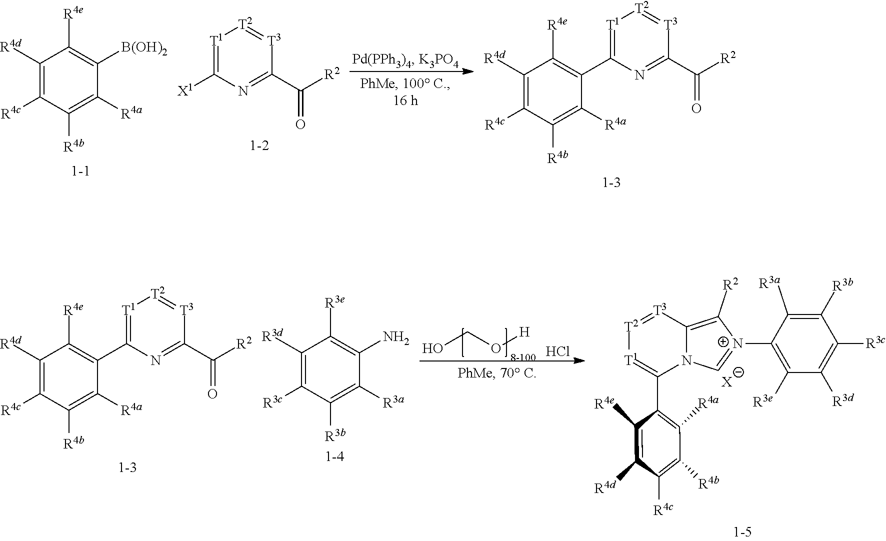

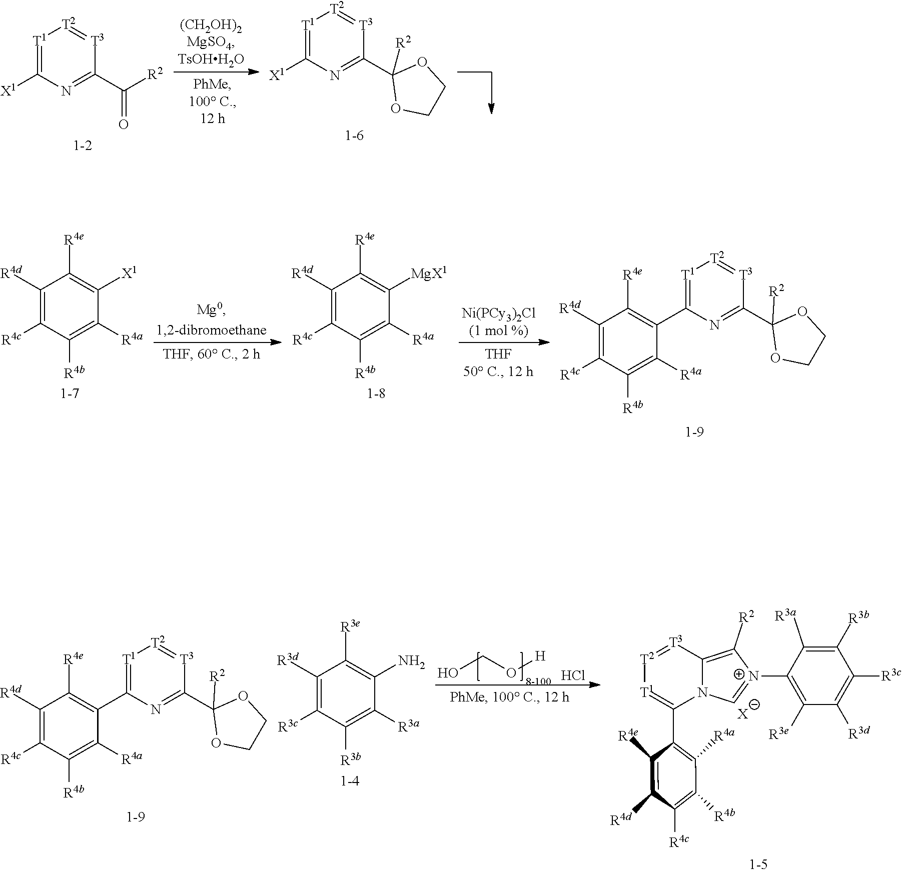

In certain illustrative embodiments, N-heterocyclic carbene (NHC) complexes of the present disclosure were prepared from the corresponding NHC ligands according to Schemes 3-6, wherein T¹, ², T³, R^{1a}, R^{1b}, R^{1c}, R², R³, R^{4a}, R^{4b}, R^{4c}, R^{4d}, R^{4e}, X, and X¹ are defined within the scope of the present disclosure.

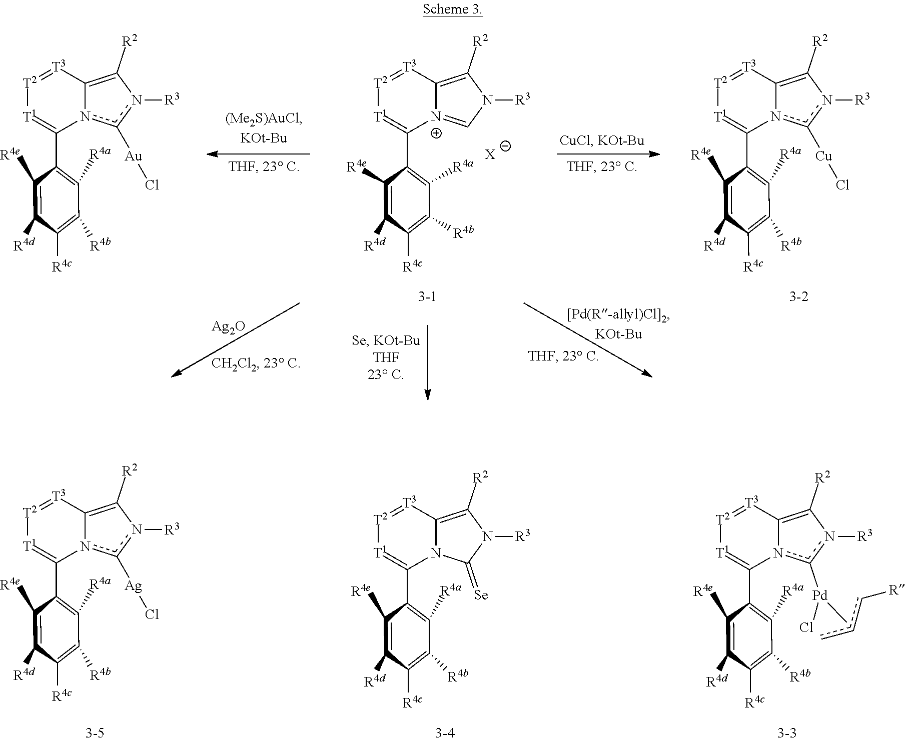

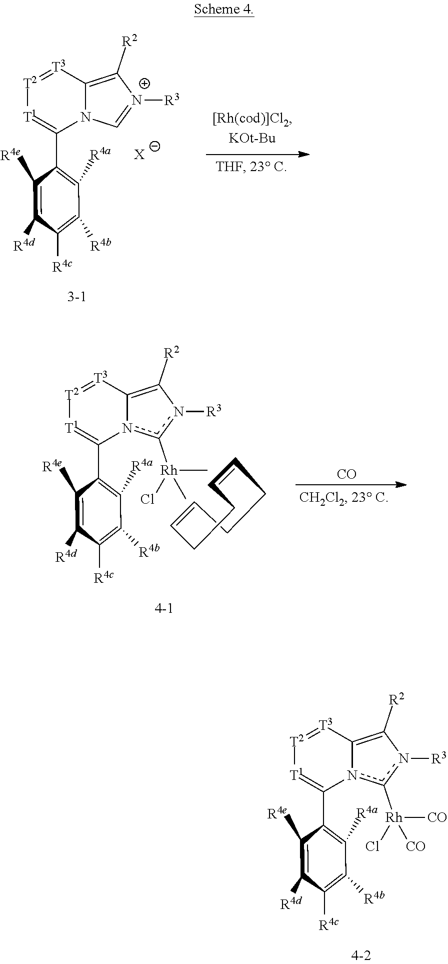

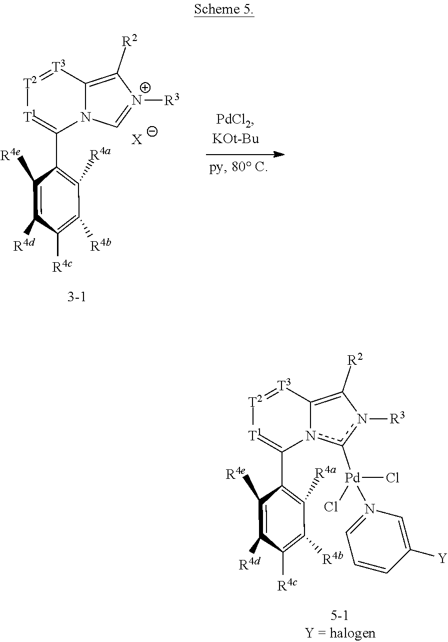

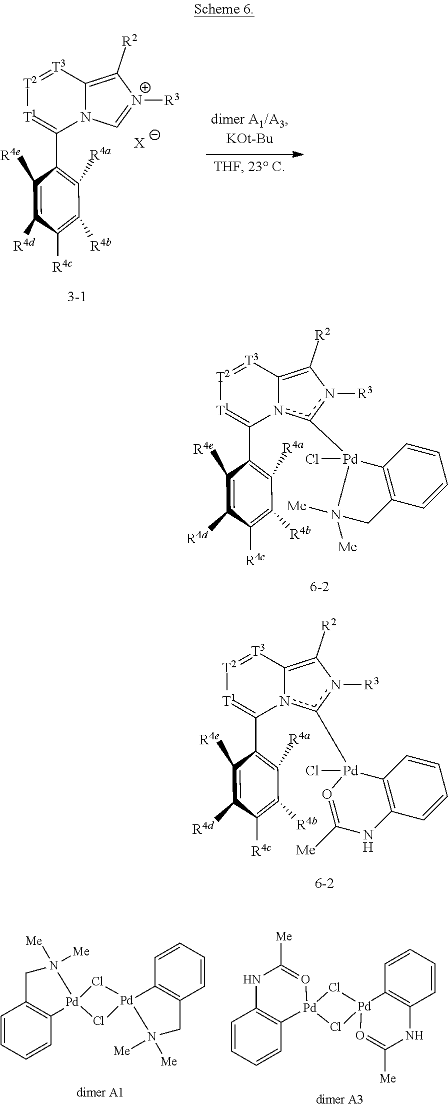

### Compounds

In one aspect, the present disclosure provides a compound of formula (I):

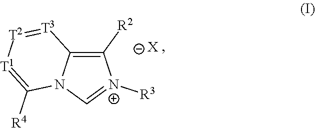

wherein:
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R³ is selected from the group consisting of optionally substituted C₆-C₁₀ aryl, optionally substituted C₂-C₈ heteroaryl, and optionally substituted C₃-C₈ cycloalkyl,
   wherein each optional substituent in R³ is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R⁴ is selected from the group consisting of optionally substituted C₃-C₁₂ cycloalkyl and phenyl substituted with at least one substituent selected from the group consisting of C₁-C₆ alkyl, C₃-C₁₂ cycloalkyl, C₁-C₆ alkoxy, phenyl, and N(R^{A})(R^{B}),
   wherein each optional substituent in the C₃-C₁₂ cycloalkyl is at least one selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
X is a counter anion;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen.

In certain embodiments, if T¹ is CH, T² is CH, T³, is CH, and R² is H, and if R⁴ is 2,4,6-triisopropylphenyl, then R³ is not 2,6-diisopropylphenyl, 2,4,6-trimethylphenyl, 2-methylphenyl, or 2,6-diethyl-4-methylphenyl.

In certain embodiments, if T¹ is CH, T² is CH, T³, is CH, and R² is H, and if R⁴ is 2,4,6-trimethylphenyl, then R³ is not 2,4,6-trimethylphenyl, 2,6-diisopropylphenyl, adamantly, 4-(2-(2-methoxyethoxy)ethoxy)-2,6-dimethylphenyl, or (2-(2-methoxyethoxy)ethoxy)phenyl.

In certain embodiments, if T¹ is CH, T² is CH, T³, is CH, and R² is H, and if R⁴ is 2-methylphenyl, then R³ is not 2,6-diisopropylphenyl.

In certain embodiments, if T¹ is CH, T² is CH, T³, is CH, and R² is H, and if R⁴ is 4-tert-butyl-2-pyridyl, then R³ is not 2,6-diisopropylphenyl.

In certain embodiments, if T¹ is CH, T² is CH, T³, is CH, and R² is H, and if R⁴ is 5-tetrahydroisoquinolinyl, then R³ is not 2,6-diisopropylphenyl.

In certain embodiments, T¹ is CR^{1a}. In certain embodiments, T² is CR^{1b}. In certain embodiments, T³ is CR^{1c}.

In certain embodiments, T¹ is CR^{1a}, T² is CR^{1b}, and T³ is CR^{1c}. In certain embodiments, T¹ is N, T² is CR^{1b}, and T³ is CR^{1c}. In certain embodiments, T¹ is CR^{1a}, T² is N, and T³ is CR^{1c}.

In certain embodiments, T¹ is CR^{1a}, T² is CR^{1b}, and T³ is N. In certain embodiments, T¹ is N, T² is N, and T³ is CR^{1c}. In certain embodiments, T¹ is N, T² is CR^{1b}, and T³ is N. In certain embodiments, T¹ is CR^{1a}, T² is N, and T³ is N.

In certain embodiments, at least one of R^{1a}, R^{1b}, and R^{1c} is H. In certain embodiments, at least two of R^{1a}, R^{1b}, and R^{1c} are H. In certain embodiments, each of R^{1a}, R^{1b}, and R^{1c} are H.

In certain embodiments, R² is H.

In certain embodiments, R³ is.

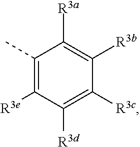

wherein:
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
   wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

In certain embodiments, R^{3a} is H. In certain embodiments, R^{3a} is methyl. In certain embodiments, R^{3a} is i-propyl. In certain embodiments, R^{3a} is diphenylmethyl. In certain embodiments, R^{3a} is methoxy. In certain embodiments, R^{3a} is fluoro.

In certain embodiments, R^{3b} is H. In certain embodiments, R^{3b} is methyl. In certain embodiments, R^{3b} is i-propyl. In certain embodiments, R^{3b} is diphenylmethyl. In certain embodiments, R^{3b} is methoxy. In certain embodiments, R^{3b} is fluoro.

In certain embodiments, R^{3c} is H. In certain embodiments, R^{3c} is methyl. In certain embodiments, R^{3′} is i-propyl. In certain embodiments, R^{3c} is diphenylmethyl. In certain embodiments, R^{3c} is methoxy. In certain embodiments, R^{3c} is fluoro.

In certain embodiments, R^{3d} is H. In certain embodiments, R^{3d} is methyl. In certain embodiments, R^{3d} is i-propyl. In certain embodiments, R^{3d} is diphenylmethyl. In certain embodiments, R^{3d} is methoxy. In certain embodiments, R^{3d} is fluoro.

In certain embodiments, R^{3e} is H. In certain embodiments, R^{3e} is methyl. In certain embodiments, R^{3e} is i-propyl. In certain embodiments, R^{3e} is diphenylmethyl. In certain embodiments, R^{3e} is methoxy. In certain embodiments, R^{3e} is fluoro.

In certain embodiments, R^{3b} and R^{3d} are each independently H. In certain embodiments, R^{3b}, R^{3c}, and R^{3d} are each independently H.

In certain embodiments, R^{3a} and R^{3e} are identical. In certain embodiments, R^{3a}, R^{3c}, and R^{3e} are identical.

In certain embodiments, R^{3a} and R^{3e} are each independently optionally substituted C₁-C₆ alkyl, and R^{3b}, R^{3c}, and R^{3d} are H. In certain embodiments, R^{3a}, R^{3c}, and R^{3e} are each independently optionally substituted C₁-C₆ alkyl, and R^{3b} and R^{3d} are H. In certain embodiments, R^{3a} and R^{3e} are each independently optionally substituted C₁-C₆ alkyl, R^{3c} is selected from the group consisting of halogen and C₁-C₆ alkoxy, and R^{3b} and R^{3d} are H.

In certain embodiments, R³ is

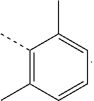

In certain embodiments, R³ is

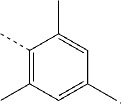

In certain embodiments, R³ is

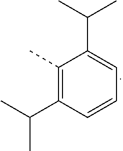

In certain embodiments, R³ is

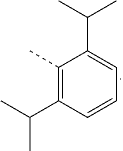

In certain embodiments, R³ is

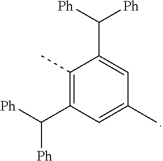

In certain embodiments, R³ is

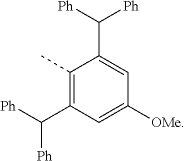

In certain embodiments, R³ is

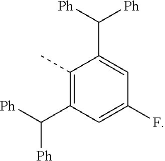

In certain embodiments, R³ is cyclohexyl.

In certain embodiments, R⁴ is:

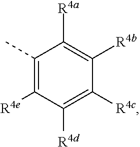

wherein:
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
   wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

In certain embodiments, R^{4a} is H. In certain embodiments, R^{4a} is methyl. In certain embodiments, R^{4a} is ethyl. In certain embodiments, R^{4a} is i-propyl. In certain embodiments, R^{4a} is cyclohexyl. In certain embodiments, R^{4a} is t-butyl. In certain embodiments, R^{4a} is methoxy. In certain embodiments, R^{4a} is i-propoxy. In certain embodiments, R^{4a} is phenyl. In certain embodiments, R^{4a} is dimethylamino.

In certain embodiments, R^{4b} is H. In certain embodiments, R^{4b} is methyl. In certain embodiments, R^{4b} is ethyl. In certain embodiments, R^{4b} is i-propyl. In certain embodiments, R^{4b} is cyclohexyl. In certain embodiments, R^{4b} is t-butyl. In certain embodiments, R^{4b} is methoxy. In certain embodiments, R^{4b} is i-propoxy. In certain embodiments, R^{4b} is phenyl. In certain embodiments, R^{4b} is dimethylamino.

In certain embodiments, R^{4c} is H. In certain embodiments, R^{4c} is methyl. In certain embodiments, R^{4c} is ethyl. In certain embodiments, R^{4c} is i-propyl. In certain embodiments, R^{4c} is cyclohexyl. In certain embodiments, R^{4c} is t-butyl. In certain embodiments, R^{4c} is methoxy. In certain embodiments, R^{4c} is i-propoxy. In certain embodiments, R^{4c} is phenyl. In certain embodiments, R^{4c} is dimethylamino.

In certain embodiments, R^{4d} is H. In certain embodiments, R^{4d} is methyl. In certain embodiments, R^{4d} is ethyl. In certain embodiments, R^{4d} is i-propyl. In certain embodiments, R^{4d} is cyclohexyl. In certain embodiments, R^{4d} is t-butyl. In certain embodiments, R^{4d} is methoxy. In certain embodiments, R^{4d} is i-propoxy. In certain embodiments, R^{4d} is phenyl. In certain embodiments, R^{4d} is dimethylamino.

In certain embodiments, R^{4e} is H. In certain embodiments, R^{4e} is methyl. In certain embodiments, R^{4e} is ethyl. In certain embodiments, R^{4e} is i-propyl. In certain embodiments, R^{4e} is cyclohexyl. In certain embodiments, R^{4e} is t-butyl. In certain embodiments, R^{4e} is methoxy. In certain embodiments, R^{4e} is i-propoxy. In certain embodiments, R^{4e} is phenyl. In certain embodiments, R^{4e} is dimethylamino.

In certain embodiments, R^{4b}, R^{4c}, R^{4d}, and R^{4e} are each independently H. In certain embodiments, R^{4b}, R^{4c}, and R^{4d} are each independently H. In certain embodiments, R^{4a}, R^{4c}, and R^{4e} are each independently H. In certain embodiments, none of R^{4a}, R^{4b}, R^{4c}, R^{4d}, and R^{4e} are H.

In certain embodiments, R^{4a} and R^{4e} are identical. In certain embodiments, R^{4b} and R^{4d} are identical. In certain embodiments, R^{4a}, R^{4c}, and R^{4e} are identical. In certain embodiments, R^{4a}, R^{4b}, R^{4c}, R^{4d}, and R^{4e} are identical.

In certain embodiments, R^{4a} is selected from the group consisting of C₁-C₆ alkyl and N(C₁-C₆ alkyl)₂, and R^{4b}, R^{4c}, R^{4d}, and R^{4e} are each independently H. In certain embodiments, R^{4a} and R^{4e} are each independently C₁-C₆ alkyl, and R^{4b}, R^{4c}, and R^{4d} are each independently H.

In certain embodiments, R^{4a} and R^{4e} are each independently C₁-C₆ alkoxy, and R^{4b}, R^{4c}, and R^{4d} are each independently H. In certain embodiments, R^{4b} and R^{4d} are each independently C₁-C₆ alkyl or phenyl, and R^{4a}, R^{4c}, and R^{4e} are each independently H. In certain embodiments, R^{4a}, R^{4c}, and R^{4e} are each independently C₁-C₆ alkyl or C₃-C₈ cycloalkyl, and R^{4b} and R^{4d} are each independently H. In certain embodiments, R^{4a}, R^{4c}, and R^{4e} are each independently C₁-C₆ alkoxy, and R^{4b} and R^{4d} are each independently H. In certain embodiments, R^{4a}, R^{4b}, R^{4c}, R^{4d}, and R^{4e} are each independently C₁-C₆ alkyl.

In certain embodiments, R⁴ is

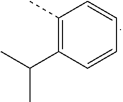

In certain embodiments, R⁴ is

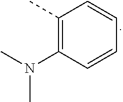

In certain embodiments, R⁴ is

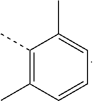

In certain embodiments, R⁴ is

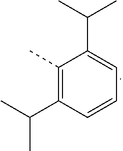

In certain embodiments, R⁴ is

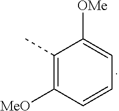

In certain embodiments, R⁴ is

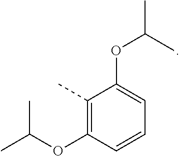

In certain embodiments, R⁴ is

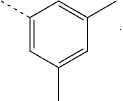

In certain embodiments, R⁴ is

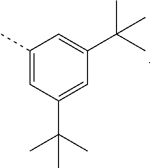

In certain embodiments, R⁴ is

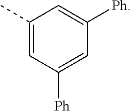

In certain embodiments, R⁴ is

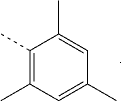

In certain embodiments, R⁴ is

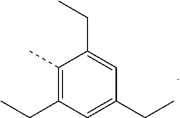

In certain embodiments, R⁴ is

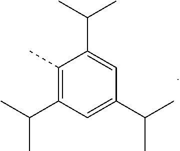

In certain embodiments, R⁴ is

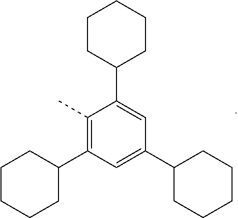

In certain embodiments, R⁴ is

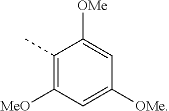

In certain embodiments, R⁴ is

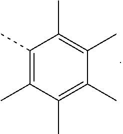

In certain embodiments, R⁴ is cyclohexyl. In certain embodiments, R⁴ is adamantyl.

In certain embodiments, X is selected from the group consisting of halogen, OS(═O)₂R^{A}, OC(═O)R^{A}, N(C(═O)R^{A})₂, tetracoordinate boronate, and hexacoordinate phosphorus. In certain embodiments, X is Cl.

In certain embodiments, the compound is selected from the group consisting of:
5-(2,6-dimethylphenyl)-2-mesitylimidazo[1,5-α]pyridin-2-ium chloride;
2-mesityl-5-(2,4,6-triethylphenyl)imidazo[1,5-α]pyridin-2-ium chloride;
2-(2,6-diisopropylphenyl)-5-(2,4,6-triethylphenyl)imidazo[1,5-α]pyridin-2-ium chloride;
2-(2,6-diisopropylphenyl)-5-(2,3,4,5,6-pentamethylphenyl)imidazo[1,5-α]pyridin-2-ium chloride;
2-mesityl-5-(2,4,6-tricyclohexylphenyl)imidazo[1,5-α]pyridin-2-ium chloride;
2-(2,6-diisopropylphenyl)-5-(2,4,6-tricyclohexylphenyl)imidazo[1,5-α]pyridin-2-ium chloride;
5-(2,6-diisopropylphenyl)-2-(2,6-dimethylphenyl)imidazo[1,5-α]pyridin-2-ium chloride;
5-(2,6-diisopropylphenyl)-2-mesitylimidazo[1,5-α]pyridin-2-ium chloride;
2,5-bis(2,6-diisopropylphenyl)imidazo[1,5-α]pyridin-2-ium chloride;
2-cyclohexyl-5-(2,6-diisopropylphenyl)imidazo[1,5-α]pyridin-2-ium chloride;
2-(2,6-diisopropylphenyl)-5-(2,6-dimethoxyphenyl)imidazo[1,5-α]pyridin-2-ium chloride;
2-(2,6-diisopropylphenyl)-5-(2,4,6-trimethoxyphenyl)imidazo[1,5-α]pyridin-2-ium chloride;
5-(2,6-diisopropoxyphenyl)-2-(2,6-diisopropylphenyl)imidazo[1,5-α]pyridin-2-ium chloride;
5-(2-(dimethylamino)phenyl)-2-mesitylimidazo[1,5-α]pyridin-2-ium chloride;
2-(2,6-diisopropylphenyl)-5-(2-(dimethylamino)phenyl)imidazo[1,5-α]pyridin-2-ium chloride;
2-(2,6-dibenzhydryl-4-methylphenyl)-5-mesitylimidazo[1,5-α]pyridin-2-ium chloride;
2-(2,6-dibenzhydryl-4-methoxyphenyl)-5-mesitylimidazo[1,5-α]pyridin-2-ium chloride;
2-(2,6-dibenzhydryl-4-methylphenyl)-5-(2,4,6-triisopropylphenyl)imidazo[1,5-α]pyridin-2-ium chloride;
2-(2,6-dibenzhydryl-4-fluorophenyl)-5-(2,4,6-triisopropylphenyl)imidazo[1,5-α]pyridin-2-ium chloride;
5-cyclohexyl-2-mesitylimidazo[1,5-α]pyridin-2-ium chloride;
5-cyclohexyl-2-(2,6-diisopropylphenyl)imidazo[1,5-α]pyridin-2-ium chloride;
5-(adamantan-1-yl)-2-mesitylimidazo[1,5-α]pyridin-2-ium chloride;
5-(adamantan-1-yl)-2-(2,6-diisopropylphenyl)imidazo[1,5-α]pyridin-2-ium chloride;
5-(3,5-dimethylphenyl)-2-mesitylimidazo[1,5-α]pyridin-2-ium chloride;
2-(2,6-Diisopropylphenyl)-5-(3,5-dimethylphenyl)imidazo[1,5-α]pyridin-2-ium chloride;
5-(3,5-di-tert-butylphenyl)-2-mesitylimidazo[1,5-α]pyridin-2-ium chloride;
5-(3,5-di-tert-butylphenyl)-2-(2,6-diisopropylphenyl)imidazo[1,5-α]pyridin-2-ium chloride;
5-([1,1′:3′,1″-terphenyl]-5′-yl)-2-mesitylimidazo[1,5-α]pyridin-2-ium chloride;
5-([1,1′:3′,1″-terphenyl]-5′-yl)-2-(2,6-diisopropylphenyl)imidazo[1,5-α]pyridin-2-ium chloride;
5-(2-isopropylphenyl)-2-mesitylimidazo[1,5-α]pyridin-2-ium chloride; and
2-(2,6-diisopropylphenyl)-5-(2-isopropylphenyl)imidazo[1,5-α]pyridin-2-ium chloride.

In another aspect, the present disclosure provides a compound of formula (II):

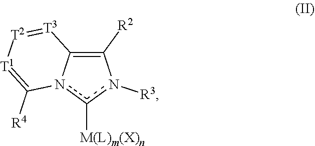

wherein:
M is a transition metal;
L is a ligand of M, wherein each occurrence of L can be the same or different;
each occurrence of [symbol] is a single or double bond,
   wherein no more than one [symbol] bonding a C atom and a N atom is a double bond;
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R¹, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R³ is selected from the group consisting of optionally substituted C₆-C₁₀ aryl, optionally substituted C₂-C₈ heteroaryl, and optionally substituted C₃-C₈ cycloalkyl,
   wherein each optional substituent in R³ is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R⁴ is selected from the group consisting of optionally substituted C₃-C₁₂ cycloalkyl and phenyl substituted with at least one substituent selected from the group consisting of C₁-C₆ alkyl, C₃-C₁₂ cycloalkyl, C₁-C₆ alkoxy, phenyl, and N(R^{A})(R^{B}),
   wherein each optional substituent in the C₃-C₁₂ cycloalkyl is at least one selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
X is a counter anion;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen;
m is an integer which is selected from the group consisting of 0, 1, 2, and 3;
n is an integer which is selected from the group consisting of 0, 1, and 2.

In certain embodiments, if T¹ is CH, T² is CH, T³, is CH, R² is H, M is selected from the group consisting of Cu, Ag, and Au, and R⁴ is 2,4,6-triisopropylphenyl, then R³ is not 2,6-diisopropylphenyl or 2,4,6-trimethylphenyl.

In certain embodiments, if T¹ is CH, T² is CH, T³, is CH, R² is H, M is Cu, and R⁴ is 2,4,6-trimethylphenyl, then R³ is not 2,4,6-trimethylphenyl, 2,6-diisopropylphenyl, adamantly, 4-(2-(2-methoxyethoxy)ethoxy)-2,6-dimethylphenyl, or (2-(2-methoxyethoxy)ethoxy)phenyl.

In certain embodiments, if T¹ is CH, T² is CH, T³ is CH, R² is H, M is Pd, R⁴ is 2,4,6-trimethylphenyl, and L is 3-chloropyridyl or pyridyl, then R³ is not 2,4,6-trimethylphenyl or 4-(2-(2-methoxyethoxy)ethoxy)-2,6-dimethylphenyl.

In certain embodiments, if T¹ is CH, T² is CH, T³ is CH, R² is H, M is Pd, R⁴ is 2,4,6-triisopropylphenyl, and X is allyl anion (i.e., vinylmethanide), then R³ is not 2,4,6-trimethylphenyl.

In certain embodiments, if T¹ is CH, T² is CH, T³, is CH, R² is H, M is Au, and R⁴ is 5-tetrahydroisoquinolinyl, then R³ is not 2,6-diisopropylphenyl.

In certain embodiments, T¹ is CR^{1a}. In certain embodiments, T² is CR^{1b}. In certain embodiments, T³ is CR^{1c}.

In certain embodiments, T¹ is CR^{1a}, T² is CR^{1b}, and T³ is CR^{1c}. In certain embodiments, T¹ is N, T² is CR^{1b}, and T³ is CR^{1c}. In certain embodiments, T¹ is CR^{1a}, T² is N, and T³ is CR^{1c}.

In certain embodiments, T¹ is CR^{1a}, T² is CR^{1b}, and T³ is N. In certain embodiments, T¹ is N, T² is N, and T³ is CR^{1c}. In certain embodiments, T¹ is N, T² is CR^{1b}, and T³ is N. In certain embodiments, T¹ is CR^{1a}, T² is N, and T³ is N.

In certain embodiments, at least one of R^{1a}, R^{1b}, and R^{1c} is H. In certain embodiments, at least two of R^{1a}, R^{1b}, and R^{1c} are H. In certain embodiments, each of R^{1a}, R^{1b}, and R^{1c} are H.

In certain embodiments, R² is H.

In certain embodiments, R³ is.

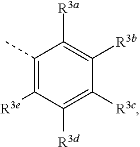

wherein:
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
   wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

In certain embodiments, R^{3a} is H. In certain embodiments, R^{3a} is methyl. In certain embodiments, R^{3a} is i-propyl. In certain embodiments, R^{3a} is diphenylmethyl. In certain embodiments, R^{3a} is methoxy. In certain embodiments, R^{3a} is fluoro.

In certain embodiments, R^{3b} is H. In certain embodiments, R^{3b} is methyl. In certain embodiments, R^{3b} is i-propyl. In certain embodiments, R^{3b} is diphenylmethyl. In certain embodiments, R^{3b} is methoxy. In certain embodiments, R^{3b} is fluoro.

In certain embodiments, R^{3c} is H. In certain embodiments, R^{3c} is methyl. In certain embodiments, R^{3e} is i-propyl. In certain embodiments, R^{3c} is diphenylmethyl. In certain embodiments, R^{3c} is methoxy. In certain embodiments, R^{3c} is fluoro.

In certain embodiments, R^{3d} is H. In certain embodiments, R^{3d} is methyl. In certain embodiments, R^{3d} is i-propyl. In certain embodiments, R^{3d} is diphenylmethyl. In certain embodiments, R^{3d} is methoxy. In certain embodiments, R^{3d} is fluoro.

In certain embodiments, R^{3e} is H. In certain embodiments, R^{3e} is methyl. In certain embodiments, R^{3e} is i-propyl. In certain embodiments, R^{3e} is diphenylmethyl. In certain embodiments, R^{3e} is methoxy. In certain embodiments, R^{3e} is fluoro.

In certain embodiments, R^{3b} and R^{3d} are each independently H. In certain embodiments, R^{3b}, R^{3c}, and R^{3d} are each independently H.

In certain embodiments, R^{3a} and R^{3e} are identical. In certain embodiments, R^{3a}, R^{3e}, and R^{3e} are identical.

In certain embodiments, R^{3a} and R^{3e} are each independently optionally substituted C₁-C₆ alkyl, and R^{3b}, R^{3c}, and R^{3d} are H. In certain embodiments, R^{3a}, R^{3c}, and R^{3e} are each independently optionally substituted C₁-C₆ alkyl, and R^{3b} and R^{3d} are H. In certain embodiments, R^{3a} and R^{3e} are each independently optionally substituted C₁-C₆ alkyl, R^{3c} is selected from the group consisting of halogen and C₁-C₆ alkoxy, and R^{3b} and R^{3d} are H.

In certain embodiments, R³ is

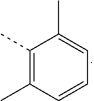

In certain embodiments, R³ is

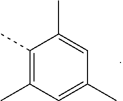

In certain embodiments, R³ is

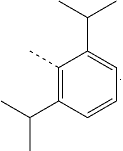

In certain embodiments, R³ is

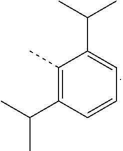

In certain embodiments, R³ is

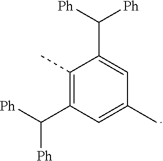

In certain embodiments, R³ is

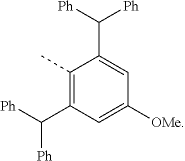

In certain embodiments, R³ is

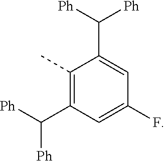

In certain embodiments, R³ is cyclohexyl.

In certain embodiments, R⁴ is:

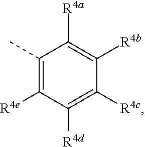

wherein:
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
   wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

In certain embodiments, R^{4a} is H. In certain embodiments, R^{4a} is methyl. In certain embodiments, R^{4a} is ethyl. In certain embodiments, R^{4a} is i-propyl. In certain embodiments, R^{4a} is cyclohexyl. In certain embodiments, R^{4a} is t-butyl. In certain embodiments, R^{4a} is methoxy.

In certain embodiments, R^{4a} is i-propoxy. In certain embodiments, R^{4a} is phenyl. In certain embodiments, R^{4a} is dimethylamino.

In certain embodiments, R^{4b} is H. In certain embodiments, R^{4b} is methyl. In certain embodiments, R^{4b} is ethyl. In certain embodiments, R^{4b} is i-propyl. In certain embodiments, R^{4b} is cyclohexyl. In certain embodiments, R^{4b} is t-butyl. In certain embodiments, R^{4b} is methoxy. In certain embodiments, R^{4b} is i-propoxy. In certain embodiments, R^{4b} is phenyl. In certain embodiments, R^{4b} is dimethylamino.

In certain embodiments, R^{4c} is H. In certain embodiments, R^{4c} is methyl. In certain embodiments, R^{4c} is ethyl. In certain embodiments, R^{4c} is i-propyl. In certain embodiments, R^{4c} is cyclohexyl. In certain embodiments, R^{4c} is t-butyl. In certain embodiments, R^{4c} is methoxy. In certain embodiments, R^{4c} is i-propoxy. In certain embodiments, R^{4c} is phenyl. In certain embodiments, R^{4c} is dimethylamino.

In certain embodiments, R^{4d} is H. In certain embodiments, R^{4d} is methyl. In certain embodiments, R^{4d} is ethyl. In certain embodiments, R^{4d} is i-propyl. In certain embodiments, R^{4d} is cyclohexyl. In certain embodiments, R^{4d} is t-butyl. In certain embodiments, R^{4d} is methoxy. In certain embodiments, R^{4d} is i-propoxy. In certain embodiments, R^{4d} is phenyl. In certain embodiments, R^{4d} is dimethylamino.

In certain embodiments, R^{4e} is H. In certain embodiments, R^{4e} is methyl. In certain embodiments, R^{4e} is ethyl. In certain embodiments, R^{4e} is i-propyl. In certain embodiments, R^{4e} is cyclohexyl. In certain embodiments, R^{4e} is t-butyl. In certain embodiments, R^{4e} is methoxy. In certain embodiments, R^{4e} is i-propoxy. In certain embodiments, R^{4e} is phenyl. In certain embodiments, R^{4e} is dimethylamino.

In certain embodiments, R^{4b}, R^{4c}, R^{4d}, and R^{4e} are each independently H. In certain embodiments, R^{4b}, R^{4c}, and R^{4d} are each independently H. In certain embodiments, R^{4a}, R^{4c}, and R^{4e} are each independently H. In certain embodiments, none of R^{4a}, R^{4b}, R^{4c}, R^{4d}, and R^{4e} are H.

In certain embodiments, R^{4a} and R^{4e} are identical. In certain embodiments, R^{4b} and R^{4d} are identical. In certain embodiments, R^{4a}, R^{4c}, and R^{4e} are identical. In certain embodiments, R^{4a}, R^{4b}, R^{4c}, R^{4d}, and R^{4e} are identical.

In certain embodiments, R^{4a} is selected from the group consisting of C₁-C₆ alkyl and N(C₁-C₆ alkyl)₂, and R^{4b}, R^{4c}, R^{4d}, and R^{4e} are each independently H. In certain embodiments, R^{4a} and R^{4e} are each independently C₁-C₆ alkyl, and R^{4b}, R^{4c}, and R^{4d} are each independently H.

In certain embodiments, R^{4a} and R^{4e} are each independently C₁-C₆ alkoxy, and R^{4b}, R^{4c}, and R^{4d} are each independently H. In certain embodiments, R^{4b} and R^{4d} are each independently C₁-C₆ alkyl or phenyl, and R^{4a}, R^{4c}, and R^{4e} are each independently H. In certain embodiments, R^{4a}, R^{4c}, and R^{4e} are each independently C₁-C₆ alkyl or C₃-C₈ cycloalkyl, and R^{4b} and R^{4d} are each independently H. In certain embodiments, R^{4a}, R^{4c}, and R^{4e} are each independently C₁-C₆ alkoxy, and R^{4b} and R^{4d} are each independently H. In certain embodiments, R^{4a}, R^{4b}, R^{4c}, R^{4d}, and R^{4e} are each independently C₁-C₆ alkyl.

In certain embodiments, R⁴ is

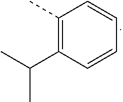

In certain embodiments, R⁴ is

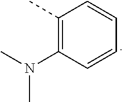

In certain embodiments, R⁴ is

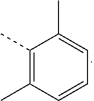

In certain embodiments, R⁴ is

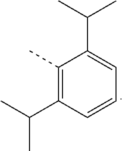

In certain embodiments, R⁴ is

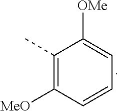

In certain embodiments, R⁴ is

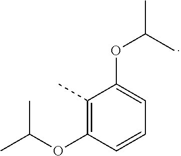

In certain embodiments, R⁴ is

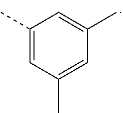

In certain embodiments, R⁴ is

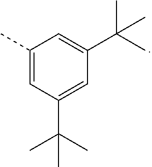

In certain embodiments, R⁴ is

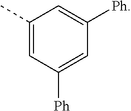

In certain embodiments, R⁴ is

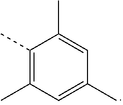

In certain embodiments, R⁴ is

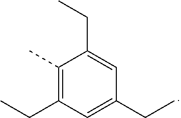

In certain embodiments, R⁴ is

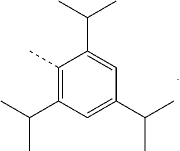

In certain embodiments, R⁴ is

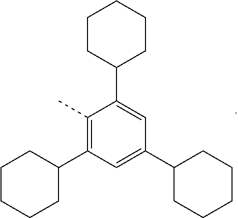

In certain embodiments, R⁴ is

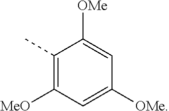

In certain embodiments, R⁴ is

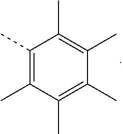

In certain embodiments, R⁴ is cyclohexyl. In certain embodiments, R⁴ is adamantyl.

In certain embodiments, X is selected from the group consisting of halogen, OS(═O)₂R^{A}, OC(═O)R^{A}, N(C(═O)R^{A})₂, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

In certain embodiments, X is Cl. In certain embodiments, X is allyl anion (i.e., vinylmethanide). In certain embodiments, X is t-butylindenyl anion (i.e., 1-t-butylinden-1-ide and/or 3-t-butylinden-1-ide). In certain embodiments, X is allylbenzene anion (i.e., 3-phenylpropen-3-ide and/or 1-phenylpropen-3-ide).

In certain embodiments, M is selected from the group consisting of Pd, Cu, Ag, Au, Ni, Pt, Co, Rh, Ir, Fe, Ru, and Os. In certain embodiments, M is Pd. In certain embodiments, M is Cu. In certain embodiments, M is Ag. In certain embodiments, M is Rh.

In certain embodiments, L is selected from the group consisting of carbon monoxide (CO), optionally substituted C₂-C₁₂ alkene, and optionally substituted C₅-C₁₂ cycloalkene, optionally substituted benzylamine, optionally substituted C₂-C₈ heteroaryl, wherein each optional substituent in the C₂-C₁₂ alkene, C₅-C₁₂ cycloalkene, benzylamine, and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

In certain embodiments, L is cyclooctadiene (COD). In certain embodiments, L is carbon monoxide (CO). In certain embodiments, L is pyridine. In certain embodiments, L is 3-chloropyridine.

In certain embodiments, the compound is selected from the group consisting of: cinnamyl [5-(2,6-diisopropylphenyl)-2-mesitylimidazo[1,5-α]pyridin-3-ylidene]chloropalladium;
cinnamyl [2-(2,6-dibenzhydryl-4-methylphenyl)-5-(2,4,6-triisopropylphenyl)imidazo[1,5-a]pyridin-3-ylidene]chloropalladium(II);
cinnamyl [5-(2,6-diisopropoxyphenyl)-2-(2,6-diisopropylphenyl)imidazo[1,5-α]pyridin-3-ylidene]chloropalladium(II);
2-dimethylaminomethylphenyl [2-(2,6-dibenzhydryl-4-methylphenyl)-5-(2,4,6-triisopropylphenyl)imidazo[1,5-α]pyridin-3-ylidene]chloropalladium(II);
2-acetamido-phenyl [2-(2,6-dibenzhydryl-4-methylphenyl)-5-(2,4,6-triisopropylphenyl)imidazo[1,5-α]pyridin-3-ylidene]chloropalladium(II);
cinnamyl [2,5-dimesitylimidazo[1,5-α]pyridin-3-ylidene]chloropalladium(II);
allyl [2,5-dimesitylimidazo[1,5-α]pyridin-3-ylidene]chloropalladium(II);
Cinnamyl [2-(2,6-diisopropylphenyl)-5-mesitylimidazo[1,5-α]pyridin-3-ylidene]chloropalladium(II);
1-tert-butyl-indenyl [2-(2,6-diisopropylphenyl)-5-mesitylimidazo[1,5-α]pyridin-3-ylidene]chloropalladium(II);
cinnamyl [2-mesityl-5-(2,4,6-triisopropylphenyl)imidazo[1,5-α]pyridin-3-ylidene]chloropalladium(II);
pyridine [2-(2,6-diisopropylphenyl)-5-mesitylimidazo[1,5-α]pyridin-3-ylidene]dichloropalladium(II);
3-chloropyridine [2-(2,6-diisopropylphenyl)-5-mesitylimidazo[1,5-α]pyridin-3-ylidene]dichloropalladium(II);
3-chloropyridine [2-mesityl-5-(2,4,6-triisopropylphenyl)imidazo[1,5-α]pyridin-3-ylidene]dichloropalladium(II);
(2,5-dimesitylimidazo[1,5-α]pyridin-3(2H)-ylidene)gold(I) chloride;
(5-(2,6-diisopropylphenyl)-2-mesitylimidazo[1,5-α]pyridin-3(2H)-ylidene)gold(I) chloride;
(2-(2,6-dibenzhydryl-4-methylphenyl)-5-mesityl-2,3-dihydroimidazo[1,5-α]pyridin-3-yl)gold(I) chloride;
(2-(2,6-dibenzhydryl-4-methoxyphenyl)-5-mesityl-2,3-dihydroimidazo[1,5-α]pyridin-3-yl)gold(I) chloride;
(2-(2,6-dibenzhydryl-4-methylphenyl)-5-(2,4,6-triisopropylphenyl)-2,3-dihydroimidazo[1,5-a]pyridin-3-yl)gold(I) chloride;
(5-(2,6-diisopropoxyphenyl)-2-(2,6-diisopropylphenyl)-2,3-dihydroimidazo[1,5-α]pyridin-3-yl)gold(I) chloride;
(2-(2,6-dibenzhydryl-4-methylphenyl)-5-mesityl-2,3-dihydroimidazo[1,5-α]pyridin-3-yl)copper(I) chloride;
(2-(2,6-dibenzhydryl-4-methoxyphenyl)-5-mesityl-2,3-dihydroimidazo[1,5-α]pyridin-3-yl)copper(I) chloride;
(2-(2,6-dibenzhydryl-4-methylphenyl)-5-(2,4,6-triisopropylphenyl)-2,3-dihydroimidazo[1,5-a]pyridin-3-yl)copper(I) chloride;
(2-(2,6-dibenzhydryl-4-methylphenyl)-5-mesityl-2,3-dihydroimidazo[1,5-α]pyridin-3-yl)silver(I) chloride;
(2-(2,6-dibenzhydryl-4-methoxyphenyl)-5-mesityl-2,3-dihydroimidazo[1,5-α]pyridin-3-yl)silver(I) chloride;
(2-(2,6-dibenzhydryl-4-methylphenyl)-5-(2,4,6-triisopropylphenyl)-2,3-dihydroimidazo[1,5-a]pyridin-3-yl)silver(I) chloride;
cyclooctadiene [5-(2,6-diisopropylphenyl)-2-mesitylimidazo[1,5-α]pyridin-3-ylidene]rhodium(I) chloride;
dicarbonyl [5-(2,6-diisopropylphenyl)-2-mesitylimidazo[1,5-α]pyridin-3-ylidene]rhodium(I) chloride;
cyclooctadiene [2,5-dimesitylimidazo[1,5-α]pyridin-2-ylidene]rhodium(I) chloride;
dicarbonyl [2,5-dimesitylimidazo[1,5-α]pyridin-2-ylidene]rhodium(I) chloride;
cyclooctadiene [2-(2,6-dibenzhydryl-4-methylphenyl)-5-(2,4,6-triisopropylphenyl)imidazo[1,5-α]pyridin-3-ylidene]rhodium(I) chloride;
dicarbonyl [2-(2,6-dibenzhydryl-4-methylphenyl)-5-(2,4,6-triisopropylphenyl)imidazo[1,5-a]pyridin-3-ylidene]rhodium(I) chloride;
(5-(2,4,6-triethylphenyl)-2-(2,6-diisopropylphenyl)-2,3-dihydroimidazo[1,5-α]pyridin-3-yl)gold(I) chloride;
cinnamyl (5-(2,4,6-triethylphenyl)-2-(2,6-diisopropylphenyl)-2,3-dihydroimidazo[1,5-a]pyridin-3-yl)chloropalladium (II);
(5-(2,4,6-trimethylphenyl)-2-(2,6-diisopropylphenyl)-2,3-dihydroimidazo[1,5-α]pyridin-3-yl)gold(I) chloride;
(5-(2,6-diisopropylphenyl)-2-(2,6-diisopropylphenyl)-2,3-dihydroimidazo[1,5-α]pyridin-3-yl)gold(I) chloride; and
(5-(2,6-diisopropylphenyl)-2-cyclohexyl-2,3-dihydroimidazo[1,5-α]pyridin-3-yl)gold(I) chloride.

TABLE 1
N-Heterocyclic Carbene (NHC) Ligands and Catalyst Complexes

| Cmpd | Structure | Name |
|---|---|---|
| 1 | 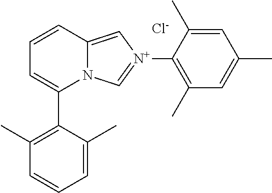 | 5-(2,6-dimethylphenyl)-2- mesitylimidazo[1,5-a]pyridin-2-ium chloride |
| 2 | 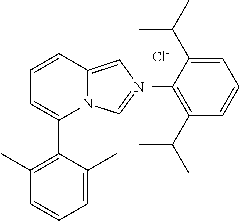 [SMILES: Cc1cccc(C)c1-c1cccc2c[n+](-c3c(C(C)C)cccc3C(C)C)cn12 ; [Cl-]] | 2-(2,6-diisopropylphenyl)-5-(2,6- dimethylphenyl)imidazo[1,5- a]pyridin-2-ium chloride |
| 3 | 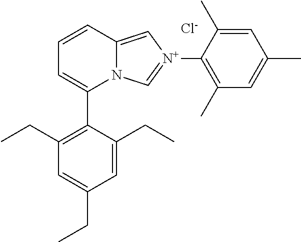 [SMILES: CCc1cc(CC)c(-c2cccc3c[n+](-c4c(C)cc(C)cc4C)cn23)c(CC)c1 ; [Cl-]] | 2-mesityl-5-(2,4,6- triethylphenyl)imidazo[1,5- a]pyridin-2-ium chloride |
| 4 | 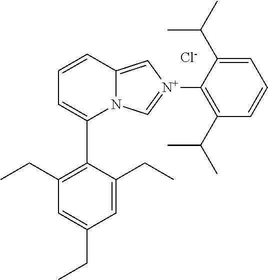 [SMILES: CCc1cc(CC)c(-c2cccc3c[n+](-c4c(C(C)C)cccc4C(C)C)cn23)c(CC)c1 ; [Cl-]] | 2-(2,6-diisopropylphenyl)-5-(2,4,6- triethylphenyl)imidazo[1,5- a]pyridin-2-ium chloride |
| 5 | 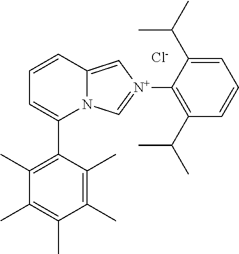 [SMILES: Cc1c(C)c(C)c(-c2cccc3c[n+](-c4c(C(C)C)cccc4C(C)C)cn23)c(C)c1C ; [Cl-]] | 2-(2,6-diisopropylphenyl)-5- (2,3,4,5,6- pentamethylphenyl)imidazo[1,5- a]pyridin-2-ium chloride |
| 6 | 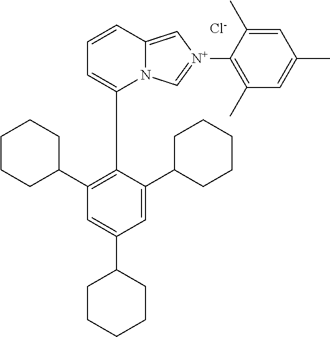 [SMILES: Cc1cc(C)c(-[n+]2cc3cccc(-c4c(C5CCCCC5)cc(C5CCCCC5)cc4C4CCCCC4)n3c2)c(C)c1 ; [Cl-]] | 2-mesityl-5-(2,4,6- tricyclohexylphenyl)imidazo[1,5- a]pyridin-2-ium chloride |
| 7 | 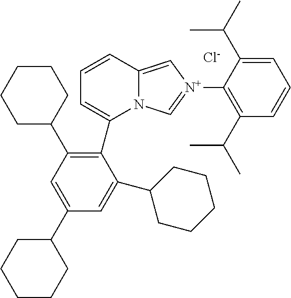 [SMILES: CC(C)c1cccc(C(C)C)c1-[n+]1cc2cccc(-c3c(C4CCCCC4)cc(C4CCCCC4)cc3C3CCCCC3)n2c1 ; [Cl-]] | 2-(2,6-diisopropylphenyl)-5-(2,4,6- tricyclohexylphenyl)imidazo[1,5- a]pyridin-2-ium chloride |
| 8 | 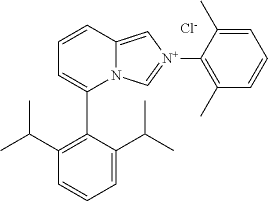 [SMILES: Cc1cccc(C)c1-[n+]1cc2cccc(-c3c(C(C)C)cccc3C(C)C)n2c1 ; [Cl-]] | 5-(2,6-diisopropylphenyl)-2-(2,6- dimethylphenyl)imidazo[1,5- a]pyridin-2-ium chloride |
| 9 | 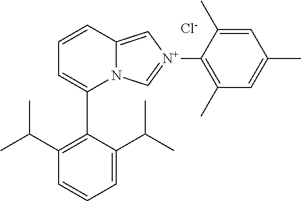 [SMILES: Cc1cc(C)c(-[n+]2cc3cccc(-c4c(C(C)C)cccc4C(C)C)n3c2)c(C)c1 ; [Cl-]] | 5-(2,6-diisopropylphenyl)-2- mesitylimidazo[1,5-a]pyridin-2-ium chloride |
| 10 | 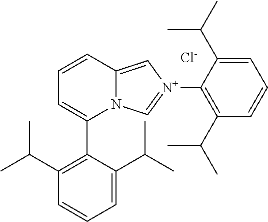 [SMILES: CC(C)c1cccc(C(C)C)c1-c1cccc2c[n+](-c3c(C(C)C)cccc3C(C)C)cn12 ; [Cl-]] | 2,5-bis(2,6- diisopropylphenyl)imidazo[1,5- a]pyridin-2-ium chloride |
| 11 | 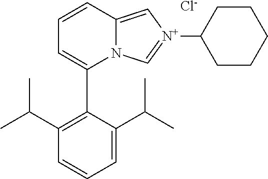 [SMILES: CC(C)c1cccc(C(C)C)c1-c1cccc2c[n+](C3CCCCC3)cn12 ; [Cl-]] | 2-cyclohexyl-5-(2,6- diisopropylphenyl)imidazo[1,5- a]pyridin-2-ium chloride |
| 12 | 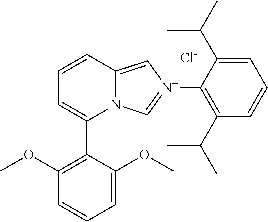 [SMILES: COc1cccc(OC)c1-c1cccc2c[n+](-c3c(C(C)C)cccc3C(C)C)cn12 ; [Cl-]] | 2-(2,6-diisopropylphenyl)-5-(2,6- dimethoxyphenyl)imidazo[1,5- a]pyridin-2-ium chloride |
| 13 | 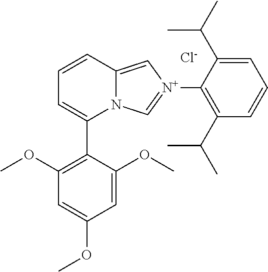 [SMILES: COc1cc(OC)c(-c2cccc3c[n+](-c4c(C(C)C)cccc4C(C)C)cn23)c(OC)c1 ; [Cl-]] | 2-(2,6-diisopropylphenyl)-5-(2,4,6- trimethoxyphenyl)imidazo[1,5- a]pyridin-2-ium chloride |
| 14 | 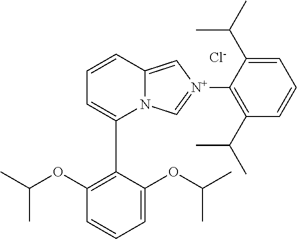 [SMILES: CC(C)Oc1cccc(OC(C)C)c1-c1cccc2c[n+](-c3c(C(C)C)cccc3C(C)C)cn12 ; [Cl-]] | 5-(2,6-diisopropoxyphenyl)-2-(2,6- diisopropylphenyl)imidazo[1,5- a]pyridin-2-ium chloride |
| 15 | 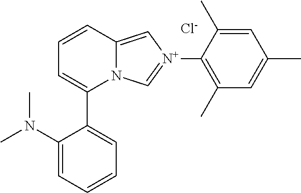 | 5-(2-(dimethylamino)phenyl)-2- mesitylimidazo[1,5-a]pyridin-2-ium chloride |
| 16 | 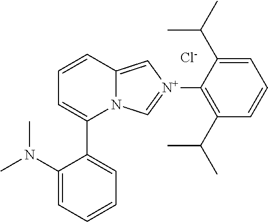 [SMILES: CC(C)c1cccc(C(C)C)c1-[n+]1cc2cccc(-c3ccccc3N(C)C)n2c1 ; [Cl-]] | 2-(2,6-diisopropylphenyl)-5-(2- (dimethylamino)phenyl)imidazo[1,5- a]pyridin-2-ium chloride |
| 17 | 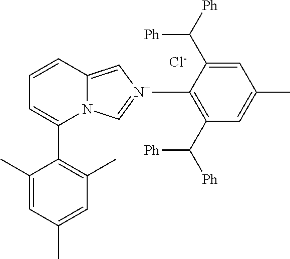 | 2-(2,6-dibenzhydryl-4- methylphenyl)-5- mesitylimidazo[1,5-a]pyridin-2-ium chloride |
| 18 | 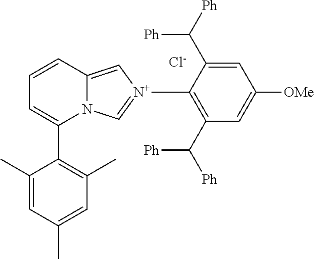 [SMILES: COc1cc(C(c2ccccc2)c2ccccc2)c(-[n+]2cc3cccc(-c4c(C)cc(C)cc4C)n3c2)c(C(c2ccccc2)c2ccccc2)c1 ; [Cl-]] | 2-(2,6-dibenzhydryl-4- methoxyphenyl)-5- mesitylimidazo[1,5-a]pyridin-2-ium chloride |
| 19 | 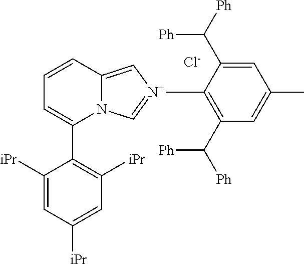 [SMILES: Cc1cc(C(c2ccccc2)c2ccccc2)c(-[n+]2cc3cccc(-c4c(C(C)C)cc(C(C)C)cc4C(C)C)n3c2)c(C(c2ccccc2)c2ccccc2)c1 ; [Cl-]] | 2-(2,6-dibenzhydryl-4- methylphenyl)-5-(2,4,6- triisopropylphenyl)imidazo[1,5- a]pyridin-2-ium chloride |
| 20 | 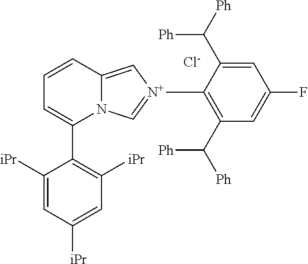 [SMILES: CC(C)c1cc(C(C)C)c(-c2cccc3c[n+](-c4c(C(c5ccccc5)c5ccccc5)cc(F)cc4C(c4ccccc4)c4ccccc4)cn23)c(C(C)C)c1 ; [Cl-]] | 2-(2,6-dibenzhydryl-4- fluorophenyl)-5-(2,4,6- triisopropylphenyl)imidazo[1,5- a]pyridin-2-ium chloride |
| 21 | 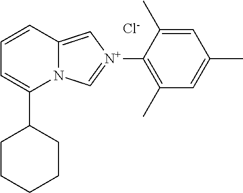 [SMILES: Cc1cc(C)c(-[n+]2cc3cccc(C4CCCCC4)n3c2)c(C)c1 ; [Cl-]] | 5-cyclohexyl-2-mesitylimidazo[1,5- a]pyridin-2-ium chloride |
| 22 | 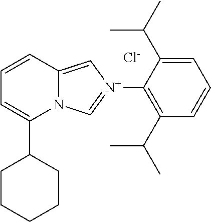 [SMILES: CC(C)c1cccc(C(C)C)c1-[n+]1cc2cccc(C3CCCCC3)n2c1 ; [Cl-]] | 5-cyclohexyl-2-(2,6- diisopropylphenyl)imidazo[1,5- a]pyridin-2-ium chloride |
| 23 | 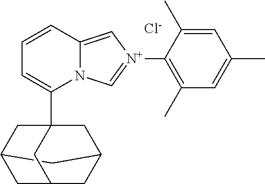 [SMILES: Cc1cc(C)c(-[n+]2cc3cccc(C45CC6CC(CC(C6)C4)C5)n3c2)c(C)c1 ; [Cl-]] | 5-(adamantan-1-yl)-2- mesitylimidazo[1,5-a]pyridin-2-ium chloride |
| 24 | 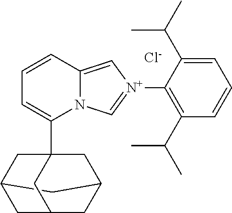 [SMILES: CC(C)c1cccc(C(C)C)c1-[n+]1cc2cccc(C34CC5CC(CC(C5)C3)C4)n2c1 ; [Cl-]] | 5-(adamantan-1-yl)-2-(2,6- diisopropylphenyl)imidazo[1,5- a]pyridin-2-ium chloride |
| 25 | 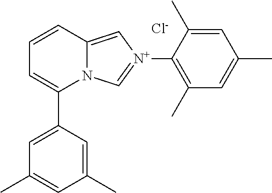 | 5-(3,5-dimethylphenyl)-2- mesitylimidazo[1,5-a]pyridin-2-ium chloride |
| 26 | 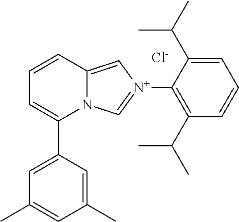 [SMILES: Cc1cc(C)cc(-c2cccc3c[n+](-c4c(C(C)C)cccc4C(C)C)cn23)c1 ; [Cl-]] | 2-(2,6-Diisopropylphenyl)-5-(3,5- dimethylphenyl)imidazo[1,5- a]pyridin-2-ium chloride |
| 27 | 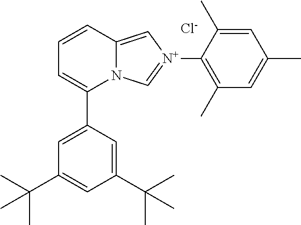 [SMILES: Cc1cc(C)c(-[n+]2cc3cccc(-c4cc(C(C)(C)C)cc(C(C)(C)C)c4)n3c2)c(C)c1 ; [Cl-]] | 5-(3,5-di-tert-butylphenyl)-2- mesitylimidazo[1,5-a]pyridin-2-ium chloride |
| 28 | 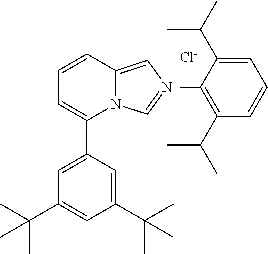 [SMILES: CC(C)c1cccc(C(C)C)c1-[n+]1cc2cccc(-c3cc(C(C)(C)C)cc(C(C)(C)C)c3)n2c1 ; [Cl-]] | 5-(3,5-di-tert-butylphenyl)-2-(2,6- diisopropylphenyl)imidazo[1,5- a]pyridin-2-ium chloride |
| 29 | 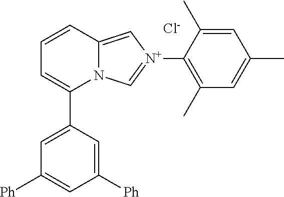 [SMILES: Cc1cc(C)c(-[n+]2cc3cccc(-c4cc(-c5ccccc5)cc(-c5ccccc5)c4)n3c2)c(C)c1 ; [Cl-]] | 5-([1,1′:3′,1″-terphenyl]-5′-yl)-2- mesitylimidazo[1,5-a]pyridin-2-ium chloride |
| 30 | 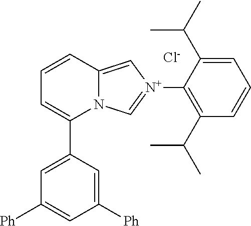 [SMILES: CC(C)c1cccc(C(C)C)c1-[n+]1cc2cccc(-c3cc(-c4ccccc4)cc(-c4ccccc4)c3)n2c1 ; [Cl-]] | 5-([1,1′:3′,1″-terphenyl]-5′-yl)-2- (2,6-diisopropylphenyl)imidazo[1,5- a]pyridin-2-ium chloride |
| 31 | 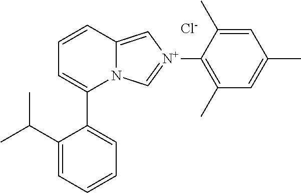 [SMILES: Cc1cc(C)c(-[n+]2cc3cccc(-c4ccccc4C(C)C)n3c2)c(C)c1 ; [Cl-]] | 5-(2-isopropylphenyl)-2- mesitylimidazo[1,5-a]pyridin-2-ium chloride |
| 32 | 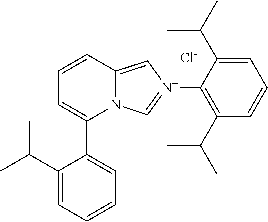 [SMILES: CC(C)c1ccccc1-c1cccc2c[n+](-c3c(C(C)C)cccc3C(C)C)cn12 ; [Cl-]] | 2-(2,6-diisopropylphenyl)-5-(2- isopropylphenyl)imidazo[1,5- a]pyridin-2-ium chloride |
| 33 | 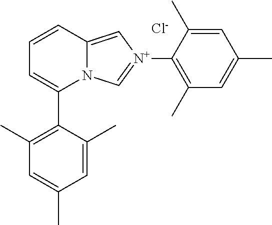 | 2,5-dimesitylimidazo[1,5-a]pyridin- 2-ium chloride |
| 34 | 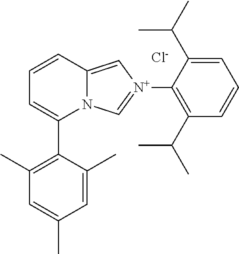 [SMILES: Cc1cc(C)c(-c2cccc3c[n+](-c4c(C(C)C)cccc4C(C)C)cn23)c(C)c1 ; [Cl-]] | 2-(2,6-diisopropylphenyl)-5- mesitylimidazo[1,5-a]pyridin-2-ium chloride |
| 35 | 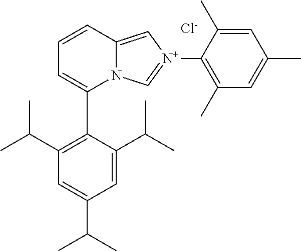 [SMILES: Cc1cc(C)c(-[n+]2cc3cccc(-c4c(C(C)C)cc(C(C)C)cc4C(C)C)n3c2)c(C)c1 ; [Cl-]] | 2-mesityl-5-(2,4,6- triisopropylphenyl)imidazo[1,5- a]pyridin-2-ium chloride |
| 36 | 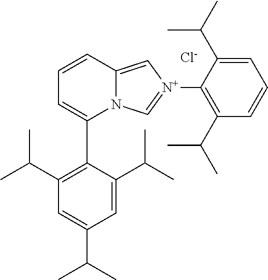 [SMILES: CC(C)c1cc(C(C)C)c(-c2cccc3c[n+](-c4c(C(C)C)cccc4C(C)C)cn23)c(C(C)C)c1 ; [Cl-]] | 2-(2,6-diisopropylphenyl)-5-(2,4,6- triisopropylphenyl)imidazo[1,5- a]pyridin-2-ium chloride |
| 37 | 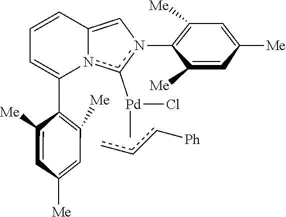 | cinnamyl [2,5-dimesitylimidazo[1,5- a]pyridin-3- ylidene]chloropalladium(II) |
| 38 | 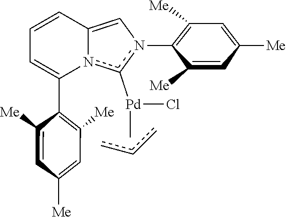 | allyl [2,5-dimesitylimidazo[1,5- a]pyridin-3- ylidene]chloropalladium(II) |
| 39 | 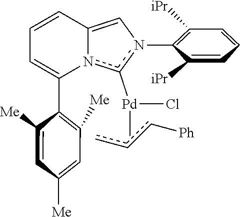 | Cinnamyl [2-(2,6- diisopropylphenyl)-5- mesitylimidazo[1,5-a]pyridin-3- ylidene] chloropalladium(II) |
| 40 | 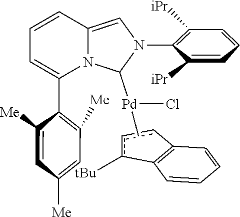 | 1-tert-butyl-indenyl [2-(2,6- diisopropylphenyl)-5- mesitylimidazo[1,5-a]pyridin-3- ylidene] chloropalladium(II) |
| 41 | 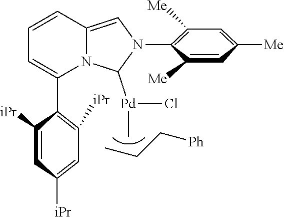 | cinnamyl [2-mesityl-5-(2,4,6- triisopropylphenyl)imidazo[1,5- a]pyridin-3-ylidene] chloropalladium(II) |
| 42 | 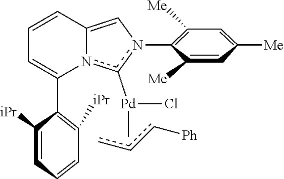 | cinnamyl [5-(2,6- diisopropylphenyl)-2- mesitylimidazo[1,5-a]pyridin-3- ylidene] chloropalladium |
| 43 | 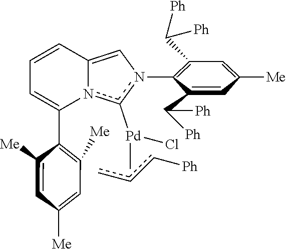 | cinnamyl [2-(2,6-dibenzhydryl-4- methylphenyl)-5- mesitylimidazo[1,5-a]pyridine-3- ylidene] chloropalladium(II) |
| 44 | 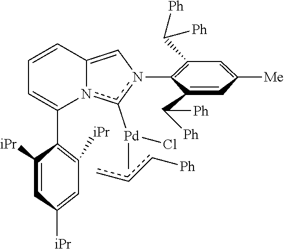 | Cinnamyl [2-(2,6-dibenzhydryl-4- methylphenyl)-5-(2,4,6- triisopropylphenyl)imidazo[1,5- a]pyridin-3-ylidene] chloropalladium(II) |
| 45 | 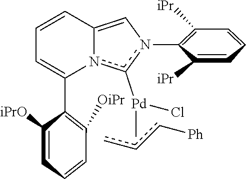 | Cinnamyl [5-(2,6- diisopropoxyphenyl)-2-(2,6- diisopropylphenyl)imidazo[1,5- a]pyridin-3-ylidene] chloropalladium(II) |
| 46 | 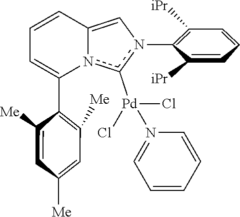 | pyridine [2-(2,6-diisopropylphenyl)- 5-mesitylimidazo[1,5-a]pyridin-3- ylidene] dichloropalladium(II) |
| 47 | 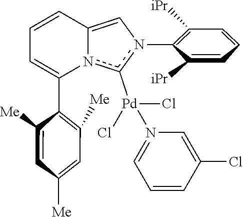 | 3-chloropyridine [2-(2,6- diisopropylphenyl)-5- mesitylimidazo[1,5-a]pyridin-3- ylidene] dichloropalladium(II) |
| 48 | 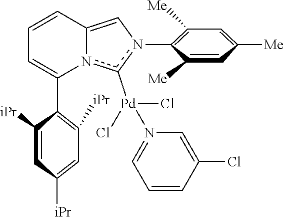 | 3-chloropyridine [2-mesityl-5-(2,4,6- triisopropylphenyl)imidazo[1,5- a]pyridin-3-ylidene] dichloropalladium(II) |
| 49 | 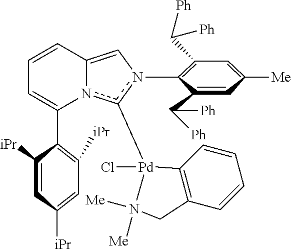 | 2-dimethylaminomethylphenyl [2- (2,6-dibenzhydryl-4-methylphenyl)- 5-(2,4,6- triisopropylphenyl)imidazo[1,5- a]pyridin-3-ylidene] chloropalladium(II) |
| 50 | 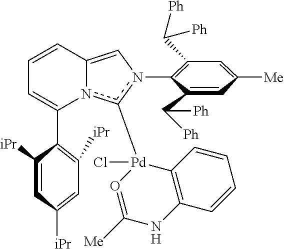 | 2-acetamido-phenyl [2-(2,6- dibenzhydryl-4-methylphenyl)-5- (2,4,6- triisopropylphenyl)imidazo[1,5- a]pyridin-3-ylidene] chloropalladium(II) |
| 51 | 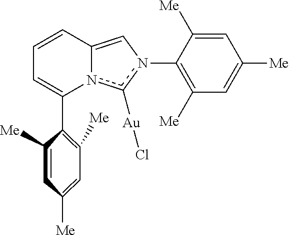 | (2,5-dimesitylimidazo[1,5-a]pyridin- 3(2H)-ylidene)gold(I) chloride |
| 52 | 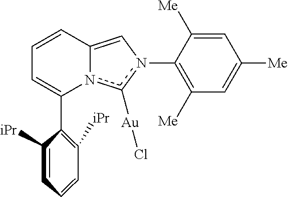 | (5-(2,6-diisopropylphenyl)-2- mesitylimidazo[1,5-a]pyridin-3(2H)- ylidene)gold(I) chloride |
| 53 | 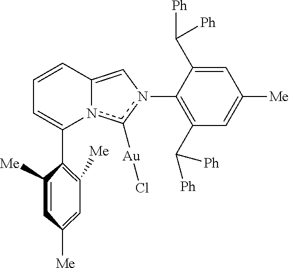 | (2-(2,6-dibenzhydryl-4- methylphenyl)-5-mesityl-2,3- dihydroimidazo[1,5-a]pyridin-3- yl)gold(I) chloride |
| 54 | 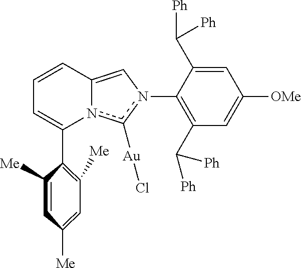 | (2-(2,6-dibenzhydryl-4- methoxyphenyl)-5-mesityl-2,3- dihydroimidazo[1,5-a]pyridin-3- yl)gold(I) chloride |
| 55 | 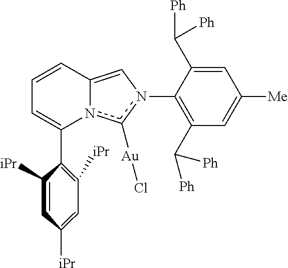 | (2-(2,6-dibenzhydryl-4- methylphenyl)-5-(2,4,6- triisopropylphenyl)-2,3- dihydroimidazo[1,5-a]pyridin-3- yl)gold(I) chloride |
| 56 | 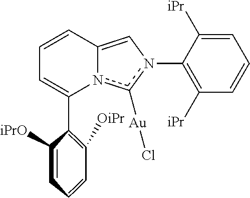 | (5-(2,6-diisopropoxyphenyl)-2-(2,6- diisopropylphenyl)-2,3- dihydroimidazo[1,5-a]pyridin-3- yl)gold(I) chloride |
| 57 | 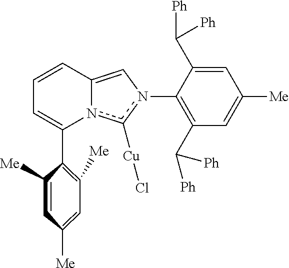 | (2-(2,6-dibenzhydryl-4- methylphenyl)-5-mesityl-2,3- dihydroimidazo[1,5-a]pyridin-3- yl)copper(I) chloride |
| 58 | 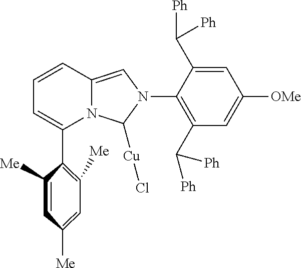 | (2-(2,6-dibenzhydryl-4- methoxyphenyl)-5-mesityl-2,3- dihydroimidazo[1,5-a]pyridin-3- yl)copper(I) chloride |
| 59 | 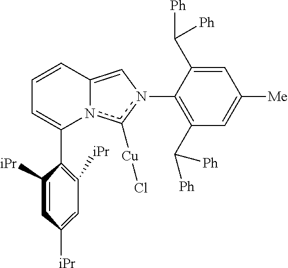 | (2-(2,6-dibenzhydryl-4- methylphenyl)-5-(2,4,6- triisopropylphenyl)-2,3- dihydroimidazo[1,5-a]pyridin-3- yl)copper(I) chloride |
| 60 | 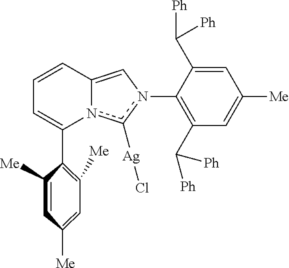 | (2-(2,6-dibenzhydryl-4- methylphenyl)-5-mesityl-2,3- dihydroimidazo[1,5-a]pyridin-3- yl)silver(I) chloride |
| 61 | 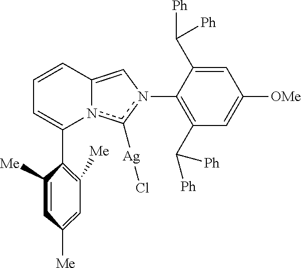 | (2-(2,6-dibenzhydryl-4- methoxyphenyl)-5-mesityl-2,3- dihydroimidazo[1,5-a]pyridin-3- yl)silver(I) chloride |
| 62 | 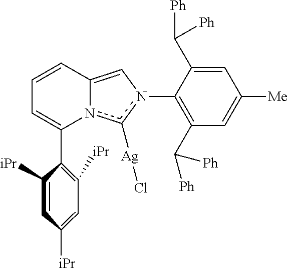 | (2-(2,6-dibenzhydryl-4- methylphenyl)-5-(2,4,6- triisopropylphenyl)-2,3- dihydroimidazo[1,5-a]pyridin-3- yl)silver(I) chloride |
| 63 | 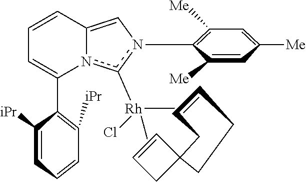 | cyclooctadiene [5-(2,6- diisopropylphenyl)-2- mesitylimidazo[1,5-a]pyridin-3- ylidene] rhodium(I) chloride |
| 64 | 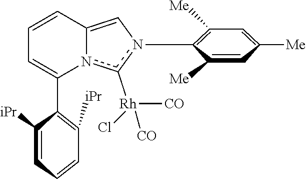 | dicarbonyl [5-(2,6- diisopropylphenyl)-2- mesitylimidazo[1,5-a]pyridin-3- ylidene] rhodium(I) chloride |
| 65 | 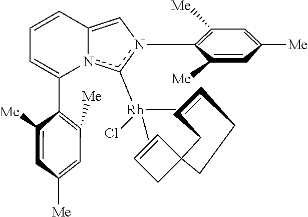 | cyclooctadiene [2,5- dimesitylimidazo[1,5-a]pyridin-2- ylidene] rhodium(I) chloride |
| 66 | 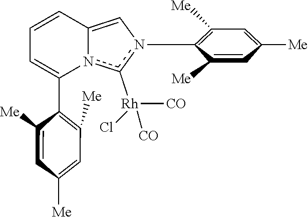 | dicarbonyl [2,5- dimesitylimidazo[1,5-a]pyridin-2- ylidene] rhodium(I) chloride |
| 67 | 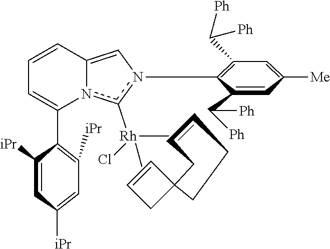 | cyclooctadiene [2-(2,6- dibenzhydryl-4-methylphenyl)-5- (2,4,6- triisopropylphenyl)imidazo[1,5- a]pyridin-3-ylidene] rhodium(I) chloride |
| 68 | 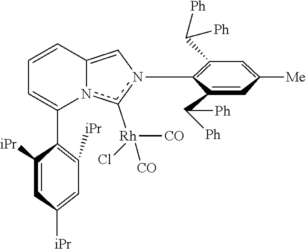 | dicarbonyl [2-(2,6-dibenzhydryl-4- methylphenyl)-5-(2,4,6- triisopropylphenyl)imidazo[1,5- a]pyridin-3-ylidene] rhodium(I) chloride |
| 69 | 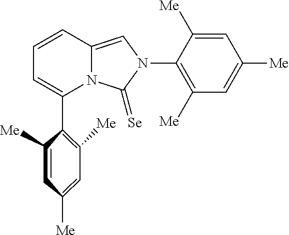 | 2,5-dimesitylimidazo[1,5-a]pyridine- 3(2H)-selenone |
| 70 | 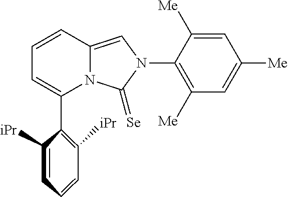 | 5-(2,6-diisopropylphenyl)-2- mesitylimidazo[1,5-a]pyridine- 3(2H)-selenone |
| 71 | 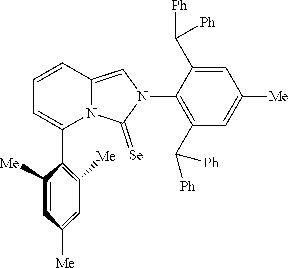 | 2-(2,6-dibenzhydryl-4- methylphenyl)-5- mesitylimidazo[1,5-a]pyridine- 3(2H)-selenone |
| 72 | 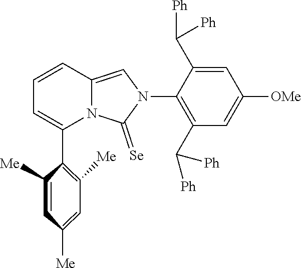 | 2-(2,6-dibenzhydryl-4- methoxyphenyl)-5- mesitylimidazo[1,5-a]pyridine- 3(2H)-selenone |
| 73 | 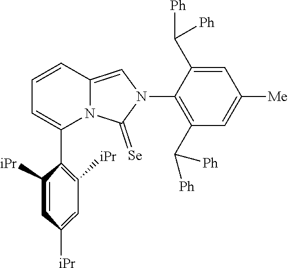 | 2-(2,6-dibenzhydryl-4- methylphenyl)-5-(2,4,6- triisopropylphenyl)imidazo[1,5- a]pyridine-3(2H)-selenone |
| 74 | 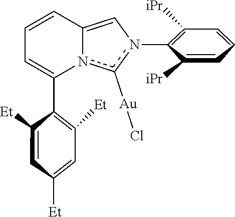 | (5-(2,4,6-triethylphenyl)-2-(2,6- diisopropylphenyl)-2,3- dihydroimidazo[1,5-a]pyridin-3- yl)gold(I) chloride |
| 75 | 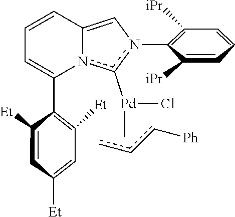 | Cinnamyl (5-(2,4,6-triethylphenyl)- 2-(2,6-diisopropylphenyl)-2,3- dihydroimidazo[1,5-a]pyridin-3- yl)chloropalladium (II) |
| 76 | 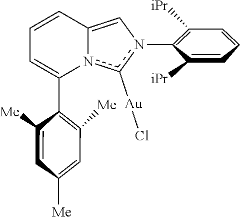 | (5-(2,4,6-trimethylphenyl)-2-(2,6- diisopropylphenyl)-2,3- dihydroimidazo[1,5-a]pyridin-3- yl)gold(I) chloride |
| 77 | 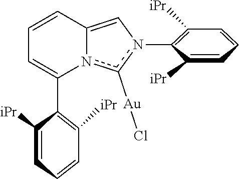 | (5-(2,6-diisopropylphenyl)-2-(2,6- diisopropylphenyl)-2,3- dihydroimidazo[1,5-a]pyridin-3- yl)gold(I) chloride |
| 78 | 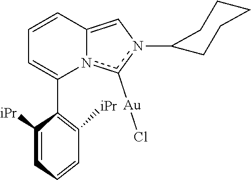 | (5-(2,6-diisopropylphenyl)-2- cyclohexyl-2,3-dihydroimidazo[1,5- a]pyridin-3-yl)gold(I) chloride |

### Methods

In one aspect, the present disclosure provides a method of promoting a reaction between a boronic acid and a nitroarene, the method comprising contacting the boronic acid and the nitroarene in the presence of the compound of the present disclosure and a base, and optionally in the presence of a phase transfer catalyst. In certain embodiments, the base is K₃PO₄. In certain embodiments, the method further comprises water. In certain embodiments, the phase transfer catalyst is TDA. In certain embodiments, the reaction occurs at a temperature of about 130° C. In certain embodiments, the reaction occurs in the presence of a solvent. In certain embodiments, the solvent is 1,4-dioxane.

In one aspect, the present disclosure provides a method of promoting a reaction between an alkyl sulfoxide and an aniline, the method comprising contacting the alkyl sulfoxide and the aniline in the presence of the compound of the present disclosure and a base. In certain embodiments, the base is LiHMDS. In certain embodiments, the reaction occurs in the presence of a solvent. In certain embodiments, the solvent is THF. In certain embodiments, the reaction occurs at a temperature of about 100° C.

In one aspect, the present disclosure provides a method of promoting a reaction between a thiophenol and an aniline, the method comprising contacting the thiophenol and the aniline in the presence of the compound of the present disclosure and a base. In certain embodiments, the base is LiHMDS. In certain embodiments, the reaction occurs in the presence of a solvent. In certain embodiments, the solvent is toluene. In certain embodiments, the reaction occurs at a temperature of about 130° C.

In one aspect, the present disclosure provides a method of promoting a reaction between an aryl halide and hydrazine, the method comprising contacting the aryl halide and the hydrazine in the presence of the compound of the present disclosure and a Lewis acid to provide an aryl hydrazine. In certain embodiments, the Lewis acid is rubidium carbonate. In certain embodiments, the reaction is performed in the presence of a solvent. In certain embodiments, the solvent is 1,4-dioxane. In certain embodiments, the hydrazine is hydrazine monohydrate. In certain embodiments, the reaction occurs at a temperature of about 100° C. In certain embodiments, the reaction further comprises contacting the aryl hydrazine and a 1,3-dione to provide a 1-phenylpyrazole. In certain embodiments, the 1,3-dione is acetylacetone.

In one aspect, the present disclosure provides a method of promoting a reaction of an aryl mesylate or aryl tosylate with a boronic acid, the method comprising contacting the aryl mesylate or aryl tosylate with the boronic acid in the presence of the compound of the present disclosure and a base. In certain embodiments, the base is potassium phosphate. In certain embodiments, the reaction occurs in the presence of a solvent. In certain embodiments, the solvent is isopropanol. In certain embodiments, the reaction occurs at a temperature of about 130° C.

In one aspect, the present disclosure provides a method of promoting a reaction of an aryl mesylate or tosylate with an aniline, the method comprising contacting the aryl mesylate or aryl tosylate with the aniline in the presence of the compound of the present disclosure and a base. In certain embodiments, the base is potassium phosphate. In certain embodiments, the reaction occurs in the presence of a solvent. In certain embodiments, the solvent is tert-amyl alcohol. In certain embodiments, the reaction occurs at a temperature of about 120° C.

In one aspect, the present disclosure provides a method of promoting a reaction between an aryl sulfamate and a boronic acid, the method comprising contacting the aryl sulfamate and the boronic acid in the presence of the compound of the present disclosure and a base. In certain embodiments, the base is potassium phosphate. In certain embodiments, the reaction occurs in the presence of a solvent. In certain embodiments, the solvent is isopropanol. In certain embodiments, the reaction occurs at a temperature of about 130° C.

In one aspect, the present disclosure provides a method of promoting a reaction between an aryl sulfamate and an aniline, the method comprising contacting the aryl sulfamate and the aniline in the presence of the compound of the present disclosure and a base. In certain embodiments, the base is potassium phosphate. In certain embodiments, the reaction occurs in the presence of a solvent. In certain embodiments, the solvent is tert-amyl alcohol. In certain embodiments, the reaction occurs at a temperature of about 120° C.

In one aspect, the present disclosure provides a method of promoting a reaction between an aryl halide and a hydroxide salt, the method comprising contacting the aryl halide and the hydroxide salt in the presence of the compound of the present disclosure. In certain embodiments, the hydroxide salt is cesium hydroxide. In certain embodiments, the cesium hydroxide is cesium hydroxide monohydrate. In certain embodiments, the reaction is performed in the presence of a solvent. In certain embodiments, the solvent is 1,4-dioxane. In certain embodiments, the reaction occurs at a temperature of about 120° C. In certain embodiments, the aryl halide is an aryl chloride. In certain embodiments, the aryl halide is an aryl bromide.

In one aspect, the present disclosure provides a method of promoting a reaction between an aryl methyl thioether and an aniline, the method comprising contacting the aryl methyl thioether and the aniline in the presence of the compound of the present disclosure and a base. In certain embodiments, the base is NaHMDS. In certain embodiments, the reaction occurs in the presence of a solvent. In certain embodiments, the solvent is toluene. In certain embodiments, the reaction occurs at a temperature of about 100° C.

In one aspect, the present disclosure provides a method of promoting a reaction between an aryl sulfoxide and an aniline, the method comprising contacting the aryl sulfoxide and the aniline in the presence of the compound of the present disclosure and a base. In certain embodiments, the base is LiHMDS. In certain embodiments, the reaction occurs in the presence of a solvent. In certain embodiments, the solvent is toluene. In certain embodiments, the reaction occurs at a temperature of about 80° C.

In one aspect, the present disclosure provides a method of promoting a reaction between an amine and an aryl chloride, the method comprising contacting the amine and the aryl chloride in the presence of the compound of the present disclosure and a base. In certain embodiments, the amine is an aniline. In certain embodiments, the amine is a C₂-C₈ heterocyclyl with at least one secondary nitrogen. In certain embodiments, the reaction occurs in the presence of a solvent. In certain embodiments, the solvent is cyclopentyl methyl ether. In certain embodiments, the reaction occurs at a temperature of about 60° C.

In one aspect, the present disclosure provides a method of promoting a reaction between an aryl chloride and a boronic acid, the method comprising contacting the aryl chloride and the boronic acid in the presence of the compound of the present disclosure and a base. In certain embodiments, the base is NaOtBu. In certain embodiments, the reaction occurs in the presence of a solvent. In certain embodiments, the solvent is THF. In certain embodiments, the reaction occurs at a temperature of about 60° C.

In one aspect, the present disclosure provides a method of promoting a reaction between an aryl fluoride and an aniline, the method comprising contacting the aryl fluoride and the aniline in the presence of the compound of the present disclosure and a base. In certain embodiments, the base is LiHMDS. In certain embodiments, the reaction occurs in the presence of a solvent.

In certain embodiments, the solvent is toluene. In certain embodiments, the reaction occurs at a temperature of about 130° C.

In one aspect, the present disclosure provides a method of promoting a reaction between an alkyne and an aniline, the method comprising contacting the alkyne and the aniline in the presence of the compound of the present disclosure and a reducing agent. In certain embodiments, the reducing agent is a silane (e.g., phenylsilane). In certain embodiments, the reaction occurs in the presence of a Lewis acid. In certain embodiments, the Lewis acid is [Ag(MeCN)₂]⁺BARF⁻. In certain embodiments, the reaction occurs in the presence of a solvent.

In certain embodiments, the solvent is toluene. In certain embodiments, the solvent is trifluorotoluene. In certain embodiments, the solvent further comprises water. In certain embodiments, the reaction occurs at a temperature of about 40° C.

In certain embodiments, at least one of the following applies:
(a) the boronic acid is a C₆-C₁₀ aryl or C₂-C₈ heteroaryl, wherein the C₆-C₁₀ aryl or C₂-C₈ heteroaryl is substituted with at least one boronate or boronic acid moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(b) the nitroarene is a C₆-C₁₀ aryl or C₂-C₈ heteroaryl, wherein the C₆-C₁₀ aryl or C₂-C₈ heteroaryl is substituted with at least one NO₂ moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(c) the alkyl sulfoxide is a C₆-C₁₀ aryl or C₂-C₈ heteroaryl, wherein the C₆-C₁₀ aryl or C₂-C₈ heteroaryl is substituted with at least one S(═O)(optionally substituted C₁-C₆ alkyl) moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(d) the aniline is a C₆-C₁₀ aryl or C₂-C₈ heteroaryl, wherein the C₆-C₁₀ aryl or C₂-C₈ heteroaryl is substituted with at least one NH₂ moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(e) the thiophenol is a C₆-C₁₀ aryl or C₂-C₈ heteroaryl, wherein the C₆-C₁₀ aryl or C₂-C₈ heteroaryl is substituted with at least one thiol moiety and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(f) the aryl halide is a C₆-C₁₀ aryl or C₂-C₈ heteroaryl, wherein the C₆-C₁₀ aryl or C₂-C₈ heteroaryl is substituted with at least one halogen selected from the group consisting of C₁, Br, and I, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(g) the aryl tosylate is a C₆-C₁₀ aryl or C₂-C₈ heteroaryl, wherein the C₆-C₁₀ aryl or C₂-C₈ heteroaryl is substituted with at least one toluenesulfonoxy moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(h) the aryl mesylate is a C₆-C₁₀ aryl or C₂-C₈ heteroaryl, wherein the C₆-C₁₀ aryl or C₂-C₈ heteroaryl is substituted with at least one methanesulfonoxy moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(i) the aryl sulfamate is a C₆-C₁₀ aryl or C₂-C₈ heteroaryl, wherein the C₆-C₁₀ aryl or C₂-C₈ heteroaryl is substituted with at least one sulfamate moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(j) the aryl chloride is a C₆-C₁₀ aryl or C₂-C₈ heteroaryl, wherein the C₆-C₁₀ aryl or C₂-C₈ heteroaryl is substituted with at least one C₁, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(k) the aryl methyl thioether is a C₆-C₁₀ aryl or C₂-C₈ heteroaryl, wherein the C₆-C₁₀ aryl or C₂-C₈ heteroaryl is substituted with at least one methylthio moiety (i.e., —SCH₃), and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(l) the aryl sulfoxide is C₆-C₁₀ aryl or C₂-C₈ heteroaryl, wherein the C₆-C₁₀ aryl or C₂-C₈ heteroaryl is substituted with at least one substituent selected from the group consisting of S(═O)(optionally substituted C₁-C₆ alkyl), S(═O)(optionally substituted C₆-C₁₀ aryl), and S(═O)(optionally substituted C₂-C₈ heteroaryl), and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(m) the amine is selected from the group consisting of a N (optionally substituted C₁-C₆ alkyl)₂, N (optionally substituted C₁-C₆ alkyl)(optionally substituted C₆-C₁₀ aryl), N (optionally substituted C₆-C₁₀ aryl)₂, and optionally substituted C₂-C₈ heterocycloalkyl, wherein the heterocycloalkyl comprises at least one secondary amine;
(n) the aryl bromide is a C₆-C₁₀ aryl or C₂-C₈ heteroaryl, wherein the C₆-C₁₀ aryl or C₂-C₈ heteroaryl is substituted with at least one Br, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂; and
(o) the aryl fluoride is a C₆-C₁₀ aryl or C₂-C₈ heteroaryl, wherein the C₆-C₁₀ aryl or C₂-C₈ heteroaryl is substituted with at least one Br, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the compound of the present disclosure is a compound of formula (III):

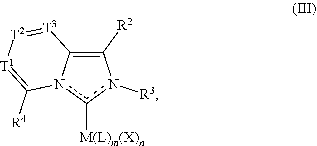

wherein:
M is a transition metal;
L is a ligand of M, wherein each occurrence of L can be the same or different;
each occurrence of [symbol] is a single or double bond,
   wherein no more than one [symbol] bonding a C atom and a N atom is a double bond; [symbol]
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R³ is selected from the group consisting of optionally substituted C₆-C₁₀ aryl, optionally substituted C₂-C₈ heteroaryl, and optionally substituted C₃-C₈ cycloalkyl,
   wherein each optional substituent in R³ is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R⁴ is selected from the group consisting of optionally substituted C₃-C₁₂ cycloalkyl and phenyl substituted with at least one substituent selected from the group consisting of C₁-C₆ alkyl, C₃-C₁₂ cycloalkyl, C₁-C₆ alkoxy, phenyl, and N(R^{A})(R^{B}),
   wherein each optional substituent in the C₃-C₁₂ cycloalkyl is at least one selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
X is a counter anion;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen;
m is an integer which is selected from the group consisting of 0, 1, 2, and 3; and
n is an integer which is selected from the group consisting of 0, 1, and 2.

In certain embodiments, M in the compound of formula (III) is Pd. In certain embodiments, the compound of formula (III) has a concentration of about 1, 2, 3, 4, 5, 6, 7, 8, 9, or 10 mol %. In certain embodiments, the compound of formula (III) is selected from the group consisting of compound 37, 38, 39, 40, 41, 42, 43, 44, 45, 46, 47, 48, 49, and 50.

In certain embodiments, the compound of the present disclosure is a compound of formula (II). In certain embodiments, M in the compound of formula (II) is Pd. In certain embodiments, the compound of formula (II) has a concentration of about 1, 2, 3, 4, 5, 6, 7, 8, 9, or 10 mol %.

In another aspect, the present disclosure provides a method of preparing a compound of formula (IV):

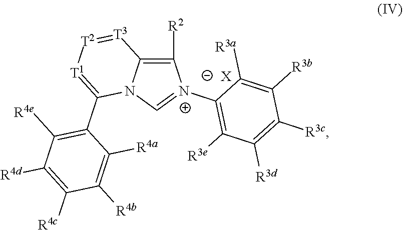

wherein:
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
   wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
   wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; and
X is a counter ion;
the method comprising contacting (Z):

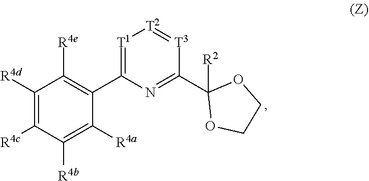

and (Y):

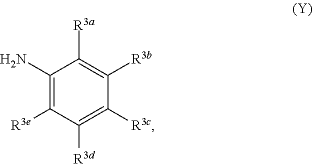

in the presence of paraformaldehyde and an acid.

In certain embodiments, Z is

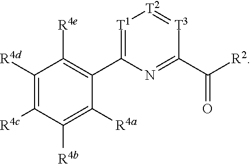

In certain embodiments, Z is

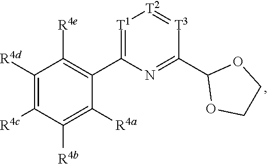

In certain embodiments, Z is

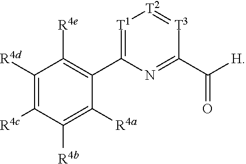

In certain embodiments, the acid is HCl in 1,4-dioxane, such as but not limited to 4 M HCl in 1,4-dioxane. In certain embodiments, the reaction occurs at a temperature of about 100° C. In certain embodiments, the reaction occurs in the presence of a solvent. In certain embodiments, the solvent is toluene.

In certain embodiments, the (Z) is prepared by reacting (X):

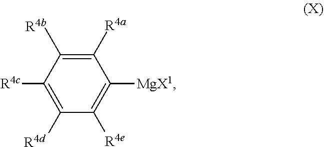

### and (W):

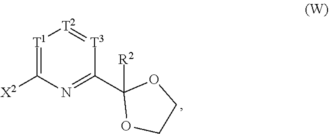

in the presence of a transition metal catalyst;
wherein X¹ and X² are each independently selected from the group consisting of Cl, Br, and I.

In certain embodiments, W is

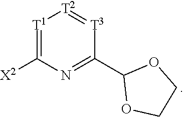

In certain embodiments, X¹ is Cl. In certain embodiments, X² is Br. In certain embodiments, the transition metal catalyst is Ni(PCy₃)Cl₂. In certain embodiments, the reaction occurs in the presence of a solvent. In certain embodiments, the solvent is THF. In certain embodiments, the reaction occurs at a temperature of about 50° C.

In certain embodiments, T¹ is CR^{1a}, T² is CR^{1b}, and T³ is CR^{1c}. In certain embodiments, T¹ is N, T² is CR^{1b}, and T³ is CR^{1c}. In certain embodiments, T¹ is CR^{1a}, T² is N, and T³ is CR^{1c}. In certain embodiments, T¹ is CR^{1a}, T² is CR^{1b}, and T³ is N. In certain embodiments, T¹ is N, T² is N, and T³ is CR^{1c}. In certain embodiments, T¹ is N, T² is CR^{1b}, and T³ is N. In certain embodiments, T¹ is CR^{1a}, T² is N, and T³ is N.

### EXAMPLES

Various embodiments of the present application can be better understood by reference to the following Examples which are offered by way of illustration. The scope of the present application is not limited to the Examples given herein.

### Example 1: Synthesis of Imidazo[1,5-α]pyridine N-Heterocyclic Carbene (NHC) Ligands General Procedure A: Suzuki Cross-Coupling

### Step 1: Suzuki Cross-Coupling

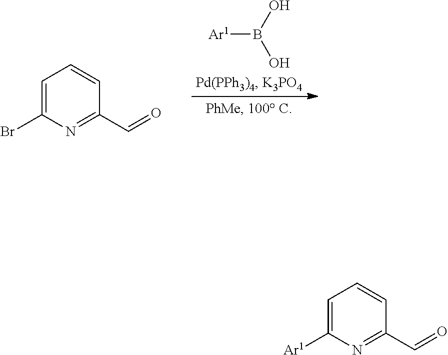

A mixture of 6-bromopyridine-2-carboxaldehyde (930 mg, 5 mmol, 1 equiv), arylboronic acid (Ar¹—B(OH)₂) (6.5 mmol, 1.3 equiv), Pd(PPh₃)₄ (289 mg, 0.25 mmol, 5 mol %), and K₃PO₄ (3.18 g, 15 mmol, 3 equiv) in toluene (20 mL, 0.25 M) was heated at 100° C. for 12 h under argon. Upon cooling, the organic layer was separated, and the water layer was extracted with ethyl acetate (50 mL×2). The organic layers were combined and dried over Na₂SO₄. The solvent was removed in vacuo, the residue was chromatographed on silica gel with elution of ethyl acetate/hexane (1/20) to give the desired product.

### Step 2: Synthesis of NHC Salts

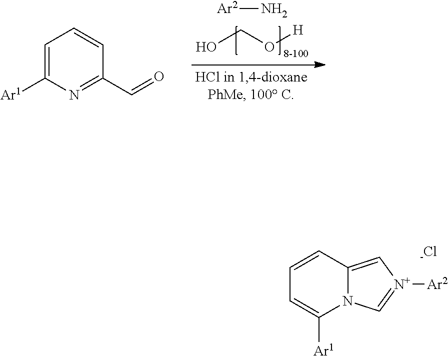

To a mixture of the above obtained picolinaldehyde (1 mmol, 1 equiv), aniline (1 mmol, 1 equiv), paraformaldehyde (45 mg, 1.5 mmol, 1.5 equiv) in toluene (2 mL, 0.5 M) was added 4 M HCl in dioxane (0.5 ml, 2 mmol, 2 equiv). The mixture was then heated at 100° C. for 12 h. Solvent was removed under reduced pressure. The residue was chromatographed on silica gel with elution of dichloromethane/methanol (50/1 to 30/1 to 15/1) to give the desired product.

### General Procedure B: Kumada Cross-Coupling

### Step 1: 2-Bromo-6-(1,3-Dioxolan-2-Yl)Pyridine

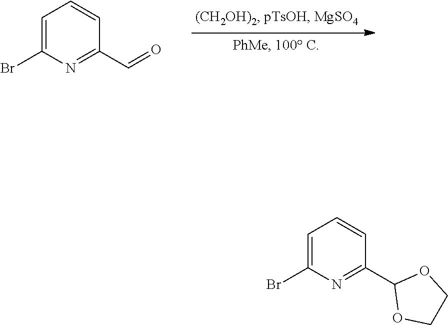 [SMILES: O=Cc1cccc(Br)n1 ; Brc1cccc(C2OCCO2)n1]

A mixture of 6-bromopyridine-2-carboxaldehyde (18.6 g, 100 mmol, 1 equiv), ethylene glycol (11.2 mL, 12.4 g, 200 mmol, 2 equiv), p-toluenesulfonic acid monohydrate (1.72 g, 10 mmol, 10 mol %) and MgSO₄ (12 g, 100 mmol, 1 equiv) in toluene (100 mL) was heated at 100° C. for 12 h. Upon cooling, aqueous NaHCO₃ solution was added to the reaction mixture. The organic layer was separated, and the water layer was extracted with ethyl acetate (100 mL×2). The combined organic layers were washed with brine and dried over Na₂SO₄. The solvent was removed in vacuo to give the product as colorless oil in 95% yield (21.9 g). ¹H NMR (500 MHz, CDCl₃) δ 7.57 (t, J=7.7 Hz, 1H), 7.51-7.43 (m, 2H), 5.79 (s, 1H), 4.16-4.11 (m, 2H), 4.08-4.02 (m, 2H). ¹³C NMR (125 MHz, CDCl₃) δ 158.61, 141.74, 139.18, 128.58, 119.49, 102.85, 65.70.

### Step 2: Kumada Cross-Coupling Reaction

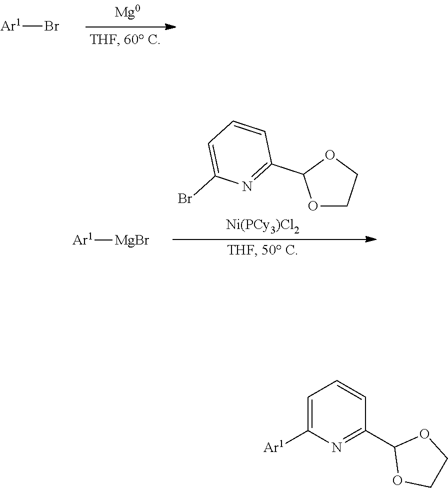

Activated magnesium turnings (173 mg, 7.2 mmol, 1.44 equiv) was suspended in anhydrous THF (10 mL). To the mixture at ambient temperature was slowly added a solution of aryl bromide (6 mmol, 1.2 equiv) in anhydrous THF (5 mL). The Grignard reaction was initiated by the addition of catalytic 1,2-dibromoethane (50 L). After complete addition, the reaction mixture was heated at 60° C. for 2 h.

To a well-stirred suspension of the 2-bromo-6-(1,3-dioxolan-2-yl)pyridine (1.15 g, 5 mmol, 1.0 equiv) and Ni(PCy₃)Cl₂ (34.5 mg, 0.05 mmol, 1 mol %) in anhydrous THF (10 mL) was slowly added the above Grignard solution over 10 minutes. The resultant brown solution was heated at 50° C. for 12 h, after which the mixture was poured over aqueous NH₄Cl solution.

The aqueous layer was extracted with ethyl acetate, and the combined organic extracts were washed with brine and dried over anhydrous Na₂SO₄, filtered and concentrated in vacuo. The crude residue was purified by flash chromatography on silica gel (hexane/ethyl acetate 10/1 to 6/1).

### Step 3: Synthesis of NHC Salts

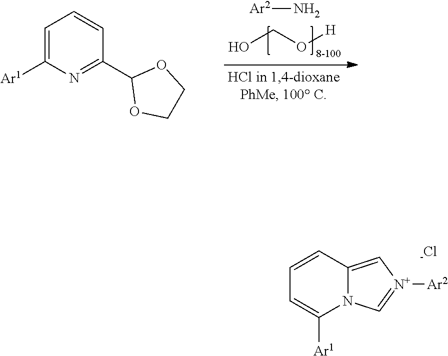

An oven-dried 100 mL round-bottomed flask equipped with a stir bar was charged with the intermediate described above (2.0 mmol, 1.0 equiv), aniline (Ar²—NH₂) (2 mmol, 1.0 equiv), paraformaldehyde (90 mg, 3.0 mmol, 1.5 equiv) and toluene (10 mL). The reaction mixture was stirred at 100° C. and 4 M HCl in dioxane (2 mL, 8.0 mmol, 4 equiv) was added. The resulting reaction mixture was stirred at 100° C. for 12 h. The crude product was purified by column chromatography (CH₂Cl₂/MeOH 50/1 to 15/1). The title product was obtained by trituration from diethyl ether/ethyl acetate as white solid.

### Synthesis of Intermediates

### General Procedure A

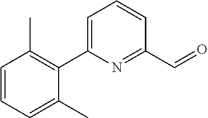 [SMILES: Cc1cccc(C)c1-c1cccc(C=O)n1]

6-(2,6-Dimethylphenyl)picolinaldehyde. Prepared according to Step 1 in General Procedure A, the reaction of 6-bromopyridine-2-carboxaldehyde (930 mg, 5 mmol, 1 equiv), 2,6-dimethylphenylboronic acid (975 mg, 6.5 mmol, 1.3 equiv), Pd(PPh₃)₄ (289 mg, 0.25 mmol, 5 mol %), and K₃PO₄ (3.18 g, 15 mmol, 3 equiv) in toluene (20 mL) gave the title compound as colorless oil in 94% yield (992 mg). ¹H NMR (500 MHz, CDCl₃) δ 10.13 (s, 1H), 7.98-7.94 (m, 2H), 7.50-7.46 (m, 1H), 7.26-7.23 (m, 1H), 7.15 (d, J=7.6 Hz, 2H), 2.06 (s, 6H). ¹³C NMR (125 MHz, CDCl₃) δ 194.03, 160.85, 153.00, 139.41, 137.56, 135.88, 129.05, 128.60, 127.99, 119.74, 20.39.

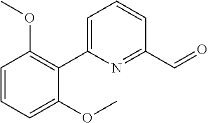 [SMILES: COc1cccc(OC)c1-c1cccc(C=O)n1]

6-(2,6-dimethoxyphenyl)picolinaldehyde. Prepared according to Step 1 in General Procedure A, the reaction of 6-bromopyridine-2-carboxaldehyde (1.86 g, 10 mmol, 1 equiv), 2,6-dimethoxyphenylboronic acid (2.37 g, 13 mmol, 1.3 equiv), Pd(PPh₃)₄ (578 mg, 0.25 mmol, 5 mol %), and K₃PO₄ (6.36 g, 15 mmol, 3 equiv) in toluene (40 mL) gave the title compound as white solid in 76% yield (1.85 g). Spectroscopic data matched literature values.

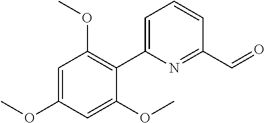 [SMILES: COc1cc(OC)c(-c2cccc(C=O)n2)c(OC)c1]

6-(2,4,6-trimethoxyphenyl)picolinaldehyde. Prepared according to Step 1 in General Procedure A, the reaction of 6-bromopyridine-2-carboxaldehyde (1.86 g, 10 mmol, 1 equiv), 2,4,6-trimethoxyphenylboronic acid (2.75 g, 13 mmol, 1.3 equiv), Pd(PPh₃)₄ (578 mg, 0.25 mmol, 5 mol %), and K₃PO₄ (6.36 g, 15 mmol, 3 equiv) in toluene (40 mL) gave the title compound as white solid in 67% yield (1.83 g). ¹H NMR (500 MHz, CDCl₃) δ 10.14 (s, 1H), 7.92-7.83 (m, 2H), 7.51 (d, J=7.5 Hz, 1H), 6.23 (s, 2H), 3.87 (s, 4H), 3.72 (s, 6H). ¹³C NMR (125 MHz, CDCl₃) δ 194.43, 161.97, 158.98, 155.60, 152.79, 136.71, 131.33, 119.58, 111.24, 91.14, 56.06, 55.62. HRMS calcd for C₃₀H₃₀O₈N₂Na (2M⁺+Na) 569.1894, found 569.1921.

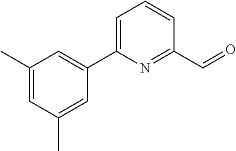 [SMILES: Cc1cc(C)cc(-c2cccc(C=O)n2)c1]

6-(3,5-dimethylphenyl)picolinaldehyde. Prepared according to Step 1 in General Procedure A, the reaction of 6-bromopyridine-2-carboxaldehyde (1.49 g, 8 mmol, 1 equiv), 3,5-dimethylphenylboronic acid (1.56 g, 10.4 mmol), Pd(PPh₃)₄ (0.46 g, 0.4 mmol, 5 mol %), and K₃PO₄ (3.4 g, 16 mmol, 2 equiv) in toluene (27 mL) gave the title compound as white solid (1.61 g, 95%). ¹H NMR (500 MHz, CDCl₃) δ 10.18 (s, 1H), 7.97-7.87 (m, 3H), 7.69 (s, 2H), 7.12 (s, 1H), 2.43 (s, 6H). ¹³C NMR (125 MHz, CDCl₃) S 194.17, 158.48, 152.84, 138.69, 138.28, 137.78, 131.47, 125.02, 124.79, 119.76, 21.58. Spectroscopic data matched literature values.

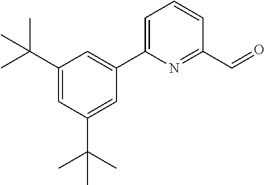 [SMILES: CC(C)(C)c1cc(-c2cccc(C=O)n2)cc(C(C)(C)C)c1]

6-(3,5-di-tert-butylphenyl)picolinaldehyde. Prepared according to Step 1 in General Procedure A, the reaction of 6-bromopyridine-2-carboxaldehyde (0.56 g, 3 mmol, 1 equiv), 3,5-di-tert-butylphenylboronic acid (0.92 g, 3.9 mmol, 1.3 equiv), Pd(PPh₃)₄ (0.17 g, 0.15 mmol, 5 mol %), and K₃PO₄ (1.3 g, 6 mmol, 2 equiv) in toluene (10 mL) gave the title compound as white solid (0.78 g, 88%). ¹H NMR (500 MHz, CDCl₃) δ 10.14 (s, 1H), 7.91-7.81 (m, 5H), 7.52 (s, 1H), 1.36 (s, 18H). ¹³C NMR (125 MHz, CDCl₃) δ 194.19, 159.25, 152.76, 151.51, 137.78, 137.66, 124.96, 123.95, 121.55, 119.42, 35.14, 31.60. HRMS calcd for C₂₀H₂₆NO (M⁺+H) 296.2009, found 296.2028.

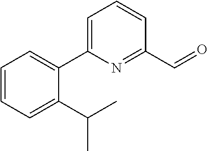 [SMILES: CC(C)c1ccccc1-c1cccc(C=O)n1]

6-(2-isopropylphenyl)picolinaldehyde. Prepared according to Step 1 in General Procedure A, the reaction of 6-bromopyridine-2-carboxaldehyde (930 mg, 5 mmol, 1 equiv), 2-isopropylphenylboronic acid (1.07 g, 6.5 mmol, 1.3 equiv), Pd(PPh₃)₄ (289 mg, 0.25 mmol, 5 mol %), and K₃PO₄ (3.18 g, 15 mmol, 3 equiv) in toluene (20 mL) gave the title compound as colorless oil in 91% yield (1.02 g). ¹H NMR (500 MHz, CDCl₃) δ 10.13 (s, 1H), 7.98-7.91 (m, 2H), 7.61 (dd, J=7.0, 2.0 Hz, 1H), 7.48-7.42 (m, 2H), 7.34 (d, J=6.0 Hz, 1H), 7.29 (t, J=6.4 Hz, 1H), 3.14 (p, J=6.9 Hz, 1H), 1.22 (d, J=6.9 Hz, 6H). ¹³C NMR (125 MHz, CDCl₃) δ 194.06, 161.21, 152.44, 146.90, 138.69, 137.32, 129.75, 129.34, 128.62, 126.19, 125.93, 119.55, 29.57, 24.28. HRMS calcd for C₁₅H₁₆NO (M⁺+H) 226.1226, found 226.1203.

### General Procedure B

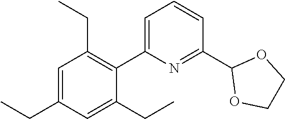 [SMILES: CCc1cc(CC)c(-c2cccc(C3OCCO3)n2)c(CC)c1]

2-(1,3-dioxolan-2-yl)-6-(2,4,6-triethylphenyl)pyridine. Prepared according to Step 2 in General Procedure B, the reaction of 2-bromo-1,3,5-triethylbenzene (1.45 g, 6 mmol, 1 equiv), magnesium turnings (173 mg, 7.2 mmol, 1.44 equiv), Ni(PCy₃)Cl₂ (34.5 mg, 0.05 mmol, 1 mol %), and 2-bromo-6-(1,3-dioxolan-2-yl)pyridine 6 (1.15 g, 5 mmol, 1.0 equiv) in THF (10 mL) gave the title compound as yellow oil in 92% yield (1.71 g). ¹H NMR (500 MHz, CDCl₃) δ 7.80 (t, J=7.7 Hz, 1H), 7.54 (d, J=7.8 Hz, 1H), 7.28 (d, J=7.6 Hz, 1H), 6.97 (s, 2H), 5.87 (s, 1H), 4.24-4.17 (m, 2H), 4.10-4.05 (m, 2H), 2.67-2.62 (m, 2H), 2.36-2.25 (m, 4H), 1.25 (t, J=7.6 Hz, 3H), 1.02 (t, J=7.6 Hz, 6H). ¹³C NMR (125 MHz, CDCl₃) δ 159.29, 156.76, 144.43, 142.05, 136.87, 126.21, 125.59, 125.56, 118.58, 104.30, 65.73, 28.96, 26.79, 15.83, 15.56. HRMS calcd for C₂₀H₂₉N₂O₂ (M⁺+NH₄) 329.2224, found 329.2167.

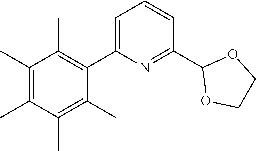 [SMILES: Cc1c(C)c(C)c(-c2cccc(C3OCCO3)n2)c(C)c1C]

2-(1,3-dioxolan-2-yl)-6-(2,3,4,5,6-pentamethylphenyl)pyridine. Prepared according to Step 2 in General Procedure B, the reaction of 1-bromo-2,3,4,5,6-pentamethylbenzene (1.36 g, 6 mmol, 1 equiv), magnesium turnings (173 mg, 7.2 mmol, 1.44 equiv), Ni(PCy₃)Cl₂ (34.5 mg, 0.05 mmol, 1 mol %), and 2-bromo-6-(1,3-dioxolan-2-yl)pyridine (1.15 g, 5 mmol, 1.0 equiv) in THF (10 mL) gave the title compound as yellow oil in 91% yield (1.35 g). ¹H NMR (500 MHz, CDCl₃) δ 7.95 (t, J=7.7 Hz, 1H), 7.68 (d, J=7.9 Hz, 1H), 7.37 (d, J=7.6 Hz, 1H), 6.02 (s, 1H), 4.39-4.34 (m, 2H), 4.25-4.22 (m, 2H), 2.44 (s, 3H), 2.39 (s, 6H), 2.06 (s, 6H). ¹³C NMR (125 MHz, CDCl₃) δ 161.35, 156.88, 138.36, 136.99, 132.58, 131.27, 125.40, 118.30, 104.41, 65.66, 18.00, 16.88, 16.48. HRMS calcd for C₃₈H₄₆O₂N₄Na (2M⁺+Na) 613.3513, found 613.3475.

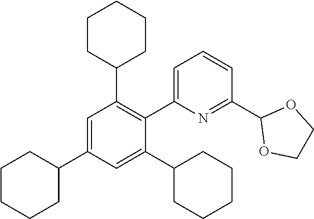 [SMILES: c1cc(-c2c(C3CCCCC3)cc(C3CCCCC3)cc2C2CCCCC2)nc(C2OCCO2)c1]

2-(1,3-dioxolan-2-yl)-6-(2,4,6-tricyclohexylphenyl)pyridine. Prepared according to Step 2 in General Procedure B, the reaction of (2-bromobenzene-1,3,5-triyl)tricyclohexane (2.42 g, 6 mmol, 1 equiv), magnesium turnings (173 mg, 7.2 mmol, 1.44 equiv), Ni(PCy₃)Cl₂ (34.5 mg, 0.05 mmol, 1 mol %), and 2-bromo-6-(1,3-dioxolan-2-yl)pyridine 6 (1.15 g, 5 mmol, 1.0 equiv) in THF (10 mL) gave the title compound as white solid in 34% yield (805 mg). ¹H NMR (500 MHz, CDCl₃) δ 7.78 (t, J=7.7 Hz, 1H), 7.53 (d, J=7.8 Hz, 1H), 7.26-7.24 (m, 1H), 7.01 (s, 2H), 5.87 (s, 1H), 4.22-4.14 (m, 2H), 4.10-4.04 (m, 2H), 2.55-2.46 (m, 1H), 2.07-1.99 (m, 2H), 1.88-1.75 (m, 8H), 1.67-1.56 (m, 8H), 1.43-1.31 (m, 8H), 1.19-1.12 (m, 2H), 1.03-0.94 (m, 4H). ¹³C NMR (125 MHz, CDCl₃) δ 159.67, 156.62, 147.89, 145.47, 136.50, 133.49, 125.44, 122.13, 118.35, 104.18, 65.62, 45.11, 41.41, 34.69, 34.63, 34.36, 27.16, 27.08, 26.92, 26.39, 26.31. HRMS calcd for C₃₂H₄₇N₂O₂ (M⁺+NH₄) 491.3632, found 491.3654.

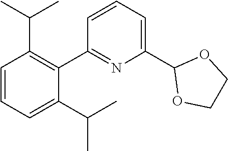 [SMILES: CC(C)c1cccc(C(C)C)c1-c1cccc(C2OCCO2)n1]

2-(2,6-diisopropylphenyl)-6-(1,3-dioxolan-2-yl)pyridine. Prepared according to Step 2 in General Procedure B, the reaction of 2-bromo-1,3-diisopropylbenzene (1.45 g, 6 mmol, 1 equiv), magnesium turnings (173 mg, 7.2 mmol, 1.44 equiv), Ni(PCy₃)Cl₂ (34.5 mg, 0.05 mmol, 1 mol %), and 2-bromo-6-(1,3-dioxolan-2-yl)pyridine 6 (1.15 g, 5 mmol, 1.0 equiv) in THF (10 mL) gave the title compound as white solid in 93% yield (1.45 g). ¹H NMR (500 MHz, CDCl₃) δ 7.80 (t, J=7.7 Hz, 1H), 7.56 (d, J=7.8 Hz, 1H), 7.35 (t, J=7.8 Hz, 1H), 7.28-7.26 (m, 1H), 7.21 (d, J=7.7 Hz, 2H), 5.87 (s, 1H), 4.24-4.17 (m, 2H), 4.10-4.05 (m, 2H), 2.51-2.42 (m, 2H), 1.12 (d, J=6.8 Hz, 6H), 1.08 (d, J=6.9 Hz, 6H). ¹³C NMR (125 MHz, CDCl₃) δ 159.39, 156.73, 146.63, 138.49, 136.67, 128.71, 125.41, 122.81, 118.63, 104.24, 65.71, 30.46, 24.27, 24.04. HRMS calcd for C₂₀H₂₉N₂O₂ (M⁺+NH₄) 329.2224, found 329.2257.

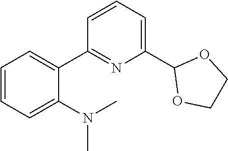 [SMILES: CN(C)c1ccccc1-c1cccc(C2OCCO2)n1]

2-(6-(1,3-dioxolan-2-yl)pyridin-2-yl)-N,N-dimethylaniline. Prepared according to Step 2 in General Procedure B, the reaction of 2-bromo-N,N-dimethylaniline (1.2 g, 6 mmol, 1 equiv), magnesium turnings (173 mg, 7.2 mmol, 1.44 equiv), Pd-PEPPSI-IPr (102 mg, 0.15 mmol, 3 mol %), and 2-bromo-6-(1,3-dioxolan-2-yl)pyridine 6 (1.15 g, 5 mmol, 1.0 equiv) in THF (10 mL) gave the title compound as yellow oil in 73% yield (987 mg). ¹H NMR (500 MHz, CDCl₃) δ 7.92 (d, J=7.3 Hz, 1H), 7.72 (t, J=7.8 Hz, 1H), 7.61 (d, J=7.1 Hz, 1H), 7.45 (d, J=7.5 Hz, 1H), 7.30 (t, J=7.4 Hz, 1H), 7.07-7.00 (m, 2H), 5.91 (s, 1H), 4.25-4.18 (m, 2H), 4.12-4.07 (m, 2H), 2.58 (s, 6H). ¹³C NMR (125 MHz, CDCl₃) δ 158.93, 156.96, 151.51, 136.66, 132.39, 132.16, 129.37, 124.54, 121.89, 118.31, 117.58, 104.41, 65.72, 44.08. HRMS calcd for C₃₂H₄₀N₅O₄ (2M⁺+NH₄) 561.3310, found 561.3244.

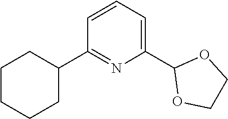 [SMILES: c1cc(C2CCCCC2)nc(C2OCCO2)c1]

2-cyclohexyl-6-(1,3-dioxolan-2-yl)pyridine. Prepared according to Step 2 in General Procedure B, the reaction of bromocyclohexane (978 mg, 6 mmol, 1 equiv), magnesium turnings (173 mg, 7.2 mmol, 1.44 equiv), Ni(PCy₃)Cl₂ (34.5 mg, 0.05 mmol, 1 mol %), and 2-bromo-6-(1,3-dioxolan-2-yl)pyridine (1.15 g, 5 mmol, 1.0 equiv) in THF (10 mL) gave the title compound as white solid in 90% yield (1.05 g). ¹H NMR (500 MHz, CDCl₃) δ 7.64 (t, J=7.8 Hz, 1H), 7.35 (d, J=7.6 Hz, 1H), 7.14 (d, J=7.8 Hz, 1H), 5.81 (s, 1H), 4.19-4.14 (m, 2H), 4.09-4.04 (m, 2H), 2.79-2.71 (m, 1H), 2.00-1.93 (m, 2H), 1.86-1.80 (m, 2H), 1.76-1.71 (m, 1H), 1.51-1.36 (m, 4H), 1.31-1.24 (m, 1H). ¹³C NMR (125 MHz, CDCl₃) δ 166.42, 156.17, 137.14, 121.04, 117.72, 104.11, 65.65, 46.59, 33.11, 26.65, 26.20. HRMS calcd for C₂₈H₄₂N₃O₄ (2M⁺+NH₄) 484.3170, found 484.3091.

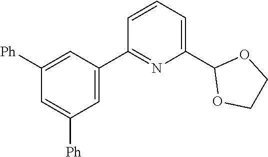 [SMILES: c1ccc(-c2cc(-c3ccccc3)cc(-c3cccc(C4OCCO4)n3)c2)cc1]

2-([1,1′:3′,1″-terphenyl]-5′-yl)-6-(1,3-dioxolan-2-yl)pyridine. Prepared according to Step 2 in General Procedure B, the reaction of 5′-Bromo-1,1′:3′,1″-terphenyl (1.86 g, 6 mmol, 1 equiv), magnesium turnings (173 mg, 7.2 mmol, 1.44 equiv), Ni(PCy₃)Cl₂ (34.5 mg, 0.05 mmol, 1 mol %), and 2-bromo-6-(1,3-dioxolan-2-yl)pyridine 6 (1.15 g, 5 mmol, 1.0 equiv) in THF (10 mL) gave the title compound as colorless oil in 81% yield (1.54 g). ¹H NMR (500 MHz, CDCl₃) δ 8.13 (d, J=1.8 Hz, 2H), 7.77-7.71 (m, 3H), 7.63 (d, J=7.0 Hz, 4H), 7.55-7.49 (m, 1H), 7.46-7.42 (m, 1H), 7.39 (t, J=7.7 Hz, 4H), 7.32-7.28 (m, 1H), 5.89 (s, 1H), 4.17-4.10 (m, 2H), 4.06-3.99 (m, 2H). ¹³C NMR (125 MHz, CDCl₃) δ 157.33, 157.03, 142.39, 141.22, 137.73, 128.92, 127.64, 127.51, 126.95, 125.20, 121.19, 119.23, 104.27, 65.78. HRMS calcd for C₂₆H₂₅N₂O₂ (M⁺+NH₄) 397.1911, found 397.1966.

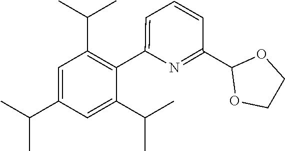 [SMILES: CC(C)c1cc(C(C)C)c(-c2cccc(C3OCCO3)n2)c(C(C)C)c1]

2-(1,3-dioxolan-2-yl)-6-(2,4,6-triisopropylphenyl)pyridine. Prepared according to Step 2 in General Procedure B, the reaction of 2-bromo-1,3,5-triisopropylbenzene (1.36 g, 6 mmol, 1 equiv), magnesium turnings (173 mg, 7.2 mmol, 1.44 equiv), Ni(PCy₃)Cl₂ (34.5 mg, 0.05 mmol, 1 mol %), and 2-bromo-6-(1,3-dioxolan-2-yl)pyridine 6 (1.15 g, 5 mmol, 1.0 equiv) in THF (10 mL) gave the title compound as yellow oil in 94% yield (1.66 g). ¹H NMR (500 MHz, CDCl₃) δ 7.78 (t, J=7.7 Hz, 1H), 7.54 (d, J=7.8 Hz, 1H), 7.29 (d, J=7.1 Hz, 1H), 5.85 (s, 1H), 4.26-4.17 (m, 2H), 4.12-4.03 (m, 2H), 2.92 (p, J=6.9 Hz, 1H), 2.47 (p, J=6.8 Hz, 2H), 1.27 (d, J=6.9 Hz, 6H), 1.12 (d, J=6.9 Hz, 6H), 1.08 (d, J=6.9 Hz, 6H). ¹³C NMR (125 MHz, CDCl₃) δ 159.67, 156.68, 148.97, 146.39, 136.58, 136.21, 125.56, 120.86, 118.47, 104.33, 65.69, 34.59, 30.49, 24.32, 24.25, 24.07.

### Imine Intermediate Syntheses

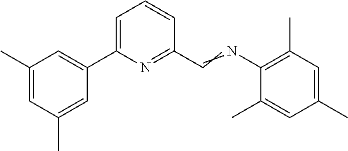

1-(6-(3,5-dimethylphenyl)pyridin-2-yl)-N-mesitylmethanimine. The reaction of 6-(3,5-dimethylphenyl)picolinaldehyde (422 mg, 2 mmol), 2,4,6-trimethylaniline (270 mg, 2 mmol) and formic acid (3 drops) in methanol (10 mL) at room temperature for 12 hours, followed by solvent concentration to 3 mL, filtration and washing with methanol gave the title compound as yellow solid (575 mg, 85%). ¹H NMR (500 MHz, CDCl₃) δ 8.43 (s, 1H), 8.23 (d, J=7.2 Hz, 1H), 7.87 (t, J=7.7 Hz, 1H), 7.80 (d, J=6.8 Hz, 1H), 7.66 (s, 2H), 7.08 (s, 1H), 6.90 (s, 2H), 2.41 (s, 6H), 2.30 (s, 3H), 2.15 (s, 6H). ¹³C NMR (125 MHz, CDCl₃) δ 164.28, 157.83, 154.64, 148.16, 139.05, 138.53, 137.36, 133.40, 131.02, 128.89, 126.92, 125.01, 122.25, 119.22, 21.59, 20.92, 18.41. HRMS calcd for C₂₃H₂₅N₂ (M⁺+H) 329.2012, found 329.2025.

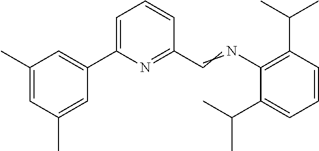 [SMILES: Cc1cc(C)cc(-c2cccc(C=Nc3c(C(C)C)cccc3C(C)C)n2)c1]

N-(2,6-diisopropylphenyl)-1-(6-(3,5-dimethylphenyl)pyridin-2-yl)methanimine. The reaction of 6-(3,5-dimethylphenyl)picolinaldehyde (422 mg, 2 mmol), 2,6-diisopropylaniline (354 mg, 2 mmol) and formic acid (3 drops) in methanol (10 mL) at room temperature for 12 hours, followed by solvent concentration to 3 mL, filtration and washing with methanol gave the title compound as yellow solid (570 mg, 77%). ¹H NMR (500 MHz, CDCl₃) δ 8.41 (s, 1H), 8.23 (d, J=7.5 Hz, 1H), 7.89 (t, J=7.8 Hz, 1H), 7.82 (d, J=7.8 Hz, 1H), 7.67 (s, 2H), 7.20-7.16 (m, 2H), 7.15-7.07 (m, 2H), 3.01 (p, J=6.8 Hz, 2H), 2.42 (s, 6H), 1.19 (d, J=6.9 Hz, 12H). ¹³C NMR (125 MHz, CDCl₃) δ 163.77, 157.92, 154.46, 148.69, 139.03, 138.53, 137.41, 137.33, 131.05, 125.04, 124.47, 123.12, 122.33, 119.34, 28.11, 23.56, 21.60. HRMS calcd for C₂₆H₃₁N₂ (M⁺+H) 371.2482, found 371.2500.

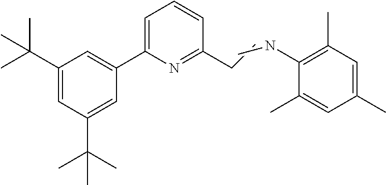

1-(6-(3,5-di-tert-butylphenyl)pyridin-2-yl)-N-mesitylmethanimine. The reaction 6-(3,5-di-tert-butylphenyl)picolinaldehyde (295 mg, 1 mmol), 2,4,6-trimethylaniline (135 mg, 1 mmol) and formic acid (3 drops) in methanol (5 mL) at room temperature for 12 hours, followed by solvent concentration to 3 mL, filtration and washing with methanol gave the title compound as yellow solid (373 mg, 90%). ¹H NMR (500 MHz, CDCl₃) δ 8.44 (s, 1H), 8.24 (d, J=7.7 Hz, 1H), 7.89 (t, J=7.8 Hz, 1H), 7.86 (d, J=1.7 Hz, 2H), 7.81 (d, J=7.7 Hz, 1H), 7.53 (s, 1H), 6.91 (s, 2H), 2.30 (s, 3H), 2.16 (s, 6H), 1.40 (s, 12H). ¹³C NMR (125 MHz, CDCl₃) δ 164.64, 158.73, 154.55, 151.41, 148.31, 138.56, 137.32, 133.39, 128.88, 126.94, 123.60, 122.48, 121.63, 118.95, 35.18, 31.66, 20.91, 18.42. HRMS calcd for C₂₉H₃₇N₂ (M⁺+H) 413.2951, found 413.2969.

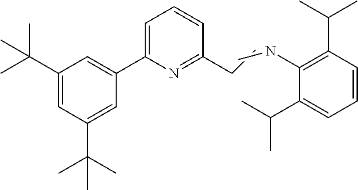

1-(6-(3,5-di-tert-butylphenyl)pyridin-2-yl)-N-(2,6-diisopropylphenyl)methanimine. The reaction 6-(3,5-di-tert-butylphenyl)picolinaldehyde (295 mg, 1 mmol), 2,6-diisopropylaniline (177 mg, 1 mmol) and formic acid (3 drops) in methanol (5 mL) at room temperature for 12 hours, followed by solvent concentration to 3 mL, filtration and washing with methanol gave the title compound as yellow solid (358 mg, 79%). ¹H NMR (500 MHz, CDCl₃) δ 8.42 (s, 1H), 8.23 (d, J=7.2 Hz, 1H), 7.90 (t, J=7.7 Hz, 1H), 7.87 (d, J=1.8 Hz, 2H), 7.83 (d, J=7.9 Hz, 1H), 7.53 (s, 1H), 7.21-7.16 (m, 2H), 7.16-7.10 (m, 1H), 3.03 (p, J=6.8 Hz, 2H), 1.40 (s, 19H), 1.20 (d, J=6.9 Hz, 12H). ¹³C NMR (125 MHz, CDCl₃) δ 164.05, 158.78, 154.41, 151.42, 148.77, 138.53, 137.40, 137.39, 124.48, 123.64, 123.15, 122.54, 121.65, 119.09, 35.18, 31.65, 28.10, 23.56. HRMS calcd for C₃₂H₄₃N₂ (M⁺+H) 455.3421, found 455.3443.

### Additional Intermediate Syntheses

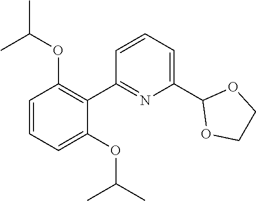 [SMILES: CC(C)Oc1cccc(OC(C)C)c1-c1cccc(C2OCCO2)n1]

2-(2,6-diisopropoxyphenyl)-6-(1,3-dioxolan-2-yl)pyridine. A mixture of nBuLi (2.5 M in hexane, 18 mL, 45 mmol, 1.5 equiv) and 2,6-diisopropoxybenzene (8.74 g, 45 mmol, 1.5 equiv) was stirred for 14 h at 50° C. for 36 h to give 2,6-diisopropoxyphenyllithium. In another flask, a few drops of 1,2-dibromoethane were added to Mg (1.08 g, 45 mmol, 1.5 equiv) in THF (50 mL). After initiation of the reaction, the remaining 1,2-dibromoethane (3.9 mL, 45 mmol) was added drop-wise, maintaining a steady exothermal reaction. Stirring was continued after the addition for 1 h at 50° C. The suspension of the magnesium bromide was poured into the aryllithium quickly. The mixture was stirred for additional 15 min at 50° C. The mixture was cooled to room temperature. Afterward, 2-bromo-6-(1,3-dioxolan-2-yl)pyridine (6.9 g, 30 mmol, 1.0 equiv) and Ni(PCy₃)Cl₂ (518 mg, 0.75 mmol, 2.5 mol %) was added to the mixture. The mixture was stirred at room temperature for 12 h, after which the mixture was poured over aqueous NH₄Cl solution. The aqueous layer was extracted with ethyl acetate, and the combined organic extracts were washed with brine and dried over anhydrous Na₂SO₄, filtered and concentrated in vacuo. The residue was loaded onto a short pad of silica gel. After washing with hexane, the residue was eluted with hexane/ethyl acetate (4/1) till fully recovery of the product. The filtrate was concentrated in vacuo to give the desired product as colorless solid in 97% yield (10 g). ¹H NMR (500 MHz, CDCl₃) δ 7.63 (t, J=7.7 Hz, 1H), 7.37 (d, J=7.1 Hz, 1H), 7.21 (d, J=7.7 Hz, 1H), 7.11 (t, J=8.3 Hz, 1H), 6.52 (d, J=8.4 Hz, 2H), 5.80 (s, 1H), 4.34-4.25 (m, 2H), 4.20-4.12 (m, 2H), 4.09-3.99 (m, 2H), 1.05 (d, J=6.2 Hz, 12H). ¹³C NMR (125 MHz, CDCl₃) δ 156.86, 155.90, 154.45, 135.77, 129.13, 126.59, 122.32, 117.89, 107.89, 104.23, 71.30, 65.30, 22.02. HRMS calcd for C₂₀H₂₅O₄NK (2M⁺+K) 382.1415, found 382.1450.

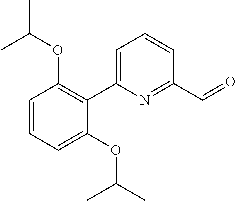 [SMILES: CC(C)Oc1cccc(OC(C)C)c1-c1cccc(C=O)n1]

6-(2,6-diisopropoxyphenyl)picolinaldehyde. A mixture of 2-(2,6-diisopropoxyphenyl)-6-(1,3-dioxolan-2-yl)pyridine (9.96 g, 29 mmol), 10% HCl solution (50 mL) was heated for 12 h at 60° C. in THF (100 mL). The mixture was quenched with aqueous NaHCO₃ solution. The aqueous layer was extracted with ethyl acetate, and the combined organic extracts were washed with brine and dried over anhydrous Na₂SO₄, filtered and concentrated in vacuo to give the desired product as white solid in 99% yield (8.59 g). ¹H NMR (500 MHz, CDCl₃) δ 10.11 (s, 1H), 7.89-7.80 (m, 2H), 7.50 (dd, J=7.5, 1.4 Hz, 1H), 7.24 (t, J=4.2 Hz, 1H), 6.62 (d, J=8.4 Hz, 2H), 4.48-4.37 (m, 2H), 1.13 (d, J=6.1 Hz, 14H). ¹³C NMR (125 MHz, CDCl₃) δ 194.65, 156.97, 156.23, 152.44, 136.24, 131.05, 130.02, 121.05, 119.30, 107.48, 71.40, 22.22.

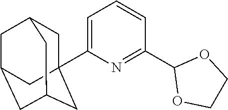 [SMILES: c1cc(C2OCCO2)nc(C23CC4CC(CC(C4)C2)C3)c1]

2-(adamantan-1-yl)-6-(1,3-dioxolan-2-yl)pyridine. Preparation of ZnCl₂ Solution: A dry and argon flushed 250 mL Schlenk-flask, equipped with a magnetic stirring bar and a septum, was charged with ZnCl₂ (2.72 g, 20 mmol). The salt was heated to 140° C. under high vacuum for 4 h. After cooling to 25° C., dry THF (20 mL) was added and stirring was continued until the salt was dissolved (4 h).

Preparation of 1-Adamantyl Zinc Chloride Solution: In an argon flushed 25 mL Schlenk flask were placed LiCl (466 mg, 11 mmol, 1.1 equiv) and dried by heating to −500° C. with a heat gun for 3 min in full vacuum. After cooling to room temperature, Mg turnings (480 mg, 20 mmol, 2.0 equiv), 15 mL of THF and 1 M ZnCl₂ solution in THF (11 mL, 11 mmol, 1.1 equiv) were put in the Schlenk flask. The magnesium was activated using 1,2-dibromoethane (5 mol %) and TMSCl (5 mol %). Then 1-adamantyl bromide (2.15 g, 10 mmol, 1 equiv) were added neat. The solids turned black, the mixture started boiling and was cooled below reflux with an ice/water bath. The reaction mixture was stirred at 60° C. until GC-analysis of hydrolyzed reaction aliquot showed full consumption of the starting material (typically 4 h). Titration against 12 showed a molarities of M=0.2 mol/L (52% yield).

Negishi Cross-Coupling of Adamantylzinc Reagents: In an argon flushed 25 mL Schlenk flask were placed palladium acetate (22 mg, 0.1 mmol, 2 mol %), SPhos (82 mg, 0.2 mmol, 4 mol %) and 2-bromo-6-(1,3-dioxolan-2-yl)pyridine (1.15 g, 5 mmol, 1.0 equiv) and dissolved in 10 mL of dry THF. Then the above 1-adamantyl zinc chloride solution in THF were added at 65° C. over 15 min. After 12 h further stirring at 65° C., the mixture was poured over aqueous NH₄Cl solution. The aqueous layer was extracted with ethyl acetate, and the combined organic extracts were washed with brine and dried over anhydrous Na₂SO₄, filtered and concentrated in vacuo. The crude residue was purified by flash chromatography on silica gel (hexane/ethyl acetate 10/1 to 6/1) to give the desired product as white solid in 53% yield (756 mg). ¹H NMR (500 MHz, CDCl₃) δ 7.65 (t, J=7.8 Hz, 1H), 7.31 (d, J=7.6 Hz, 1H), 7.24 (d, J=7.9 Hz, 1H), 5.84 (s, 1H), 4.26-4.17 (m, 2H), 4.12-4.04 (m, 2H), 2.10 (s, 3H), 2.01 (s, 6H), 1.78 (s, 6H). ¹³C NMR (125 MHz, CDCl₃) δ 168.77, 156.00, 136.90, 119.27, 117.40, 104.55, 65.73, 41.99, 39.10, 36.96, 28.92. HRMS calcd for C₃₆H₅₀N₃O₄ (2M⁺+NH₄) 588.3796, found 588.3716.

### Synthesis of Ligands

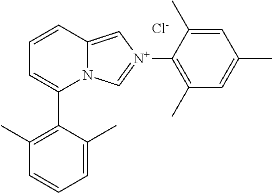

5-(2,6-dimethylphenyl)-2-mesitylimidazo[1,5-α]pyridin-2-ium chloride (1). Prepared according to Step 2 in General Procedure A, the reaction of 6-(2,6-dimethylphenyl) picolinaldehyde (211 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (0.5 mL, 2.0 mmol, 2 equiv) in toluene (5 mL) gave the title compound as white solid in 76% yield (286 mg). ¹H NMR (500 MHz, CDCl₃) δ 8.42 (s, 1H), 8.04 (d, J=9.5 Hz, 1H), 7.87 (s, 1H), 7.02 (t, J=8.2 Hz, 1H), 6.80 (t, J=7.6 Hz, 1H), 6.73-6.56 (m, 3H), 6.44 (s, 2H), 1.73 (s, 3H), 1.53 (s, 6H), 1.44 (s, 6H). ¹³C NMR (125 MHz, CDCl₃) δ 140.93, 136.67, 133.32, 132.91, 131.55, 130.87, 130.67, 129.30, 128.81, 128.51, 125.60, 122.01, 120.02, 119.23, 117.80, 20.71, 18.97, 16.97. HRMS calcd for C₂₄H₂₅N₂ (M⁺−Cl) 341.2012, found 341.2028.

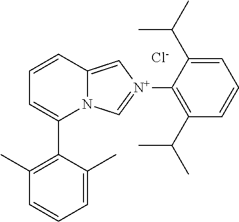 [SMILES: Cc1cccc(C)c1-c1cccc2c[n+](-c3c(C(C)C)cccc3C(C)C)cn12 ; [Cl-]]

2-(2,6-diisopropylphenyl)-5-(2,6-dimethylphenyl)imidazo[1,5-α]pyridin-2-ium chloride (2). Prepared according to Step 2 in General Procedure A, the reaction of 6-(2,6-dimethylphenyl)picolinaldehyde (211 mg, 1 mmol, 1 equiv), 2,6-diisopropylaniline (177 mg, 1 mmol, 1.0 equiv), paraformaldehyde (45 mg, 1.5 mmol, 1.5 equiv) and 4 M HCl in dioxane (0.5 mL, 2.0 mmol, 2 equiv) in toluene (5 mL) gave the title compound as white solid in 87% yield (365 mg). ¹H NMR (500 MHz, CDCl₃) δ 9.25 (s, 1H), 8.93 (d, J=9.5 Hz, 1H), 7.94 (s, 1H), 7.59-7.50 (m, 2H), 7.39 (t, J=7.6 Hz, 1H), 7.29 (d, J=8.1 Hz, 2H), 7.25 (d, J=7.6 Hz, 2H), 7.15 (d, J=7.0 Hz, 1H), 2.09-2.02 (m, 8H), 1.23 (d, J=6.9 Hz, 6H), 1.03 (d, J=6.9 Hz, 6H). ¹³C NMR (125 MHz, CDCl₃) δ 143.65, 135.97, 132.00, 131.19, 130.99, 130.52, 129.41, 128.06, 127.95, 125.44, 123.58, 121.17, 119.74, 119.08, 118.46, 27.70, 23.49, 22.93, 18.10. HRMS calcd for C₂₇H₃₁N₂ (M⁺−Cl) 383.2482, found 383.2503.

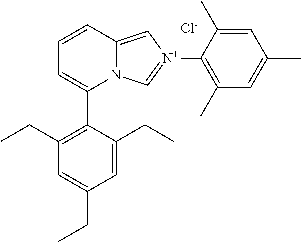 [SMILES: CCc1cc(CC)c(-c2cccc3c[n+](-c4c(C)cc(C)cc4C)cn23)c(CC)c1 ; [Cl-]]

2-mesityl-5-(2,4,6-triethylphenyl)imidazo[1,5-α]pyridin-2-ium chloride (3). Prepared according to Step 3 in General Procedure B, the reaction of 2-(1,3-dioxolan-2-yl)-6-(2,4,6-triethylphenyl)pyridine (311 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 69% yield (299 mg). ¹H NMR (500 MHz, CDCl₃) δ 9.37 (s, 1H), 8.96 (d, J=9.3 Hz, 1H), 7.86 (s, 1H), 7.51-7.42 (m, 1H), 7.11 (s, 2H), 7.08 (d, J=6.9 Hz, 1H), 6.98 (s, 2H), 2.68 (q, J=7.6 Hz, 2H), 2.37-2.28 (m, 5H), 2.22-2.16 (m, 2H), 1.95 (s, 6H), 1.27 (t, J=7.6 Hz, 3H), 1.01 (t, J=7.6 Hz, 6H). ¹³C NMR (125 MHz, CDCl₃) δ 148.34, 143.45, 141.91, 133.99, 132.69, 132.66, 131.21, 129.90, 127.20, 125.60, 125.35, 121.45, 120.97, 120.88, 119.84, 28.93, 26.49, 21.23, 17.40, 16.16, 15.32. HRMS calcd for C₂₈H₃₃N₂ (M⁺−Cl) 397.2638, found 397.2658.

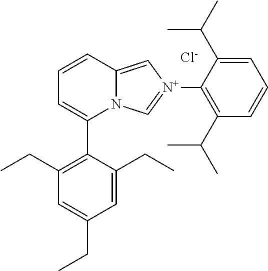 [SMILES: CCc1cc(CC)c(-c2cccc3c[n+](-c4c(C(C)C)cccc4C(C)C)cn23)c(CC)c1 ; [Cl-]]

2-(2,6-diisopropylphenyl)-5-(2,4,6-triethylphenyl)imidazo[1,5-α]pyridin-2-ium chloride (4). Prepared according to Step 3 in General Procedure B, the reaction of 2-(1,3-dioxolan-2-yl)-6-(2,4,6-triethylphenyl)pyridine (311 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 85% yield (404 mg). ¹H NMR (500 MHz, CDCl₃) δ 9.10 (s, 1H), 8.82 (d, J=9.3 Hz, 1H), 7.88 (s, 1H), 7.53-7.42 (m, 2H), 7.24-7.19 (m, 2H), 7.07 (d, J=6.9, 1H), 7.04 (s, 2H), 2.61 (q, J=7.6 Hz, 2H), 2.30-2.21 (m, 2H), 2.18-2.08 (m, 2H), 2.02-1.92 (m, 2H), 1.20 (t, 3H), 1.15 (d, J=6.7 Hz, 6H), 0.97-0.91 (m, 12H). ¹³C NMR (125 MHz, CDCl₃) δ 147.93, 144.43, 142.84, 132.50, 131.92, 131.80, 130.08, 126.72, 126.08, 124.93, 124.31, 121.97, 120.89, 119.82, 119.05, 28.43, 25.93, 24.14, 23.75, 15.65, 14.77. HRMS calcd for C₃₁H₃₉N₂ (M⁺−Cl) 439.3108, found 439.3133.

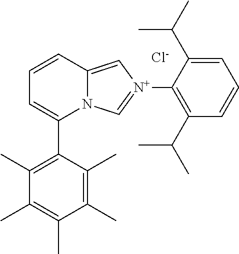 [SMILES: Cc1c(C)c(C)c(-c2cccc3c[n+](-c4c(C(C)C)cccc4C(C)C)cn23)c(C)c1C ; [Cl-]]

2-(2,6-diisopropylphenyl)-5-(2,3,4,5,6-pentamethylphenyl)imidazo[1,5-α]pyridin-2-ium chloride (5). Prepared according to Step 3 in General Procedure B, the reaction of 2-(1,3-dioxolan-2-yl)-6-(2,3,4,5,6-pentamethylphenyl)pyridine (595 mg, 2 mmol, 1 equiv), 2,6-diisopropylaniline (354 mg, 2 mmol, 1.0 equiv), paraformaldehyde (90 mg, 3 mmol, 1.5 equiv) and 4 M HCl in dioxane (2 mL, 8.0 mmol, 4 equiv) in toluene (10 mL) gave the title compound as white solid in 68% yield (627 mg). ¹H NMR (500 MHz, CDCl₃) δ 9.38 (s, 1H), 9.10-8.95 (m, 1H), 7.80 (s, 1H), 7.55-7.43 (m, 2H), 7.26-7.19 (m, 2H), 7.03 (d, J=5.7 Hz, 1H), 2.26 (s, 3H), 2.20 (s, 6H), 2.13-2.02 (m, 2H), 1.91 (s, 7H), 1.22 (d, J=5.6 Hz, 6H), 0.99 (d, J=5.7 Hz, 6H). ¹³C NMR (125 MHz, CDCl₃) δ 144.95, 138.99, 134.68, 134.62, 132.43, 132.23, 132.10, 130.76, 127.33, 126.02, 124.73, 121.88, 121.40, 120.78, 120.73, 28.89, 24.81, 24.04, 17.34, 17.19, 16.71. HRMS calcd for C₃₀H₃₇N₂ (M⁺−Cl) 425.2951, found 425.2966.

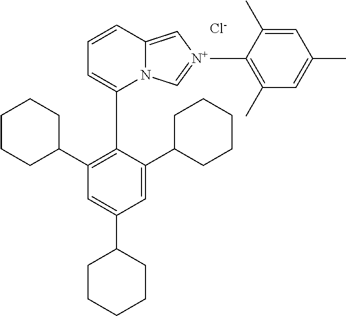 [SMILES: Cc1cc(C)c(-[n+]2cc3cccc(-c4c(C5CCCCC5)cc(C5CCCCC5)cc4C4CCCCC4)n3c2)c(C)c1 ; [Cl-]]

2-mesityl-5-(2,4,6-tricyclohexylphenyl)imidazo[1,5-α]pyridin-2-ium chloride (6). Prepared according to Step 3 in General Procedure B, the reaction of 2-(1,3-dioxolan-2-yl)-6-(2,4,6-tricyclohexylphenyl)pyridine (474 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 73% yield (435 mg). ¹H NMR (500 MHz, CDCl₃) δ 9.37 (d, J=2.0 Hz, 1H), 9.03 (d, J=9.4 Hz, 1H), 7.88 (d, J=1.8 Hz, 1H), 7.45 (dd, J=9.4, 6.8 Hz, 1H), 7.10 (s, 2H), 6.98 (d, J=7.1 Hz, 1H), 6.96 (s, 2H), 2.56-2.46 (m, 1H), 2.30 (s, 3H), 1.94-1.82 (m, 10H), 1.79-1.75 (m, 2H), 1.73-1.67 (m, 4H), 1.64-1.57 (m, 4H), 1.51-1.22 (m, 12H), 1.18-1.09 (m, 2H), 1.06-0.97 (m, 2H), 0.89-0.80 (m, 2H). ¹³C NMR (125 MHz, CDCl₃) δ 151.95, 146.84, 141.90, 133.97, 132.97, 132.63, 131.13, 129.78, 125.31, 124.66, 123.80, 121.37, 121.08, 120.68, 119.61, 44.99, 42.47, 35.16, 35.00, 34.36, 26.83, 26.57, 26.07, 25.63, 21.18, 16.88. HRMS calcd for C₄₀H₅₁N₂ (M⁺−Cl) 559.4047, found 559.4071.

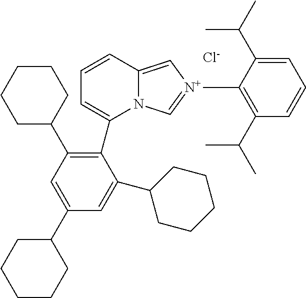 [SMILES: CC(C)c1cccc(C(C)C)c1-[n+]1cc2cccc(-c3c(C4CCCCC4)cc(C4CCCCC4)cc3C3CCCCC3)n2c1 ; [Cl-]]

2-(2,6-diisopropylphenyl)-5-(2,4,6-tricyclohexylphenyl)imidazo[1,5-α]pyridin-2-ium chloride (7). Prepared according to Step 3 in General Procedure B, the reaction of 2-(1,3-dioxolan-2-yl)-6-(2,4,6-tricyclohexylphenyl)pyridine (474 mg, 1 mmol, 1 equiv), 2,6-diisopropylaniline (177 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 73% yield (465 mg). ¹H NMR (500 MHz, CDCl₃) δ 9.50 (s, 1H), 9.17 (d, J=9.3 Hz, 1H), 7.93 (s, 1H), 7.54-7.49 (m, 2H), 7.28 (d, J=7.8 Hz, 2H), 7.12 (s, 2H), 7.02 (d, J=6.7 Hz, 1H), 2.55-2.49 (m, 1H), 2.03-1.99 (m, 2H), 1.89-1.84 (m, 4H), 1.77-1.64 (m, 12H), 1.57-1.50 (m, 2H), 1.43-1.32 (m, 8H), 1.22 (d, J=6.7 Hz, 6H), 1.18-1.13 (m, 2H), 1.11-1.06 (m, 2H), 1.03 (d, J=6.9 Hz, 6H), 0.93-0.88 (m, 2H). ¹³C NMR (125 MHz, CDCl₃) δ 152.01, 146.95, 145.04, 132.82, 132.59, 132.26, 130.67, 125.61, 124.73, 123.83, 121.75, 121.50, 121.14, 120.93, 45.02, 42.57, 35.59, 34.61, 34.39, 28.98, 26.88, 26.72, 26.59, 26.12, 25.70, 24.89, 23.97. HRMS calcd for C₄₃H₅₇N₂ (M⁺−Cl) 601.4516, found 601.4543.

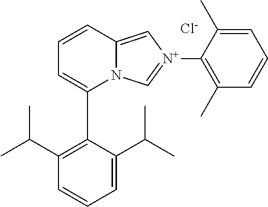 [SMILES: Cc1cccc(C)c1-[n+]1cc2cccc(-c3c(C(C)C)cccc3C(C)C)n2c1 ; [Cl-]]

5-(2,6-diisopropylphenyl)-2-(2,6-dimethylphenyl)imidazo[1,5-α]pyridin-2-ium chloride (8). Prepared according to Step 3 in General Procedure B, the reaction of 2-(2,6-diisopropylphenyl)-6-(1,3-dioxolan-2-yl)pyridine (311 mg, 1 mmol, 1 equiv), 2,6-dimethylaniline (121 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 75% yield (314 mg). ¹H NMR (500 MHz, CDCl₃) δ 9.43 (s, 1H), 9.01 (d, J=9.6 Hz, 1H), 7.91 (s, 1H), 7.56 (t, J=7.9 Hz, 1H), 7.49 (dd, J=9.3, 6.9 Hz, 1H), 7.39-7.32 (m, 3H), 7.19 (d, J=7.8 Hz, 2H), 7.07 (d, J=6.9 Hz, 1H), 2.38-2.26 (m, 2H), 1.98 (s, 6H), 1.16 (d, J=6.7 Hz, 6H), 1.04 (d, J=6.9 Hz, 6H). ¹³C NMR (125 MHz, CDCl₃) δ 148.23, 134.42, 133.58, 132.78, 132.52, 132.42, 131.64, 129.32, 126.85, 125.44, 124.70, 121.60, 120.95, 120.73, 119.80, 31.35, 24.98, 24.09, 17.31. HRMS calcd for C₂₇H₃₁N₂ (M⁺−Cl) 383.2482, found 383.2503.

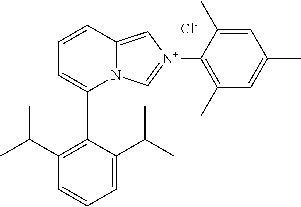 [SMILES: Cc1cc(C)c(-[n+]2cc3cccc(-c4c(C(C)C)cccc4C(C)C)n3c2)c(C)c1 ; [Cl-]]

5-(2,6-diisopropylphenyl)-2-mesitylimidazo[1,5-α]pyridin-2-ium chloride (9). Prepared according to Step 3 in General Procedure B, the reaction of 2-(2,6-diisopropylphenyl)-6-(1,3-dioxolan-2-yl)pyridine (311 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 89% yield (385 mg). ¹H NMR (500 MHz, CDCl₃) δ 9.01 (s, 1H), 8.76 (d, J=9.4 Hz, 1H), 7.90 (s, 1H), 7.61-7.46 (m, 2H), 7.35 (d, J=7.8 Hz, 2H), 7.08 (d, J=6.7 Hz, 1H), 6.98 (s, 2H), 2.39-2.26 (m, 5H), 1.93 (s, 6H), 1.15 (d, J=6.6 Hz, 6H), 1.03 (d, J=6.7 Hz, 6H). ¹³C NMR (125 MHz, CDCl₃) δ 148.24, 142.04, 134.01, 132.71, 132.53, 132.46, 131.09, 129.92, 126.81, 125.71, 124.72, 121.24, 120.96, 120.89, 119.30, 31.36, 25.02, 24.11, 21.25, 17.25. HRMS calcd for C₂₈H₃₃N₂ (M⁺−Cl) 397.2683, found 397.2660.

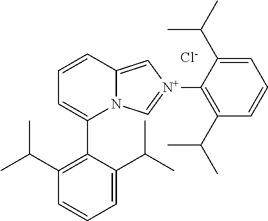 [SMILES: CC(C)c1cccc(C(C)C)c1-c1cccc2c[n+](-c3c(C(C)C)cccc3C(C)C)cn12 ; [Cl-]]

2,5-bis(2,6-diisopropylphenyl)imidazo[1,5-α]pyridin-2-ium chloride (10). Prepared according to Step 3 in General Procedure B, the reaction of 2-(2,6-diisopropylphenyl)-6-(1,3-dioxolan-2-yl)pyridine (311 mg, 1 mmol, 1 equiv), 2,6-diisopropylaniline (177 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 81% yield (385 mg). ¹H NMR (500 MHz, CDCl₃) δ 9.34 (s, 1H), 9.12-8.95 (m, 1H), 7.89 (s, 1H), 7.59-7.49 (m, 3H), 7.35 (d, J=7.8 Hz, 2H), 7.28 (d, J=7.8 Hz, 2H), 7.13-7.07 (m, 1H), 2.39-2.24 (m, 2H), 2.09-1.96 (m, 2H), 1.26-1.14 (m, 12H), 1.08-0.99 (m, 12H). ¹³C NMR (125 MHz, CDCl₃) δ 148.31, 145.08, 132.61, 132.46, 132.36, 130.50, 126.87, 125.86, 124.76, 124.65, 121.85, 121.79, 121.58, 121.53, 121.32, 31.49, 28.98, 25.18, 24.83, 24.43, 23.96. HRMS calcd for C₃₁H₃₉N₂ (M⁺−Cl) 439.3108, found 439.3134.

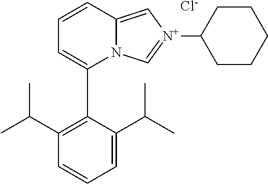 [SMILES: CC(C)c1cccc(C(C)C)c1-c1cccc2c[n+](C3CCCCC3)cn12 ; [Cl-]]

2-cyclohexyl-5-(2,6-diisopropylphenyl)imidazo[1,5-α]pyridin-2-ium chloride (11). Prepared according to Step 3 in General Procedure B, the reaction of 2-(2,6-diisopropylphenyl)-6-(1,3-dioxolan-2-yl)pyridine (311 mg, 1 mmol, 1 equiv), cyclohexylamine (177 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 61% yield (242 mg). ¹H NMR (500 MHz, CDCl₃) δ 9.37 (s, 1H), 8.26 (s, 1H), 8.20 (d, J=9.3 Hz, 1H), 7.59 (t, J=7.9 Hz, 1H), 7.37 (d, J=7.9 Hz, 2H), 7.35-7.32 (m, 1H), 6.91 (d, J=6.7 Hz, 1H), 5.02-4.94 (m, 1H), 2.27-2.20 (m, 4H), 1.90-1.84 (m, 2H), 1.74-1.66 (m, 3H), 1.60-1.50 (m, 2H), 1.27-1.21 (m, 1H), 1.11 (d, J=6.9 Hz, 6H), 1.05 (d, J=6.9 Hz, 6H). ¹³C NMR (125 MHz, CDCl₃) δ 148.16, 133.17, 132.21, 131.12, 126.84, 124.69, 124.60, 119.96, 119.54, 119.27, 116.37, 61.41, 34.23, 31.20, 25.07, 24.93, 24.59, 24.07. HRMS calcd for C₂₅H₃₃N₂ (M⁺−Cl) 361.2638, found 361.2654.

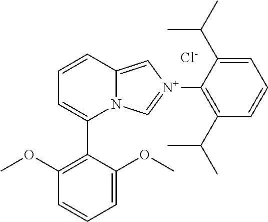 [SMILES: COc1cccc(OC)c1-c1cccc2c[n+](-c3c(C(C)C)cccc3C(C)C)cn12 ; [Cl-]]

2-(2,6-diisopropylphenyl)-5-(2,6-dimethoxyphenyl)imidazo[1,5-α]pyridin-2-ium chloride (12). Prepared according to Step 2 in General Procedure A, the reaction of 6-(2,6-dimethoxyphenyl)picolinaldehyde (487 mg, 2 mmol, 1 equiv), 2,6-diisopropylaniline (354 mg, 2 mmol, 1.0 equiv), paraformaldehyde (90 mg, 3 mmol, 1.5 equiv) and 4 M HCl in dioxane (1 mL, 4.0 mmol, 2 equiv) in toluene (10 mL) gave the title compound as white solid in 83% yield (749 mg). ¹H NMR (500 MHz, CDCl₃) δ 8.87 (s, 1H), 8.72-8.60 (m, 1H), 8.15 (s, 1H), 7.58-7.42 (m, 3H), 7.30 (d, J=7.9 Hz, 2H), 7.14 (d, J=6.9 Hz, 1H), 6.73 (d, J=8.4 Hz, 2H), 3.75 (s, 6H), 2.22-2.11 (m, 2H), 1.21 (d, J=6.7 Hz, 7H), 1.09 (d, J=6.7 Hz, 6H). ¹³C NMR (125 MHz, CDCl₃) δ 158.42, 145.09, 133.86, 133.78, 132.16, 130.79, 128.52, 125.90, 124.66, 122.94, 122.10, 120.13, 118.61, 107.30, 104.65, 56.34, 56.32, 28.67, 24.50. HRMS calcd for C₂₇H₃₁N₂O₂ (M⁺−Cl) 415.2380, found 415.2397.

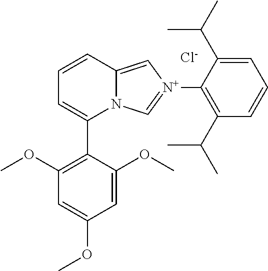 [SMILES: COc1cc(OC)c(-c2cccc3c[n+](-c4c(C(C)C)cccc4C(C)C)cn23)c(OC)c1 ; [Cl-]]

2-(2,6-diisopropylphenyl)-5-(2,4,6-trimethoxyphenyl)imidazo[1,5-α]pyridin-2-ium chloride (13). Prepared according to Step 2 in General Procedure A, the reaction of 6-(2,4,6-trimethoxyphenyl)picolinaldehyde (547 mg, 2 mmol, 1 equiv), 2,6-diisopropylaniline (354 mg, 2 mmol, 1.0 equiv), paraformaldehyde (90 mg, 3 mmol, 1.5 equiv) and 4 M HCl in dioxane (1 mL, 4.0 mmol, 2 equiv) in toluene (10 mL) gave the title compound as white solid in 54% yield (519 mg). ¹H NMR (500 MHz, MeOD) δ 9.50 (s, 1H), 8.33 (d, J=1.8 Hz, 1H), 7.90 (d, J=9.3 Hz, 1H), 7.63 (t, J=7.9 Hz, 1H), 7.50 (dd, J=9.3, 7.0 Hz, 1H), 7.45 (d, J=7.8 Hz, 2H), 7.18 (d, J=7.0 Hz, 1H), 6.41 (s, 2H), 3.90 (s, 3H), 3.78 (s, 7H), 2.22-2.12 (m, 2H), 1.21 (d, J=6.8 Hz, 6H), 1.18 (d, J=6.9 Hz, 6H). ¹³C NMR (125 MHz, MeOD) δ 165.97, 160.86, 146.44, 133.04, 132.71, 132.65, 131.59, 128.72, 127.42, 125.63, 122.70, 117.78, 117.57, 101.44, 92.32, 56.71, 56.20, 29.90, 24.64, 24.19. HRMS calcd for C₂₈H₃₃N₂O₃ (M⁺−Cl) 445.2486, found 445.2503.

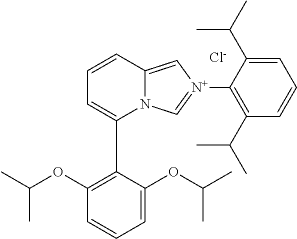 [SMILES: CC(C)Oc1cccc(OC(C)C)c1-c1cccc2c[n+](-c3c(C(C)C)cccc3C(C)C)cn12 ; [Cl-]]

5-(2,6-Diisopropoxyphenyl)-2-(2,6-diisopropylphenyl)imidazo[1,5-α]pyridin-2-ium chloride (14). To a mixture of 6-(2,6-diisopropoxyphenyl)picolinaldehyde (6 g, 20 mmol, 1 equiv), 2,6-diisopropylaniline (3.54 g, 20 mmol, 1 equiv), paraformaldehyde (900 mg, 30 mmol, 1.5 equiv) in toluene (100 mL, 0.2 M) was added 4 M HCl in dioxane (10 ml, 40 mmol, 2 equiv). The mixture was then heated at 100° C. for 3 h. Solvent was removed under reduced pressure. The residue was triturated with ethyl acetate to give the desired product as white solid in 89% yield (9.02 g). ¹H NMR (500 MHz, DMSO) δ 9.89 (s, 1H), 8.79-8.71 (m, 1H), 8.02 (d, J=8.9 Hz, 1H), 7.64 (t, J=7.8 Hz, 1H), 7.53 (dd, J=9.3, 6.9 Hz, 1H), 7.48 (t, J=8.5 Hz, 1H), 7.45 (d, J=7.8 Hz, 2H), 7.19 (d, J=6.9 Hz, 1H), 6.86 (d, J=8.5 Hz, 2H), 4.58-4.49 (m, 2H), 2.04-1.93 (m, 2H), 1.12 (d, J=6.9 Hz, 6H), 1.08 (d, J=6.3 Hz, 12H), 0.97 (d, J=6.0 Hz, 6H). ¹³C NMR (125 MHz, DMSO) δ 156.91, 144.49, 132.83, 131.50, 131.11, 130.25, 130.03, 127.40, 126.19, 124.22, 120.60, 117.28, 116.82, 110.74, 107.70, 71.08, 28.26, 24.34, 23.32, 21.76, 21.63. HRMS calcd for C₃₁H₃₉N₂O₂ (M⁺−Cl) 471.3006, found 471.3024.

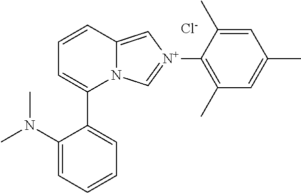

5-(2-(dimethylamino)phenyl)-2-mesitylimidazo[1,5-α]pyridin-2-ium chloride (15). Prepared according to Step 3 in General Procedure B, the reaction of 2-(6-(1,3-dioxolan-2-yl)pyridin-2-yl)-N,N-dimethylaniline (270 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 68% yield (266 mg). ¹H NMR (500 MHz, CDCl₃) δ 9.02 (s, 1H), 8.76 (d, J=9.3 Hz, 1H), 8.16 (s, 1H), 7.56-7.50 (m, 2H), 7.48-7.44 (m, 1H), 7.27 (s, 1H), 7.19 (t, J=8.4 Hz, 2H), 7.04-6.99 (m, 2H), 2.53 (s, 6H), 2.35 (s, 3H), 2.06 (s, 3H), 1.97 (s, 3H). ¹³C NMR (125 MHz, CDCl₃) δ 150.77, 141.82, 136.17, 134.39, 133.85, 133.29, 132.52, 131.52, 131.32, 130.05, 129.77, 125.41, 124.20, 122.89, 120.59, 119.46, 118.63, 117.94, 43.32, 21.28, 17.70, 16.99. HRMS calcd for C₂₄H₂₆N₃ (M⁺−Cl) 356.2121, found 356.2139.

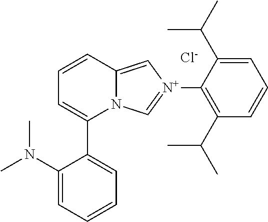 [SMILES: CC(C)c1cccc(C(C)C)c1-[n+]1cc2cccc(-c3ccccc3N(C)C)n2c1 ; [Cl-]]

2-(2,6-diisopropylphenyl)-5-(2-(dimethylamino)phenyl)imidazo[1,5-α]pyridin-2-ium chloride (16). Prepared according to Step 3 in General Procedure B, the reaction of 2-(6-(1,3-dioxolan-2-yl)pyridin-2-yl)-N,N-dimethylaniline (270 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 70% yield (304 mg). ¹H NMR (500 MHz, CDCl₃) δ 9.19 (s, 1H), 8.95 (d, J=9.9 Hz, 1H), 8.06 (s, 1H), 7.58-7.49 (m, 3H), 7.48 (d, J=6.0 Hz, 1H), 7.33 (d, J=7.8 Hz, 1H), 7.30 (d, J=7.2 Hz, 2H), 7.22-7.13 (m, 2H), 2.54 (s, 6H), 2.39-2.30 (m, 1H), 1.97-1.93 (m, 1H), 1.25 (d, J=6.7 Hz, 3H), 1.20 (d, J=6.7 Hz, 3H), 1.17 (d, J=6.9 Hz, 3H), 0.97 (d, J=6.7 Hz, 3H). ¹³C NMR (125 MHz, CDCl₃) δ 149.47, 143.91, 143.49, 135.06, 132.57, 131.71, 131.26, 130.27, 129.56, 125.67, 124.44, 123.80, 123.43, 121.92, 121.06, 120.11, 119.59, 119.44, 117.70, 43.14, 27.78, 27.71, 24.42, 24.04, 23.57, 23.36. HRMS calcd for C₂₇H₃₂N₃ (M⁺−Cl) 398.2591, found 398.2608.

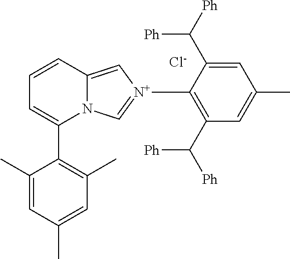

2-(2,6-dibenzhydryl-4-methylphenyl)-5-mesitylimidazo[1,5-α]pyridin-2-ium chloride (17). An oven-dried 100 mL round-bottomed flask equipped with a stir bar was charged with 6-mesitylpicolinaldehyde (450 mg, 2.0 mmol, 1.0 equiv), 2,6-dibenzhydryl-4-methylaniline (879 mg, 2 mmol, 1.0 equiv), paraformaldehyde (90 mg, 3.0 mmol, 1.5 equiv) and toluene (10 mL). The reaction mixture was stirred at 100° C. and 4 M HCl in dioxane (1 mL, 4.0 mmol, 2 equiv) was added. The resulting reaction mixture was stirred at 100° C. for 12 h. The crude product was purified by column chromatography (CH₂Cl₂/MeOH 50/1 to 15/1). The title product was obtained by trituration from diethyl ether/ethyl acetate as white solid in 87% yield (1.21 g). ¹H NMR (500 MHz, CDCl₃) δ 9.19 (s, 1H), 8.65 (d, J=9.3 Hz, 1H), 7.40 (dd, J=9.3, 6.8 Hz, 1H), 7.29-7.25 (m, 4H), 7.18-7.09 (m, 6H), 7.06-6.99 (m, 5H), 6.87 (d, J=6.7 Hz, 1H), 6.84 (s, 2H), 6.81-6.72 (m, 6H), 5.08 (s, 2H), 2.35 (s, 3H), 2.21 (s, 3H), 1.57 (s, 6H). ¹³C NMR (125 MHz, CDCl₃) δ 142.15, 141.89, 141.75, 141.00, 140.18, 136.64, 132.96, 131.48, 130.87, 130.74, 129.80, 129.31, 128.81, 128.75, 128.53, 127.24, 127.19, 125.91, 125.43, 122.89, 120.50, 120.44, 119.95, 51.31, 21.93, 21.33, 19.54. HRMS calcd for C₄₉H₄₃N₂ (M⁺−Cl) 659.3421, found 659.3466.

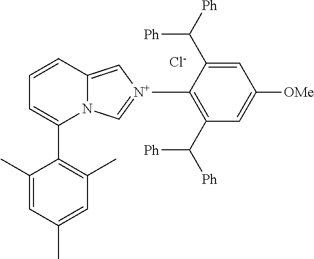 [SMILES: COc1cc(C(c2ccccc2)c2ccccc2)c(-[n+]2cc3cccc(-c4c(C)cc(C)cc4C)n3c2)c(C(c2ccccc2)c2ccccc2)c1 ; [Cl-]]

2-(2,6-dibenzhydryl-4-methoxyphenyl)-5-mesitylimidazo[1,5-α]pyridin-2-ium chloride (18). An oven-dried 100 mL round-bottomed flask equipped with a stir bar was charged with 6-mesitylpicolinaldehyde (450 mg, 2.0 mmol, 1.0 equiv), 2,6-dibenzhydryl-4-methoxyaniline (911 mg, 2 mmol, 1.0 equiv), paraformaldehyde (90 mg, 3.0 mmol, 1.5 equiv) and toluene (10 mL). The reaction mixture was stirred at 100° C. and 4 M HCl in dioxane (1 mL, 4.0 mmol, 2 equiv) was added. The resulting reaction mixture was stirred at 100° C. for 12 h. The crude product was purified by column chromatography (CH₂Cl₂/MeOH 50/1 to 15/1). The title product was obtained by trituration from diethyl ether/ethyl acetate as white solid in 89% yield (1.26 g). ¹H NMR (500 MHz, CDCl₃) δ 9.28 (s, 1H), 8.68 (d, J=9.4 Hz, 1H), 7.45 (dd, J=9.4, 6.8 Hz, 1H), 7.35-7.31 (m, 4H), 7.30-7.26 (m, 2H), 7.24-7.17 (m, 6H), 7.13-7.06 (m, 5H), 6.94-6.83 (m, 7H), 6.54 (s, 2H), 5.15 (s, 2H), 3.59 (s, 3H), 2.41 (s, 3H), 1.62 (s, 6H). ¹³C NMR (125 MHz, CDCl₃) δ 161.10, 142.23, 141.95, 141.57, 141.00, 136.62, 132.99, 131.40, 129.72, 129.33, 128.85, 128.78, 128.48, 127.37, 127.27, 125.94, 125.91, 125.32, 123.21, 120.47, 120.34, 115.72, 55.41, 51.56, 21.34, 19.54. HRMS calcd for C₄₉H₄₃N₂O (M⁺−Cl) 675.3370, found 675.3413.

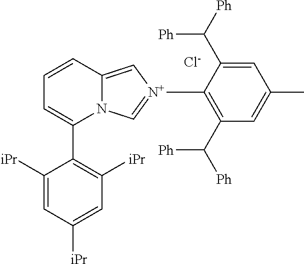 [SMILES: Cc1cc(C(c2ccccc2)c2ccccc2)c(-[n+]2cc3cccc(-c4c(C(C)C)cc(C(C)C)cc4C(C)C)n3c2)c(C(c2ccccc2)c2ccccc2)c1 ; [Cl-]]

2-(2,6-dibenzhydryl-4-methoxyphenyl)-5-(2,4,6-triisopropylphenyl)imidazo[1,5-a]pyridin-2-ium chloride (19). An oven-dried 100 mL round-bottomed flask equipped with a stir bar was charged with 6-(2,4,6-triisopropylphenyl)picolinaldehyde (450 mg, 2.0 mmol, 1.0 equiv), 2,6-dibenzhydryl-4-methylaniline (879 mg, 2 mmol, 1.0 equiv), paraformaldehyde (90 mg, 3.0 mmol, 1.5 equiv) and toluene (10 mL). The reaction mixture was stirred at 100° C. and 4 M HCl in dioxane (1 mL, 4.0 mmol, 2 equiv) was added. The resulting reaction mixture was stirred at 100° C. for 12 h. The crude product was purified by column chromatography (CH₂Cl₂/MeOH 50/1 to 15/1). The title product was obtained by trituration from diethyl ether/ethyl acetate as white solid in 71% yield (1.11 g). ¹H NMR (500 MHz, CDCl₃) δ 8.71 (s, 1H), 7.76 (s, 2H), 7.26-7.17 (m, 14H), 7.14-7.09 (m, 1H), 6.90 (d, J=6.7 Hz, 4H), 6.79-6.71 (m, 6H), 6.40 (s, 1H), 4.89 (s, 2H), 3.05-2.97 (m, 1H), 2.35-2.27 (m, 2H), 2.21 (s, 3H), 1.34 (d, J=7.0 Hz, 6H), 1.15 (d, J=6.7 Hz, 6H), 1.08 (d, J=6.9 Hz, 6H). ¹³C NMR (125 MHz, CDCl₃) δ 153.24, 147.98, 142.50, 142.05, 141.76, 140.85, 140.81, 133.14, 130.76, 130.50, 129.29, 129.23, 128.85, 128.70, 127.90, 127.36, 127.32, 124.18, 122.74, 122.52, 121.95, 119.02, 118.73, 52.17, 34.63, 31.64, 29.84, 25.18, 24.55, 24.01, 22.01. HRMS calcd for C₅₅H₅₅N₂ (M⁺−Cl) 743.4360, found 743.4390.

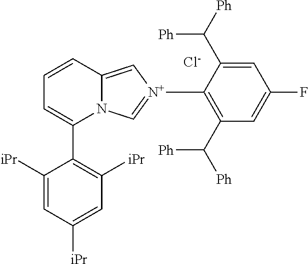 [SMILES: CC(C)c1cc(C(C)C)c(-c2cccc3c[n+](-c4c(C(c5ccccc5)c5ccccc5)cc(F)cc4C(c4ccccc4)c4ccccc4)cn23)c(C(C)C)c1 ; [Cl-]]

2-(2,6-Dibenzhydryl-4-fluorophenyl)-5-(2,4,6-triisopropylphenyl)imidazo[1,5-a]pyridin-2-ium chloride (20). Prepared according to Step 3 in General Procedure B, the reaction of 2-(1,3-dioxolan-2-yl)-6-(2,4,6-triisopropylphenyl)pyridine (354 mg, 1 mmol, 1 equiv), 2,6-dibenzhydryl-4-fluoroaniline (444 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 87% yield (682 mg). ¹H NMR (500 MHz, CDCl₃) δ 9.56 (s, 1H), 7.65-7.47 (m, 2H), 7.26-7.11 (m, 14H), 7.04-7.01 (m, 1H), 7.01-6.90 (m, 4H), 6.75 (s, 4H), 6.69-6.58 (m, 2H), 6.39-6.19 (m, 1H), 5.01 (s, 2H), 3.06-2.96 (m, 1H), 2.36-2.25 (m, 2H), 1.34 (d, J=4.8 Hz, 6H), 1.20-1.08 (m, 12H). ¹³C NMR (125 MHz, CDCl₃) δ 163.48 (d, J=253.9 Hz), 153.15, 147.80, 144.45 (d, J=5.4 Hz), 141.21, 140.18 (d, J=8.7 Hz), 133.77, 129.33, 129.27, 128.98, 128.64, 127.94, 127.55, 124.15, 122.76, 121.52, 118.81, 118.20, 117.44 (d, J=24.5 Hz), 52.24, 34.55, 31.69, 25.13, 24.56, 24.00. ¹⁹F NMR (471 MHz, CDCl₃) δ −105.11. HRMS calcd for C₅₄H₅₂FN2 (M⁺−Cl) 747.4109, found 747.4136.

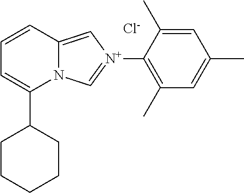 [SMILES: Cc1cc(C)c(-[n+]2cc3cccc(C4CCCCC4)n3c2)c(C)c1 ; [Cl-]]

5-cyclohexyl-2-mesitylimidazo[1,5-α]pyridin-2-ium chloride (21). Prepared according to Step 3 in General Procedure B, the reaction of 2-cyclohexyl-6-(1,3-dioxolan-2-yl)pyridine (233 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 70% yield (248 mg). ¹H NMR (500 MHz, CDCl₃) δ 11.65 (s, 1H), 7.63 (d, J=8.9 Hz, 2H), 7.27-7.25 (m, 1H), 7.02 (s, 2H), 6.90 (d, J=7.0 Hz, 1H), 4.07-3.99 (m, 1H), 2.33 (s, 3H), 2.21-2.16 (m, 2H), 2.09 (s, 6H), 2.01-1.92 (m, 2H), 1.85-1.77 (m, 3H), 1.46-1.38 (m, 2H), 1.32-1.19 (m, 1H). ¹³C NMR (125 MHz, CDCl₃) δ 144.36, 141.34, 134.16, 131.54, 131.10, 129.95, 128.32, 126.21, 115.36, 113.54, 113.12, 38.29, 31.45, 26.33, 25.41, 21.28, 17.86. HRMS calcd for C₂₂H₂₇N₂ (M⁺−Cl) 319.2169, found 319.2184.

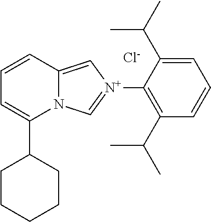 [SMILES: CC(C)c1cccc(C(C)C)c1-[n+]1cc2cccc(C3CCCCC3)n2c1 ; [Cl-]]

5-cyclohexyl-2-(2,6-diisopropylphenyl)imidazo[1,5-α]pyridin-2-ium chloride (22). Prepared according to Step 3 in General Procedure B, the reaction of 2-cyclohexyl-6-(1,3-dioxolan-2-yl)pyridine (233 mg, 1 mmol, 1 equiv), 2,6-diisopropylaniline (177 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 78% yield (310 mg). ¹H NMR (500 MHz, CDCl₃) δ 11.94 (s, 0.2H), 11.58 (s, 0.8H), 7.80-7.67 (m, 2H), 7.54 (t, J=7.9 Hz, 1H), 7.38-7.27 (m, 3H), 6.95 (d, J=7.0 Hz, 1H), 4.05 (t, J=11.5 Hz, 1H), 2.19-2.14 (m, 3H), 2.01-1.92 (m, 2H), 1.86-1.76 (m, 3H), 1.48-1.38 (m, 2H), 1.33-1.24 (m, 7H), 1.15 (d, J=6.9 Hz, 6H). ¹³C NMR (125 MHz, CDCl₃) δ 145.09, 144.10, 131.99, 131.05, 130.99, 128.58, 126.45, 124.66, 115.76, 115.04, 113.43, 38.28, 31.34, 28.97, 26.33, 25.39, 24.66, 24.48. HRMS calcd for C₂₅H₃₃N₂ (M⁺−Cl) 361.2638, found 361.2656.

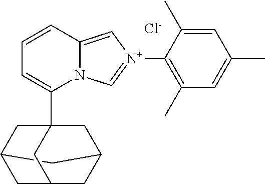 [SMILES: Cc1cc(C)c(-[n+]2cc3cccc(C45CC6CC(CC(C6)C4)C5)n3c2)c(C)c1 ; [Cl-]]

5-(adamantan-1-yl)-2-mesitylimidazo[1,5-α]pyridin-2-ium chloride (23). Prepared according to Step 3 in General Procedure B, the reaction of 2-(adamantan-1-yl)-6-(1,3-dioxolan-2-yl)pyridine (285 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 82% yield (334 mg). ¹H NMR (500 MHz, CDCl₃) δ 9.40-9.26 (m, 1H), 8.15 (s, 1H), 7.80 (d, J=9.2 Hz, 1H), 6.86-6.74 (m, 1H), 6.60-6.49 (m, 3H), 1.84 (s, 3H), 1.71 (s, 3H), 1.67 (s, 6H), 1.54 (s, 6H), 1.41 (d, J=11.9 Hz, 3H), 1.35-1.27 (m, 3H). ¹³C NMR (125 MHz, CDCl₃) δ 143.59, 140.62, 133.46, 132.35, 130.84, 129.12, 124.95, 124.62, 117.72, 115.43, 115.34, 37.64, 37.43, 35.56, 27.33, 20.60, 17.10, 17.05. HRMS calcd for C₂₆H₃₁N₂ (M⁺−Cl) 371.2482, found 371.2501.

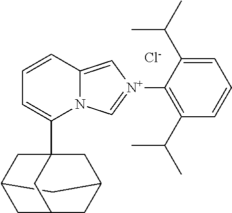 [SMILES: CC(C)c1cccc(C(C)C)c1-[n+]1cc2cccc(C34CC5CC(CC(C5)C3)C4)n2c1 ; [Cl-]]

5-(adamantan-1-yl)-2-(2,6-diisopropylphenyl)imidazo[1,5-α]pyridin-2-ium chloride (24). Prepared according to Step 3 in General Procedure B, the reaction of 2-(adamantan-1-yl)-6-(1,3-dioxolan-2-yl)pyridine (285 mg, 1 mmol, 1 equiv), 2,6-diisopropylaniline (177 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 86% yield (386 mg). ¹H NMR (500 MHz, CDCl₃) δ 9.29 (s, 1H), 8.73 (s, 1H), 8.33 (d, J=9.2 Hz, 1H), 7.39 (t, J=7.9 Hz, 1H), 7.20-7.13 (m, 3H), 6.96 (d, J=7.2 Hz, 1H), 2.03-1.99 (m, 3H), 1.97-1.89 (m, 8H), 1.64 (d, J=12.4 Hz, 7H), 1.00 (d, J=6.9 Hz, 6H), 0.97 (d, J=6.9 Hz, 6H). ¹³C NMR (125 MHz, CDCl₃) δ 144.43, 142.95, 132.73, 131.75, 130.33, 125.48, 124.27, 123.92, 118.89, 117.48, 116.22, 37.88, 37.59, 35.81, 28.31, 27.45, 24.33, 23.64. HRMS calcd for C₂₉H₃₇N₂ (M⁺−Cl) 413.2951, found 413.2971.

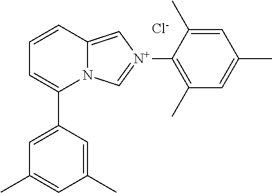

5-(3,5-dimethylphenyl)-2-mesitylimidazo[1,5-α]pyridin-2-ium chloride (25). The reaction of the corresponding imine (225 mg, 0.68 mmol), paraformaldehyde (21 mg, 0.68 mmol), trimethylchlorosilane (146 mg, 1.36 mmol) in toluene (4 mL) at 70° C. for 15 h, followed by solvent removal and trituration of the residue with ethyl acetate and diethyl ether gave the title compound as white solid (238 mg, 93%). ¹H NMR (500 MHz, CDCl₃) δ 9.05 (s, 1H), 8.84 (s, 1H), 8.64 (d, J=9.2 Hz, 1H), 7.49-7.42 (m, 1H), 7.34-7.28 (m, 3H), 7.14 (d, J=6.9 Hz, 1H), 7.07 (s, 2H), 2.45 (s, 6H), 2.40 (s, 3H), 2.11 (s, 6H). ¹³C NMR (125 MHz, CDCl₃) δ 141.79, 140.27, 135.49, 134.13, 133.21, 132.61, 131.31, 130.87, 129.99, 126.05, 125.78, 122.23, 120.16, 119.20, 118.47, 21.52, 21.26, 17.79. HRMS calcd for C₂₄H₂₅N₂ (M⁺−Cl) 341.2012, found 341.2026.

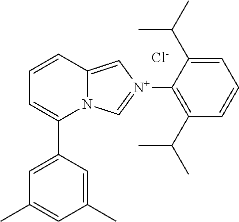 [SMILES: Cc1cc(C)cc(-c2cccc3c[n+](-c4c(C(C)C)cccc4C(C)C)cn23)c1 ; [Cl-]]

2-(2,6-diisopropylphenyl)-5-(3,5-dimethylphenyl)imidazo[1,5-α]pyridin-2-ium chloride (26). The reaction of the corresponding imine (370 mg, 1 mmol), paraformaldehyde (30 mg, 1 mmol), trimethylchlorosilane (217 mg, 2 mmol) in toluene (4 mL) at 70° C. for 15 h, followed by solvent removal and trituration of the residue with ethyl acetate and diethyl ether gave the title compound as white solid (369 mg, 88%). ¹H NMR (500 MHz, CDCl₃) δ 9.34 (s, 1H), 8.92 (d, J=9.3 Hz, 1H), 8.58 (s, 1H), 7.56 (t, J=7.9 Hz, 1H), 7.50-7.43 (m, 1H), 7.33 (d, J=7.8 Hz, 2H), 7.20 (s, 3H), 7.14 (d, J=6.9 Hz, 1H), 2.38 (s, 6H), 2.17 (p, J=6.8 Hz, 2H), 1.26 (d, J=6.9 Hz, 6H), 1.12 (d, J=6.8 Hz, 6H). ¹³C NMR (125 MHz, CDCl₃) δ 145.11, 140.35, 134.82, 133.20, 132.76, 132.29, 130.94, 130.73, 126.01, 125.80, 124.82, 122.04, 121.15, 120.08, 119.58, 28.86, 24.86, 24.26, 21.46. HRMS calcd for C₂₇H₃₁N₂ (M⁺−Cl) 383.2482, found 383.2501.

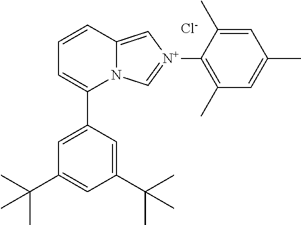 [SMILES: Cc1cc(C)c(-[n+]2cc3cccc(-c4cc(C(C)(C)C)cc(C(C)(C)C)c4)n3c2)c(C)c1 ; [Cl-]]

5-(3,5-di-tert-butylphenyl)-2-mesitylimidazo[1,5-α]pyridin-2-ium chloride (27). The reaction of the corresponding imine (350 mg, 0.85 mmol), paraformaldehyde (26 mg, 0.85 mmol), trimethylchlorosilane (185 mg, 1.7 mmol) in toluene (4 mL) at 70° C. for 15 h, followed by solvent removal and trituration of the residue with ethyl acetate and diethyl ether gave the title compound as white solid (372 mg, 95%). ¹H NMR (500 MHz, CDCl₃) δ 9.26 (s, 1H), 8.73 (d, J=9.3 Hz, 1H), 8.49 (s, 1H), 7.63 (s, 1H), 7.45-7.37 (m, 3H), 7.12 (d, J=5.0 Hz, 1H), 7.01 (s, 2H), 2.34 (s, 3H), 2.05 (s, 6H), 1.35-1.31 (m, 18H). ¹³C NMR (125 MHz, CDCl₃) δ 153.35, 141.82, 135.68, 133.95, 132.86, 131.26, 130.55, 130.03, 125.73, 125.64, 122.37, 121.39, 120.80, 119.37, 119.32, 35.30, 31.45, 21.24, 17.67. HRMS calcd for C₃₀H₃₇N₂ (M⁺−Cl) 425.2951, found 425.2970.

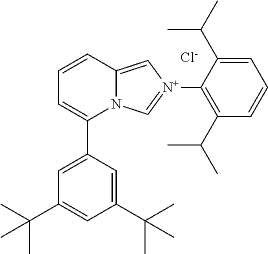 [SMILES: CC(C)c1cccc(C(C)C)c1-[n+]1cc2cccc(-c3cc(C(C)(C)C)cc(C(C)(C)C)c3)n2c1 ; [Cl-]]

5-(3,5-di-tert-butylphenyl)-2-(2,6-diisopropylphenyl)imidazo[1,5-α]pyridin-2-ium chloride (28). The reaction of the corresponding imine (330 mg, 0.72 mmol), paraformaldehyde (22 mg, 0.72 mmol), trimethylchlorosilane (156 mg, 1.44 mmol) in toluene (4 mL) at 70° C. for 15 h, followed by solvent removal and trituration of the residue with ethyl acetate and diethyl ether gave the title compound as white solid (315 mg, 87%). ¹H NMR (500 MHz, CDCl₃) δ 8.89 (s, 1H), 8.65 (d, J=9.4 Hz, 1H), 8.39 (s, 1H), 7.64 (s, 1H), 7.59-7.51 (m, 2H), 7.43 (d, J=1.8 Hz, 2H), 7.32 (d, J=7.9 Hz, 2H), 7.17 (d, J=6.9 Hz, 1H), 2.23 (p, J=6.8 Hz, 2H), 1.34 (s, 18H), 1.25 (d, J=6.8 Hz, 6H), 1.11 (d, J=6.8 Hz, 6H). ¹³C NMR (125 MHz, CDCl₃) δ 153.38, 145.29, 135.72, 132.69, 132.32, 130.71, 130.57, 126.46, 125.74, 124.78, 122.52, 121.99, 120.47, 119.91, 119.47, 35.33, 31.46, 28.83, 24.77, 24.46. HRMS calcd for C₃₃H₄₃N₂ (M⁺−Cl) 467.3421, found 467.3445.

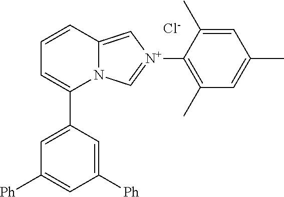 [SMILES: Cc1cc(C)c(-[n+]2cc3cccc(-c4cc(-c5ccccc5)cc(-c5ccccc5)c4)n3c2)c(C)c1 ; [Cl-]]

5-([1,1′:3′,1″-terphenyl]-5′-yl)-2-mesitylimidazo[1,5-α]pyridin-2-ium chloride (29). Prepared according to Step 3 in General Procedure B, the reaction of 2-([1,1′:3′,1″-terphenyl]-5′-yl)-6-(1,3-dioxolan-2-yl)pyridine (379 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 87% yield (436 mg). ¹H NMR (500 MHz, CDCl₃) δ 9.01 (s, 1H), 8.86 (s, 1H), 8.51 (d, J=9.3 Hz, 1H), 7.97 (s, 1H), 7.88 (s, 2H), 7.69 (d, J=7.8 Hz, 4H), 7.53-7.44 (m, 5H), 7.37 (t, J=7.2 Hz, 2H), 7.32 (d, J=6.9 Hz, 1H), 6.99 (s, 2H), 2.30 (s, 3H), 2.07 (s, 6H). ¹³C NMR (125 MHz, CDCl₃) δ 142.95, 140.72, 138.59, 134.12, 133.18, 131.62, 131.37, 130.39, 129.10, 128.42, 127.95, 127.61, 126.53, 125.43, 125.05, 121.84, 119.20, 118.99, 117.09, 20.40, 16.99. HRMS calcd for C₃₄H₂₉N₂ (M⁺−Cl) 465.2325, found 465.2344.

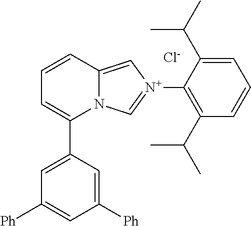 [SMILES: CC(C)c1cccc(C(C)C)c1-[n+]1cc2cccc(-c3cc(-c4ccccc4)cc(-c4ccccc4)c3)n2c1 ; [Cl-]]

5-([1,1′:3′,1″-terphenyl]-5′-yl)-2-(2,6-diisopropylphenyl)imidazo[1,5-α]pyridin-2-ium chloride (30). Prepared according to Step 3 in General Procedure B, the reaction of 2-([1,1′:3′,1″-terphenyl]-5′-yl)-6-(1,3-dioxolan-2-yl)pyridine (379 mg, 1 mmol, 1 equiv), 2,6-diisopropylaniline (177 mg, 1 mmol, 1.0 equiv), paraformaldehyde (45 mg, 1.5 mmol, 1.5 equiv) and 4 M HCl in dioxane (1 mL, 4.0 mmol, 4 equiv) in toluene (5 mL) gave the title compound as white solid in 92% yield (500 mg). ¹H NMR (500 MHz, CDCl₃) δ 9.30 (s, 1H), 9.20 (s, 1H), 8.90 (d, J=9.5 Hz, 1H), 8.24 (s, 1H), 8.11 (s, 2H), 7.97-7.90 (m, 4H), 7.87-7.77 (m, 2H), 7.73-7.66 (m, 5H), 7.62 (d, J=7.6 Hz, 2H), 7.55 (d, J=7.9 Hz, 2H), 2.58-2.44 (m, 2H), 1.48 (d, J=6.9 Hz, 6H), 1.35 (d, J=6.9 Hz, 6H). ¹³C NMR (125 MHz, CDCl₃) δ 144.08, 143.00, 138.33, 133.72, 131.61, 131.43, 131.19, 129.65, 128.31, 127.82, 127.61, 126.36, 125.88, 124.67, 123.87, 121.91, 119.54, 119.22, 118.19, 27.88, 23.66, 23.55. HRMS calcd for C₃₇H₃₅N₂ (M⁺−Cl) 507.2795, found 507.2818.

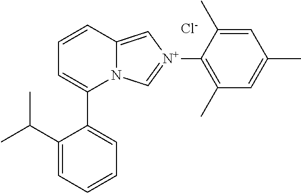 [SMILES: Cc1cc(C)c(-[n+]2cc3cccc(-c4ccccc4C(C)C)n3c2)c(C)c1 ; [Cl-]]

5-(2-isopropylphenyl)-2-mesitylimidazo[1,5-α]pyridin-2-ium chloride (31). Prepared according to Step 2 in General Procedure A, the reaction of 6-(2-isopropylphenyl) picolinaldehyde (225 mg, 1 mmol, 1 equiv), 2,4,6-trimethylaniline (135 mg, 1 mmol, 1.0 equiv), paraformaldehyde (45 mg, 1.5 mmol, 1.5 equiv) and 4 M HCl in dioxane (0.5 mL, 2.0 mmol, 2 equiv) in toluene (5 mL) gave the title compound as white solid in 81% yield (317 mg). ¹H NMR (500 MHz, CDCl₃) δ 9.23 (s, 1H), 8.85 (d, J=9.4 Hz, 1H), 8.02 (s, 1H), 7.58 (t, J=6.7 Hz, 1H), 7.52 (d, J=7.7 Hz, 1H), 7.47-7.38 (m, 3H), 7.08 (d, J=6.8 Hz, 1H), 6.99-6.95 (m, 2H), 2.50-2.43 (m, 1H), 2.31 (s, 3H), 2.00 (s, 3H), 1.94 (s, 3H), 1.15 (d, J=6.7 Hz, 3H), 1.12 (d, J=6.9 Hz, 3H). ¹³C NMR (125 MHz, CDCl₃) δ 147.90, 141.86, 134.17, 133.84, 133.79, 132.54, 132.22, 131.13, 129.99, 129.96, 129.80, 128.74, 127.82, 127.12, 125.48, 121.41, 121.19, 120.29, 119.39, 30.94, 25.20, 23.44, 21.21, 17.63, 17.25. HRMS calcd for C₂₅H₂₇N₂ (M⁺−Cl) 355.2169, found 355.2186.

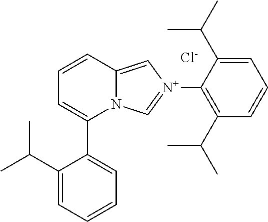 [SMILES: CC(C)c1ccccc1-c1cccc2c[n+](-c3c(C(C)C)cccc3C(C)C)cn12 ; [Cl-]]

2-(2,6-diisopropylphenyl)-5-(2-isopropylphenyl)imidazo[1,5-α]pyridin-2-ium chloride (32). Prepared according to Step 2 in General Procedure A, the reaction of 6-(2-isopropylphenyl)picolinaldehyde (225 mg, 1 mmol, 1 equiv), 2,6-diisopropylaniline (177 mg, 1 mmol, 1.0 equiv), paraformaldehyde (45 mg, 1.5 mmol, 1.5 equiv) and 4 M HCl in dioxane (0.5 mL, 2.0 mmol, 2 equiv) in toluene (5 mL) gave the title compound as white solid in 81% yield (351 mg). ¹H NMR (500 MHz, CDCl₃) δ 9.14 (s, 1H), 8.85 (d, J=9.3 Hz, 1H), 7.97 (s, 1H), 7.58-7.48 (m, 4H), 7.38 (t, J=7.5 Hz, 1H), 7.31 (d, J=7.6 Hz, 1H), 7.27-7.23 (m, 2H), 7.11 (d, J=6.9 Hz, 1H), 2.55-2.49 (m, 1H), 2.11-1.97 (m, 2H), 1.20-1.06 (m, 12H), 1.04-0.97 (m, 6H). ¹³C NMR (125 MHz, CDCl₃) δ 147.95, 145.02, 144.89, 133.56, 132.36, 132.27, 130.51, 129.51, 128.62, 127.78, 127.19, 126.02, 124.72, 124.64, 121.92, 121.02, 120.59, 120.00, 30.93, 28.83, 28.74, 24.96, 24.58, 24.26, 24.16, 23.58. HRMS calcd for C₂₈H₃₃N₂ (M⁺−Cl) 397.2638, found 397.2658.

### 2,5-dimesitylimidazo[1,5-α]pyridin-2-ium chloride (33)

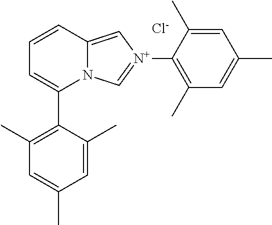

Step 1. Activated magnesium turnings (346 mg, 14.4 mol, 1.44 equiv) was suspended in anhydrous THF (10 mL). To the mixture at 60° C. was slowly added 2-bromo-1,3,5-trimethylbenzene (478 mg, 2.4 mmol). The Grignard reaction was then initiated by the addition of catalytic 1,2-dibromoethane (ca. 80 μL). The remaining 2-bromo-1,3,5-trimethylbenzene (1.91 g, 9.6 mmol, total 1.2 equiv) was added slowly. After complete addition, the reaction mixture was heated at 60° C. for 2 h. To a well-stirred suspension of 2-bromo-6-(1,3-dioxolan-2-yl)pyridine (2.3 g, 10 mmol, 1.0 equiv) and Ni(PCy₃)Cl₂ (69 mg, 0.1 mmol, 1 mol %) in anhydrous THF (5 mL) was slowly added the above Grignard solution over 10 minutes. The resultant brown solution was heated at 50° C. for 12 h, after which the mixture was poured over aqueous NH₄Cl solution (30 mL). The aqueous layer was extracted with ethyl acetate (30 mL×2), and the combined organic extracts were washed with brine (30 mL×2) and dried over anhydrous Na₂SO₄, filtered and concentrated in vacuo. The residue was loaded onto a short pad of silica gel. After washing with hexane, the residue was eluted with hexane/ethyl acetate (4/1) till fully recovery of the product. The filtrate was concentrated in vacuo to give the desired product as white solid in 91% yield (2.47 g). ¹H NMR (500 MHz, CDCl₃) δ 7.80 (t, J=7.7 Hz, 1H), 7.52 (d, J=7.8 Hz, 1H), 7.22 (d, J=7.6 Hz, 1H), 5.86 (s, 1H), 4.28-4.15 (m, 2H), 4.15-4.03 (m, 2H), 2.31 (s, 3H), 2.02 (s, 6H). ¹³C NMR (125 MHz, CDCl₃) δ 159.54, 157.03, 137.64, 137.50, 137.14, 135.93, 128.46, 125.28, 118.55, 104.33, 65.72, 21.21, 20.36.

Step 2. To a mixture of 2-(2,4,6-trimethylphenyl)-6-(1,3-dioxolan-2-yl)pyridine (2.15 g, 8 mmol, 1 equiv), 2,4,6-trimethylaniline (1.08 g, 8 mmol, 1 equiv), paraformaldehyde (360 mg, 12 mmol, 1.5 equiv) in toluene (16 mL, 0.5 M) was added 4 M HCl in dioxane (8 mL, 32 mmol, 4 equiv). The mixture was then heated at 100° C. for 12 h. Solvent was removed under reduced pressure. The residue was dried under high vacuum and then was triturated with ethyl acetate (15 mL)/hexane (5 mL) to give the desired product as white solid in 95% yield (3.06 g). Spectroscopic data matched literature values.

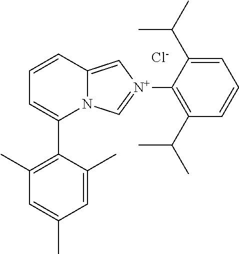 [SMILES: Cc1cc(C)c(-c2cccc3c[n+](-c4c(C(C)C)cccc4C(C)C)cn23)c(C)c1 ; [Cl-]]

2-(2,6-diisopropylphenyl)-5-mesitylimidazo[1,5-α]pyridin-2-ium chloride (34). Prepared according to Step 3 in General Procedure B, the reaction of 2-(2,4,6-trimethylphenyl)-6-(1,3-dioxolan-2-yl)pyridine (539 g, 2 mmol, 1 equiv), 2,6-diisopropylaniline (354 mg, 2 mmol, 1.0 equiv), paraformaldehyde (90 mg, 3 mmol, 1.5 equiv) and 4 M HCl in dioxane (2 mL, 8.0 mmol, 4 equiv) in toluene (10 mL) gave the title compound as white solid in 85% yield (736 mg). Spectroscopic data matched literature values.

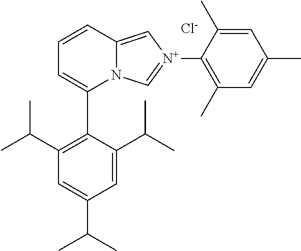 [SMILES: Cc1cc(C)c(-[n+]2cc3cccc(-c4c(C(C)C)cc(C(C)C)cc4C(C)C)n3c2)c(C)c1 ; [Cl-]]

2-Mesityl-5-(2,4,6-triisopropylphenyl)imidazo[1,5-α]pyridin-2-ium chloride (35). Prepared according to Step 3 in General Procedure B, the reaction of 2-(1,3-dioxolan-2-yl)-6-(2,4,6-triisopropylphenyl)pyridine (708 mg, 2 mmol, 1.0 equiv), 2,4,6-trimethylaniline (270 mg, 2 mmol, 1 equiv), paraformaldehyde (90 mg, 3 mmol, 1.5 equiv) and 4 M HCl in dioxane (2 mL, 8.0 mmol, 4 equiv) in toluene (10 mL) gave the title compound as white solid in 82% yield (784 mg). Spectroscopic data matched literature values.

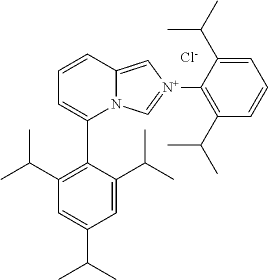 [SMILES: CC(C)c1cc(C(C)C)c(-c2cccc3c[n+](-c4c(C(C)C)cccc4C(C)C)cn23)c(C(C)C)c1 ; [Cl-]]

2-(2,6-Diisopropylphenyl)-5-(2,4,6-triisopropylphenyl)imidazo[1,5-α]pyridin-2-ium chloride (36). Prepared according to Step 3 in General Procedure B, the reaction of 2-(1,3-dioxolan-2-yl)-6-(2,4,6-triisopropylphenyl)pyridine (708 mg, 2 mmol, 1.0 equiv), 2,6-diisopropylaniline (354 mg, 2 mmol, 1.0 equiv), paraformaldehyde (90 mg, 3 mmol, 1.5 equiv) and 4 M HCl in dioxane (2 mL, 8.0 mmol, 4 equiv) in toluene (10 mL) gave the title compound as white solid in 88% yield (915 mg). Spectroscopic data matched literature values.

### Example 2: Representative Gram-Scale Synthesis of NHC Ligands

### 5-(2,6-diisopropylphenyl)-2-mesitylimidazo[1,5-α]pyridin-2-ium chloride (9)

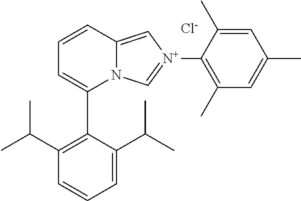 [SMILES: Cc1cc(C)c(-[n+]2cc3cccc(-c4c(C(C)C)cccc4C(C)C)n3c2)c(C)c1 ; [Cl-]]

Step 1. Activated magnesium turnings (1.38 g, 57.6 mol, 1.44 equiv) were suspended in anhydrous THF (30 mL). To the mixture at 60° C. was slowly added 1-bromo-2,6-diisopropylbenzene (2.32 g, 9.6 mmol). The Grignard reaction was then initiated by the addition of catalytic 1,2-dibromoethane (ca. 100 μL). The remaining 1-bromo-2,6-diisopropylbenzene (9.26 g, 38.4 mmol, total 1.2 equiv) was added slowly. After complete addition, the reaction mixture was heated at 60° C. for 2 h. To a well-stirred suspension of 2-bromo-6-(1,3-dioxolan-2-yl)pyridine (9.20 g, 40 mmol, 1.0 equiv) and Ni(PCy₃)Cl₂ (276 mg, 0.4 mmol, 1 mol %) in anhydrous THF (20 mL) was slowly added the above Grignard solution over 15 minutes. The resultant brown solution was heated at 50° C. for 12 h, after which the mixture was poured over aqueous NH₄Cl solution (50 mL). The aqueous layer was extracted with ethyl acetate (100 mL×2), and the combined organic extracts were washed with brine (100 mL×2), dried over anhydrous Na₂SO₄, filtered and concentrated in vacuo. The residue was loaded onto a short pad of silica gel. After washing with hexane, the residue was eluted with hexane/ethyl acetate (4/1) till fully recovery of the product. The filtrate was concentrated in vacuo to give the desired product as white solid in 96% yield (12.10 g).

Step 2. To a mixture of 2-(2,6-diisopropylphenyl)-6-(1,3-dioxolan-2-yl)pyridine (9.34 g, mmol, 1 equiv), 2,4,6-trimethylaniline (4.05 g, 30 mmol, 1 equiv), paraformaldehyde (1.35 g, mmol, 1.5 equiv) in toluene (60 mL, 0.5 M) was added 4 M HCl in dioxane (30 mL, 120 mmol, 4 equiv). The mixture was then heated at 100° C. for 12 h. Solvent was removed under reduced pressure. The residue was dried under high vacuum and then was triturated with ethyl acetate (45 mL)/hexane (15 mL) to give the desired product as white solid in 89% yield (11.09 g). Spectroscopic data matched values reported elsewhere herein.

### 5-(2,6-diisopropoxyphenyl)-2-(2,6-diisopropylphenyl)imidazo[1,5-α]pyridin-2-ium chloride (14)

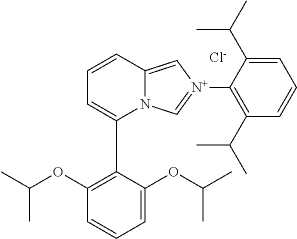 [SMILES: CC(C)Oc1cccc(OC(C)C)c1-c1cccc2c[n+](-c3c(C(C)C)cccc3C(C)C)cn12 ; [Cl-]]

See the synthesis of 5-(2,6-Diisopropoxyphenyl)-2-(2,6-diisopropylphenyl)imidazo[1,5-a]pyridin-2-ium chloride (14), as described elsewhere herein.

### 2-(2,6-dibenzhydryl-4-methylphenyl)-5-(2,4,6-triisopropylphenyl)imidazo[1,5-α]pyridin-2-ium chloride (19)

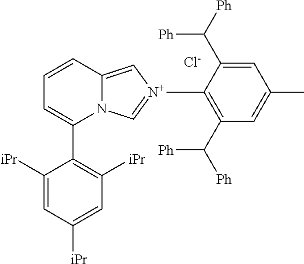 [SMILES: Cc1cc(C(c2ccccc2)c2ccccc2)c(-[n+]2cc3cccc(-c4c(C(C)C)cc(C(C)C)cc4C(C)C)n3c2)c(C(c2ccccc2)c2ccccc2)c1 ; [Cl-]]

Step 1. Activated magnesium turnings (1.38 g, 57.6 mol, 1.44 equiv) were suspended in anhydrous THF (30 mL). To the mixture at 60° C. was slowly added 1-bromo-2,4,6-triisopropylbenzene (2.72 g, 9.6 mmol). The Grignard reaction was then initiated by the addition of catalytic 1,2-dibromoethane (ca. 100 μL). The remaining 1-bromo-2,4,6-triisopropylbenzene (10.88 g, 38.4 mmol, total 1.2 equiv) was added slowly. After complete addition, the reaction mixture was heated at 60° C. for 2 h. To a well-stirred suspension of 2-bromo-6-(1,3-dioxolan-2-yl)pyridine (9.20 g, 40 mmol, 1.0 equiv) and Ni(PCy₃)Cl₂ (276 mg, 0.4 mmol, 1 mol %) in anhydrous THF (20 mL) was slowly added the above Grignard solution over 15 minutes. The resultant brown solution was heated at 50° C. for 12 h, after which the mixture was poured over aqueous NH₄Cl solution (50 mL). The aqueous layer was extracted with ethyl acetate (100 mL×2), and the combined organic extracts were washed with brine (100 mL×2), dried over anhydrous Na₂SO₄, filtered and concentrated in vacuo. The residue was loaded onto a short pad of silica gel. After washing with hexane, the residue was eluted with hexane/ethyl acetate (4/1) till fully recovery of the product. The filtrate was concentrated in vacuo to give the desired product as white solid in 85% yield (12.08 g).

Step 2. To a mixture of 2-(2,4,6-triisopropylphenyl)-6-(1,3-dioxolan-2-yl)pyridine (7.07 g, 20 mmol, 1 equiv), 2,6-dibenzhydryl-4-methylaniline (8.79 g, 20 mmol, 1 equiv), paraformaldehyde (0.9 g, 30 mmol, 1.5 equiv) in toluene (40 mL, 0.5 M) was added 4 M HCl in dioxane (20 mL, 80 mmol, 4 equiv). The mixture was then heated at 100° C. for 12 h. Solvent was removed under reduced pressure. The residue was dried under high vacuum and then was triturated with ethyl acetate (60 mL)/hexane (20 mL) to give the desired product as white solid in 89% yield (14.15 g). Spectroscopic data matched values reported in the section above.

### Example 3: Synthesis of Imidazo[1,5-α]pyridine NHC Ligand-Metal Complexes

### cinnamyl [2,5-dimesitylimidazo[1,5-α]pyridin-3-ylidene]chloropalladium(II) (37)

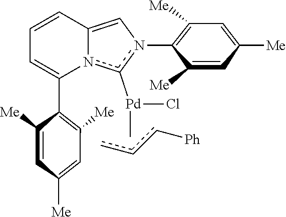

An oven-dried flask equipped with a stir bar was charged with the corresponding NHC·HCl salt (391 mg, 1.0 mmol, 1.0 equiv), KOtBu (157 mg, 1.4 mmol, 1.4 equiv), and [Pd(cin)Cl]₂ (256 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 3 h. The reaction mixture was diluted with CH₂Cl₂ and filtered. The solution was collected and concentrated. The product was purified by column chromatography on silica gel to give the desired product as yellow solid. Yield 65% (398 mg). ¹H NMR (500 MHz, CDCl₃) δ 7.37 (s, 1H), 7.33 (d, J=7.5 Hz, 1H), 7.21-7.17 (m, 3H), 7.06-7.01 (m, 4H), 6.93-6.87 (m, 3H), 6.35 (d, J=6.6 Hz, 1H), 4.50-4.40 (m, 1H), 4.04 (d, J=12.5 Hz, 1H), 3.06-3.00 (m, 1H), 2.55 (s, 1H), 2.44 (s, 3H), 2.40 (s, 3H), 2.34 (s, 6H), 1.91 (s, 3H), 1.88 (s, 3H). ¹³C NMR (125 MHz, CDCl₃) δ 174.24, 139.78, 139.11, 139.05, 138.53, 138.46, 137.43, 136.84, 134.05, 133.72, 132.46, 129.95, 129.52, 128.22, 128.06, 127.52, 126.83, 122.66, 116.77, 115.70, 113.54, 109.88, 90.08, 60.53, 46.38, 21.82, 21.50, 21.29, 20.08, 19.46, 17.58. HRMS calcd for C₃₄H₃₅N₂Pd (M⁺−Cl) 577.1842, found 577.1855. Crystals suitable for X-ray crystallography were obtained from saturated hexane/DCM solution.

### allyl [2,5-dimesitylimidazo[1,5-α]pyridin-3-ylidene]chloropalladium(II) (38)

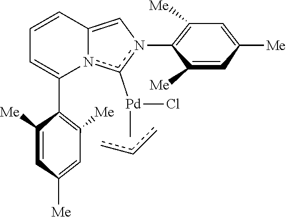

An oven-dried flask equipped with a stir bar was charged with the corresponding NHC·HCl salt (391 mg, 1.0 mmol, 1.0 equiv), KOtBu (157 mg, 1.4 mmol, 1.4 equiv), and [Pd(allyl)Cl]₂ (183 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 3 h. The reaction mixture was diluted with CH₂Cl₂ and filtered. The solution was collected and concentrated. The product was purified by column chromatography on silica gel to give the desired product as yellow solid. Yield 78% (452 mg). NMR spectroscopic were too complicated to analyze due to the existence of different conformers. HRMS calcd for C₃₁H₃₇N₂Pd (M⁺−Cl) 543.1986, found 543.1969. Crystals suitable for X-ray crystallography were obtained from saturated hexane/DCM solution.

### cinnamyl [2-(2,6-diisopropylphenyl)-5-mesitylimidazo[1,5-α]pyridin-3-ylidene]chloropalladium(II) (39)

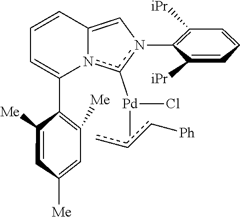

An oven-dried flask equipped with a stir bar was charged with the corresponding NHC·HCl salt (433 mg, 1.0 mmol, 1.0 equiv), KOtBu (157 mg, 1.4 mmol, 1.4 equiv), and [Pd(cin)Cl]₂ (256 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 3 h. The reaction mixture was diluted with CH₂Cl₂ and filtered. The solution was collected and concentrated. The product was purified by column chromatography on silica gel to give the desired product as yellow solid. Yield 73% (478 mg). NMR spectroscopic were too complicated to analyze due to the existence of different conformers. HRMS calcd for C₃₇H₄₁N₂Pd (M⁺−Cl) 619.2313, found 619.2333. Crystals suitable for X-ray crystallography were obtained from saturated hexane/DCM solution.

### 1-tert-butyl-indenyl [2-(2,6-diisopropylphenyl)-5-mesitylimidazo[1,5-α]pyridin-3-ylidene]chloropalladium(II) (40)

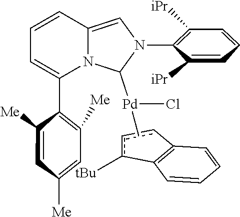

An oven-dried flask equipped with a stir bar was charged with the corresponding NHC·HCl salt (433 mg, 1.0 mmol, 1.0 equiv), KOtBu (157 mg, 1.4 mmol, 1.4 equiv), and [(1-tBu-ind)PdCl]₂ (313 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 3 h. The reaction mixture was diluted with CH₂Cl₂ and filtered. The solution was collected and concentrated. The product was purified by column chromatography on silica gel to give the desired product as yellow solid. Yield 37% (262 mg). ¹H NMR (500 MHz, CDCl₃) δ 7.47 (t, J=7.7 Hz, 1H), 7.32-7.28 (m, 3H), 7.12 (s, 1H), 7.06 (d, J=7.7 Hz, 2H), 6.95-6.89 (m, 2H), 6.61 (t, J=7.6 Hz, 1H), 6.45-6.38 (m, 1H), 6.23 (t, J=7.2 Hz, 1H), 5.76 (d, J=7.0 Hz, 1H), 5.22 (d, J=3.0 Hz, 1H), 4.77 (s, 1H), 3.40-3.28 (m, 1H), 2.66 (s, 3H), 2.46 (s, 3H), 1.86-1.75 (m, 4H), 1.41 (d, J=6.8 Hz, 3H), 1.29 (s, 9H), 1.15 (d, J=6.8 Hz, 3H), 0.94 (d, J=6.9 Hz, 3H), 0.73 (d, J=6.6 Hz, 3H). ¹³C NMR (125 MHz, CDCl₃) δ 167.42, 147.15, 145.55, 141.64, 139.60, 139.52, 139.12, 138.08, 136.84, 136.76, 133.49, 133.31, 130.59, 130.13, 127.27, 125.49, 125.02, 123.16, 122.92, 122.77, 119.55, 119.36, 117.09, 116.71, 116.36, 116.01, 108.73, 65.99, 61.22, 34.39, 29.44, 28.21, 27.79, 26.15, 26.10, 24.09, 22.86, 22.16, 21.34, 19.46, 15.43. HRMS calcd for C₄₁H₄₇N₂Pd (M⁺−Cl) 673.2784, found 673.2762. Crystals suitable for X-ray crystallography were obtained from saturated hexane/DCM solution.

### cinnamyl [2-mesityl-5-(2,4,6-triisopropylphenyl)imidazo[1,5-α]pyridin-3-ylidene]chloropalladium(II) (41)

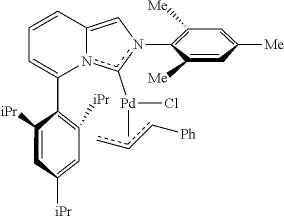

An oven-dried flask equipped with a stir bar was charged with the corresponding NHC·HCl salt (475 mg, 1.0 mmol, 1.0 equiv), KOtBu (157 mg, 1.4 mmol, 1.4 equiv), and [Pd(cin)Cl]₂ (256 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 3 h. The reaction mixture was diluted with CH₂Cl₂ and filtered. The solution was collected and concentrated. The product was purified by column chromatography on silica gel to give the desired product as yellow solid. Yield 71% (495 mg). NMR spectroscopic were too complicated to analyze due to the existence of different conformers. HRMS calcd for C₄₀H₄₇N₂Pd (M⁺−Cl) 661.2783, found 661.2807. Crystals suitable for X-ray crystallography were obtained from saturated hexane/DCM solution.

### cinnamyl [5-(2,6-diisopropylphenyl)-2-mesitylimidazo[1,5-α]pyridin-3-ylidene]chloropalladium (42)

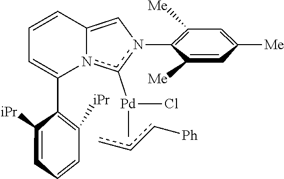

An oven-dried flask equipped with a stir bar was charged with the corresponding NHC·HCl salt (433 mg, 1.0 mmol, 1.0 equiv), KOtBu (157 mg, 1.4 mmol, 1.4 equiv), and [Pd(cin)Cl]₂ (256 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 3 h. The reaction mixture was diluted with CH₂Cl₂ and filtered. The solution was collected and concentrated. The product was purified by column chromatography on silica gel to give the desired product as yellow solid. Yield 68% (445 mg). NMR spectroscopic were too complicated to analyze due to the existence of different conformers. HRMS calcd for C₃₇H₄₁N₂Pd (M⁺−Cl) 619.2313, found 619.2331. Crystals suitable for X-ray crystallography were obtained from saturated hexane/DCM solution.

### Cinnamyl [2-(2,6-dibenzhydryl-4-methylphenyl)-5-mesitylimidazo[1,5-α]pyridine-3-ylidene]chloropalladium(II) (43)

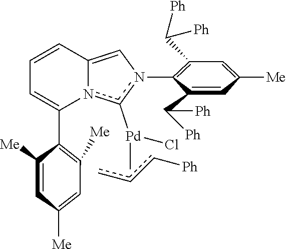

An oven-dried flask equipped with a stir bar was charged with the corresponding NHC·HCl salt (695 mg, 1.0 mmol, 1.0 equiv), KOtBu (157 mg, 1.4 mmol, 1.4 equiv), and [Pd(cin)Cl]₂ (256 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 12 h. The reaction mixture was diluted with CH₂Cl₂ and passed through a short pad of silica gel covered with a pad of Celite eluting with CH₂Cl₂ until the product was completely recovered. The solution was collected and concentrated. The desired product was obtained as yellow solid after triturating with hexane and drying under high vacuum. Yield 85% (780 mg). NMR spectroscopic were too complicated to analyze due to the existence of different conformers. HRMS calcd for C₅₈H₅₂N₂Pd (M⁺−Cl) 881.3101, found 881.3170. Crystals suitable for X-ray crystallography were obtained from saturated hexane/DCM solution.

### Cinnamyl [2-(2,6-dibenzhydryl-4-methylphenyl)-5-(2,4,6-triisopropylphenyl)imidazo[1,5-a]pyridin-3-ylidene]chloropalladium(II) (44)

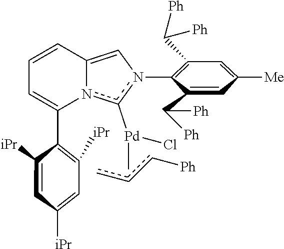

An oven-dried flask equipped with a stir bar was charged with the corresponding NHC·HCl salt (780 mg, 1.0 mmol, 1.0 equiv), KOtBu (157 mg, 1.4 mmol, 1.4 equiv), and [Pd(cin)Cl]₂ (256 mg, 0.5 mmol, 0.5 equiv), placed under a positive pressure of argon and subjected to three evacuation/backfilling cycles under high vacuum. THF (10 mL, 0.1 M) was added, and the resulting reaction mixture was stirred at room temperature for 12 h. The reaction mixture was diluted with CH₂Cl₂ and passed through a short pad of silica gel covered with a pad of Celite eluting with CH₂Cl₂ until the product was completely recovered. The solution was collected and concentrated. The desired product was obtained as yellow solid after triturating with hexane and drying under high vacuum. Yield 83% (832 mg). NMR spectroscopic were too complicated to analyze due to the existence of different conformers. HRMS calcd for C₆₄H₆₃N₂Pd (M⁺−Cl) 965.4042, found 965.4097. Crystals suitable for X-ray crystallography were obtained from saturated hexane/DCM solution.

### Cinnamyl [5-(2,6-diisopropoxyphenyl)-2-(2,6-diisopropylphenyl)imidazo[1,5-α]pyridin-3-ylidene]chloropalladium(II) (45)

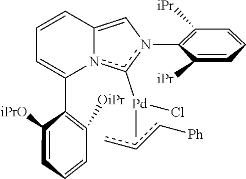

An oven-dried flask equipped with a stir bar was charged with the corresponding NHC·HCl salt (51 mg, 0.1 mmol, 1.0 equiv), KOtBu (15.7 mg, 0.14 mmol, 1.4 equiv), and [Pd(cin)Cl]₂ (25.6 mg, 0.05 mmol, 0.5 equiv), placed under a positive pressure of argon and subjected to three evacuation/backfilling cycles under high vacuum. THF (1 mL, 0.1 M) was added, and the resulting reaction mixture was stirred at room temperature for 3 h. The reaction mixture was diluted with CH₂Cl₂ and filtered. The solution was collected and concentrated. The product was purified by column chromatography on silica gel to give the desired product as yellow solid. Yield 72% (52.5 mg). NMR spectroscopic were too complicated to analyze due to the existence of different conformers. HRMS calcd for C₄₀H₄₇N₂O₂Pd (M⁺−Cl) 693.2667, found 693.2689.

### pyridine [2-(2,6-diisopropylphenyl)-5-mesitylimidazo[1,5-α]pyridin-3-ylidene]dichloropalladium(II) (46)

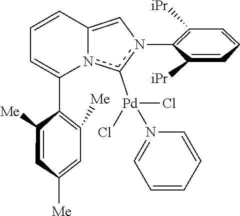

An oven-dried flask equipped with a stir bar was charged with the corresponding NHC·HCl salt (433 mg, 1.0 mmol, 1.0 equiv), KOtBu (157 mg, 1.4 mmol, 1.4 equiv), and PdCl₂ (177 mg, 1.0 mmol, 1.0 equiv), placed under a positive pressure of argon and subjected to three evacuation/backfilling cycles under high vacuum. Pyridine (5 mL) was added, and the resulting reaction mixture was stirred 80° C. for 12 h. After cooling to room temperature, the reaction mixture was diluted with CH₂Cl₂ and passed through a short pad of silica gel covered with a pad of Celite eluting with CH₂Cl₂ until the product was completely recovered. The filtrate was concentrated in vacuo and purified via silica gel flash chromatography (hexane/ethyl acetate 10:1 to 4:1). The catalyst was obtained as yellow solid (496 mg, 76%). ¹H NMR (500 MHz, CDCl₃) δ 8.15 (d, J=4.9 Hz, 2H), 7.54 (t, J=7.6 Hz, 1H), 7.49 (t, J=7.8 Hz, 1H), 7.39 (s, 1H), 7.31 (d, J=7.6 Hz, 3H), 7.10-7.02 (m, 4H), 6.98 (dd, J=9.2, 6.7 Hz, 1H), 6.60 (dd, J=6.5, 1.3 Hz, 1H), 2.70 (hept, J=6.7 Hz, 2H), 2.30 (s, 9H), 1.41 (d, J=6.7 Hz, 6H), 1.06 (d, J=6.9 Hz, 6H). ¹³C NMR (125 MHz, CDCl₃) δ 151.25, 146.85, 142.16, 139.82, 138.85, 138.63, 137.21, 135.71, 132.89, 132.62, 130.61, 128.63, 123.75, 123.70, 117.23, 117.11, 116.61, 28.79, 26.91, 23.24, 21.60, 21.34. HRMS calcd for C₃₃H₃₇N₃PdCl (M⁺−Cl) 616.1714, found 616.1742.

### 3-chloropyridine [2-(2,6-diisopropylphenyl)-5-mesitylimidazo[1,5-α]pyridin-3-ylidene]dichloropalladium(II) (47)

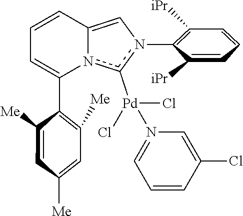

An oven-dried flask equipped with a stir bar was charged with the corresponding NHC·HCl salt (433 mg, 1.0 mmol, 1.0 equiv), KOtBu (157 mg, 1.4 mmol, 1.4 equiv), and PdCl₂ (177 mg, 1.0 mmol, 1.0 equiv), placed under a positive pressure of argon and subjected to three evacuation/backfilling cycles under high vacuum. 3-Chloropyridine (5 mL) was added, and the resulting reaction mixture was stirred 80° C. for 12 h. After cooling to room temperature, the reaction mixture was diluted with CH₂Cl₂ and passed through a short pad of silica gel covered with a pad of Celite eluting with CH₂Cl₂ until the product was completely recovered. The filtrate was concentrated in vacuo and purified via silica gel flash chromatography (hexane/ethyl acetate 10:1 to 4:1). The catalyst was obtained as yellow solid (371 mg, 54%). ¹H NMR (500 MHz, CDCl₃) δ 8.19-8.13 (m, 2H), 7.57-7.53 (m, 1H), 7.50 (t, J=7.8 Hz, 1H), 7.40 (s, 1H), 7.34-7.30 (m, 3H), 7.06 (s, 2H), 7.04-6.98 (m, 2H), 6.61 (dd, J=6.7, 1.3 Hz, 1H), 2.72-2.63 (m, 2H), 2.32 (s, 3H), 2.29 (s, 6H), 1.40 (d, J=6.6 Hz, 6H), 1.06 (d, J=6.8 Hz, 6H). ¹³C NMR (125 MHz, CDCl₃) δ 150.30, 149.26, 146.84, 140.58, 139.81, 138.90, 138.78, 137.30, 135.61, 132.97, 132.53, 131.68, 130.68, 128.65, 124.12, 123.78, 122.98, 117.35, 117.24, 116.64, 28.80, 26.90, 23.22, 21.60, 21.34. HRMS calcd for C₃₃H₃₆N₃PdCl₂ (M⁺−Cl) 650.1322, found 650.1354. Crystals suitable for X-ray crystallography were obtained from saturated hexane/DCM solution.

### 3-chloropyridine [2-mesityl-5-(2,4,6-triisopropylphenyl)imidazo[1,5-α]pyridin-3-ylidene]dichloropalladium(II) (48)

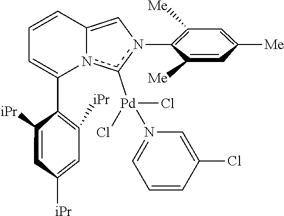

An oven-dried flask equipped with a stir bar was charged with the corresponding NHC·HCl salt (475 mg, 1.0 mmol, 1.0 equiv), KOtBu (157 mg, 1.4 mmol, 1.4 equiv), and PdCl₂ (177 mg, 1.0 mmol, 1.0 equiv), placed under a positive pressure of argon and subjected to three evacuation/backfilling cycles under high vacuum. 3-Chloropyridine (5 mL) was added, and the resulting reaction mixture was stirred 80° C. for 12 h. After cooling to room temperature, the reaction mixture was diluted with CH₂Cl₂ and passed through a short pad of silica gel covered with a pad of Celite eluting with CH₂Cl₂ until the product was completely recovered. The filtrate was concentrated in vacuo and purified via silica gel flash chromatography (hexane/ethyl acetate 10:1 to 4:1). The catalyst was obtained as yellow solid. (453 mg, 62%). ¹H NMR (500 MHz, CDCl₃) δ 8.26 (d, J=2.4 Hz, 1H), 8.12 (d, J=4.1 Hz, 1H), 7.53 (d, J=8.3 Hz, 1H), 7.35 (s, 1H), 7.33 (d, J=9.3 Hz, 1H), 7.19 (s, 2H), 7.02-6.97 (m, 3H), 6.96-6.90 (m, 1H), 6.68 (d, J=6.2 Hz, 1H), 3.05-2.97 (m, 1H), 2.73-2.63 (m, 2H), 2.35 (s, 3H), 2.21 (s, 6H), 1.32-1.26 (m, 12H), 1.05 (d, J=6.7 Hz, 6H). ¹³C NMR (125 MHz, CDCl₃) δ 150.36, 150.29, 149.55, 149.09, 140.10, 139.44, 137.72, 137.35, 136.28, 136.16, 134.31, 131.65, 131.18, 129.20, 123.98, 122.09, 121.26, 119.35, 117.10, 115.35, 34.42, 31.52, 27.01, 24.27, 22.72, 21.39, 19.03. HRMS calcd for C₃₆H₄₂N₃PdCl₂ (M⁺−Cl) 694.1788, found 694.1825.

### 2-dimethylaminomethylphenyl [2-(2,6-dibenzhydryl-4-methylphenyl)-5-(2,4,6-triisopropylphenyl)imidazo[1,5-α]pyridin-3-ylidene]chloropalladium(II) (49)

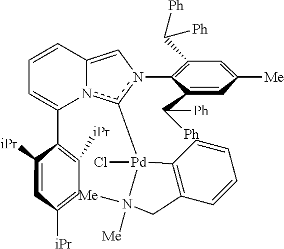

An oven-dried flask equipped with a stir bar was charged with the corresponding NHC·HCl salt (390 mg, 0.5 mmol, 1.0 equiv), KOtBu (79 mg, 0.7 mmol, 1.4 equiv), and dimer [Pd(C₆H₄—CH₂—NMe₂)Cl]₂ (138 mg, 0.25 mmol, 0.5 equiv), placed under a positive pressure of argon and subjected to three evacuation/backfilling cycles under high vacuum. THF (5 mL, 0.1 M) was added, and the resulting reaction mixture was stirred at room temperature for 12 h. The reaction mixture was diluted with CH₂Cl₂ and passed through a short pad of silica gel covered with a pad of Celite eluting with CH₂Cl₂ until the product was completely recovered. The solution was collected and concentrated. The product was purified by column chromatography on silica gel to give the desired product as yellow solid. Yield 81% (413 mg). ¹H NMR (500 MHz, CDCl₃) δ 7.74 (d, J=6.7 Hz, 2H), 7.26-7.22 (m, 3H), 7.19-7.13 (m, 4H), 7.06-7.01 (m, 3H), 6.99 (s, 1H), 6.96 (d, J=7.1 Hz, 1H), 6.91 (t, J=7.4 Hz, 2H), 6.73-6.63 (m, 8H), 6.61-6.55 (m, 2H), 6.50-6.46 (m, 1H), 6.43 (d, J=5.7 Hz, 1H), 6.35 (d, J=7.1 Hz, 2H), 5.79 (s, 1H), 5.50-5.44 (m, 2H), 5.00 (s, 1H), 3.64 (d, J=14.3 Hz, 1H), 3.36 (d, J=14.4 Hz, 1H), 2.97-2.91 (m, 1H), 2.75-2.63 (m, 2H), 2.43 (s, 3H), 2.18 (s, 3H), 2.13 (s, 3H), 1.69 (d, J=6.6 Hz, 3H), 1.29-1.27 (m, 6H), 1.05 (d, J=6.7 Hz, 3H), 0.85 (d, J=6.7 Hz, 3H), 0.14 (d, J=6.6 Hz, 3H). ¹³C NMR (125 MHz, CDCl₃) δ 163.56, 149.54, 149.40, 149.16, 148.08, 147.39, 145.71, 144.85, 143.91, 143.63, 142.83, 141.44, 138.43, 137.62, 136.48, 136.17, 131.61, 131.39, 131.16, 130.13, 129.63, 129.46, 129.41, 128.07, 128.04, 127.28, 126.25, 126.01, 125.61, 125.58, 124.08, 121.63, 121.59, 120.92, 119.84, 119.78, 119.50, 117.56, 72.73, 51.73, 51.05, 50.08, 49.87, 34.50, 32.08, 31.88, 31.41, 29.85, 29.81, 29.51, 28.06, 25.27, 24.75, 23.71, 22.84, 22.44, 22.04, 20.75, 14.27. Crystals suitable for X-ray crystallography were obtained from saturated hexane/DCM solution. HRMS calcd for C₆₄H₆₆N₃Pd (M⁺−Cl) 982.4386, found 982.4417.

### 2-acetamido-phenyl [2-(2,6-dibenzhydryl-4-methylphenyl)-5-(2,4,6-triisopropylphenyl)imidazo[1,5-α]pyridin-3-ylidene]chloropalladium(II) (50)

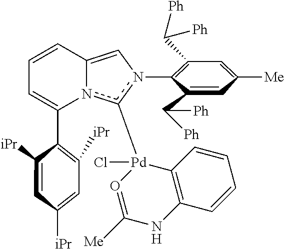

An oven-dried flask equipped with a stir bar was charged with the corresponding NHC·HCl salt (390 mg, 0.5 mmol, 1.0 equiv), KOtBu (79 mg, 0.7 mmol, 1.4 equiv), and dimer [Pd(C₆H₄—NHC(O)Me)Cl]₂ (138 mg, 0.25 mmol, 0.5 equiv), placed under a positive pressure of argon and subjected to three evacuation/backfilling cycles under high vacuum. THF (5 mL, 0.1 M) was added, and the resulting reaction mixture was stirred at room temperature for 12 h. The reaction mixture was diluted with CH₂Cl₂ and passed through a short pad of silica gel covered with a pad of Celite eluting with CH₂Cl₂ until the product was completely recovered. The solution was collected and concentrated. The product was purified by column chromatography on silica gel to give the desired product as yellow solid. Yield 84% (428 mg). NMR spectroscopic were too complicated to analyze due to the existence of different conformers. HRMS calcd for C₆₃H₆₂N₃OPd (M⁺−Cl) 982.3922, found 982.3963.

### (2,5-dimesitylimidazo[1,5-α]pyridin-3(2H)-ylidene)gold(I) chloride (51)

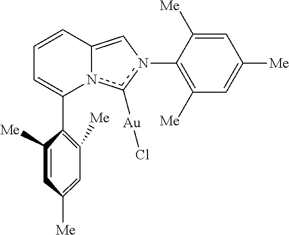

An oven-dried flask equipped with a stir bar was charged with 2,5-dimesitylimidazo[1,5-a]pyridin-2-ium chloride (39 mg, 0.10 mmol, 1.0 equiv), chloro(dimethyl sulfide)gold(I) (30 mg, 0.10 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 93% yield (54.6 mg). ¹H NMR (500 MHz, CDCl₃) δ 7.45 (d, J=9.2 Hz, 1H), 7.26 (s, 1H), 7.10-7.02 (m, 3H), 6.92 (s, 2H), 6.55 (dd, J=6.6, 1.4 Hz, 1H), 2.41 (s, 3H), 2.31 (s, 3H), 2.07 (s, 6H), 1.94 (s, 6H). ¹³C NMR (125 MHz, CDCl₃) δ 164.49, 140.95, 139.78, 138.74, 136.90, 136.25, 134.21, 131.67, 130.80, 129.44, 129.08, 123.61, 116.83, 116.34, 112.45, 21.58, 21.29, 20.03, 17.80. HRMS calcd for C₂₅H₃₀N₃AuClN₂ (M⁺+NH₄) 604.1788, found 604.1826.

### (5-(2,6-diisopropylphenyl)-2-mesitylimidazo[1,5-α]pyridin-3(2H)-ylidene)gold(I) chloride (52)

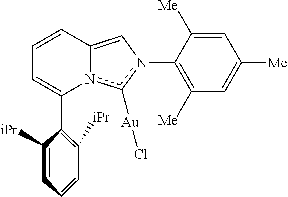

An oven-dried flask equipped with a stir bar was charged with the corresponding NHC·HCl salt (43 mg, 0.10 mmol, 1.0 equiv), chloro(dimethyl sulfide)gold(I) (30 mg, 0.10 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 82% yield (51.8 mg). ¹H NMR (500 MHz, CDCl₃) δ 7.62 (t, J=7.8 Hz, 1H), 7.49 (d, J=9.2 Hz, 1H), 7.35-7.28 (m, 3H), 7.10-7.04 (m, 1H), 6.92 (s, 2H), 6.57 (d, J=6.6 Hz, 1H), 2.42 (p, J=6.9 Hz, 2H), 2.29 (s, 3H), 1.93 (s, 6H), 1.28 (d, J=6.9 Hz, 6H), 1.14 (d, J=6.8 Hz, 6H). ¹³C NMR (125 MHz, CDCl₃) δ 165.10, 147.46, 139.68, 138.02, 136.33, 134.20, 131.61, 131.59, 131.00, 129.47, 123.95, 123.22, 117.04, 116.94, 112.44, 31.77, 25.30, 23.69, 21.25, 17.69. HRMS calcd for C₂₈H₃₃N₂AuClN₂ (M⁺+H) 629.1992, found 629.2015.

### (2-(2,6-dibenzhydryl-4-methylphenyl)-5-mesityl-2,3-dihydroimidazo[1,5-α]pyridin-3-yl)gold(I) chloride (53)

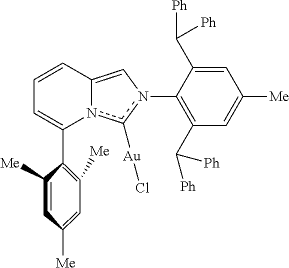

An oven-dried flask equipped with a stir bar was charged with the corresponding NHC·HCl salt (70 mg, 0.10 mmol, 1.0 equiv), chloro(dimethyl sulfide)gold(I) (30 mg, 0.10 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 96% yield (86 mg). ¹H NMR (500 MHz, CDCl₃) δ 7.25-7.21 (m, 4H), 7.19-7.14 (m, 8H), 7.10 (s, 2H), 7.07-7.04 (m, 4H), 6.86-6.84 (m, 2H), 6.80-6.75 (m, 4H), 6.66 (s, 2H), 6.49-6.45 (m, 1H), 5.70 (s, 1H), 5.31 (s, 2H), 2.47 (s, 3H), 2.19 (s, 3H), 2.08 (s, 6H). ¹³C NMR (125 MHz, CDCl₃) δ 164.05, 142.59, 142.08, 141.03, 140.93, 139.78, 138.09, 136.90, 135.75, 130.73, 130.20, 129.87, 129.65, 129.24, 129.07, 128.59, 128.46, 126.71, 126.69, 122.71, 116.83, 116.41, 115.50, 51.66, 21.95, 21.60, 20.08. HRMS calcd for C₄₉H₄₃N₂AuC1 (M+H) 891.2775, found 891.2803.

### (2-(2,6-dibenzhydryl-4-methoxyphenyl)-5-mesityl-2,3-dihydroimidazo[1,5-α]pyridin-3-yl)gold(I) chloride (54)

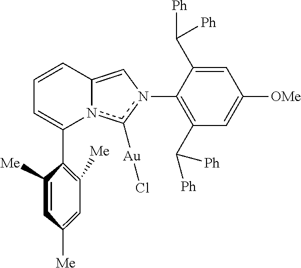

An oven-dried flask equipped with a stir bar was charged with the corresponding NHC·HCl salt (71 mg, 0.10 mmol, 1.0 equiv), chloro(dimethyl sulfide)gold(I) (30 mg, 0.10 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 96% yield (87 mg). ¹H NMR (500 MHz, CDCl₃) δ 7.26-7.21 (m, 4H), 7.20-7.14 (m, 8H), 7.11 (s, 2H), 7.07 (d, J=7.4 Hz, 4H), 6.88-6.84 (m, 2H), 6.83-6.78 (m, 4H), 6.50-6.45 (m, 1H), 6.38 (s, 2H), 5.69 (s, 1H), 5.34 (s, 2H), 3.53 (s, 3H), 2.48 (s, 3H), 2.09 (s, 6H). ¹³C NMR (125 MHz, CDCl₃) δ 164.39, 159.83, 142.90, 142.32, 141.82, 140.87, 137.99, 136.87, 131.22, 130.71, 129.79, 129.58, 129.14, 129.03, 128.62, 128.47, 126.80, 126.76, 122.67, 116.81, 116.40, 115.74, 114.98, 55.16, 51.89, 21.58, 20.06. HRMS calcd for C₄₉H₄₃N₂OAuC1 (M+H) 907.2724, found 907.2763.

### (2-(2,6-dibenzhydryl-4-methylphenyl)-5-(2,4,6-triisopropylphenyl)-2,3-dihydroimidazo[1,5-a]pyridin-3-yl)gold(I) chloride (55)

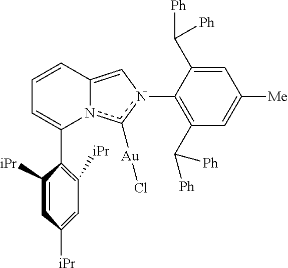

An oven-dried flask equipped with a stir bar was charged with the corresponding NHC·HCl salt (78 mg, 0.10 mmol, 1.0 equiv), chloro(dimethyl sulfide)gold(I) (30 mg, 0.10 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 93% yield (91 mg). ¹H NMR (500 MHz, CDCl₃) δ 7.21-7.13 (m, 14H), 7.07-7.03 (m, 4H), 6.86-6.81 (m, 2H), 6.80-6.74 (m, 4H), 6.65 (s, 2H), 6.53-6.48 (m, 1H), 5.58 (s, 1H), 5.24 (s, 2H), 3.06-2.98 (m, 1H), 2.53-2.42 (m, 2H), 2.18 (s, 3H), 1.41 (d, J=7.0 Hz, 6H), 1.29 (d, J=7.0 Hz, 6H), 1.15 (d, J=6.7 Hz, 6H). ¹³C NMR (125 MHz, CDCl₃) δ 164.66, 152.36, 147.03, 142.74, 142.34, 140.99, 139.82, 137.72, 135.83, 130.17, 129.73, 129.58, 129.18, 129.16, 128.56, 128.48, 126.68, 126.63, 122.34, 122.18, 117.15, 116.86, 115.71, 51.55, 34.87, 32.00, 25.31, 24.59, 24.07, 21.99. HRMS calcd for C₅₅H₅₅N₂AuCl (M⁺+H) 975.3714, found 975.3729. (5-(2,6-diisopropoxyphenyl)-2-(2,6-diisopropylphenyl)-2,3-dihydroimidazo[1,5-α]pyridin-3-yl)gold(I) chloride (56)

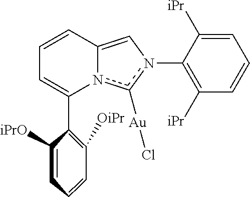

An oven-dried flask equipped with a stir bar was charged with the corresponding NHC·HCl salt (51 mg, 0.10 mmol, 1.0 equiv), chloro(dimethyl sulfide)gold(I) (30 mg, 0.10 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 87% yield (61 mg). ¹H NMR (500 MHz, CDCl₃) δ 7.50-7.40 (m, 3H), 7.21 (d, J=7.8 Hz, 2H), 7.07 (dd, J=9.1, 6.7 Hz, 1H), 6.64 (d, J=8.5 Hz, 2H), 6.58 (d, J=6.1 Hz, 1H), 4.57-4.49 (m, 2H), 2.28-2.18 (m, 2H), 1.22-1.18 (m, 12H), 1.14-1.09 (m, 12H). ¹³C NMR (125 MHz, CDCl₃) δ 165.00, 157.52, 145.23, 135.93, 134.43, 131.77, 131.53, 130.49, 124.12, 123.97, 117.52, 116.28, 114.83, 113.04, 106.97, 70.98, 28.42, 24.80, 24.32, 22.79, 22.47. HRMS calcd for C₃₁H₃₈AuN₂O₂ (M⁺−Cl) 667.2593, found 667.2624.

### (2-(2,6-dibenzhydryl-4-methylphenyl)-5-mesityl-2,3-dihydroimidazo[1,5-α]pyridin-3-yl)copper(I) chloride (57)

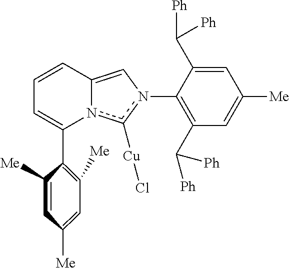

An oven-dried flask equipped with a stir bar was charged with the corresponding NHC·HCl salt (70 mg, 0.10 mmol, 1.0 equiv), copper(I) chloride (19.8 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 94% yield (71 mg). ¹H NMR (500 MHz, CDCl₃) δ 7.24-7.20 (m, 4H), 7.18-7.13 (m, 8H), 7.06 (s, 2H), 7.00 (d, J=7.5 Hz, 4H), 6.95-6.91 (m, 1H), 6.87-6.83 (m, 1H), 6.82-6.75 (m, 4H), 6.67 (s, 2H), 6.42 (d, J=6.4 Hz, 1H), 5.93 (s, 1H), 5.25 (s, 2H), 2.44 (s, 3H), 2.17 (s, 3H), 2.01 (s, 6H). ¹³C NMR (125 MHz, CDCl₃) δ 169.45, 142.80, 142.06, 141.00, 140.88, 139.61, 138.47, 136.22, 135.74, 130.11, 130.05, 130.00, 129.58, 129.54, 129.26, 128.67, 128.42, 126.64, 126.62, 122.87, 116.54, 115.23, 115.13, 51.68, 21.89, 21.58, 19.71. HRMS calcd for C₄₉H₄₂N₂Cu (M⁺−Cl) 721.2139, found 721.2676.

### (2-(2,6-dibenzhydryl-4-methoxyphenyl)-5-mesityl-2,3-dihydroimidazo[1,5-α]pyridin-3-yl)copper(I) chloride (58)

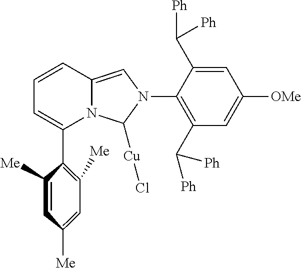

An oven-dried flask equipped with a stir bar was charged with the corresponding NHC·HCl salt (71 mg, 0.10 mmol, 1.0 equiv), copper(I) chloride (19.8 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 93% yield (72 mg). ¹H NMR (500 MHz, CDCl₃) δ 7.25-7.20 (m, 4H), 7.19-7.13 (m, 8H), 7.07 (s, 2H), 7.02 (d, J=7.5 Hz, 4H), 6.92 (d, J=9.2 Hz, 1H), 6.87-6.83 (m, 1H), 6.82-6.76 (m, 4H), 6.43 (d, J=6.6 Hz, 1H), 6.38 (s, 2H), 5.91 (s, 1H), 5.27 (s, 2H), 3.52 (s, 3H), 2.45 (s, 3H), 2.02 (s, 6H). ¹³C NMR (125 MHz, CDCl₃) δ 168.91, 158.77, 141.79, 141.63, 140.82, 140.06, 137.50, 135.24, 130.27, 129.07, 128.97, 128.62, 128.53, 128.23, 127.77, 127.47, 125.77, 125.74, 121.84, 115.55, 114.42, 114.23, 113.95, 54.14, 50.97, 20.61, 18.75. HRMS calcd for C₄₉H₄₂N₂OCu (M⁺
−Cl) 737.2588, found 737.2630.

### (2-(2,6-dibenzhydryl-4-methylphenyl)-5-(2,4,6-triisopropylphenyl)-2,3-dihydroimidazo[1,5-a]pyridin-3-yl)copper(I) chloride (59)

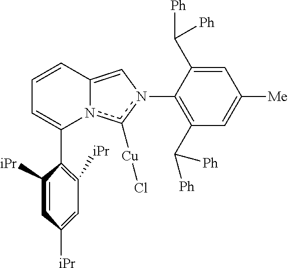

An oven-dried flask equipped with a stir bar was charged with the corresponding NHC·HCl salt (78 mg, 0.10 mmol, 1.0 equiv), copper(I) chloride (19.8 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 88% yield (74 mg). ¹H NMR (500 MHz, CDCl₃) δ 7.25-7.14 (m, 14H), 7.04-7.00 (m, 4H), 6.87-6.83 (m, 2H), 6.80-6.76 (m, 4H), 6.67 (s, 2H), 6.53-6.49 (m, 1H), 5.64 (s, 1H), 5.19 (s, 2H), 3.09-2.99 (m, 1H), 2.54-2.44 (m, 2H), 2.18 (s, 3H), 1.41 (d, J=6.9 Hz, 6H), 1.23 (d, J=6.9 Hz, 6H), 1.17 (d, J=6.8 Hz, 6H). ¹³C NMR (125 MHz, CDCl₃) δ 169.52, 152.43, 146.68, 142.85, 142.38, 140.99, 139.64, 138.12, 135.81, 130.09, 129.74, 129.59, 129.14, 128.47, 128.44, 128.17, 126.65, 126.54, 122.75, 122.47, 116.61, 115.99, 115.82, 51.60, 34.80, 31.75, 25.21, 24.46, 24.43, 21.95. HRMS calcd for C₅₅H₅₄N₂Cu (M⁺−Cl) 805.3578, found 805.3613. (2-(2,6-dibenzhydryl-4-methylphenyl)-5-mesityl-2,3-dihydroimidazo[1,5-α]pyridin-3-yl)silver(I) chloride (60)

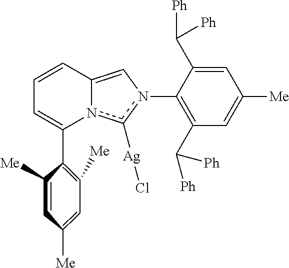

An oven-dried flask equipped with a stir bar was charged with the corresponding NHC·HCl salt (70 mg, 0.10 mmol, 1.0 equiv), silver(I) oxide (46 mg, 0.20 mmol, 2.0 equiv), placed under a positive pressure of argon and subjected to three evacuation/backfilling cycles under high vacuum. Dichloromethane (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 88% yield (71 mg). ¹H NMR (500 MHz, CDCl₃) δ 7.26-7.23 (m, 4H), 7.21-7.16 (m, 8H), 7.06-7.03 (m, 3H), 6.97-6.90 (m, 5H), 6.82-6.76 (m, 4H), 6.69 (s, 2H), 6.48 (d, J=6.4 Hz, 1H), 6.19 (s, 1H), 5.16 (s, 2H), 2.45 (s, 3H), 2.20 (s, 3H), 1.96 (s, 6H). ¹³C NMR (125 MHz, CDCl₃) δ 173.84 (d, J=272.8 Hz), 173.84 (d, J=237.0 Hz), 142.83, 141.81, 141.39, 140.87, 139.88, 138.46, 136.13, 135.98, 130.24, 129.53, 129.41, 129.30, 128.88, 128.48, 126.77, 126.73, 122.94, 116.74, 115.68, 115.47, 115.41, 51.61, 21.90, 21.65, 19.83. HRMS calcd for C₄₉H₄₂N₂Ag (M⁺−Cl) 765.2393, found 765.2427.

### (2-(2,6-dibenzhydryl-4-methoxyphenyl)-5-mesityl-2,3-dihydroimidazo[1,5-a]pyridin-3-yl)silver(I) chloride (61)

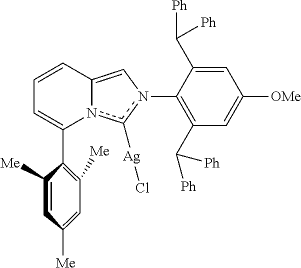

An oven-dried flask equipped with a stir bar was charged with the corresponding NHC·HCl salt (71 mg, 0.10 mmol, 1.0 equiv), silver(I) oxide (46 mg, 0.20 mmol, 2.0 equiv), placed under a positive pressure of argon and subjected to three evacuation/backfilling cycles under high vacuum. Dichloromethane (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 87% yield (71 mg). ¹H NMR (500 MHz, CDCl₃) δ 7.26-7.23 (m, 4H), 7.20-7.16 (m, 8H), 7.07-7.02 (m, 3H), 6.99-6.95 (m, 4H), 6.93-6.88 (m, 1H), 6.83-6.77 (m, 4H), 6.48 (d, J=5.2 Hz, 1H), 6.40 (s, 2H), 6.17 (d, J=1.8 Hz, 1H), 5.18 (s, 2H), 3.54 (s, 3H), 2.45 (s, 3H), 1.97 (s, 6H). ¹³C NMR (125 MHz, CDCl₃) δ 174.24 (d, J=273.4 Hz), 174.24 (d, J=237.0 Hz), 159.85, 142.76, 142.60, 141.57, 141.37, 138.39, 136.13, 131.46, 130.60, 130.54, 129.52, 129.37, 129.22, 128.93, 128.51, 126.87, 126.81, 122.89, 116.73, 115.73, 115.66, 115.04, 55.17, 51.85, 21.65, 19.83. HRMS calcd for C₄₉H₄₂N₂OAg (M⁺−Cl) 781.2343, found 781.2372.

### (2-(2,6-dibenzhydryl-4-methylphenyl)-5-(2,4,6-triisopropylphenyl)-2,3-dihydroimidazo[1,5-a]pyridin-3-yl)silver(I) chloride (62)

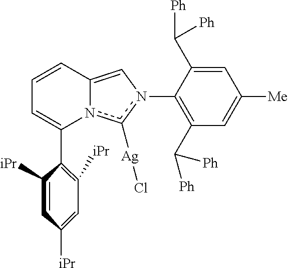

An oven-dried flask equipped with a stir bar was charged with the corresponding NHC·HCl salt (78 mg, 0.10 mmol, 1.0 equiv), silver(I) oxide (46 mg, 0.20 mmol, 2.0 equiv), placed under a positive pressure of argon and subjected to three evacuation/backfilling cycles under high vacuum. Dichloromethane (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as white solid in 90% yield (80 mg). ¹H NMR (500 MHz, CDCl₃) δ 7.29-7.19 (m, 12H), 7.18-7.13 (m, 2H), 7.04-6.97 (m, 5H), 6.96-6.90 (m, 1H), 6.86-6.79 (m, 4H), 6.74 (s, 2H), 6.61-6.56 (m, 1H), 5.89 (s, 1H), 5.14 (s, 2H), 3.12-3.03 (m, 1H), 2.56-2.44 (m, 2H), 2.22 (s, 3H), 1.45 (d, J=7.2 Hz, 6H), 1.18 (d, J=4.1 Hz, 12H). ¹³C NMR (125 MHz, CDCl₃) δ 173.39 (d, J=270.5 Hz), 173.39 (d, J=234.7 Hz), 172.38 (d, J=17.9 Hz), 152.43, 146.72, 142.78, 142.11, 140.89, 139.88, 137.89, 136.01, 130.37, 130.32, 130.14, 129.43, 129.10, 128.55, 128.46, 127.67, 126.72, 126.58, 122.59, 122.51, 116.82, 116.52, 116.24, 116.19, 51.40, 34.68, 31.64, 25.12, 24.45, 24.32, 21.87. HRMS calcd for C₅₅H₅₄N₂Ag (M⁺−Cl) 849.3332, found 849.3330.

### cyclooctadiene [5-(2,6-diisopropylphenyl)-2-mesitylimidazo[1,5-α]pyridin-3-ylidene]rhodium(I) chloride (63)

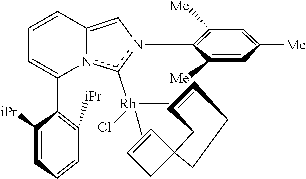

An oven-dried flask equipped with a stir bar was charged with the corresponding NHC·HCl salt (43 mg, 0.10 mmol, 1.0 equiv), [Rh(COD)Cl]₂ (24.6 mg, 0.05 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was purified by column chromatography on silica gel to give the desired product as yellow solid. Yield 53% (34 mg). ¹H NMR (500 MHz, CDCl₃) δ 7.58-7.48 (m, 2H), 7.26-7.22 (m, 2H), 7.18 (d, J=7.5 Hz, 1H), 7.13 (s, 1H), 6.93 (s, 1H), 6.79 (dd, J=9.1, 6.5 Hz, 1H), 6.40 (d, J=6.0 Hz, 1H), 4.60-4.52 (m, 1H), 4.26-4.17 (m, 1H), 3.76-3.67 (m, 1H), 3.19-3.10 (m, 1H), 2.88-2.80 (m, 1H), 2.58 (s, 3H), 2.38 (s, 3H), 1.94-1.83 (m, 1H), 1.77-1.72 (m, 1H), 1.72 (s, 3H), 1.61 (d, J=6.6 Hz, 3H), 1.56-1.51 (m, 1H), 1.47-1.41 (m, 1H), 1.36-1.29 (m, 2H), 1.25 (d, J=6.7 Hz, 3H), 1.21-1.16 (m, 1H), 1.14-1.03 (m, 2H), 1.00 (d, J=6.9 Hz, 3H), 0.81 (d, J=6.7 Hz, 3H). ¹³C NMR (125 MHz, CDCl₃) δ 174.92 (d, J=50.4 Hz), 149.92, 147.13, 139.00, 138.03, 137.96, 137.20, 134.99, 134.45, 134.06, 129.99, 129.83, 127.79, 124.62, 122.35, 121.68, 117.80, 117.19, 114.72, 93.54 (d, J=9.1 Hz), 91.51 (d, J=7.7 Hz), 69.68 (d, J=14.1 Hz), 69.45 (d, J=14.1 Hz), 32.58, 31.73, 31.15, 30.23, 27.96, 27.79, 27.73, 26.04, 23.89, 22.02, 21.20, 20.36, 17.34. HRMS calcd for C₃₆H₄₄N₂Rh (M⁺−Cl) 607.2554, found 607.2559.

### dicarbonyl [5-(2,6-diisopropylphenyl)-2-mesitylimidazo[1,5-α]pyridin-3-ylidene]rhodium(I) chloride (64)

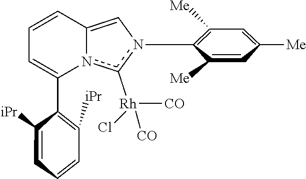

In a 10 mL vial, the corresponding (NHC)Rh(cod)C₁ (32 mg, 0.05 mmol) was dissolved in dichloromethane (0.5 mL). Carbon monoxide was bubbled into the solution for 3 h at room temperature. Dichloromethane was then removed under vacuo. The product was obtained by trituration from hexane as yellow solid. Yield 88% (26 mg). ¹H NMR (500 MHz,) δ 7.49 (t, J=7.8 Hz, 1H), 7.44-7.39 (m, 2H), 7.38 (s, 1H), 7.28 (d, J=7.6 Hz, 1H), 7.02-6.93 (m, 3H), 6.66 (d, J=6.6 Hz, 1H), 3.19-3.06 (m, 1H), 2.35 (s, 3H), 2.24 (s, 3H), 2.00-1.93 (m, 1H), 1.90 (s, 3H), 1.50 (d, J=6.6 Hz, 3H), 1.22-1.16 (m, 6H), 0.94 (d, J=6.7 Hz, 3H). ¹³C NMR (125 MHz, CDCl₃) δ 185.49 (d, J=55.4 Hz), 183.07 (d, J=75.8 Hz), 169.69 (d, J=45.0 Hz), 149.03, 147.54, 139.30, 137.30, 136.55, 135.52, 133.67, 132.71, 130.23, 129.87, 128.65, 124.07, 122.67, 122.16, 118.55, 117.10, 114.47, 31.83, 30.58, 27.30, 26.50, 23.63, 21.16, 19.17, 17.47. HRMS calcd for C₃₀H₃₂N₂O₂Rh (M⁺−Cl) 555.1513, found 555.1541.

### cyclooctadiene [2,5-dimesitylimidazo[1,5-α]pyridin-2-ylidene]rhodium(I) chloride (65)

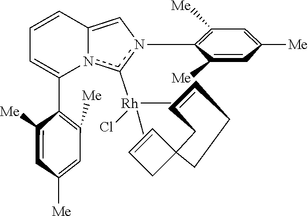

An oven-dried flask equipped with a stir bar was charged with the corresponding NHC·HCl salt (39 mg, 0.10 mmol, 1.0 equiv), [Rh(COD)Cl]₂ (24.6 mg, 0.05 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was purified by column chromatography on silica gel to give the desired product as yellow solid. Yield 61% (37 mg). ¹H NMR (500 MHz, CDCl₃) δ 7.24-7.18 (m, 3H), 7.14 (s, 1H), 6.93 (s, 1H), 6.89 (s, 1H), 6.82 (dd, J=9.2, 6.6 Hz, 1H), 6.32 (d, J=6.0 Hz, 1H), 4.56-4.49 (m, 1H), 4.28-4.23 (m, 1H), 3.29-3.22 (m, 1H), 2.87-2.81 (m, 1H), 2.69 (s, 3H), 2.60 (s, 3H), 2.43 (s, 3H), 2.40 (s, 3H), 1.75 (s, 3H), 1.70 (s, 3H), 1.58-1.54 (m, 2H), 1.48-1.40 (m, 2H), 1.31-1.25 (m, 2H), 1.13-1.05 (m, 2H). ¹³C NMR (125 MHz, CDCl₃) δ 173.67 (d, J=50.9 Hz), 139.77, 138.97, 138.95, 138.27, 137.66, 137.11, 136.90, 134.83, 133.65, 133.56, 129.74, 129.51, 127.67, 127.58, 122.41, 116.45, 116.19, 114.53, 93.45 (d, J=2.7 Hz), 93.38 (d, J=4.5 Hz), 71.07 (d, J=14.5 Hz), 67.11 (d, J=14.5 Hz), 34.13, 29.88, 28.31, 26.47, 22.15, 21.28, 21.10, 20.23, 19.79, 17.47. HRMS calcd for C₃₃H₃₈N₂Rh (M⁺−Cl) 565.2085, found 565.2093.

### dicarbonyl [2,5-dimesitylimidazo[1,5-α]pyridin-2-ylidene]rhodium(I) chloride (66)

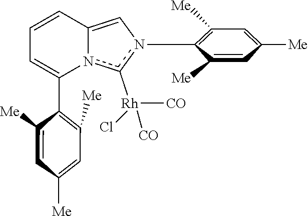

In a 10 mL vial, the corresponding (NHC)Rh(cod)C₁ (30 mg, 0.05 mmol) was dissolved in dichloromethane (0.5 mL). Carbon monoxide was bubbled into the solution for 3 h at room temperature. Dichloromethane was then removed under vacuo. The product was obtained by trituration from hexane as yellow solid. Yield 91% (25 mg). ¹H NMR (500 MHz, CDCl₃) δ 7.38 (d, J=9.2 Hz, 1H), 7.36 (s, 1H), 7.12 (s, 1H), 7.01-6.95 (m, 4H), 6.53 (d, J=6.6 Hz, 1H), 2.38 (s, 3H), 2.35 (s, 3H), 2.33 (s, 3H), 2.19 (s, 3H), 1.98 (s, 3H), 1.92 (s, 3H). ¹³C NMR (125 MHz, CDCl₃) δ 185.11 (d, J=55.4 Hz), 183.13 (d, J=75.8 Hz), 168.48 (d, J=45.0 Hz), 139.64, 139.55, 139.11, 138.89, 137.42, 136.68, 135.88, 133.74, 133.68, 131.57, 129.80, 129.51, 128.75, 128.15, 123.12, 116.72, 116.47, 114.38, 21.87, 21.33, 21.32, 20.25, 19.17, 17.61. HRMS calcd for C₂₇H₂₆N₂O₂Rh (M⁺−Cl) 513.1044, found 513.1067.

### cyclooctadiene [2-(2,6-dibenzhydryl-4-methylphenyl)-5-(2,4,6-triisopropylphenyl)imidazo[1,5-a]pyridin-3-ylidene]rhodium(I) chloride (67)

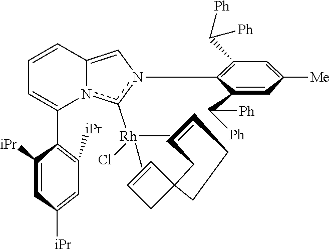

An oven-dried flask equipped with a stir bar was charged with the corresponding NHC·HCl salt (78 mg, 0.10 mmol, 1.0 equiv), [Rh(COD)Cl]₂ (24.6 mg, 0.05 mmol, 1.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was purified by column chromatography on silica gel to give the desired product as yellow solid. Yield 46% (46 mg). ¹H NMR (500 MHz, CDCl₃) δ 7.51-7.43 (m, 3H), 7.25-7.19 (m, 4H), 7.19-7.02 (m, 9H), 6.99 (s, 2H), 6.96-6.88 (m, 4H), 6.63 (s, 1H), 6.50 (d, J=6.7 Hz, 2H), 6.46 (d, J=7.6 Hz, 1H), 6.32 (s, 1H), 6.31-6.28 (m, 1H), 4.81-4.72 (m, 1H), 4.69 (s, 2H), 4.58-4.50 (m, 1H), 3.93-3.83 (m, 1H), 3.73-3.64 (m, 1H), 3.41-3.33 (m, 1H), 3.10-3.02 (m, 1H), 2.28 (s, 3H), 2.25-2.19 (m, 1H), 1.78-1.69 (m, 2H), 1.62 (d, J=6.6 Hz, 4H), 1.41 (d, J=7.0 Hz, 6H), 1.39-1.34 (m, 2H), 1.29 (d, J=6.7 Hz, 3H), 1.27-1.26 (m, 2H), 1.20 (d, J=6.7 Hz, 3H), 0.87-0.84 (m, 2H), 0.79 (d, J=6.7 Hz, 3H). ¹³C NMR (125 MHz, CDCl₃) δ 172.07 (d, J=50.4 Hz), 149.76, 149.73, 146.34, 145.19, 144.52, 143.86, 143.84, 142.42, 142.31, 138.61, 137.55, 137.00, 132.91, 131.52, 130.45, 130.41, 130.04, 129.82, 129.02, 128.60, 128.44, 128.10, 127.97, 127.91, 126.63, 126.59, 125.99, 125.93, 123.05, 120.14, 119.40, 119.05, 117.42, 94.01 (d, J=6.8 Hz), 93.48 (d, J=8.2 Hz), 71.46 (d, J=14.1 Hz), 66.54 (d, J=14.1 Hz), 51.99, 50.34, 34.82, 34.62, 33.98, 31.74, 31.51, 30.72, 29.86, 28.76, 27.97, 26.94, 25.45, 24.24, 24.17, 24.06, 22.80, 22.62, 21.97, 14.27. HRMS calcd for C₆₃H₆₆N₂Rh (M⁺−Cl) 953.4276, found 953.4283.

### dicarbonyl [2-(2,6-dibenzhydryl-4-methylphenyl)-5-(2,4,6-triisopropylphenyl)imidazo[1,5-α]pyridin-3-ylidene]rhodium(I) chloride (68)

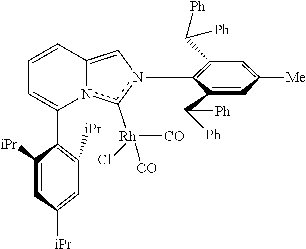

In a 10 mL vial, the corresponding (NHC)Rh(cod)C₁ (40 mg, 0.04 mmol) was dissolved in dichloromethane (0.4 mL). Carbon monoxide was bubbled into the solution for 24 h at room temperature. Dichloromethane was then removed under vacuo. The product was obtained by trituration from hexane as yellow solid. Yield 90% (38 mg). ¹H NMR (500 MHz, CDCl₃) δ 7.32 (s, 1H), 7.26-7.05 (m, 15H), 6.99 (d, J=7.5 Hz, 2H), 6.86-6.79 (m, 2H), 6.73 (s, 1H), 6.71-6.67 (m, 1H), 6.65 (s, 1H), 6.59 (d, J=6.6 Hz, 1H), 6.57-6.48 (m, 3H), 6.40 (s, 1H), 5.03 (s, 1H), 4.86 (s, 1H), 3.27-3.16 (m, 1H), 3.05-2.96 (m, 1H), 2.26-2.17 (m, 4H), 1.55 (d, J=6.6 Hz, 3H), 1.34 (dd, J=7.0, 3.8 Hz, 6H), 1.23-1.16 (m, 6H), 0.94 (d, J=6.7 Hz, 3H). ¹³C NMR (125 MHz, CDCl₃) δ 185.90 (d, J=55.9 Hz), 183.79 (d, J=75.4 Hz), 167.85 (d, J=44.1 Hz), 151.03, 149.31, 147.07, 144.50, 143.51, 143.25, 142.26, 141.81, 139.07, 136.94, 136.69, 131.29, 130.78, 130.46, 130.32, 129.91, 129.87, 129.80, 129.16, 128.25, 128.12, 128.06, 127.92, 126.78, 126.57, 126.24, 126.04, 122.33, 121.02, 120.75, 119.22, 118.77, 117.19, 51.62, 50.03, 34.86, 31.69, 31.00, 27.57, 26.46, 24.27, 23.89, 21.93, 21.90. HRMS calcd for C₅₇H₅₄N₂O₂Rh (M⁺−Cl) 901.3235, found 901.3260.

### 2,5-dimesitylimidazo[1,5-α]pyridine-3(2H)-selenone (69)

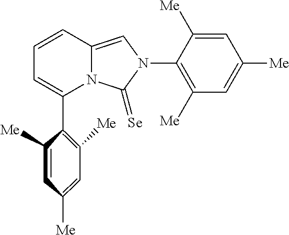

An oven-dried flask equipped with a stir bar was charged with 2,5-dimesitylimidazo[1,5-α]pyridin-2-ium chloride (39 mg, 0.10 mmol, 1.0 equiv), selenium (16 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as yellow solid in 91% yield (39.4 mg). ¹H NMR (500 MHz, CDCl₃) δ 7.23 (d, J=9.3 Hz, 1H), 7.13 (s, 1H), 6.96 (s, 2H), 6.87 (s, 2H), 6.84-6.78 (m, 1H), 6.27 (d, J=6.4 Hz, 1H), 2.33 (s, 3H), 2.32 (s, 3H), 2.09 (s, 6H), 1.95 (s, 6H). ¹³C NMR (125 MHz, CDCl₃) δ 147.92, 139.68, 139.18, 138.56, 137.69, 135.22, 134.99, 131.59, 131.18, 129.29, 127.31, 122.59, 117.24, 115.77, 109.81, 21.46, 21.37, 20.97, 18.29. ⁷⁷Se NMR (95 MHz, CDCl₃) δ 100.66. HRMS calcd for C₂₅H₂₇N₂Se (M⁺+H) 434.1257, found 434.1274.

### 5-(2,6-diisopropylphenyl)-2-mesitylimidazo[1,5-α]pyridine-3(2H)-selenone (70)

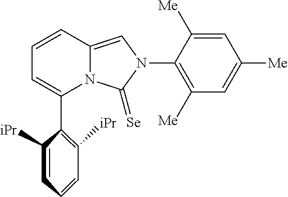

An oven-dried flask equipped with a stir bar was charged with the corresponding NHC·HCl salt (43 mg, 0.10 mmol, 1.0 equiv), selenium (16 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as yellow solid in 94% yield (44.8 mg). ¹H NMR (500 MHz, CDCl₃) δ 7.40 (t, J=7.7 Hz, 1H), 7.25 (d, J=11.4 Hz, 1H), 7.18-7.08 (m, 3H), 6.96 (s, 2H), 6.77 (dd, J=9.1, 6.6 Hz, 1H), 6.26 (d, J=6.3 Hz, 1H), 2.68-2.53 (m, 2H), 2.31 (s, 3H), 1.94 (s, 6H), 1.28 (d, J=6.9 Hz, 6H), 1.13 (d, J=6.8 Hz, 6H). ¹³C NMR (125 MHz, CDCl₃) δ 147.84, 139.09, 139.06, 135.11, 135.09, 131.84, 130.96, 129.48, 129.25, 121.96, 117.48, 116.16, 109.67, 31.76, 25.26, 22.53, 21.33, 18.25. ⁷⁷Se NMR (95 MHz, CDCl₃) δ 144.89. HRMS calcd for C₂₈H₃₃N₂Se (M⁺+H) 477.1805, found 477.1825.

### 2-(2,6-dibenzhydryl-4-methylphenyl)-5-mesitylimidazo[1,5-α]pyridine-3(2H)-selenone (71)

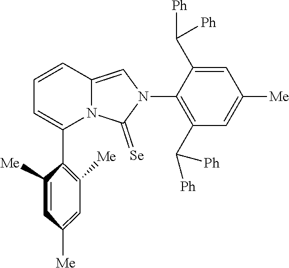

An oven-dried flask equipped with a stir bar was charged with the corresponding NHC·HCl salt (70 mg, 0.10 mmol, 1.0 equiv), selenium (16 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as yellow solid in 91% yield (67 mg). ¹H NMR (500 MHz, CDCl₃) δ 7.25-7.21 (m, 8H), 7.18-7.13 (m, 8H), 6.97 (s, 2H), 6.82 (d, J=5.7 Hz, 4H), 6.70 (s, 2H), 6.57-6.52 (m, 1H), 6.51-6.47 (m, 1H), 6.18 (dd, J=6.3, 1.5 Hz, 1H), 5.27 (s, 1H), 5.23 (s, 2H), 2.42 (s, 3H), 2.21 (s, 6H), 2.20 (s, 3H). ¹³C NMR (125 MHz, CDCl₃) δ 146.98, 143.03, 142.34, 142.02, 138.87, 138.78, 138.55, 137.88, 134.50, 131.88, 130.10, 129.96, 129.32, 129.14, 128.27, 128.24, 127.32, 126.46, 126.41, 121.20, 117.33, 115.68, 113.20, 52.21, 21.99, 21.57, 21.06. ⁷⁷Se NMR (95 MHz, CDCl₃) δ 119.35. HRMS calcd for C₄₉H₄₃N₂Se (M⁺+H) 739.2586, found 739.2603.

### 2-(2,6-dibenzhydryl-4-methoxyphenyl)-5-mesitylimidazo[1,5-α]pyridine-3(2H)-selenone (72)

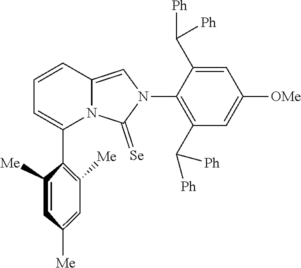

An oven-dried flask equipped with a stir bar was charged with the corresponding NHC·HCl salt (71 mg, 0.10 mmol, 1.0 equiv), selenium (16 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as yellow solid in 92% yield (69 mg). ¹H NMR (500 MHz, CDCl₃) δ 7.23-7.19 (m, 8H), 7.17-7.12 (m, 8H), 6.96 (s, 2H), 6.84-6.79 (m, 4H), 6.55-6.50 (m, 1H), 6.49-6.45 (m, 1H), 6.40 (s, 2H), 6.16 (d, J=6.3 Hz, 1H), 5.24 (s, 1H), 5.21 (s, 2H), 3.53 (s, 3H), 2.41 (s, 3H), 2.19 (s, 6H). ¹³C NMR (125 MHz, CDCl₃) δ 159.39, 147.40, 143.88, 142.79, 142.13, 138.88, 138.58, 137.90, 131.92, 130.03, 129.93, 129.27, 129.08, 128.31, 127.34, 126.61, 126.49, 121.17, 117.34, 115.69, 114.86, 113.47, 55.11, 52.42, 21.58, 21.07. ⁷⁷Se NMR (95 MHz, CDCl₃) δ 118.02. HRMS calcd for C₄₉H₄₃N₂SeO (M⁺+H) 755.2535, found 755.2544.

### 2-(2,6-dibenzhydryl-4-methylphenyl)-5-(2,4,6-triisopropylphenyl)imidazo[1,5-α]pyridine-3(2H)-selenone (73)

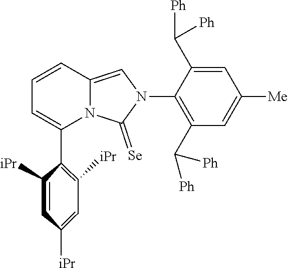

An oven-dried flask equipped with a stir bar was charged with the corresponding NHC·HCl salt (78 mg, 0.10 mmol, 1.0 equiv), selenium (16 mg, 0.20 mmol, 2.0 equiv) and KOtBu (16.8 mg, 0.15 mmol, 1.5 equiv), placed under a positive pressure of argon and subjected to three evacuation/backfilling cycles under high vacuum. THF (1.0 mL, 0.10 M) were added and the resulting solution was stirred at room temperature for 12 h. The reaction mixture was diluted with CH₂Cl₂ and filtered through Celite. The solution was collected and concentrated. The product was obtained by trituration from hexane as yellow solid in 94% yield (77 mg). ¹H NMR (500 MHz, CDCl₃) δ 7.23-7.17 (m, 8H), 7.16-7.10 (m, 8H), 7.02 (s, 2H), 6.81 (d, J=6.5 Hz, 4H), 6.68 (s, 2H), 6.48 (s, 2H), 6.24-6.19 (m, 1H), 5.26 (s, 1H), 5.19 (s, 2H), 3.01 (p, J=7.0 Hz, 1H), 2.73 (p, J=6.7 Hz, 2H), 2.18 (s, 3H), 1.37 (d, J=6.9 Hz, 6H), 1.35 (d, J=6.9 Hz, 6H), 1.16 (d, J=6.8 Hz, 6H). ¹³C NMR (125 MHz, CDCl₃) δ 150.01, 148.34, 147.55, 143.05, 142.67, 141.93, 138.72, 138.68, 134.59, 130.03, 129.94, 129.39, 129.28, 129.01, 128.27, 128.19, 126.39, 126.34, 120.68, 120.10, 117.31, 116.13, 112.80, 52.22, 34.45, 31.99, 25.20, 24.35, 22.88, 22.01. ⁷⁷Se NMR (95 MHz, CDCl₃) δ 170.40. HRMS calcd for C₅₅H₅₅N₂Se (M⁺+H) 823.3525, found 822.3549.

### (5-(2,4,6-triethylphenyl)-2-(2,6-diisopropylphenyl)-2,3-dihydroimidazo[1,5-α]pyridin-3-yl)gold(I) chloride (74)

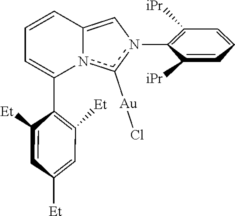

An oven-dried flask equipped with a stir bar was charged with the corresponding NHC·HCl salt (47.5 mg, 0.10 mmol, 1.0 equiv), chloro(dimethylsulfide)gold(I) (29.5 mg, 0.1 mmol, 1.0 equiv) and potassium carbonate (27.6 mg, 0.2 mmol, 2 equiv), placed under a positive pressure of argon and subjected to three evacuation/backfilling cycles under high vacuum. Acetone (1.0 mL, 0.10 M) were added and the resulting solution was heated and stirred at 60° C. for 24 h. The reaction mixture was diluted with CH₂Cl₂ and filtered through Celite. The solution was collected and concentrated. The residue was loaded onto a short pad of silica gel and eluted with CH₂Cl₂ until the product was completely recovered. After triturating with pentane and drying under high vacuum, the desired product was obtained as white solid. Yield 83% (55.8 mg). ¹H NMR (500 MHz, CDCl₃) δ 7.48 (d, J=8.4 Hz, 1H), 7.44 (t, J=7.8 Hz, 1H), 7.32 (s, 1H), 7.20 (d, J=7.8 Hz, 2H), 7.11 (s, 2H), 7.10-7.06 (m, 1H), 6.62 (dd, J=6.6, 1.1 Hz, 1H), 2.74 (q, J=7.6 Hz, 2H), 2.50-2.42 (m, 2H), 2.32-2.25 (m, 2H), 2.17-2.10 (m, 2H), 1.34 (t, J=7.6 Hz, 3H), 1.21 (d, J=6.8 Hz, 6H), 1.15-1.10 (m, 12H). ¹³C NMR (125 MHz, CDCl₃) δ 166.05, 147.47, 145.16, 142.60, 138.29, 135.68, 131.35, 130.71, 129.83, 126.39, 124.16, 123.59, 117.05, 116.90, 113.62, 29.09, 28.62, 26.82, 24.46, 15.50. HRMS calcd for C₃₁H₃₉AuN₂Cl (M⁺+H) 670.2384, found 670.2415; 672.2357, found 672.2383.

### Cinnamyl (5-(2,4,6-triethylphenyl)-2-(2,6-diisopropylphenyl)-2,3-dihydroimidazo[1,5-α]pyridin-3-yl)chloropalladium (II) (75)

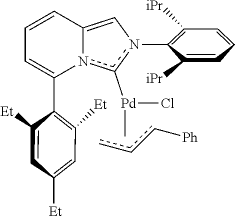

An oven-dried flask equipped with a stir bar was charged with the corresponding NHC·HCl salt (998 mg, 2.1 mmol, 1.05 equiv), KOtBu (314 mg, 2.8 mmol, 1.4 equiv), and [Pd(cin)Cl]₂ (518 mg, 1 mmol, 0.5 equiv), placed under a positive pressure of argon and subjected to three evacuation/backfilling cycles under high vacuum. Anhydrous tetrahydrofuran (20 mL) was added, and the resulting reaction mixture was stirred at room temperature for 4 h. The reaction mixture was diluted with CH₂Cl₂ and passed through a short pad of Celite. The solution was collected and concentrated. The residue was loaded onto a short pad of silica gel and eluted with hexane/ethyl acetate (4:1) until the product was completely recovered. After triturating with pentane and drying under high vacuum, the desired product was obtained as yellow solid. Yield 79% (1.1 g). NMR data were acquired at −30° C. ¹H NMR showed the presence of two isomers, the ratio between two isomers is 3:7. ¹H NMR (500 MHz, CDCl₃, 243 K) δ 7.52-7.39 (m, 2H), 7.39-7.30 (m, 2H), 7.28-7.12 (m, 5H), 7.03-6.84 (m, 4H), 6.57 (d, J=6.4 Hz, 0.3H), 6.47 (d, J=6.5 Hz, 0.7H), 4.79-4.68 (m, 0.3H), 4.31-4.21 (m, 0.7H), 3.90 (d, J=12.5 Hz, 0.7H), 3.22 (d, J=12.6 Hz, 0.3H), 3.20-3.13 (m, 0.7H), 3.12-3.06 (m, 0.3H), 3.04-2.98 (m, 0.3H), 2.96-2.88 (m, 0.7H), 2.86-2.65 (m, 4H), 2.26-2.15 (m, 1H), 2.14-2.08 (m, 0.7H), 2.06-2.00 (m, 0.3H), 1.97-1.86 (m, 1H), 1.47-1.38 (m, 1H), 1.36 (t, J=7.6 Hz, 2H), 1.33-1.19 (m, 8H), 1.15 (t, J=7.5 Hz, 2H), 1.10-1.03 (m, 8H), 1.01-0.92 (m, 1H). ¹³C NMR (126 MHz, CDCl₃, 243 K) δ 175.56, 175.28, 147.17, 146.06, 145.19, 144.88, 144.80, 144.60, 144.45, 142.46, 141.92, 138.43, 138.30, 138.03, 137.84, 136.84, 136.73, 132.54, 131.42, 131.04, 129.98, 129.86, 128.25, 128.02, 127.33, 126.88, 126.58, 126.08, 125.73, 124.29, 124.01, 122.94, 122.79, 122.63, 122.56, 116.69, 116.08, 115.69, 115.37, 109.55, 108.14, 90.16, 89.54, 47.66, 46.25, 28.99, 28.93, 28.59, 28.36, 28.24, 28.10, 27.89, 27.27, 26.96, 26.67, 25.61, 25.48, 23.32, 22.76, 22.29, 22.03, 16.49, 15.50, 15.23, 14.24, 14.10. HRMS calcd for C₄₀H₄₈N₂PdCl (M⁺+H) 719.2366, found 719.2341; 721.2359, found 719.2328.

### (5-(2,4,6-trimethylphenyl)-2-(2,6-diisopropylphenyl)-2,3-dihydroimidazo[1,5-α]pyridin-3-yl)gold(I) chloride (76)

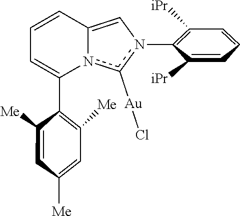

An oven-dried flask equipped with a stir bar was charged with the corresponding NHC·HCl salt (43 mg, 0.1 mmol, 1.0 equiv), chloro(dimethylsulfide)gold(I) (30 mg, 1 mmol, 1.0 equiv) and potassium carbonate (42 mg, 0.3 mmol, 3 equiv), placed under a positive pressure of argon and subjected to three evacuation/backfilling cycles under high vacuum. Acetone (1 mL, 0.10 M) were added and the resulting solution was heated and stirred at 60° C. for 3 h. The reaction mixture was diluted with CH₂Cl₂ and filtered through Celite. The solution was collected and concentrated. The residue was loaded onto a short pad of silica gel and eluted with CH₂Cl₂ until the product was completely recovered. After triturating with pentane and drying under high vacuum, the desired product was obtained as white solid. Yield 74% (46 mg). ¹H NMR (500 MHz, CDCl₃) δ 7.49-7.43 (m, 2H), 7.32 (s, 1H), 7.21 (d, J=7.8 Hz, 2H), 7.11-7.07 (m, 1H), 7.05 (s, 2H), 6.58 (dd, J=6.6, 1.2 Hz, 1H), 2.41 (s, 3H), 2.18-2.12 (m, 2H), 2.08 (s, 6H), 1.23 (d, J=6.9 Hz, 6H), 1.12 (d, J=6.9 Hz, 6H). ¹³C NMR (126 MHz, CDCl₃) δ 165.55, 145.18, 141.00, 138.73, 136.91, 135.60, 131.34, 130.75, 129.09, 124.17, 123.88, 116.81, 116.51, 113.70, 28.60, 24.51, 24.47, 21.55, 19.95.

### (5-(2,6-diisopropylphenyl)-2-(2,6-diisopropylphenyl)-2,3-dihydroimidazo[1,5-α]pyridin-3-yl)gold(I) chloride (77)

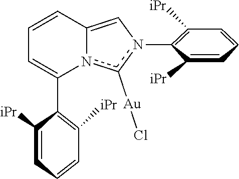

An oven-dried flask equipped with a stir bar was charged with the corresponding NHC·HCl salt (48 mg, 0.1 mmol, 1.0 equiv), chloro(dimethylsulfide)gold(I) (30 mg, 1 mmol, 1.0 equiv) and potassium carbonate (42 mg, 0.3 mmol, 3 equiv), placed under a positive pressure of argon and subjected to three evacuation/backfilling cycles under high vacuum. Acetone (1 mL, 0.10 M) were added and the resulting solution was heated and stirred at 60° C. for 4 h. The reaction mixture was diluted with CH₂Cl₂ and filtered through Celite. The solution was collected and concentrated. The residue was loaded onto a short pad of silica gel and eluted with CH₂Cl₂ until the product was completely recovered. After triturating with pentane and drying under high vacuum, the desired product was obtained as white solid. Yield 78% (52 mg). ¹H NMR (500 MHz, CDCl₃) δ 7.62 (t, J=7.8 Hz, 1H), 7.50 (dd, J=9.3, 1.1 Hz, 1H), 7.44 (t, J=7.8 Hz, 1H), 7.34 (s, 1H), 7.31 (d, J=7.9 Hz, 2H), 7.21 (d, J=7.8 Hz, 2H), 7.13-7.08 (m, 1H), 6.60 (dd, J=6.6, 1.2 Hz, 1H), 2.46-2.39 (m, 2H), 2.18-2.10 (m, 2H), 1.27 (d, J=6.9 Hz, 6H), 1.20 (d, J=6.8 Hz, 6H), 1.15 (d, J=6.8 Hz, 6H), 1.12 (d, J=6.9 Hz, 6H). ¹³C NMR (126 MHz, CDCl₃) δ 166.22, 147.46, 145.13, 138.03, 135.63, 131.53, 131.30, 131.08, 130.69, 124.20, 123.97, 123.48, 117.09, 117.03, 113.59, 31.88, 28.63, 25.34, 24.58, 24.33, 23.60.

### (5-(2,6-diisopropylphenyl)-2-cyclohexyl-2,3-dihydroimidazo[1,5-α]pyridin-3-yl)gold(I) chloride (78)

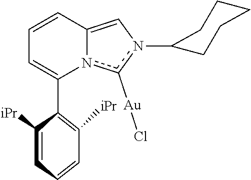

An oven-dried flask equipped with a stir bar was charged with the corresponding NHC·HCl salt (40 mg, 0.1 mmol, 1.0 equiv), chloro(dimethylsulfide)gold(I) (30 mg, 1 mmol, 1.0 equiv) and potassium carbonate (42 mg, 0.3 mmol, 3 equiv), placed under a positive pressure of argon and subjected to three evacuation/backfilling cycles under high vacuum. Acetone (1 mL, 0.10 M) were added and the resulting solution was heated and stirred at 60° C. for 8 h. The reaction mixture was diluted with CH₂Cl₂ and filtered through Celite. The solution was collected and concentrated. The residue was loaded onto a short pad of silica gel and eluted with CH₂Cl₂ until the product was completely recovered. After triturating with pentane and drying under high vacuum, the desired product was obtained as white solid. Yield 53% (31 mg). ¹H NMR (500 MHz, CDCl₃) δ 7.63 (t, J=7.8 Hz, 1H), 7.43 (s, 1H), 7.40 (d, J=9.2 Hz, 2H), 7.33 (d, J=7.9 Hz, 2H), 7.01-6.94 (m, 1H), 6.48 (d, J=6.4 Hz, 1H), 4.98-4.88 (m, 1H), 2.36-2.27 (m, 2H), 2.18-2.12 (m, 2H), 1.91-1.84 (m, 2H), 1.80-1.73 (m, 1H), 1.73-1.61 (m, 2H), 1.54-1.42 (m, 2H), 1.26 (d, J=6.9 Hz, 7H), 1.24-1.17 (m, 1H), 1.09 (d, J=6.8 Hz, 6H). ¹³C NMR (126 MHz, CDCl₃) δ 162.37, 147.58, 137.53, 131.73, 131.50, 130.93, 123.95, 122.74, 116.90, 116.76, 107.92, 63.20, 34.48, 31.63, 25.41, 25.39, 25.27, 23.77.

### Example 4: Catalysis

### General Procedure for the Suzuki-Miyaura Cross-Coupling of Nitroarenes Using [Pd(NHC (allyl)Cl] Complexes (General Procedure I)

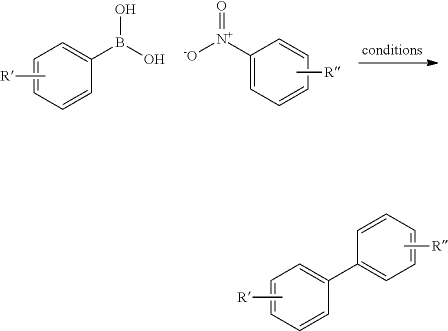

An oven-dried vial equipped with a stir bar was charged with nitroarene (neat, 1.0 equiv), boronic acid (typically, 1.5 equiv), K₃PO₄ (typically, 3.0 equiv), Pd—NHC catalyst (typically, 5 mol %), water (typically, 3 equiv), TDA (typically, 10 mol %) placed under a positive pressure of argon, and subjected to three evacuation/backfilling cycles under high vacuum. Dioxane (typically, 0.2 M) was added with vigorous stirring at room temperature, the reaction mixture was placed in a preheated oil bath (typically, 130° C.) and stirred for 15 h. After 15 h, the reaction mixture was cooled down to room temperature, diluted with CH₂Cl₂ (10 mL), filtered, and concentrated. A sample was analyzed by ¹H NMR (CDCl₃, 500 MHz) and GC-MS to obtain conversion, and yield using internal standard and comparison with authentic samples. Purification by chromatography on silica gel afforded the product.

### Synthesis of 4-methoxybiphenyl (General Procedure I)

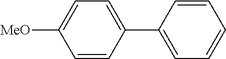 [SMILES: COc1ccc(-c2ccccc2)cc1]

According to the general procedure, the reaction of nitrobenzene (0.2 mmol), 4-methoxyphenylboronic acid (1.5 equiv), K₃PO₄ (3.0 equiv), 39 (5 mol %), water (3 equiv) and TDA (10 mol %) for 16 h at 130° C., afforded after work-up and chromatography the title compound in 88% yield (32.4 mg). White solid. ¹H NMR (500 MHz, CDCl₃) δ 7.59-7.51 (m, 4H), 7.42 (t, J=7.7 Hz, 2H), 7.30 (t, J=7.4 Hz, 1H), 6.98 (d, J=8.7 Hz, 2H), 3.86 (s, 3H). ¹³C NMR (125 MHz, CDCl₃) δ 159.30, 140.99, 133.94, 128.86, 128.30, 126.89, 126.80, 114.35, 55.50.

### General Procedure for the Suzuki-Miyaura Cross-Coupling of Nitroarenes Using [Pd(NHC)(py)Cl₂] Complexes (General Procedure II)

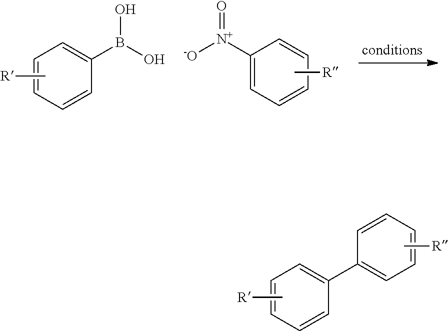

An oven-dried vial equipped with a stir bar was charged with nitroarene (neat, 1.0 equiv), boronic acid (typically, 1.5 equiv), K₃PO₄ (typically, 3.0 equiv), Pd—NHC catalyst (typically, 5 mol %), water (typically, 3 equiv), TDA (typically, 10 mol %) placed under a positive pressure of argon, and subjected to three evacuation/backfilling cycles under high vacuum. Dioxane (typically, 0.2 M) was added with vigorous stirring at room temperature, the reaction mixture was placed in a preheated oil bath (typically, 130° C.) and stirred for 15 h. After 15 h, the reaction mixture was cooled down to room temperature, diluted with CH₂Cl₂ (10 mL), filtered, and concentrated. A sample was analyzed by ¹H NMR (CDCl₃, 500 MHz) and GC-MS to obtain conversion, and yield using internal standard and comparison with authentic samples. Purification by chromatography on silica gel (EtOAc/hexanes) afforded the product.

### Synthesis of 4-methoxybiphenyl (General Procedure II)

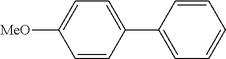 [SMILES: COc1ccc(-c2ccccc2)cc1]

According to the general procedure, the reaction of nitrobenzene (0.2 mmol), 4-methoxyphenylboronic acid (1.5 equiv), K₃PO₄ (3.0 equiv), 47 (5 mol %), water (3 equiv) and TDA (10 mol %) for 36 h at 130° C., afforded after work-up and chromatography the title compound in 71% yield (26.1 mg). White solid. ¹H NMR (500 MHz, CDCl₃) δ 7.61-7.51 (m, 4H), 7.42 (t, J=7.7 Hz, 2H), 7.31 (t, J=7.3 Hz, 1H), 6.99 (d, J=8.8 Hz, 2H), 3.86 (s, 3H). ¹³C NMR (125 MHz, CDCl₃) δ 159.30, 140.98, 133.94, 128.86, 128.30, 126.89, 126.80, 114.35, 55.49.

### General Procedure for Amination of Alkyl Sulfoxides (General Procedure III)

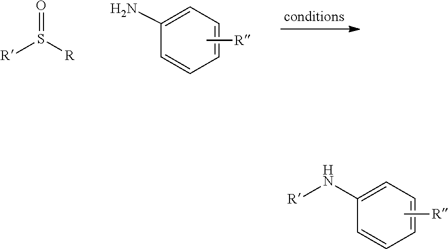

An oven-dried vial equipped with a stir bar was charged with sulfoxide (neat, 1.0 equiv), aniline (typically, 1.5 equiv), [Pd(cin)Cl]₂ (typically, 2.5 mol %), Pd—NHC catalyst (typically, 10 mol %) placed under a positive pressure of argon, and subjected to three evacuation/backfilling cycles under high vacuum. THF (typically, 0.2 M) and LiHMDS in THF (typically, 2 equiv) was added with vigorous stirring at room temperature, the reaction mixture was placed in a preheated oil bath (typically, 100° C.) and stirred for 12 h. After 12 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. A sample was analyzed by ¹H NMR (CDCl₃, 500 MHz) and GC-MS to obtain conversion, and yield using internal standard and comparison with authentic samples. Purification by chromatography on silica gel (EtOAc/hexanes) afforded the title product.

### Synthesis of 4-methyl-N-phenylaniline (General Procedure III)

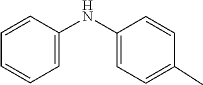

An oven-dried vial equipped with a stir bar was charged with methyl phenyl sulfoxide (28 mg, 0.2 mmol, 1.0 equiv), p-toluidine (32 mg, 0.3 mmol, 1.5 equiv), [Pd(cin)Cl]₂ (2.6 mg, 0.005 mmol, 2.5 mol %), 9 (8.7 mg, 0.02 mmol, 10 mol %) placed under a positive pressure of argon, and subjected to three evacuation/backfilling cycles under high vacuum. THF (1 mL, 0.2 M) and 1 M LiHMDS in THF (0.4 mL, 0.4 mmol, 2 equiv) was added with vigorous stirring at room temperature, the reaction mixture was placed in a preheated oil bath and stirred for 12 h at 100° C. After 12 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. Purification by chromatography on silica gel (EtOAc/hexanes) afforded the title product in 86% yield (31.5 mg). White solid. ¹H NMR (500 MHz, CDCl₃) δ 7.25-7.19 (m, 2H), 7.07 (d, J=8.2 Hz, 2H), 7.02-6.95 (m, 4H), 6.86 (t, J=7.3 Hz, 1H), 5.64 (s, 1H), 2.29 (s, 3H). ¹³C NMR (125 MHz, CDCl₃) δ 144.06, 140.39, 131.11, 130.00, 129.45, 120.47, 119.06, 117.02, 20.83.

### General Procedure for Amination of Thiophenols (General Procedure IV)

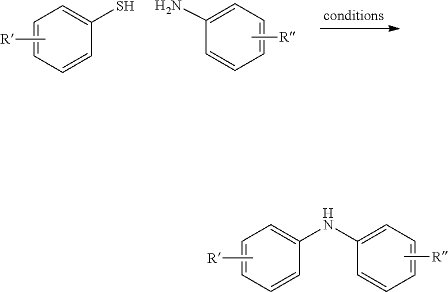

An oven-dried vial equipped with a stir bar was charged with thiophenol (neat, 1.0 equiv), aniline (typically, 2 equiv), Pd—NHC catalyst (typically, 5 mol %), placed under a positive pressure of argon, and subjected to three evacuation/backfilling cycles under high vacuum. Toluene (typically, 0.2 M) and LiHMDS in toluene (typically, 4 equiv) was added with vigorous stirring at room temperature, the reaction mixture was placed in a preheated oil bath (typically, 130° C.) and stirred for 12 h. After 12 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. A sample was analyzed by ¹H NMR (CDCl₃, 500 MHz) and GC-MS to obtain conversion, and yield using internal standard and comparison with authentic samples. Purification by chromatography on silica gel (EtOAc/hexanes) afforded the title product.

### Synthesis of 2-methyl-N-phenylaniline (General Procedure IV)

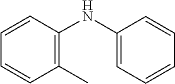

An oven-dried vial equipped with a stir bar was charged with thiophenol (22 mg, 0.2 mmol, 1.0 equiv), o-toluidine (43 mg, 0.4 mmol, 2 equiv), 37 (6.1 mg, 0.01 mmol, 5 mol %), placed under a positive pressure of argon, and subjected to three evacuation/backfilling cycles under high vacuum. Toluene (1 mL, 0.2 M) and 1 M LiHMDS in toluene (0.8 mL, 0.8 mmol, 4 equiv) was added with vigorous stirring at room temperature, the reaction mixture was placed in a preheated oil bath and stirred for 12 h at 130° C. After 12 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. Purification by chromatography on silica gel (EtOAc/hexanes) afforded the title product in 85% yield (29.7 mg). White solid. ¹H NMR (500 MHz, CDCl₃) δ 7.26-7.20 (m, 3H), 7.18 (d, J=6.8 Hz, 1H), 7.12 (t, J=7.7 Hz, 1H), 6.98-6.82 (m, 4H), 5.37 (s, 1H), 2.24 (s, 3H). ¹³C NMR (125 MHz, CDCl₃) δ 144.10, 141.34, 131.08, 129.45, 128.42, 126.90, 122.12, 120.62, 118.91, 117.60, 18.04.

### General Procedure for Monoarylation of Hydrazine (General Procedure V)

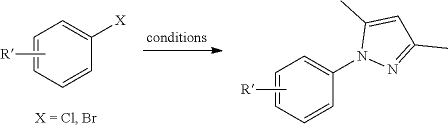

An oven dried vial equipped with a stir bar was charged with halobenzene (neat, 1.0 equiv), Pd—NHC catalyst (typically, 5 mol %), and rubidium carbonate (typically, 3 equiv), placed under a positive pressure of argon, and subjected to three evacuation/backfilling cycles under high vacuum. Dioxane (typically, 0.1 M) and hydrazine monohydrate (typically, 5 equiv) was then added to the reaction, and the reaction was heated at 120° C. for 12 h. After the indicated time, acetylacetone (typically, 9.0 equiv) and 2M TFA in EtOH (typically, 4 equiv) was added to the reaction. The reaction was then heated at 100° C. for 6 h with stirring. Afterwards, the reaction mixture was diluted with CH₂Cl₂ (10 mL), filtered, and concentrated. A sample was analyzed by ¹H NMR (CDCl₃, 500 MHz) and GC-MS to obtain conversion, and yield using internal standard and comparison with authentic samples. Purification by chromatography on silica gel (EtOAc/hexanes) afforded the title product.

### Synthesis of 1-(4-methoxyphenyl)-3,5-dimethyl-1H-pyrazole (General Procedure V)

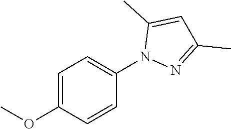

An oven dried vial equipped with a stir bar was charged with 4-chloroanisole (28.4 mg, 0.20 mmol, 1.0 equiv), 44 (9.7 mg, 0.01 mmol, 5 mol %) and rubidium carbonate (139 mg, 0.6 mmol, 3 equiv), placed under a positive pressure of argon, and subjected to three evacuation/backfilling cycles under high vacuum. Dioxane (2 mL, 0.1 M) and hydrazine monohydrate (50 μL, 1 mmol, 5 equiv) was then added to the reaction, and the reaction was heated at 120° C. for 12 h. After the indicated time, acetylacetone (184 μL, 1.8 mmol, 9.0 equiv) and 2M TFA in EtOH (0.4 mL, 0.8 mmol, 4 equiv) was added to the reaction. The reaction was then heated at 100° C. for 6 h with stirring. Afterwards, the reaction mixture was diluted with CH₂Cl₂ (10 mL), filtered, and concentrated. Purification by chromatography on silica gel (EtOAc/hexanes) afforded the title product as yellow oil. Yield 87% (35.2 mg). ¹H NMR (500 MHz, CDCl₃) δ 7.32 (d, J=8.9 Hz, 2H), 6.95 (d, J=8.9 Hz, 2H), 5.96 (s, 1H), 3.84 (s, 3H), 2.29 (s, 3H), 2.24 (s, 3H). ¹³C NMR (125 MHz, CDCl₃) δ 158.90, 148.63, 139.60, 133.21, 126.50, 114.23, 106.36, 55.66, 13.64, 12.28.

### General Procedure for Suzuki-Miyaura Cross-Coupling of Aryl Mesylates and Tosylates (General Procedure VI)

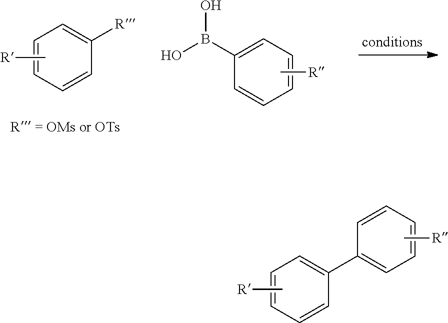

An oven-dried vial equipped with a stir bar was charged with an aryl mesylate (neat, 1.0 equiv), boronic acid (typically, 4.5 equiv), potassium phosphate (typically, 3.0 equiv), Pd—NHC catalyst (typically, 5 mol %) placed under a positive pressure of argon and subjected to three evacuation/backfilling cycles under high vacuum. Isopropanol (typically, 0.25 M) was added with vigorous stirring and the reaction mixture was placed in a 130° C. oil bath and stirred for the indicated time. After the indicated time, the reaction mixture was cooled down to room temperature, diluted with ethyl acetate (10 mL), filtered, and concentrated. A sample was analyzed by ¹H NMR (CDCl₃, 500 MHz) and GC-MS to obtain conversion, selectivity and yield using internal standard and comparison with authentic samples. Purification by chromatography on silica gel (EtOAc/hexanes) afforded the title product.

### Synthesis of 4-methoxy-4′-(trifluoromethyl)-1,1′-biphenyl (General Procedure VI)

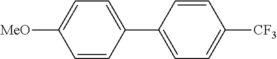 [SMILES: COc1ccc(-c2ccc(C(F)(F)F)cc2)cc1]

According to the general procedure, the reaction of 4-methoxyphenyl mesylate (0.20 mmol), 4-trifluoromethylphenylboronic acid (0.90 mmol), K₃PO₄ (0.60 mmol) and 43 (0.01 mmol) in isopropanol (0.25 M) for 12 h at 130° C., after work-up and chromatography the title compound was afforded in 99% yield (50.3 mg) as white solid. ¹H NMR (500 MHz, CDCl₃) ¹H NMR (500 MHz, CDCl₃) δ 7.68-7.64 (m, 4H), 7.55 (d, J=8.8 Hz, 2H), 7.01 (d, J=8.7 Hz, 2H), 3.87 (s, 3H). ¹³C NMR (125 MHz, CDCl₃) δ ¹³C NMR (125 MHz, CDCl₃) δ 159.84, 144.29, 132.18, 128.69 (q, J=32.4 Hz), 128.35, 126.87, 125.67 (q, J=3.8 Hz), 124.38 (q, J=271.8 Hz), 55.39.

### General Procedure for Buchwald-Hartwig Amination of Aryl Mesylates and Tosylates (General Procedure VII)

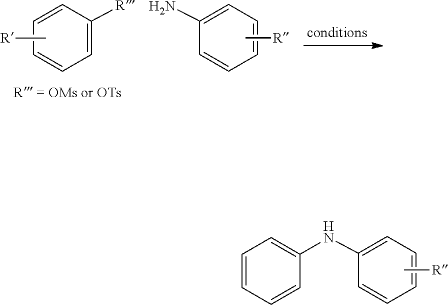

An oven-dried vial equipped with a stir bar was charged with an aryl mesylate (neat, 1.0 equiv), aniline (typically, 1.2 equiv), potassium phosphate (typically, 2.0 equiv), Pd—NHC catalyst (typically, 5 mol %) placed under a positive pressure of argon and subjected to three evacuation/backfilling cycles under high vacuum. tert-Amyl alcohol (typically, 0.25 M) was added with vigorous stirring and the reaction mixture was placed in a 120° C. oil bath and stirred for the indicated time. After the indicated time, the reaction mixture was cooled down to room temperature, diluted with ethyl acetate (10 mL), filtered, and concentrated. A sample was analyzed by ¹H NMR (CDCl₃, 500 MHz) and GC-MS to obtain conversion, selectivity and yield using internal standard and comparison with authentic samples. Purification by chromatography on silica gel (EtOAc/hexanes) afforded the title product.

### Synthesis of N-(p-tolyl)quinolin-6-amine (General Procedure VII)

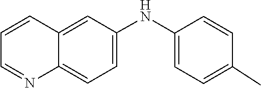 [SMILES: Cc1ccc(Nc2ccc3ncccc3c2)cc1]

According to the general procedure, the reaction of quinolin-6-yl methanesulfonate (0.20 mmol), p-toluidine (0.24 mmol), K₃PO₄ (0.40 mmol) and 42 (0.01 mmol) in tert-Amyl alcohol (0.25 M) for 12 h at 130° C., after work-up and chromatography the title compound was afforded in 99% yield (46.4 mg) as brown oil. ¹H NMR (500 MHz, CDCl₃) δ 8.60 (dd, J=4.3, 1.7 Hz, 1H), 7.88 (d, J=9.0 Hz, 1H), 7.82 (dd, J=8.3, 1.6 Hz, 1H), 7.29 (dd, J=9.1, 2.6 Hz, 1H), 7.20 (dd, J=8.3, 4.3 Hz, 1H), 7.18 (t, J=2.4 Hz, 1H), 7.10-7.02 (m, 4H), 5.89 (s, 1H), 2.27 (s, 3H). δ ¹³C NMR (125 MHz, CDCl₃) δ 147.25, 144.07, 142.53, 139.31, 134.28, 132.27, 130.49, 130.06, 129.70, 122.68, 121.47, 120.20, 108.14, 20.82.

### General Procedure for Suzuki-Miyaura Cross-Coupling of Aryl Sulfamates (General Procedure VIII)

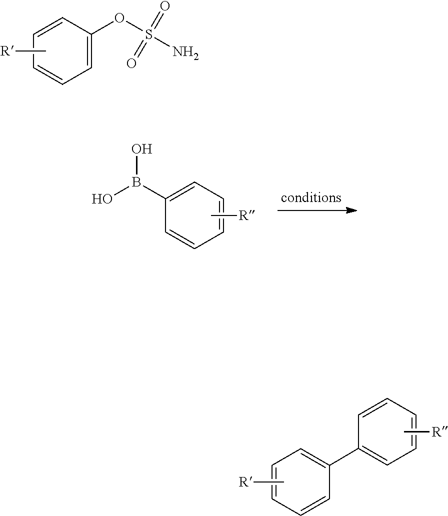

An oven-dried vial equipped with a stir bar was charged with an aryl sulfamate (neat, 1.0 equiv), boronic acid (typically, 4.5 equiv), potassium phosphate (typically, 3.0 equiv), Pd—NHC catalyst (typically, 5 mol %) placed under a positive pressure of argon and subjected to three evacuation/backfilling cycles under high vacuum. Isopropanol (typically, 0.25 M) was added with vigorous stirring and the reaction mixture was placed in a 130° C. oil bath and stirred for the indicated time. After the indicated time, the reaction mixture was cooled down to room temperature, diluted with ethyl acetate (10 mL), filtered, and concentrated. A sample was analyzed by ¹H NMR (CDCl₃, 500 MHz) and GC-MS to obtain conversion, selectivity and yield using internal standard and comparison with authentic samples. Purification by chromatography on silica gel (EtOAc/hexanes) afforded the title product.

### Synthesis of 1-(4′-methoxy-[1,1′-biphenyl]-4-yl)ethan-1-one (General Procedure VIII)

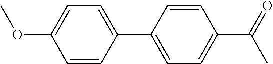 [SMILES: COc1ccc(-c2ccc(C(C)=O)cc2)cc1]

According to the general procedure, the reaction of 4-methoxy-N,N-dimethylsulfamate (0.20 mmol), (4-acetylphenyl)boronic acid (0.90 mmol), K₃PO₄ (0.60 mmol) and 42 (0.01 mmol) in isopropanol (0.25 M) for 12 h at 130° C., after work-up and chromatography the title compound was afforded in 92% yield (41.7 mg) as white solid. ¹H NMR (500 MHz, CDCl₃) ¹H NMR (500 MHz, CDCl₃) δ 8.01 (d, J=8.5 Hz, 2H), 7.65 (d, J=8.5 Hz, 2H), 7.58 (d, J=8.7 Hz, 2H), 7.00 (d, J=8.7 Hz, 2H), 3.87 (s, 3H), 2.63 (s, 3H). S ¹³C NMR (125 MHz, CDCl₃) δ 197.73, 159.92, 145.38, 135.30, 132.27, 128.95, 128.38, 126.63, 114.41, 55.40, 26.64. 55.39.

### General Procedure for Buchwald-Hartwig Amination of Aryl Sulfamates (General Procedure IX)

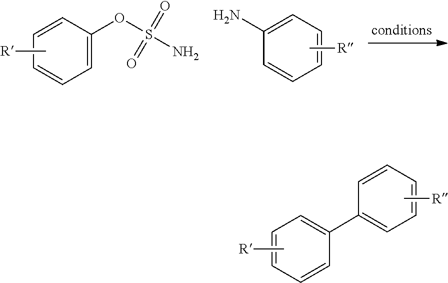

An oven-dried vial equipped with a stir bar was charged with an aryl sulfamate (neat, 1.0 equiv), aniline (typically, 1.2 equiv), potassium phosphate (typically, 2.0 equiv), Pd—NHC catalyst (typically, 5 mol %) placed under a positive pressure of argon and subjected to three evacuation/backfilling cycles under high vacuum. tert-Amyl alcohol (typically, 0.25 M) was added with vigorous stirring and the reaction mixture was placed in a 130° C. oil bath and stirred for the indicated time. After the indicated time, the reaction mixture was cooled down to room temperature, diluted with ethyl acetate (10 mL), filtered, and concentrated. A sample was analyzed by ¹H NMR (CDCl₃, 500 MHz) and GC-MS to obtain conversion, selectivity and yield using internal standard and comparison with authentic samples. Purification by chromatography on silica gel (EtOAc/hexanes) afforded the title product.

### Synthesis of N-(p-tolyl)naphthalen-2-amine (General Procedure IX)

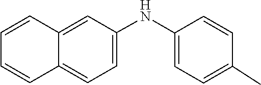 [SMILES: Cc1ccc(Nc2ccc3ccccc3c2)cc1]

According to the general procedure, the reaction of naphthalen-2-yl dimethylsulfamate (0.20 mmol), p-toluidine (0.24 mmol), K₃PO₄ (0.40 mmol) and 42 (0.01 mmol) in tert-Amyl alcohol (0.25 M) for 12 h at 130° C., after work-up and chromatography the title compound was afforded in 99% yield (46.0 mg) as brown oil. ¹H NMR (500 MHz, CDCl₃) δ 7.63 (d, J=8.8 Hz, 1H), 7.53 (d, J=8.2 Hz, 1H), 7.34-7.26 (m, 1H), 7.27 (d, J=2.2 Hz, 1H), 7.19 (t, J=7.5 Hz, 1H), 7.09 (dd, J=8.8, 2.3 Hz, 1H), 7.07-6.96 (m, 4H), 5.68 (s, 1H), 2.25 (s, 3H). S ¹³C NMR (125 MHz, CDCl₃) δ 141.71, 140.10, 134.73, 131.40, 129.97, 129.15, 128.88, 127.64, 126.42, 126.37, 123.19, 119.59, 119.37, 110.28, 20.78.

### General Procedure for Hydroxylation of Aryl Chlorides: Synthesis of 3-methoxyphenol (General Procedure X)

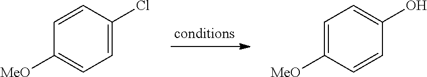 [SMILES: COc1ccc(Cl)cc1 ; COc1ccc(O)cc1]

An oven dried vial equipped with a stir bar was charged with 3-chloroanisole (28.4 mg, 0.20 mmol, 1.0 equiv), 44 (9.7 mg, 0.01 mmol, 5 mol %) and cesium hydroxide monohydrate (67 mg, 0.4 mmol, 2 equiv), placed under a positive pressure of argon, and subjected to three evacuation/backfilling cycles under high vacuum. Dioxane (1 mL, 0.2 M) was then added to the reaction, and the reaction was heated at 120° C. for 16 h. After the indicated time, quenched with saturated 1 M HCl aqueous solution (2 mL) and extracted with ethyl acetate (2×5 mL). The combined organic layers were dried over anhydrous Na₂SO₄ and concentrated under reduced pressure. Purification by chromatography on silica gel (EtOAc/hexanes) afforded the title product in 98% yield (24.3 mg). Yellow oil. Spectroscopic data matched literature values.

### General Procedure for Amination of Aryl Methyl Thioethers: Synthesis of diphenylamine (General Procedure XI)

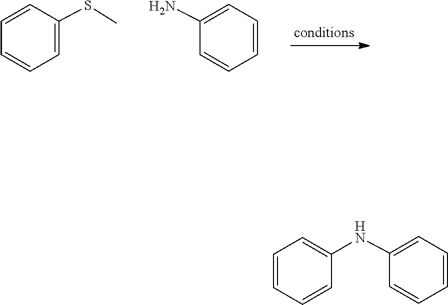

An oven-dried vial equipped with a stir bar was charged with thioanisole (25 mg, 0.2 mmol, 1.0 equiv), aniline (37.2 mg, 0.4 mmol, 2 equiv), 42 (6.6 mg, 0.01 mmol, 5 mol %), placed under a positive pressure of argon, and subjected to three evacuation/backfilling cycles under high vacuum. Toluene (1 mL, 0.2 M) and 1 M NaHMDS in toluene (0.4 mL, 0.4 mmol, 2 equiv) was added with vigorous stirring at room temperature, the reaction mixture was placed in a preheated oil bath and stirred for 12 h at 100° C. After 12 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. Purification by chromatography on silica gel (EtOAc/hexanes) afforded the title product in 94% yield (31.8 mg). White solid. ¹H NMR (500 MHz, CDCl₃) δ 7.26 (t, J=7.7 Hz, 4H), 7.07 (d, J=8.5 Hz, 4H), 6.92 (t, J=7.3 Hz, 2H), 5.78 (s, 1H). ¹³C NMR (125 MHz, CDCl₃) δ 143.22, 129.49, 121.19, 117.99.

### General Procedure for Amination of Aryl Sulfoxides: Synthesis of 4-methyl-N-phenylaniline (General Procedure XII)

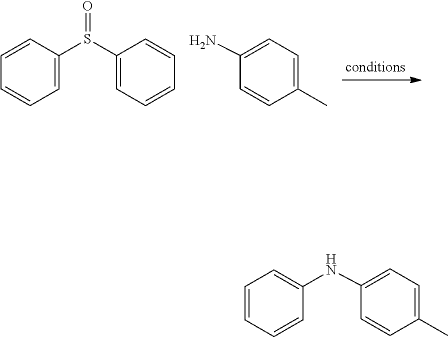

An oven-dried vial equipped with a stir bar was charged with diphenyl sulfoxide (61 mg, 0.3 mmol, 1.5 equiv), p-toluidine (21.4 mg, 0.2 mmol, 1 equiv), 42 (6.6 mg, 0.01 mmol, 5 mol %), placed under a positive pressure of argon, and subjected to three evacuation/backfilling cycles under high vacuum. THF (1 mL, 0.2 M) and 1 M LiHMDS in THF (0.4 mL, 0.4 mmol, 2 equiv) was added with vigorous stirring at room temperature, the reaction mixture was placed in a preheated oil bath and stirred for 12 h at 80° C. After 12 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. Purification by chromatography on silica gel (EtOAc/hexanes) afforded the title product in 92% yield (33.7 mg). White solid. ¹H NMR (500 MHz, CDCl₃) δ 7.25-7.19 (m, 2H), 7.07 (d, J=8.2 Hz, 2H), 7.02-6.95 (m, 4H), 6.86 (t, J=7.3 Hz, 1H), 5.64 (s, 1H), 2.29 (s, 3H). ¹³C NMR (125 MHz, CDCl₃) δ 144.06, 140.39, 131.11, 130.00, 129.45, 120.47, 119.06, 117.02, 20.83.

### General Procedure for Amination of Aryl Chlorides: Synthesis of 4-(p-tolyl)morpholine (General Procedure XIII)

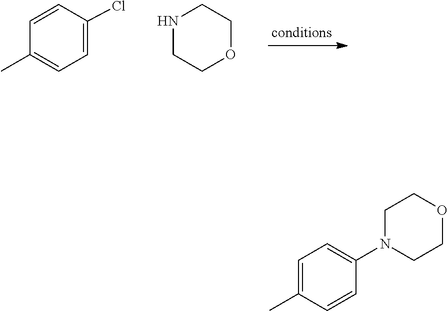

An oven-dried vial equipped with a stir bar was charged with 4-chlorotoluene (25.3 mg, 0.2 mmol, 1.5 equiv), morpholine (24.2 mg, 0.28 mmol, 1.4 equiv), 42 (1.3 mg, 0.002 mmol, 1 mol %), and KOtBu (31.4 mg, 0.28 mmol, 1.4 equiv) placed under a positive pressure of argon, and subjected to three evacuation/backfilling cycles under high vacuum. CPME (1 mL, 0.2 M) was added with vigorous stirring at room temperature, the reaction mixture was placed in a preheated oil bath and stirred for 16 h at 60° C. After 16 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. Purification by chromatography on silica gel (EtOAc/hexanes) afforded the title product in 99% yield (35 mg). White solid. Spectroscopic data matched literature values.

### General Procedure for Amination of Aryl Chlorides: Synthesis of 4-methyl-N-phenylaniline (General Procedure XIV

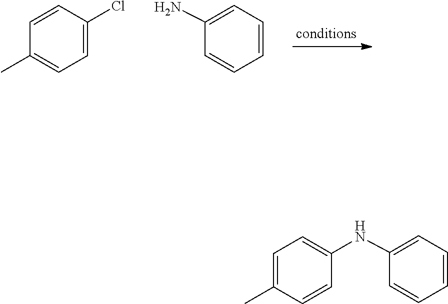

An oven-dried vial equipped with a stir bar was charged with 4-chlorotoluene (25.3 mg, 0.2 mmol, 1.5 equiv), aniline (26 mg, 0.28 mmol, 1.4 equiv), 42 (1.3 mg, 0.002 mmol, 1 mol %), and NaOtBu (26.9 mg, 0.28 mmol, 1.4 equiv) placed under a positive pressure of argon, and subjected to three evacuation/backfilling cycles under high vacuum. THF (1 mL, 0.2 M) was added with vigorous stirring at room temperature, the reaction mixture was placed in a preheated oil bath and stirred for 16 h at 60° C. After 16 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. Purification by chromatography on silica gel (EtOAc/hexanes) afforded the title product in 99% yield (36.3 mg). White solid. ¹H NMR (500 MHz, CDCl₃) δ 7.25-7.19 (m, 2H), 7.07 (d, J=8.2 Hz, 2H), 7.02-6.95 (m, 4H), 6.86 (t, J=7.3 Hz, 1H), 5.64 (s, 1H), 2.29 (s, 3H). ¹³C NMR (125 MHz, CDCl₃) δ 144.06, 140.39, 131.11, 130.00, 129.45, 120.47, 119.06, 117.02, 20.83.

### General Procedure for Suzuki-Miyaura Cross-Coupling of Aryl Chlorides: Synthesis of 4-methoxy-1,1′-biphenyl (General Procedure XV)

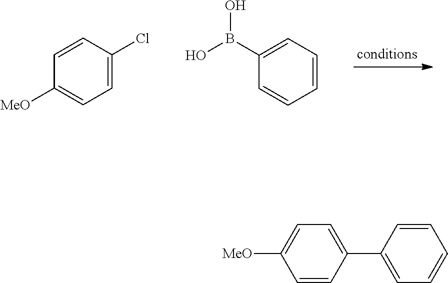 [SMILES: COc1ccc(Cl)cc1 ; OB(O)c1ccccc1 ; COc1ccc(-c2ccccc2)cc1]

An oven-dried vial equipped with a stir bar was charged with 4-chloroanisole (28.5 mg, 0.2 mmol, 1.5 equiv), phenylboronic acid (36.6 mg, 0.3 mmol, 1.5 equiv), 42 (1.3 mg, 0.002 mmol, 1 mol %), and KOtBu (33.6 mg, 0.3 mmol, 1.5 equiv) placed under a positive pressure of argon, and subjected to three evacuation/backfilling cycles under high vacuum. nBuOH (1 mL, 0.2 M) was added with vigorous stirring at room temperature, the reaction mixture was placed in a preheated oil bath and stirred for 16 h at 60° C. After 16 h, the reaction mixture was diluted with CH₂Cl₂ (10 mL), filtered, and concentrated. Purification by chromatography on silica gel (EtOAc/hexanes) afforded the title product as white solid. Yield 93% (34.2 mg). ¹H NMR (500 MHz, CDCl₃) δ 7.59-7.52 (m, 4H), 7.43 (t, J=7.8 Hz, 2H), 7.31 (t, J=7.4 Hz, 1H), 6.99 (d, J=8.8 Hz, 2H), 3.86 (s, 3H). ¹³C NMR (125 MHz, CDCl₃) δ 159.28, 140.97, 133.92, 128.86, 128.29, 126.88, 126.79, 114.34, 55.49.

### General Procedure for Amination of Aryl Fluorides (Ar—F) (General Procedure XVI)

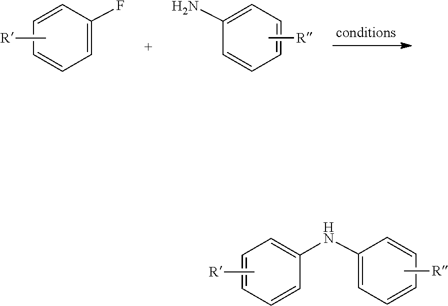

An oven-dried vial equipped with a stir bar was charged with arylfluoride (neat, 1.0 equiv), aniline (typically, 1.5 equiv), [Pd(ligand)Cl]₂ (typically, 2.5 mol %), and NHC·HCl (10 mol % or 20 mol %) placed under a positive pressure of argon, and subjected to three evacuation/backfilling cycles under high vacuum. Toluene (typically, 0.2 M) and LiHMDS in toluene (1 M solution, 2 equiv) was added with vigorous stirring at room temperature, the reaction mixture was placed in a preheated oil bath (typically, 130° C.) and stirred for 16 h. After 16 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. A sample was analyzed by ¹H NMR (CDCl₃, 500 MHz) and GC-MS to obtain conversion, and yield using internal standard and comparison with authentic samples. Purification by chromatography on silica gel afforded the title product. In certain embodiments, “ligand” is cinnamyl. In certain embodiments, NHC is 5-(2,4,6-phenyl)-2-(2,6-diisopropylphenyl)imidazo[1,5-α]pyridin-2-ium chloride. In certain embodiments, [Pd(ligand)Cl]₂ and NHC·HCl react to form compound 74 in situ.

### Synthesis of 4-methoxy-N-phenylaniline (General Procedure XVI)

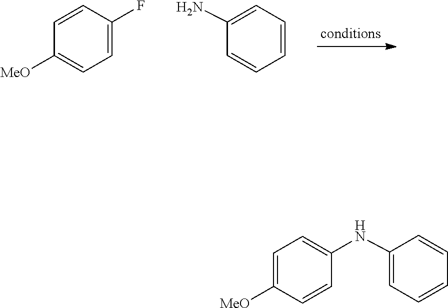 [SMILES: COc1ccc(F)cc1 ; Nc1ccccc1 ; COc1ccc(Nc2ccccc2)cc1]

An oven-dried vial equipped with a stir bar was charged with 4-fluoroanisole (25.2 mg, 0.2 mmol, 1.0 equiv), aniline (28 mg, 0.3 mmol, 1.5 equiv), [Pd(cin)Cl]₂ (2.6 mg, 0.0025 mmol, 2.5 mol %), and 5-(2,4,6-phenyl)-2-(2,6-diisopropylphenyl)imidazo[1,5-α]pyridin-2-ium chloride (9.5 mg, 0.01 mmol, 10 mol %) placed under a positive pressure of argon, and subjected to three evacuation/backfilling cycles under high vacuum. Toluene (1 mL, 0.2 M) and LiHMDS in toluene (1 M, 0.4 mL, 0.4 mmol, 2 equiv) was added with vigorous stirring at room temperature, the reaction mixture was placed in a preheated oil bath (130° C.) and stirred for 16 h. After 16 h, the reaction mixture was cooled down to room temperature, quenched with saturated ammonium chloride solution (5 mL) and extracted with ethyl acetate (2×5 mL). The combined organic layers were dried over anhydrous Na₂SO₄ and concentrated under reduced pressure. Purification by chromatography on silica gel afforded 4-methoxy-N-phenylaniline as brown solid. Yield 90% (35.8 mg). ¹H NMR (500 MHz, CDCl₃) δ 7.22 (t, J=7.5 Hz, 2H), 7.08 (d, J=8.9 Hz, 2H), 6.92 (d, J=7.6 Hz, 2H), 6.87 (d, J=8.9 Hz, 2H), 6.84 (t, J=7.4 Hz, 1H), 5.50 (s, 1H), 3.81 (s, 3H). ¹³C NMR (126 MHz, CDCl₃) δ 155.42, 145.31, 135.86, 129.44, 122.35, 119.70, 115.78, 114.81, 55.72.

### General Procedure for the Reductive Amination of Alkynes with Nitroarenes (General Procedure XVII)

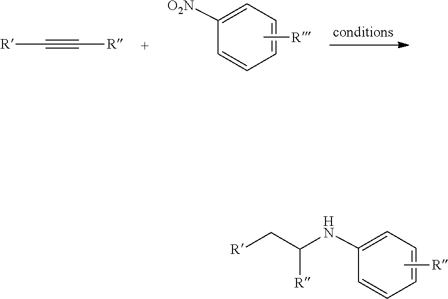

An oven-dried vial equipped with a stir bar was charged with alkyne (if solid, neat, 1.0 equiv), nitroarene (typically, 2.0 equiv), [(NHC)AuCl](typically, 5 mol %), Lewis acid (10 mol %) and 4 Å molecular sieve (MS) (typically, 100 mg). Toluene or trifluorotoluene (typically, 0.25 M), phenylsilane (typically, 10 equiv) and alkyne (if liquid, neat, 1.0 equiv) were added with vigorous stirring. Water (typically, 5 equiv) was added if needed, and the reaction mixture was placed in a preheated oil bath (typically, 40° C.) and stirred for 16 h. After 16 h, the reaction mixture was cooled down to room temperature, diluted with CH₂Cl₂ (10 mL), filtered, and concentrated. A sample was analyzed by ¹H NMR (CDCl₃, 500 MHz) and GC-MS to obtain conversion, and yield using internal standard and comparison with authentic samples. Purification by chromatography on silica gel afforded the tile product. In certain embodiments, (NHC)AuX is compound 52. In certain embodiments, Lewis acid is [Ag(MeCN)₂]⁺BARF⁻.

### Synthesis of 4-methoxy-N-(1-phenylethyl)aniline (General Procedure XVII)

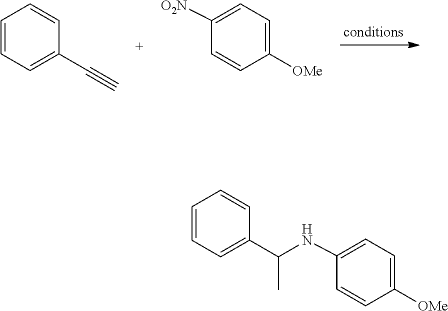

An oven-dried vial equipped with a stir bar was charged with 4-nitroanisole (61.2 mg, 0.4 mmol, 2.0 equiv), (52) [(5-(2,6-Diisopropylphenyl)-2-mesitylimidazo[1,5-α]pyridin-3(2H)-ylidene)gold(I) chloride](6.3 mg, 0.01 mg, 5 mol %), [Ag(MeCN)₂]⁺BARF⁻ (21 mg, 0.02 mmol, 10 mol %) and 4 Å molecular sieve (MS) (100 mg). Toluene (0.8 mL, 0.25 M), phenylsilane (216 mg, 0.2 mmol, 10 equiv) and phenylacetylene (20.4 mg, 0.2 mmol, 1.0 equiv) were added with vigorous stirring. The reaction mixture was placed in a preheated oil bath (40° C.) and stirred for 16 h. After 16 h, the reaction mixture was cooled down to room temperature, diluted with CH₂Cl₂ (10 mL), filtered, and concentrated. Purification by chromatography on silica gel afforded 4-methoxy-N-(1-phenylethyl)aniline as colorless oil. Yield 91% (41.4 mg).

### Example 5: Characterization of NHC Ligands and Ligand-Metal Complexes

### Electronic Characterization

NHC—Se (5 complexes) and NHC—Rh(I) complexes (3 complexes) were synthesized for characterization purposes of p-acceptance and s-donation, respectively. The dsₑ values of 119.35 ppm for (71) (CDCl₃), 118.02 ppm for (72), 170.40 ppm for (73), 143.44 ppm for (70) and 100.66 ppm for (69) can be compared with IPr (dsₑ=90 ppm), IPr* (dsₑ=106 ppm), and IMes (dsₑ=27 ppm), indicating stronger p-accepting properties of this class of ligands. The avg. n_{CO}=2031.6 cm⁻¹ for (68) (TEP of 2045.3 cm⁻¹), the avg. n_{CO}=2032.2 cm⁻¹ for (64) (TEP of 2045.8 cm⁻¹), and the avg. n_{CO}=2031.2 cm⁻¹ for (66) (TEP of 2045.0 cm⁻¹) provide a combined measure of the electronic properties of the ligand and can be compared with IPr (TEP of 2051.5 cm⁻¹), IPr* (TEP of 2052.7 cm⁻¹) and IMes (TEP of 2050.8 cm⁻¹), indicating strong σ-donation of this class of ligands. One-bond CH J coupling constants from ¹³C satellites of the ¹H NMR spectrum (NHC salt) provide good indication of σ-donating properties of an NHC ligand. The value of 225 Hz for ImPy-Dipp-IMes L-shaped ligand (CDCl₃) is consistent with this ligand being strongly σ-donating (cf., IPr: ¹J_{CH}=223.7 Hz).

### Complexation Studies

The complexation studies demonstrate that ligands of the present disclosure can be readily engaged in the synthesis of well-defined, moisture- and air-stable complexes with Pd(II), Au(I), Ag(I), Cu(I), Rh(I) and Se. All prepared complexes were found to be air- and moisture-stable in a solid state, permitting their application in catalysis.

### X-Ray Structural Analysis

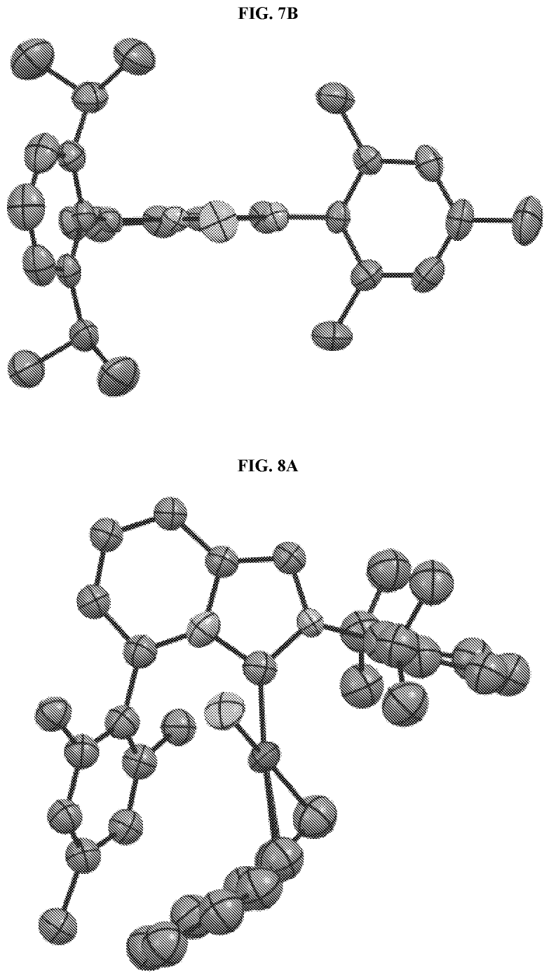

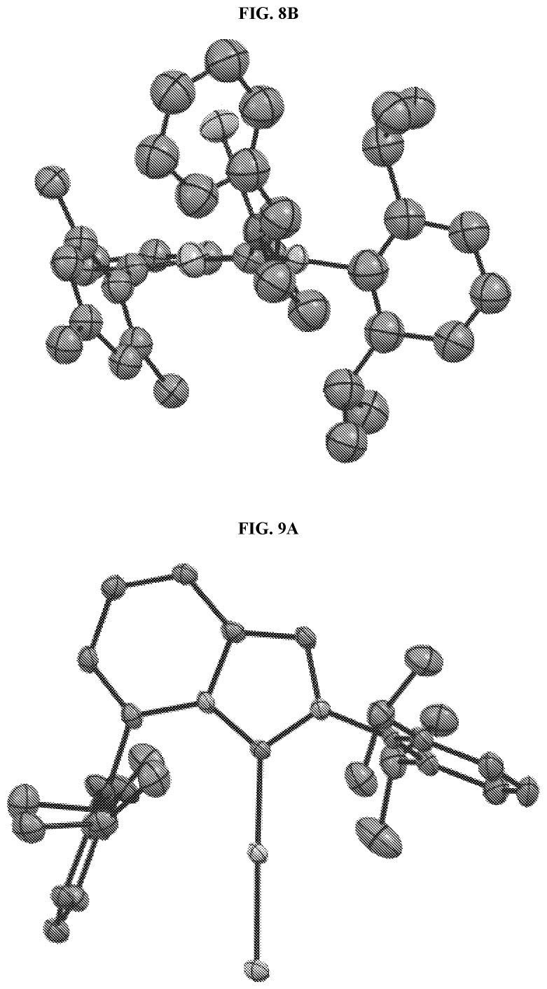

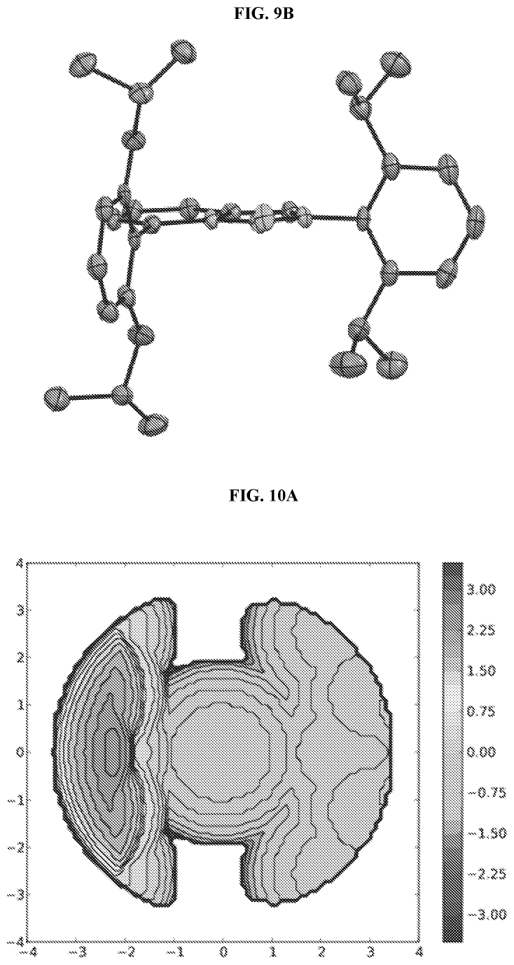

The structures of eight Pd(II) complexes, six Au(I) complexes, and representative Cu(I) and Rh(I) complexes have been fully characterized by x-ray crystallographic analysis (Table 2 and FIGS. 7A-7B, FIGS. 8A-8B, and FIGS. 9A-9B).

TABLE 2
Buried volume (% V_{bur}) of selected compounds

| Compound | Metal | % V_{bur} |
|---|---|---|
| 51^{a} | Au | 46.4% |
| 52^{a} | Au | 49.5% |
| 53^{a} | Au | 59.6% |
| 54^{a} | Au | 68.7% |
| 56^{a} | Au | 52.6% |
| [Au(34)Cl]^{a} | Au | 50.9% |
| 37^{b} | Pd | 42.0% |
| 39^{b} | Pd | 43.2% |
| 41^{b} | Pd | 42.1% |
| 43^{b} | Pd | 45.6% |
| 38^{c} | Pd | 41.7% |
| 40^{d} | Pd | 40.9% |
| 47^{e} | Pd | 43.8% |
| 49^{f} | Pd | 39.0% |
| 59^{g} | Cu | 62.0% |
| 64^{h} | Rh | 42.0% |

^{a}[Au(NHC)Cl];
^{b}[Pd(NHC)(cin)Cl];
^{c}[Pd(NHC)(allyl)Cl];
^{d}[Pd(NHC)(1-t-Bu-ind)Cl];
^{e}[Pd(NHC)(3-Cl-py)Cl₂];
^{f}[Pd(NHC)(C₆H₄—CH₂NMe₂)Cl];
^{g}[Cu(NHC)Cl];
^{h}[Rh(NHC)(CO)₂Cl].

### Structural Characterization

The % buried volume (% V_{bur}) of [Au(34)Cl], is 50.9%, which can be compared with (% V_{bur}) of 51.9% determined for [Au(35)Cl], and (% V_{bur}) of 52.6% determined for 56. These values define ideal catalytic pocket to accommodate substrates for Pd-catalyzed cross-couplings of usual substrates by this class of biaryl L-shaped ligands. In contrast, (% V_{bur}) of 54.1% determined for [Au(36)Cl], and (% V_{bur}) of 46.4% determined for 51, are respectively too large and too small for efficient catalysis in Pd-catalyzed cross-couplings of usual substrates.

Furthermore, (% V_{bur}) of 49.5% determined for 52 is in the expected range for opening of the catalytic pocket by removing 4-substitution, while maintaining 2,6-di-ortho substitution of the biaryl ring (cf. [Au(35)Cl], 51.9%). Finally, (% V_{bur}) of 59.6% determined for 53 and (% V_{bur}) of 58.7% determined for 54 are indicative of bulky-yet-flexible biaryl L-shaped ligands and can be compared with (% V_{bur}) of 50.4% determined for [Au(IPr*)Cl]. This steric range of catalytic pocket is ideal for Pd-catalyzed cross-coupling of small nucleophiles, where flexibility of the dibenzhydryl motifs provides selective steric environment of the catalytic pocket.

The comparison of (% V_{bur}) for linear metal-NHC complexes of sterically-defined biaryl L-shaped NHC ligands with their square-planar complexes in the same series of ligands may indicate that these ligand are capable of adjusting to the steric environment. For example, using (34) as a representative ligand (% V_{bur}) changes from 40.9% to 50.9% (D of 25%) from the [Pd(34)(1-t-Bu-ind)Cl] to [Au(34)Cl]complex. Furthermore, there is steric differentiation even within square-planar Pd(II) complexes with different ligands on Pd, corresponding to the decrease of (% V_{bur}) with an increasing steric demand of the ligand, [Pd(34)(1-t-Bu-ind)Cl](% V_{bur}=40.9%)<[Pd(34)(allyl)Cl](% V_{bur}=41.7%)<[Pd(34)(cin)Cl](% V_{bur}=43.2%)<[Pd(34)(3-Cl-py)Cl₂](% V_{bur}=43.8%).

Finally, the % buried volume (% V_{bur}) of sterically-demanding bulky-yet-flexible (59) is 62.0%, which represents the largest recorded % buried volume for any NHC ligand to date. The rigid steric arrangement of biaryl motif in combination with adjusting to the steric environment and flexibility match of the C₅/N₂ substituents provides unique steric environment for catalysis, which is unavailable in other classes of NHC ligands developed to date.

### DFT Analysis

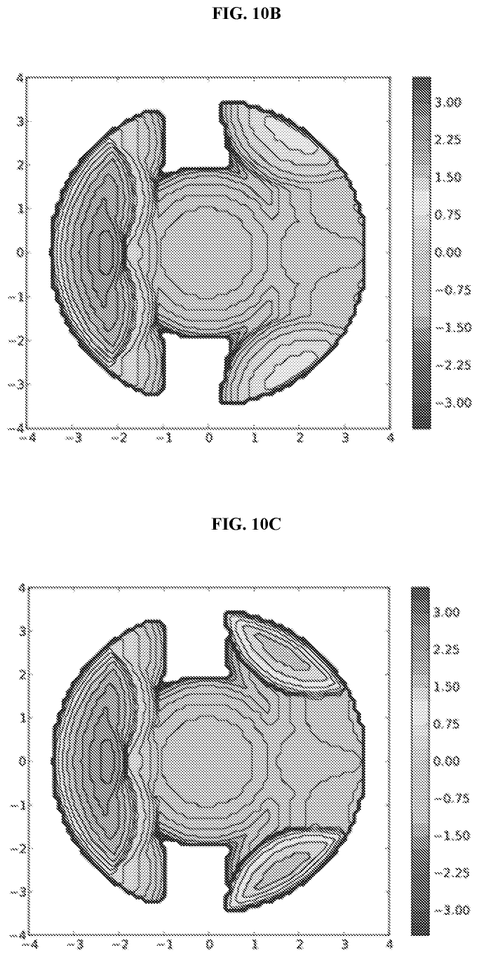

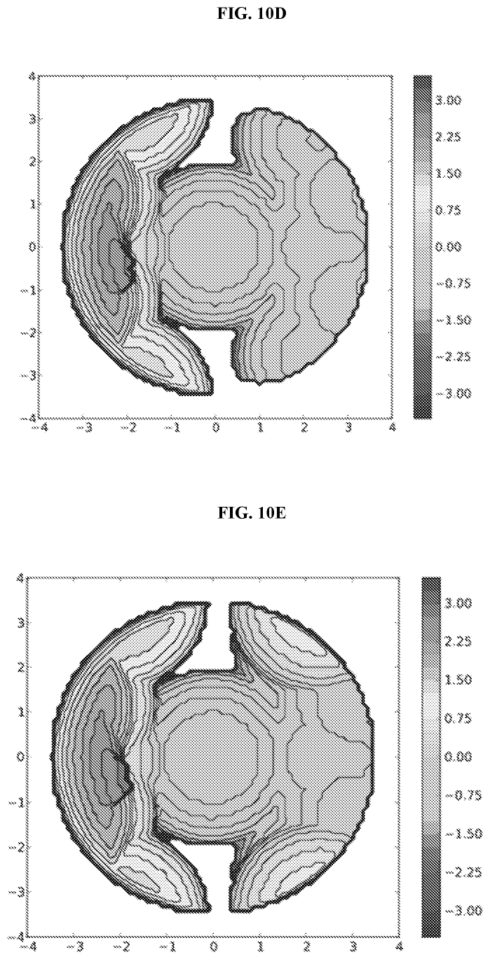

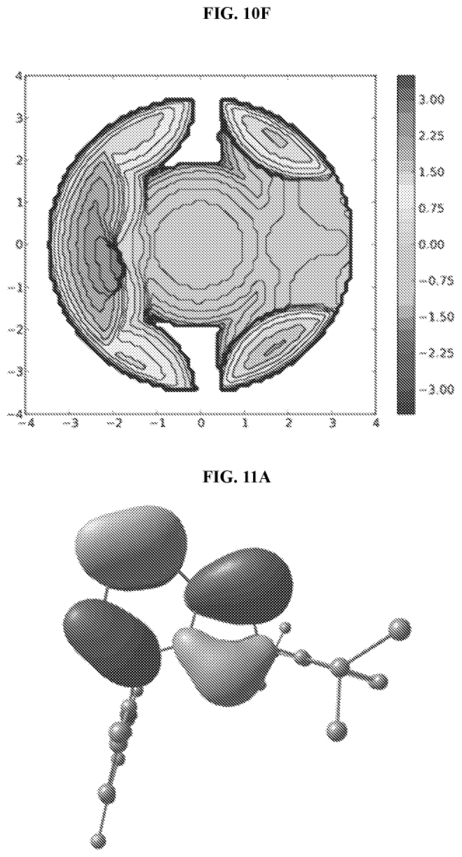

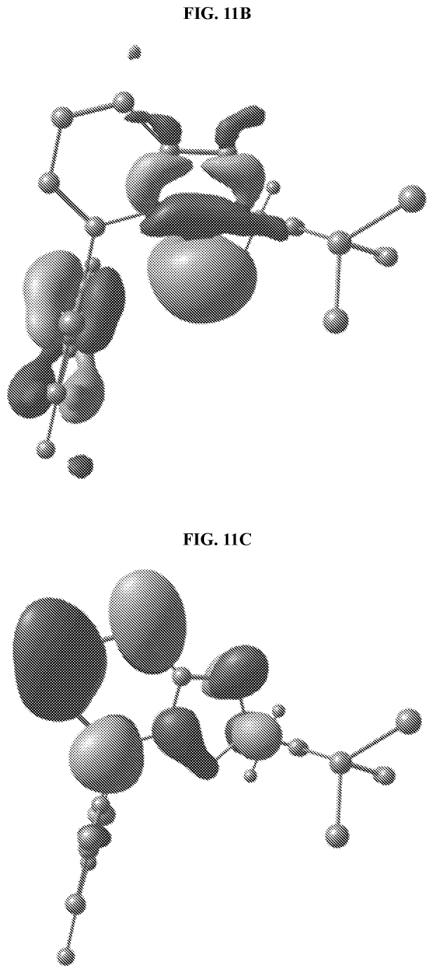

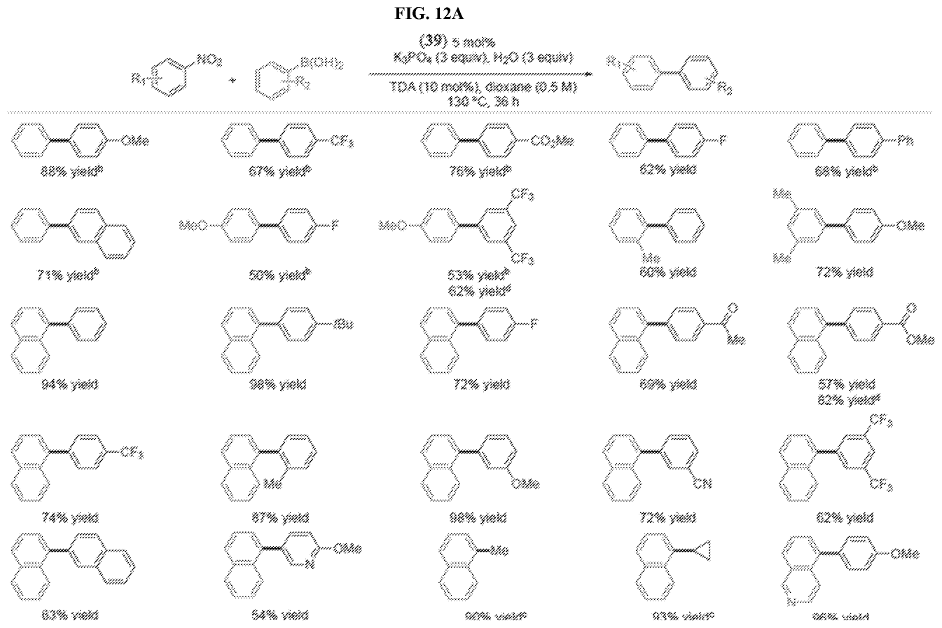

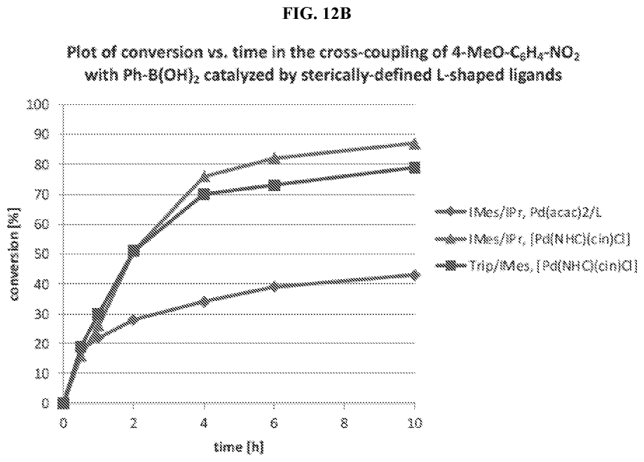

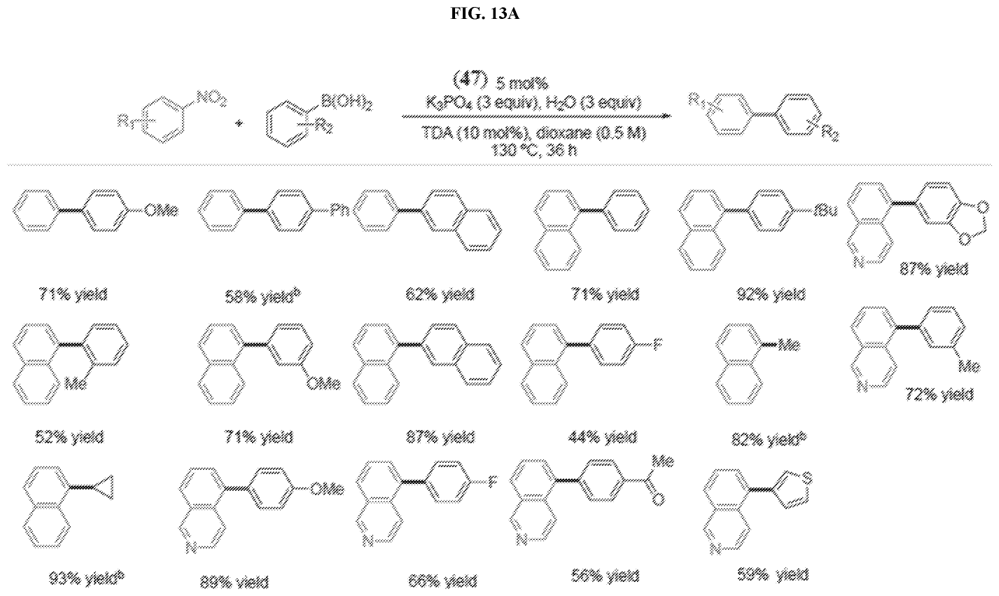

To gain insight into the steric and electronic structure of the sterically-defined L-shaped ligands, computations at the B3LYP 6-311++g(d,p) level of theory were employed (FIGS. 10A-10F).

### Steric Effect

To evaluate the steric effect, geometry and the percent buried volume (% V_{bur}) were calculated for the linear [Cu(NHC)Cl]complexes. The % buried volume (% V_{bur}) of [Cu(NHC)Cl] in the series of ligands 33, 34, and 17, is 46.8%, 50.1%, 51.8% respectively, and in the series of ligands 35, 36, and 19 is 49.8%, 52.4%, 54.2% respectively. There is an excellent linear correlation between the steric demand of the N₂-wingtip substituent as a function of steric demand of C₅-substituent (R²=0.99).

The steric matching between C₅ and N₂ substituents supports the findings from x-ray crystallographic analysis. Biaryl L-shaped ligands with C₅/N₂ Dipp/IMes combination are sterically similar (% V_{bur}=49.8%-50.1%) with catalytic pocket accommodating substrates for Pd-catalyzed cross-coupling. C₅/N₂ substitution with sterically-more demanding substituents results in a decrease in size of catalytic pocket (% V_{bur}=51.8%-54.2%), while C₅/N₂ substitution with less sterically-demanding substituents leads to low % buried volume in this series of ligands (% V_{bur}=46.8%). Quadrant distribution of 34 is 55.3%, 55.3%, 45.0%, 45.0% for each quadrant, and for 35 is 60.9%, 61.4%, 38.5%, 38.2%.

The % buried volume values can be compared with the reference imidazolylidene [Cu(IMes)Cl], [Cu(IPr)Cl] and [Cu(IPr*)Cl] with (% V_{bur}) of 36.4%, 42.6% and 49.7% determined at the same level of theory.

### Electronic Properties

To evaluate electronic properties of sterically-defined L-shaped ligands, HOMO and LUMO energy levels were determined at the B3LYP 6-311++g(d,p) level of theory (FIGS. 11A-11C). The HOMO-1 (s-bonding orbital) in the series of 51, 34, and 17 is −5.86 eV, −5.91 eV, −5.96 eV and in the series of 35, 36, and 19 is −5.87 eV, −5.91 eV, −5.95 eV, which is in the same range as for the standard imidazolylidene ligands IMes (−5.90 eV), IPr (−6.01 eV), IPr* (−6.12 eV) determined at the same level of theory. Furthermore, the LUMO (p-accepting orbital) in the series of 33, 34, and 17 is −1.24 eV, −1.29 eV, −1.25 eV and in the series of 35, 36, and 19 is −1.25 eV, −1.30 eV, −1.25 eV, which is much lower than for the standard imidazolylidene ligands IMes (−0.33 eV), IPr (−0.48 eV), IPr* (−0.90 eV) determined at the same level of theory.

In addition, the HOMO (p-donating orbital) in the series of 33, 34, and 17 is −5.43 eV, −5.49 eV, −5.42 eV and in the series of 35, 36, and 19 is −5.44 eV, −5.50 eV, −5.40 eV, which is much higher than the p-donating orbital for the standard imidazolylidene ligands IMes (−6.44 eV), IPr (−6.55 eV), IPr* (−6.28 eV) determined at the same level of theory.

This indicates that (1) sterically-defined L-shaped ligands are strongly nucleophilic ligands, as expected for sterically-bulky N—Ar NHC ligands, with Cₐ-donation matching those of IPr and IMes and significantly stronger than phosphines (e.g. dialkylbiarylphosphines), and (2) significantly better n-acceptors than the standard imidazolylidene IPr and IMes ligands. Furthermore, biaryl L-shaped ligands are characterized by strong p-donating abilities, which is not available in the standard imidazolylidene IPr and IMes ligands.

### Catalysis

Catalytic studies were conducted to evaluate the activity of the ligands, and/or catalyst complexes thereof, of the present disclosure. From the outset, these ligands have been designed to utilize the pivotal biaryl arrangement that stabilizes the reactive ligand-metal intermediates through sterically-defined rigid five-membered interaction in palladium cross-coupling catalysis. Palladium-catalyzed cross-coupling reactions represent an important application of ligands in contemporary transition-metal-catalysis with fundamental and practical significance in medicinal chemistry, drug discovery, biochemistry, agrochemistry, natural product synthesis, small molecule synthesis and polymer synthesis.

Specifically, activation of unreactive bonds was targeted, such as C—N, C—S and C—O, which is one of the most underdeveloped areas in cross-coupling chemistry. Activation of stronger C—X bonds (X═N, S, O) requires strongly σ-donating ligands to promote oxidative addition, while stabilizing interaction between the ipso position of the biaryl ring and palladium further lowers the barrier to oxidative addition. The net result is cross-coupling of typically unreactive C—X bonds, which have a number of synthetic advantages: (1) show orthogonal reactivity to the classical aryl halides and pseudohalides, (2) provide synthetic handles in molecule functionalization, (3) can be applied to the highly-desirable late-stage functionalization, (4) are derived from different pool of precursors than aryl halides and pseudohalides.

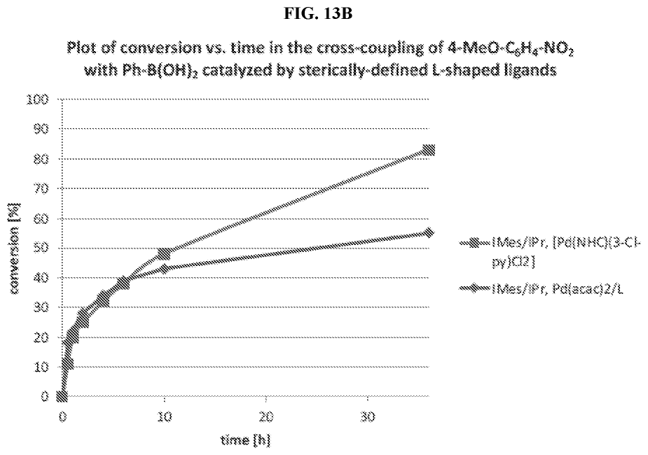

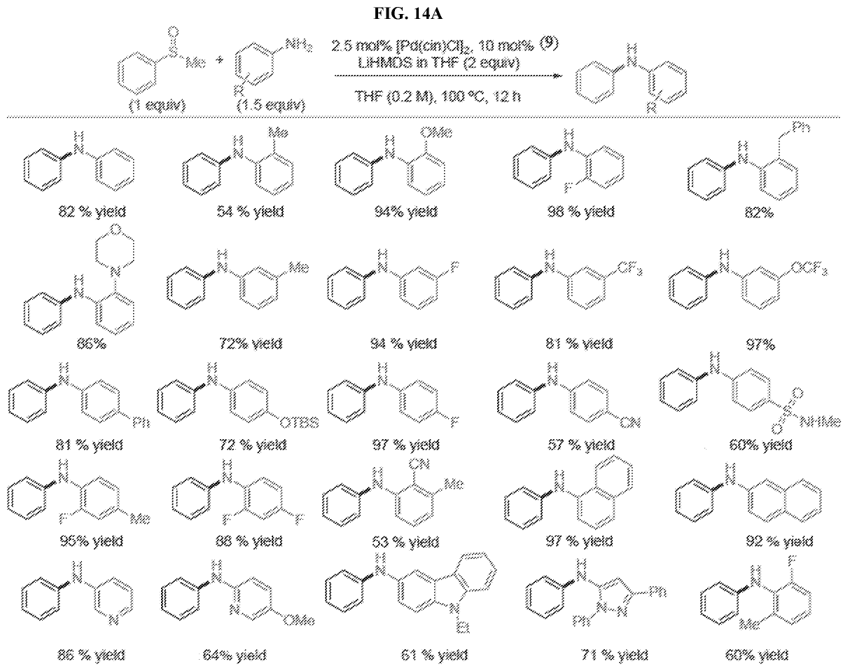

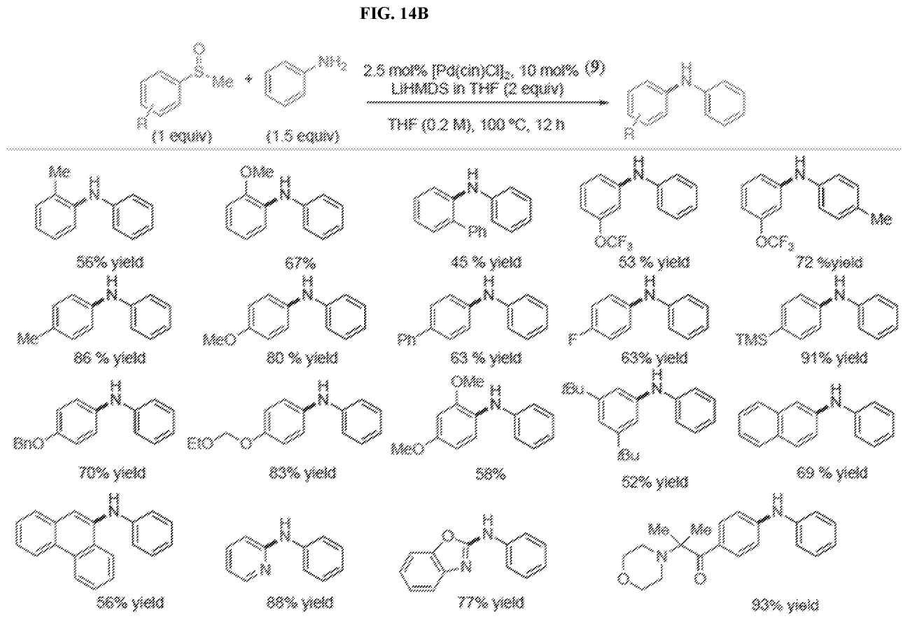

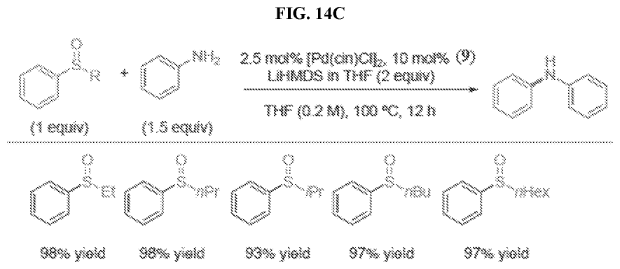

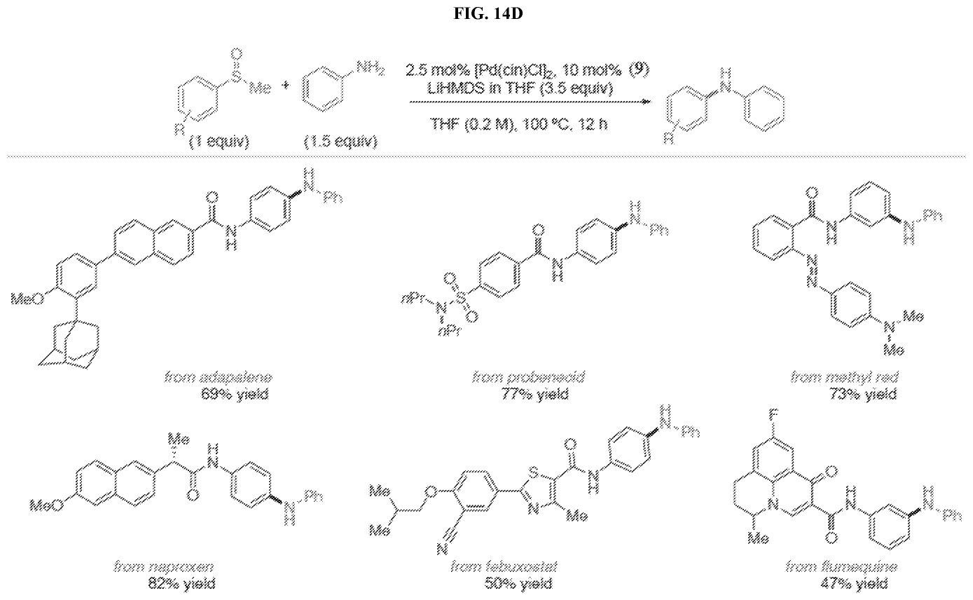

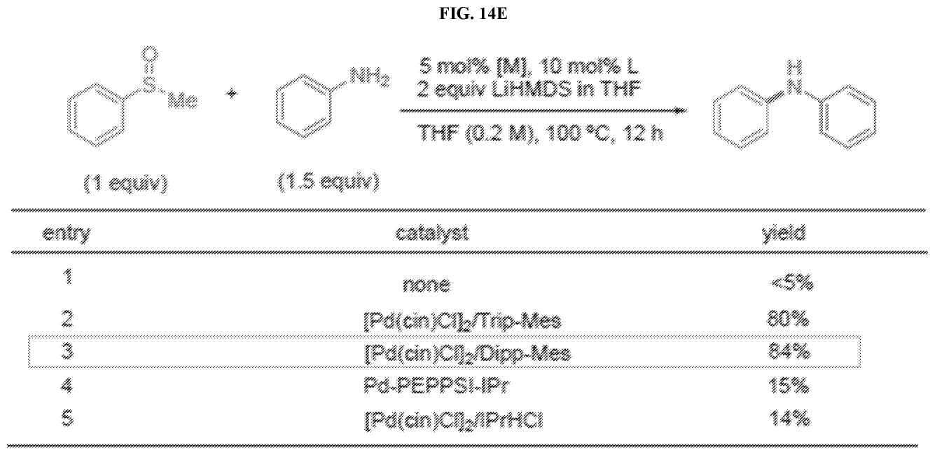

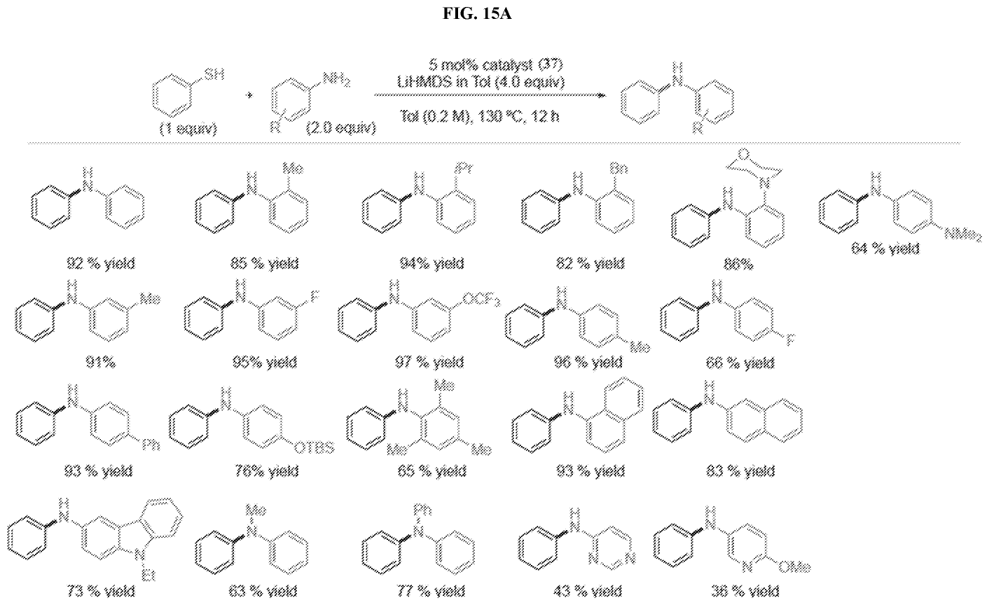

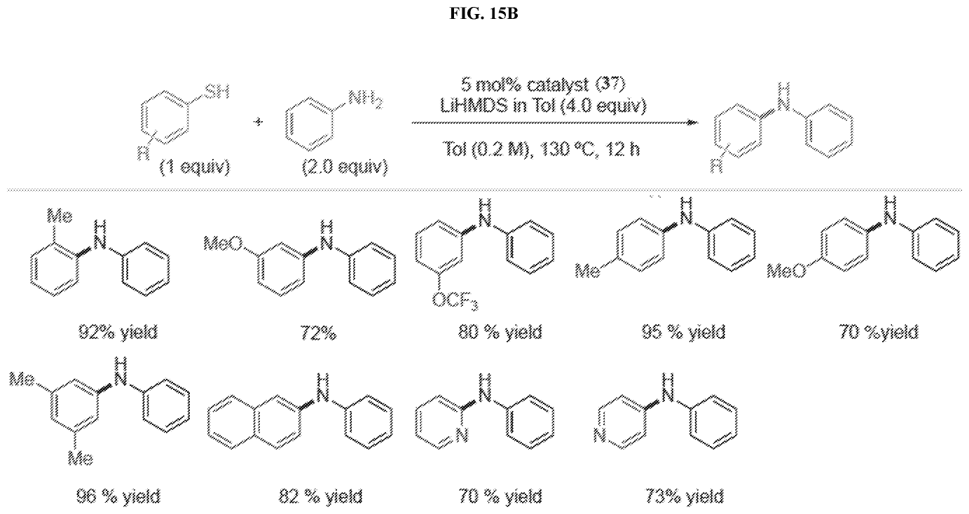

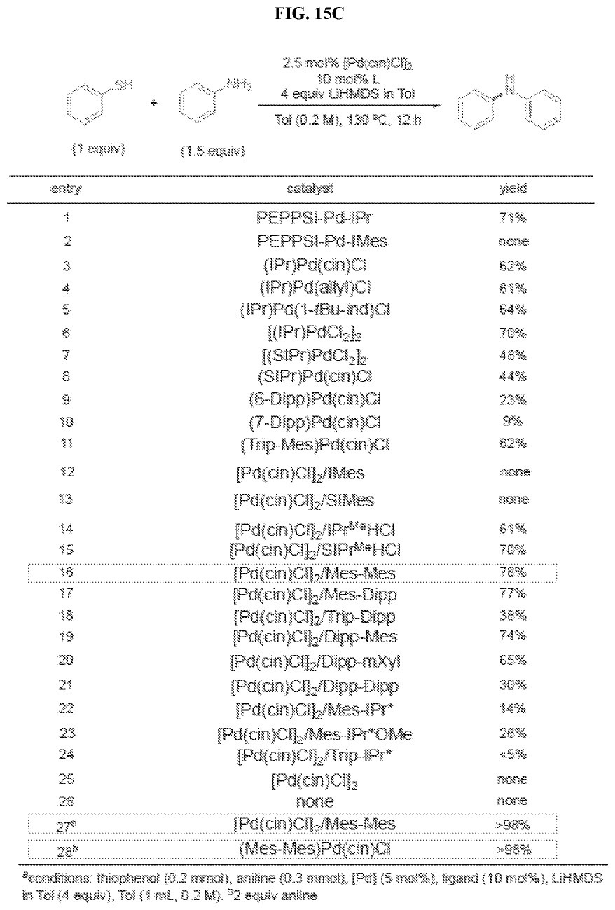

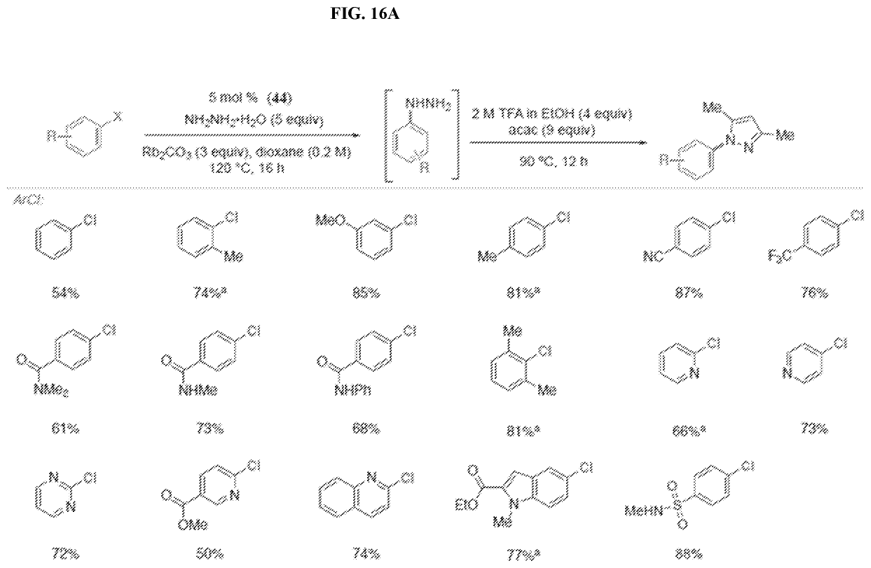

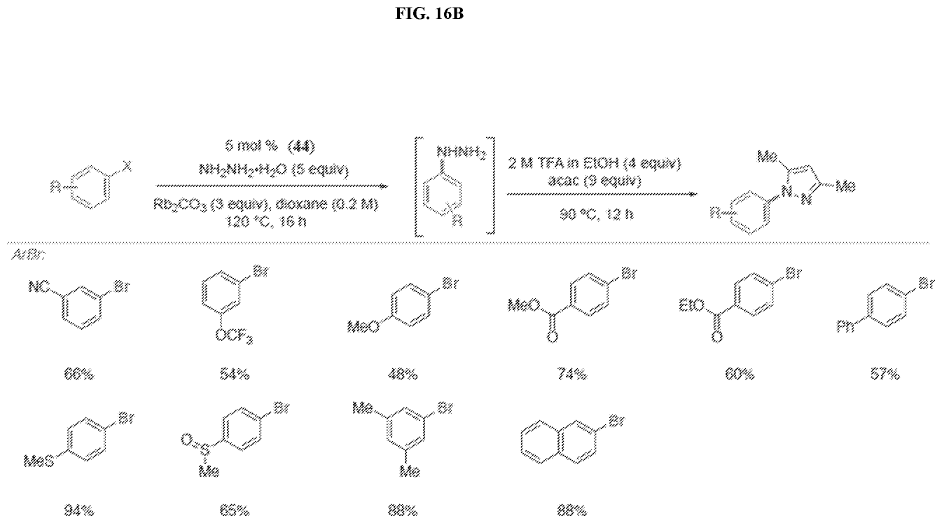

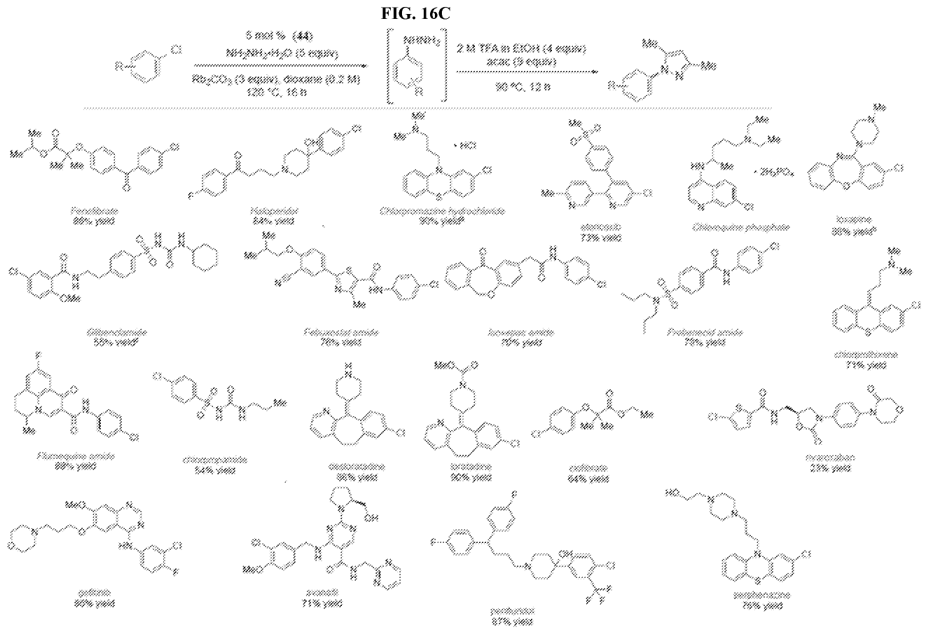

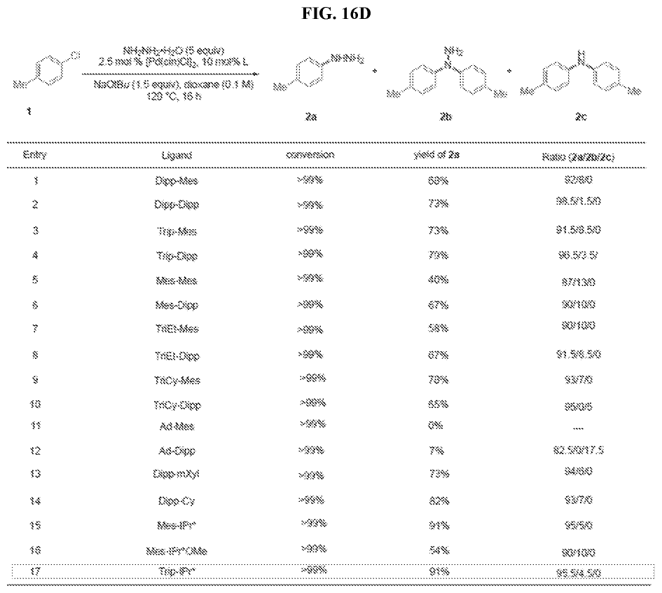

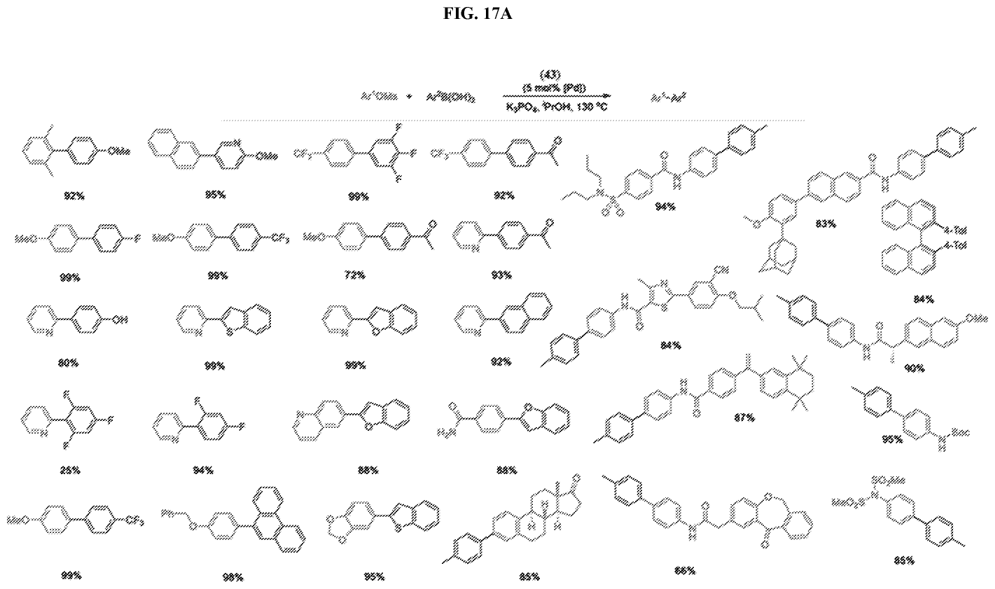

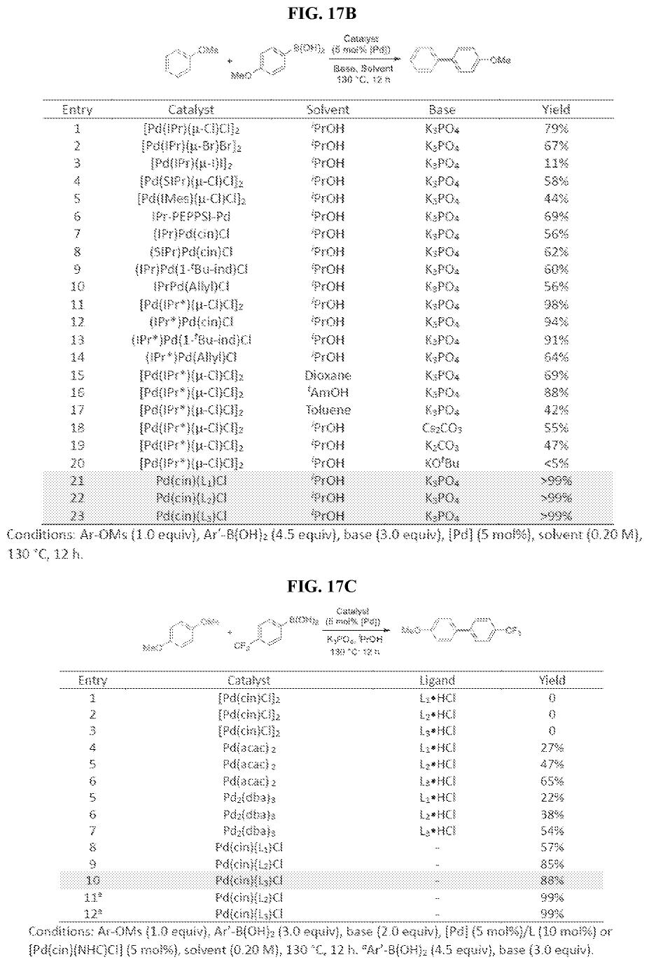

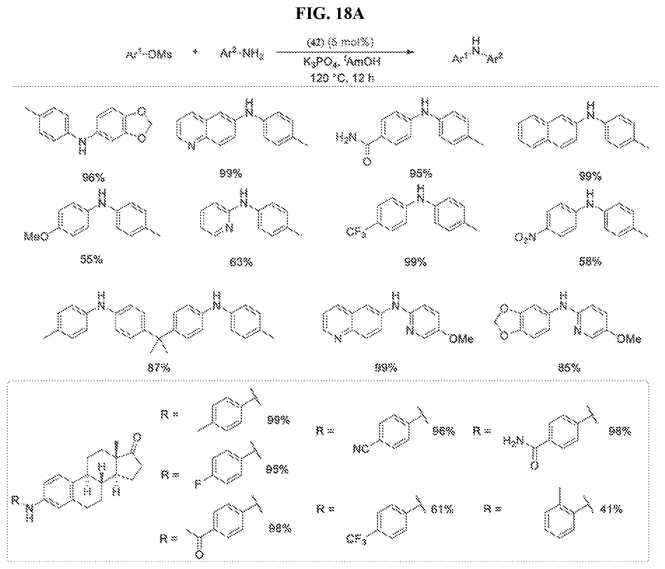

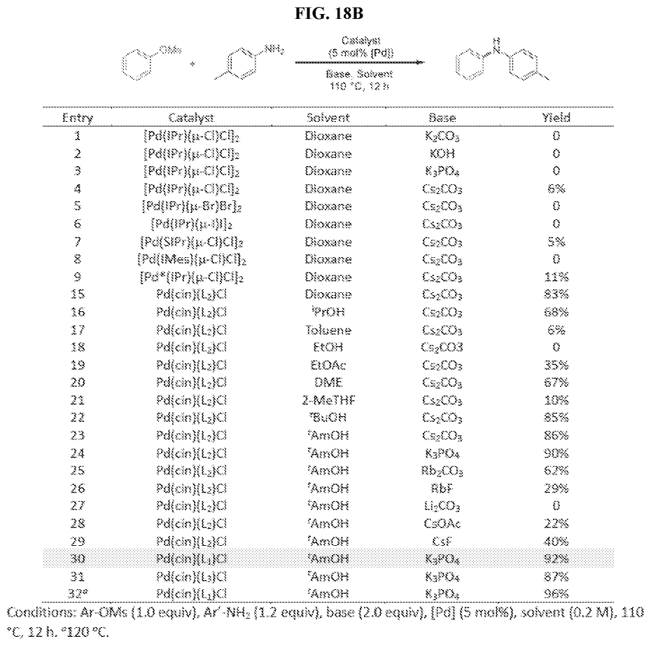

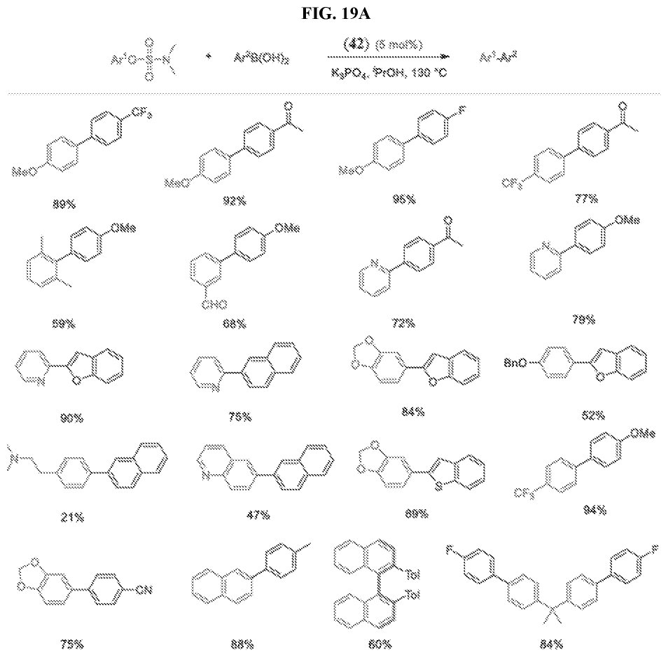

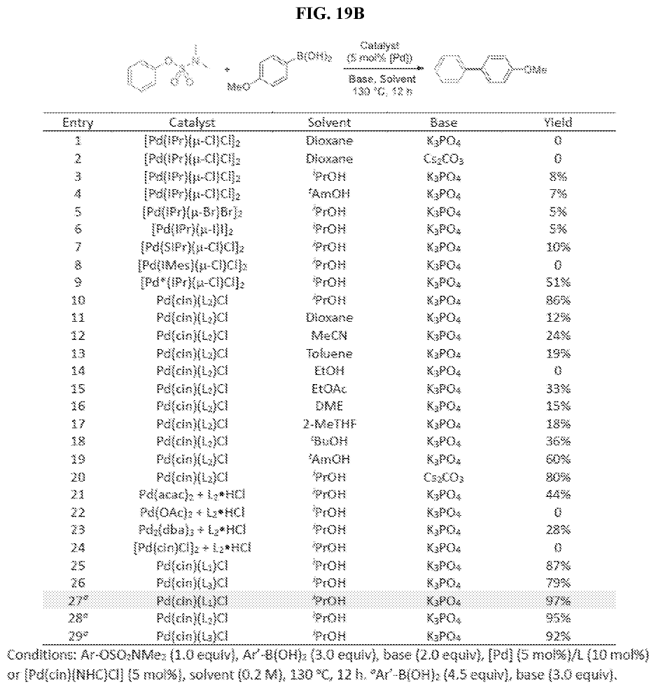

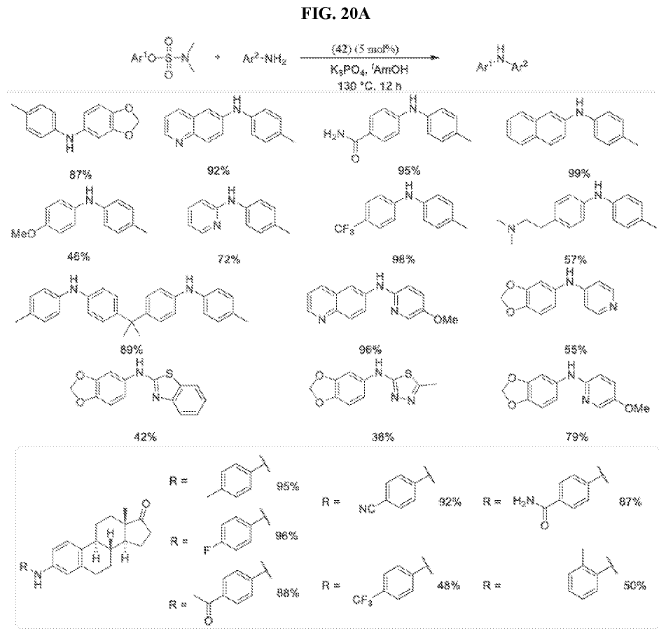

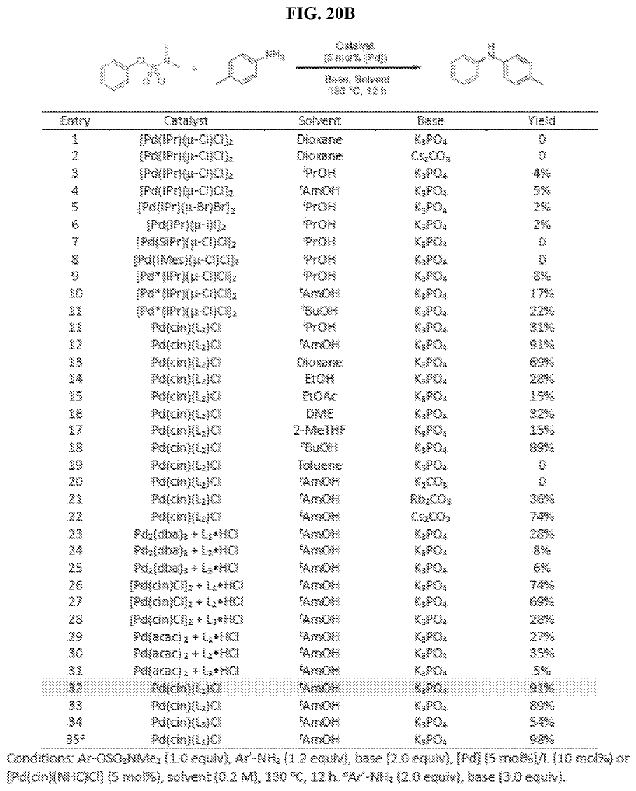

The following non-limiting reactions have been demonstrated using sterically-defined biaryl L-shaped ligands of the present disclosure:
(a) Suzuki-Miyaura cross-coupling of nitroarenes (Ar—NO₂) using well-defined air- and moisture-stable [Pd(NHC)(cin)Cl]precatalysts (FIGS. 12A-12B).
(b) Suzuki-Miyaura cross-coupling of nitroarenes (Ar—NO₂) using well-defined air- and moisture-stable [Pd-PEPPSI]precatalysts (FIGS. 13A-13B).
(c) Buchwald-Hartwig amination of alkyl sulfoxides (Ar—S(O)R, R=alkyl) (FIGS. 14A-14E). *Sterically-defined L-shaped ligands are required for this reaction. Classical NHCs show negligible reactivity (<20%). Phosphine ligands show negligible reactivity.
(d) Buchwald-Hartwig amination of thiophenols (Ar—Cl) (FIGS. 15A-15C). Sterically-defined L-shaped ligands show higher reactivity than classical NHCs. Without wishing to be bound by theory, the reactivity difference might be due to steric-tuning of the catalytic pocket not possible with imidazolylidene and related ligands. Phosphine ligands show negligible reactivity.
(e) Monoarylation of parent hydrazine with aryl chlorides (Ar—Cl) (FIGS. 16A-16D). *Sterically-defined L-shaped ligands are required for this reaction. Classical NHCs show negligible reactivity (<20%). Phosphine ligands show limited reactivity. Aryl bromides (Ar—Br) are also efficient substrates. NHC=ImPy-Trip-IPr* is the ligand of choice, as a well-defined [Pd(NHC)(cin)Cl]complex. Since the reaction requires weak bases for broad functional group tolerance, without wishing to be bound by theory, the reaction may be ideal for well-defined Pd—NHC catalysts (i.e., in situ preparation is not effective).
(f) Suzuki-Miyaura cross-coupling of aryl mesylates and tosylates (Ar—OR, R=methanesulfonoxy or 4-toluenesulfonoxy) (FIGS. 17A-C). Sterically-defined L-shaped ligands show higher reactivity than classical NHCs. IPr* is the only comparable, albeit less reactive NHC ligand, which, without wishing to be bound by theory, might suggest that the high reactivity of L-shaped biaryl ligands is due to steric-tuning of the catalytic pocket. In situ prepared catalyst (Pd(acac)₂/K₃PO₄ or Pd₂(dba)₃/K₃PO₄ also works, but is less reactive. The method represents the first general Suzuki-Miyaura cross-coupling of aryl mesylates/tosylates catalyzed by Pd—NHC systems.
(g) Buchwald-Hartwig amination of aryl mesylates and sulfonates (Ar—OR, R=methanesulfonoxy or 4-toluenesulfonoxy) (FIGS. 18A-18B). Sterically-defined L-shaped ligands are required for this reaction. Alternative NHCs show negligible reactivity (<10%). In situ prepared catalyst (Pd(acac)₂/K₃PO₄ or Pd₂(dba)₃/K₃PO₄ also works, but is less reactive. The method represents the first general Buchwald-Hartwig amination of aryl mesylates catalyzed by Pd—NHC systems.
(h) Suzuki-Miyaura cross-coupling of aryl sulfamates (Ar—OR, R=SO₂NR₂′) (FIGS. 19A-19B). Sterically-defined L-shaped ligands are required for this reaction. Alternative NHCs show negligible reactivity (<10%). IPr* is the only comparable, albeit much less reactive NHC ligand (50%), which might suggest that the high reactivity of L-shaped biaryl ligands is due to steric-tuning of the catalytic pocket. In situ prepared catalyst (Pd(acac)₂/K₃PO₄ or Pd₂(dba)₃/K₃PO₄ also works, but is less reactive. The method represents the first Suzuki-Miyaura cross-coupling of aryl sulfamates catalyzed by Pd—NHC systems.
(i) Buchwald-Hartwig amination of aryl sulfamates (Ar—OR, R=SO₂NR₂′) (FIGS. 20A-20B). Sterically-defined L-shaped ligands are required for this reaction. Classical NHCs show negligible reactivity (<20%). In situ prepared catalyst (Pd(acac)₂/K₃PO₄ or Pd₂(dba)₃/K₃PO₄ also works, but is less reactive. The method represents the first Buchwald-Hartwig amination of aryl sulfamates catalyzed by Pd—NHC systems.

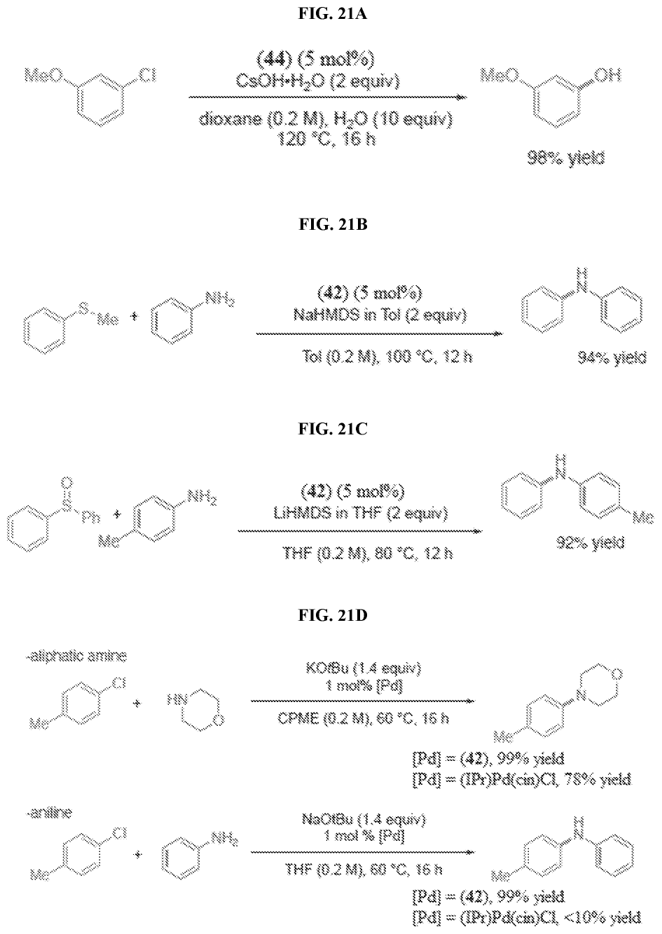

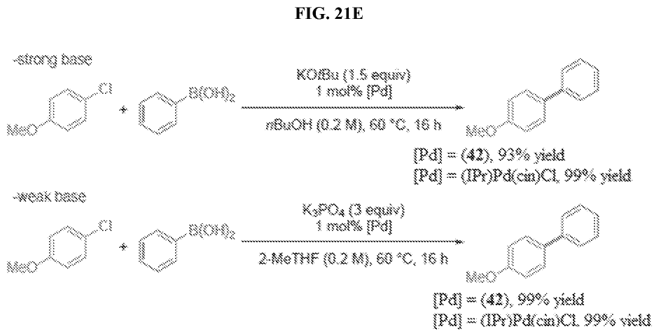

Additionally, feasibility studies have been conducted which further demonstrate the utility of the catalyst complexes described herein:
(a) Hydroxylation of aryl chlorides (Ar—Cl). Sterically-defined L-shaped ligands of the present disclosure are required for this reaction. Alternative NHCs show negligible reactivity (<5%) (FIG. 21A).
(b) Buchwald-Hartwig amination of aryl methyl thioethers (Ar—SMe) (FIG. 21B).
(c) Buchwald-Hartwig amination of aryl sulfoxides (Ar—S(O)R, R=aryl) (FIG. 21C).
(d) Comparative studies have been performed in Buchwald-Hartwig amination of aryl chlorides (Ar—Cl). Sterically-defined L-shaped ligands of the present disclosure showed better reactivity than the standard [Pd(IPr)(cin)Cl](Neolyst CX31, CAS: 884879-23-6). (conditions: KOt-Bu, CPME, 60° C.; NaOt-Bu, THF, 60° C.) (FIG. 21D).
(e) Comparative studies have been performed in Suzuki-Miyaura cross-coupling of aryl chlorides (Ar—Cl). Sterically-defined L-shaped ligands showed comparable reactivity to the standard [Pd(IPr)(cin)Cl](Neolyst CX31, CAS: 884879-23-6). (conditions: KOt-Bu, n-BuOH, 60° C.; K₃PO₄, 2-MeTHF, 60° C.) (FIG. 21E).
(f) Suzuki-Miyaura cross-coupling of aryl pivalates (Ar—OR, R=COt-Bu). Sterically-defined L-shaped ligands of the present disclosure are required for this reaction. Classical NHCs show negligible reactivity (<5%). The process represents the first activation of O-acylated phenols by Pd catalysis.

### Enumerated Embodiments

The following exemplary embodiments are provided, the numbering of which is not to be construed as designating levels of importance:

Embodiment 1 provides a compound of formula (I):

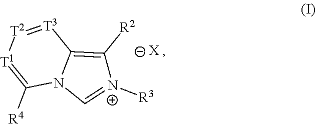

wherein:
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R³ is selected from the group consisting of optionally substituted C₆-C₁₀ aryl, optionally substituted C₂-C₈ heteroaryl, and optionally substituted C₃-C₈ cycloalkyl,
   wherein each optional substituent in R³ is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R⁴ is selected from the group consisting of optionally substituted C₃-C₁₂ cycloalkyl and phenyl substituted with at least one substituent selected from the group consisting of C₁-C₆ alkyl, C₃-C₁₂ cycloalkyl, C₁-C₆ alkoxy, phenyl, and N(R^{A})(R^{B}),
   wherein each optional substituent in the C₃-C₁₂ cycloalkyl is at least one selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
X is a counter anion;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen;
wherein if T¹ is CH, T² is CH, T³, is CH, and R² is H, then each of the following apply:
(a) if R⁴ is 2,4,6-triisopropylphenyl, then R³ is not 2,6-diisopropylphenyl, 2,4,6-trimethylphenyl, 2-methylphenyl, or 2,6-diethyl-4-methylphenyl;
(b) if R⁴ is 2,4,6-trimethylphenyl, then R³ is not 2,4,6-trimethylphenyl, 2,6-diisopropylphenyl, adamantly, 4-(2-(2-methoxyethoxy)ethoxy)-2,6-dimethylphenyl, or (2-(2-methoxyethoxy)ethoxy)phenyl;
(c) if R⁴ is 2-methylphenyl, then R³ is not 2,6-diisopropylphenyl;
(d) if R⁴ is 4-tert-butyl-2-pyridyl, then R³ is not 2,6-diisopropylphenyl; and
(e) if R⁴ is 5-tetrahydroisoquinolinyl, then R³ is not 2,6-diisopropylphenyl.

Embodiment 2 provides the compound of Embodiment 1, wherein at least one of the following applies:
(a) T¹ is CR^{1a};
(b) T² is CR^{1b}; and
(c) T³ is CR^{1c}.

Embodiment 3 provides the compound of Embodiment 1 or 2, wherein at least one of the following applies:
(a) at least one of R^{1a}, R^{1b}, and R^{1c} is H;
(b) at least two of R^{1a}, R^{1b}, and R^{1c} are H; and
(c) each of R^{1a}, R^{1b}, and R^{1c} are H.

Embodiment 4 provides the compound of any one of Embodiments 1-3, wherein R² is H.

Embodiment 5 provides the compound of any one of Embodiments 1-4, wherein R³ is:

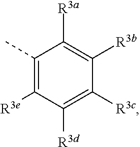

wherein:
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
   wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

Embodiment 6 provides the compound of Embodiment 5, wherein R^{3a}, R^{3b}, R^{3c}, R^{3d}, and R₃° are each independently selected from the group consisting of H, methyl, i-propyl, diphenylmethyl, methoxy, and fluoro.

Embodiment 7 provides the compound of Embodiment 5 or 6, wherein one of the following applies:
(a) R^{3b} and R^{3d} are each independently H; or
(b) R^{3b}, R^{3c}, and R^{3d} are each independently H.

Embodiment 8 provides the compound of any one of Embodiments 5-7, wherein one of the following applies:
(a) R^{3a} and R^{3e} are identical; or
(b) R^{3a}, R^{3c}, and R^{3e} are identical.

Embodiment 9 provides the compound of any one of Embodiments 5-8, wherein one of the following applies:
(a) R^{3a} and R^{3e} are each independently optionally substituted C₁-C₆ alkyl, and R^{3b}, R^{3c}, and R^{3d} are H;
(b) R^{3a}, R^{3c}, and R^{3e} are each independently optionally substituted C₁-C₆ alkyl, and R^{3b} and R^{3d} are H; or
(c) R^{3a} and R^{3e} are each independently optionally substituted C₁-C₆ alkyl, R^{3e} is selected from the group consisting of halogen and C₁-C₆ alkoxy, and R^{3b} and R^{3d} are H.

Embodiment 10 provides the compound of any one of Embodiments 1-9, wherein R³ is selected from the group consisting of:

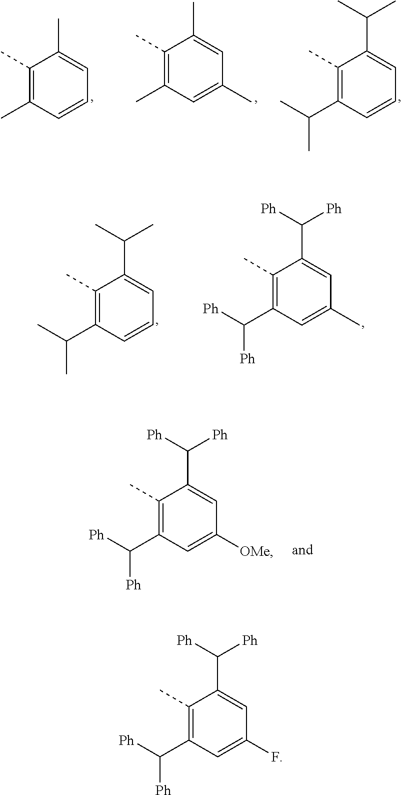

Embodiment 11 provides the compound of any one of Embodiments 1-4, wherein R³ is cyclohexyl.

Embodiment 12 provides the compound of any one of Embodiments 1-11, wherein R⁴ is:

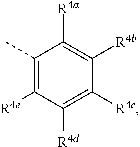

wherein:
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
   wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

Embodiment 13 provides the compound of Embodiment 12, wherein R^{4a}, R^{4b}, R^{4c}, R^{4d}, and R^{4e} are each independently selected from the group consisting of H, methyl, ethyl, i-propyl, cyclohexyl, t-butyl, methoxy, i-propoxy, phenyl, and dimethylamino.

Embodiment 14 provides the compound of Embodiment 12 or 13, wherein one of the following applies:
(a) R^{4b}, R^{4c}, R^{4d}, and R^{4e} are each independently H;
(b) R^{4b}, R^{4c}, and R^{4d} are each independently H;
(c) R^{4a}, R^{4c}, and R^{4e} are each independently H; or
(d) none of R^{4a}, R^{4b}, R^{4c}, R^{4d}, and R^{4e} are H.

Embodiment 15 provides the compound of any one of Embodiments 12-14, wherein one of the following applies:
(a) R^{4a} and R^{4e} are identical;
(b) R^{4b} and R^{4d} are identical;
(c) R^{4a}, R^{4c}, and R^{4e} are identical;
(c) R^{4a}, R^{4b}, R^{4c}, R^{4d}, and R^{4e} are identical.

Embodiment 16 provides the compound of any one of Embodiments 12-15, wherein one of the following applies:
(a) R^{4a} is selected from the group consisting of C₁-C₆ alkyl and N(C₁-C₆ alkyl)₂, and R^{4b}, R^{4c}, R^{4d}, and R^{4e} are each independently H;
(b) R^{4a} and R^{4e} are each independently C₁-C₆ alkyl, and R^{4b}, R^{4c}, and R^{4d} are each independently H;
(c) R^{4a} and R^{4e} are each independently C₁-C₆ alkoxy, and R^{4b}, R^{4c}, and R^{4d} are each independently H;
(d) R^{4b} and R^{4d} are each independently C₁-C₆ alkyl or phenyl, and R^{4a}, R^{4c}, and R^{4e} are each independently H;
(e) R^{4a}, R^{4c}, and R^{4e} are each independently C₁-C₆ alkyl or C₃-C₈ cycloalkyl, and R^{4b} and R^{4d} are each independently H;
(f) R^{4a}, R^{4c}, and R^{4e} are each independently C₁-C₆ alkoxy, and R^{4b} and R^{4d} are each independently H; or
(g) R^{4a}, R^{4b}, R^{4c}, R^{4d}, and R^{4e} are each independently C₁-C₆ alkyl.

Embodiment 17 provides the compound of any one of Embodiments 1-16, wherein R⁴ is selected from the group consisting of:

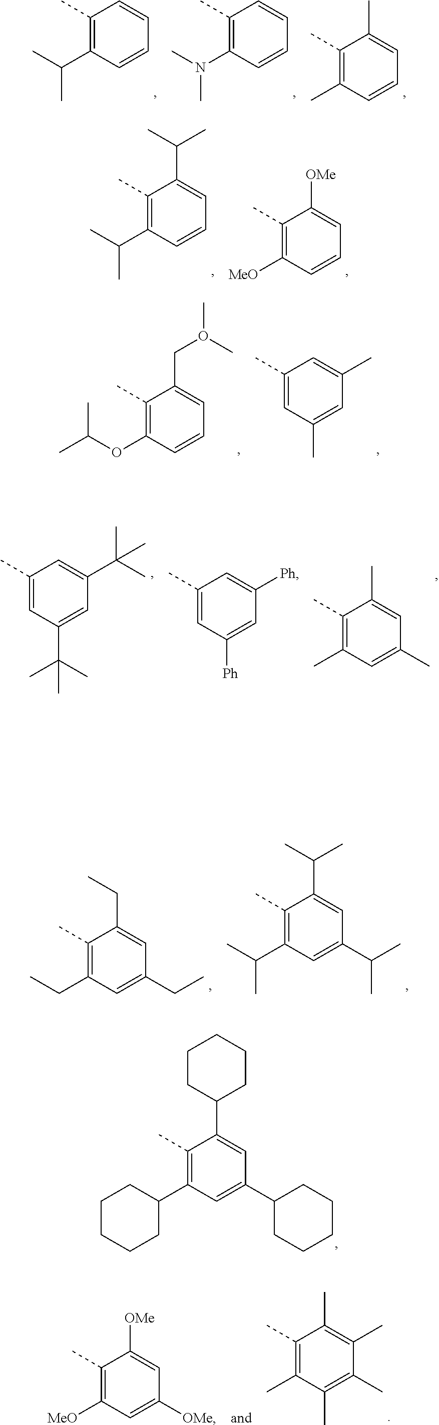

Embodiment 18 provides the compound of any one of Embodiments 1-11, wherein R⁴ is cyclohexyl or adamantyl.

Embodiment 19 provides the compound of any one of Embodiments 1-18, wherein X is selected from the group consisting of halogen, OS(═O)₂R^{A}, OC(═O)R^{A}, N(C(═O)R^{A})₂, tetracoordinate boronate, and hexacoordinate phosphorus.

Embodiment 20 provides the compound of any one of Embodiments 1-19, wherein X is C₁.

Embodiment 21 provides the compound of any one of Embodiments 1-20, which is selected from the group consisting of:
5-(2,6-dimethylphenyl)-2-mesitylimidazo[1,5-α]pyridin-2-ium chloride;
2-mesityl-5-(2,4,6-triethylphenyl)imidazo[1,5-α]pyridin-2-ium chloride;
2-(2,6-diisopropylphenyl)-5-(2,4,6-triethylphenyl)imidazo[1,5-α]pyridin-2-ium chloride;
2-(2,6-diisopropylphenyl)-5-(2,3,4,5,6-pentamethylphenyl)imidazo[1,5-α]pyridin-2-ium chloride;
2-mesityl-5-(2,4,6-tricyclohexylphenyl)imidazo[1,5-α]pyridin-2-ium chloride;
2-(2,6-diisopropylphenyl)-5-(2,4,6-tricyclohexylphenyl)imidazo[1,5-α]pyridin-2-ium chloride;
5-(2,6-diisopropylphenyl)-2-(2,6-dimethylphenyl)imidazo[1,5-α]pyridin-2-ium chloride;
5-(2,6-diisopropylphenyl)-2-mesitylimidazo[1,5-α]pyridin-2-ium chloride;
2,5-bis(2,6-diisopropylphenyl)imidazo[1,5-α]pyridin-2-ium chloride;
2-cyclohexyl-5-(2,6-diisopropylphenyl)imidazo[1,5-α]pyridin-2-ium chloride;
2-(2,6-diisopropylphenyl)-5-(2,6-dimethoxyphenyl)imidazo[1,5-α]pyridin-2-ium chloride;
2-(2,6-diisopropylphenyl)-5-(2,4,6-trimethoxyphenyl)imidazo[1,5-α]pyridin-2-ium chloride;
5-(2,6-diisopropoxyphenyl)-2-(2,6-diisopropylphenyl)imidazo[1,5-α]pyridin-2-ium chloride;
5-(2-(dimethylamino)phenyl)-2-mesitylimidazo[1,5-α]pyridin-2-ium chloride;
2-(2,6-diisopropylphenyl)-5-(2-(dimethylamino)phenyl)imidazo[1,5-α]pyridin-2-ium chloride;
2-(2,6-dibenzhydryl-4-methylphenyl)-5-mesitylimidazo[1,5-α]pyridin-2-ium chloride;
2-(2,6-dibenzhydryl-4-methoxyphenyl)-5-mesitylimidazo[1,5-α]pyridin-2-ium chloride;
2-(2,6-dibenzhydryl-4-methylphenyl)-5-(2,4,6-triisopropylphenyl)imidazo[1,5-α]pyridin-2-ium chloride;
2-(2,6-dibenzhydryl-4-fluorophenyl)-5-(2,4,6-triisopropylphenyl)imidazo[1,5-α]pyridin-2-ium chloride;
5-cyclohexyl-2-mesitylimidazo[1,5-α]pyridin-2-ium chloride;
5-cyclohexyl-2-(2,6-diisopropylphenyl)imidazo[1,5-α]pyridin-2-ium chloride;
5-(adamantan-1-yl)-2-mesitylimidazo[1,5-α]pyridin-2-ium chloride;
5-(adamantan-1-yl)-2-(2,6-diisopropylphenyl)imidazo[1,5-α]pyridin-2-ium chloride;
5-(3,5-dimethylphenyl)-2-mesitylimidazo[1,5-α]pyridin-2-ium chloride;
2-(2,6-Diisopropylphenyl)-5-(3,5-dimethylphenyl)imidazo[1,5-α]pyridin-2-ium chloride;
5-(3,5-di-tert-butylphenyl)-2-mesitylimidazo[1,5-α]pyridin-2-ium chloride;
5-(3,5-di-tert-butylphenyl)-2-(2,6-diisopropylphenyl)imidazo[1,5-α]pyridin-2-ium chloride;
5-([1,1′:3′,1″-terphenyl]-5′-yl)-2-mesitylimidazo[1,5-α]pyridin-2-ium chloride;
5-([1,1′:3′,1″-terphenyl]-5′-yl)-2-(2,6-diisopropylphenyl)imidazo[1,5-α]pyridin-2-ium chloride;
5-(2-isopropylphenyl)-2-mesitylimidazo[1,5-α]pyridin-2-ium chloride; and
2-(2,6-diisopropylphenyl)-5-(2-isopropylphenyl)imidazo[1,5-α]pyridin-2-ium chloride.

Embodiment 22 provides a compound of formula (II):

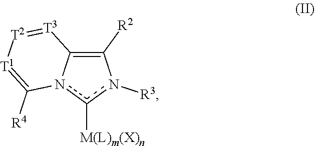

wherein:
M is a transition metal;
L is a ligand of M, wherein each occurrence of L can be the same or different; each occurrence of [symbol] is a single or double bond,
   wherein no more than one [symbol] bonding a C atom and a N atom is a double bond;
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R³ is selected from the group consisting of optionally substituted C₆-C₁₀ aryl, optionally substituted C₂-C₈ heteroaryl, and optionally substituted C₃-C₈ cycloalkyl,
   wherein each optional substituent in R³ is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(O)R^{A}, C(═O)N(R^{A})(R^{B}), C(O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R⁴ is selected from the group consisting of optionally substituted C₃-C₁₂ cycloalkyl and phenyl substituted with at least one substituent selected from the group consisting of C₁-C₆ alkyl, C₃-C₁₂ cycloalkyl, C₁-C₆ alkoxy, phenyl, and N(R^{A})(R^{B}),
   wherein each optional substituent in the C₃-C₁₂ cycloalkyl is at least one selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
X is a counter anion;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen;
m is an integer which is selected from the group consisting of 0, 1, 2, and 3;
n is an integer which is selected from the group consisting of 0, 1, and 2;
wherein if T¹ is CH, T² is CH, T³, is CH, R² is H, M is selected from the group consisting of Cu, Ag, and Au, and R⁴ is 2,4,6-triisopropylphenyl, then R³ is not 2,6-diisopropylphenyl or 2,4,6-trimethylphenyl;
wherein if T¹ is CH, T² is CH, T³, is CH, R² is H, M is Cu, and R⁴ is 2,4,6-trimethylphenyl, then R³ is not 2,4,6-trimethylphenyl, 2,6-diisopropylphenyl, adamantly, 4-(2-(2-methoxyethoxy)ethoxy)-2,6-dimethylphenyl, or (2-(2-methoxyethoxy)ethoxy)phenyl;
wherein if T¹ is CH, T² is CH, T³ is CH, R² is H, M is Pd, R⁴ is 2,4,6-trimethylphenyl, and L is 3-chloropyridyl or pyridyl, then R³ is not 2,4,6-trimethylphenyl or 4-(2-(2-methoxyethoxy)ethoxy)-2,6-dimethylphenyl;
wherein if T¹ is CH, T² is CH, T³ is CH, R² is H, M is Pd, R⁴ is 2,4,6-triisopropylphenyl, and X is allyl anion (i.e., vinylmethanide), then R³ is not 2,4,6-trimethylphenyl; and
wherein if T¹ is CH, T² is CH, T³, is CH, R² is H, M is Au, and R⁴ is 5-tetrahydroisoquinolinyl, then R³ is not 2,6-diisopropylphenyl.

Embodiment 23 provides the compound of Embodiment 22, wherein at least one of the following applies:
(a) T¹ is CR^{1a};
(b) T² is CR^{1b}; and
(c) T³ is CR^{1c}.

Embodiment 24 provides the compound of Embodiment 22 or 23, wherein at least one of the following occurs:
(a) at least one of R^{1a}, R^{1b}, and R^{1c} is H;
(b) at least two of R^{1a}, R^{1b}, and R^{1c} are H; and
(c) each of R^{1a}, R^{1b}, and R^{1c} are H.

Embodiment 25 provides the compound of any one of Embodiments 22-24, wherein R² is H.

Embodiment 26 provides the compound of any one of Embodiments 22-25, wherein R³ is:

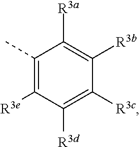

wherein:
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
   wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

Embodiment 27 provides the compound of Embodiment 26, wherein R^{3a}, R^{3b}, R^{3c}, R^{3d}, and R^{3e} are each independently selected from the group consisting of H, methyl, i-propyl, diphenylmethyl, methoxy, and fluoro.

Embodiment 28 provides the compound of Embodiment 26 or 27, wherein one of the following applies:
(a) R^{3b} and R^{3d} are each independently H; or
(b) R^{3b}, R^{3c}, and R^{3d} are each independently H.

Embodiment 29 provides the compound of any one of Embodiments 26-28, wherein one of the following applies:
(a) R^{3a} and R^{3e} are identical; or
(b) R^{3a}, R^{3c}, and R^{3e} are identical.

Embodiment 30 provides the compound of any one of Embodiments 26-29, wherein one of the following applies:
(a) R^{3a} and R^{3e} are each independently optionally substituted C₁-C₆ alkyl, and R^{3b}, R^{3c}, and R^{3d} are H;
(b) R^{3a}, R^{3c}, and R^{3e} are each independently optionally substituted C₁-C₆ alkyl, and R^{3b} and R^{3d} are H; or
(c) R^{3a} and R^{3e} are each independently optionally substituted C₁-C₆ alkyl, R^{3c} is selected from the group consisting of halogen and C₁-C₆ alkoxy, and R^{3b} and R^{3d} are H.

Embodiment 31 provides the compound of any one of Embodiments 22-30, wherein R³ is selected from the group consisting of:

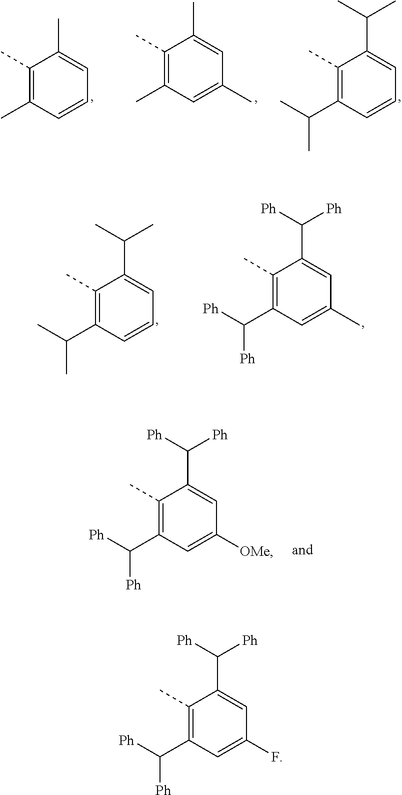

Embodiment 32 provides the compound of any one of Embodiments 22-25, wherein R³ is cyclohexyl.

Embodiment 33 provides the compound of any one of Embodiments 22-32, wherein R⁴ is:

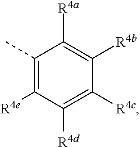

wherein:
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
   wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

Embodiment 34 provides the compound of Embodiment 33, wherein R^{4a}, R^{4b}, R^{4c}, R^{4d}, and R^{4e} are each independently selected from the group consisting of H, methyl, ethyl, i-propyl, cyclohexyl, t-butyl, methoxy, i-propoxy, phenyl, and dimethylamino.

Embodiment 35 provides the compound of Embodiment 33 or 34, wherein one of the following applies:
(a) R^{4b}, R^{4c}, R^{4d}, and R^{4e} are each independently H;
(b) R^{4b}, R^{4c}, and R^{4d} are each independently H;
(c) R^{4a}, R^{4c}, and R^{4e} are each independently H; or
(d) none of R^{4a}, R^{4b}, R^{4c}, R^{4d}, and R^{4e} are H.

Embodiment 36 provides the compound of any one of Embodiments 33-35, wherein one of the following applies:
(a) R^{4a} and R^{4e} are identical;
(b) R^{4b} and R^{4d} are identical;
(c) R^{4a}, R^{4c}, and R^{4e} are identical;
(c) R^{4a}, R^{4b}, R^{4c}, R^{4d}, and R^{4e} are identical.

Embodiment 37 provides the compound of any one of Embodiments 33-36, wherein one of the following applies:
(a) R^{4a} is selected from the group consisting of C₁-C₆ alkyl and N(C₁-C₆ alkyl)₂, and R^{4b}, R^{4c}, R^{4d}, and R^{4e} are each independently H;
(b) R^{4a} and R^{4e} are each independently C₁-C₆ alkyl, and R^{4b}, R^{4c}, and R^{4d} are each independently H;
(c) R^{4a} and R^{4e} are each independently C₁-C₆ alkoxy, and R^{4b}, R^{4c}, and R^{4d} are each independently H;
(d) R^{4b} and R^{4d} are each independently C₁-C₆ alkyl or phenyl, and R^{4a}, R^{4c}, and R^{4e} are each independently H;
(e) R^{4a}, R^{4c}, and R^{4e} are each independently C₁-C₆ alkyl or C₃-C₈ cycloalkyl, and R^{4b} and R^{4d} are each independently H;
(f) R^{4a}, R^{4c}, and R^{4e} are each independently C₁-C₆ alkoxy, and R^{4b} and R^{4d} are each independently H; or
(g) R^{4a}, R^{4b}, R^{4c}, R^{4d}, and R^{4e} are each independently C₁-C₆ alkyl.

Embodiment 38 provides the compound of any one of Embodiments 22-37, wherein R⁴ is selected from the group consisting of.

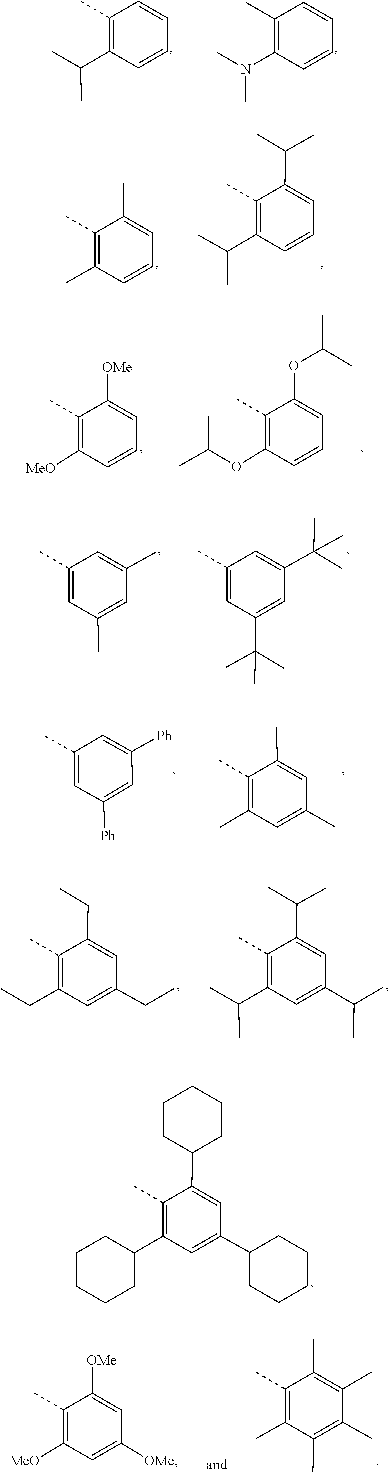

Embodiment 39 provides the compound of any one of Embodiments 22-32, wherein R⁴ is cyclohexyl or adamantyl.

Embodiment 40 provides the compound of any one of Embodiments 22-39, wherein X is selected from the group consisting of halogen, OS(═O)₂R^{A}, OC(═O)R^{A}, N(C(═O)R^{A})₂, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

Embodiment 41 provides the compound of any one of Embodiments 22-40, wherein X is selected from the group consisting of C₁, allyl anion (i.e., vinylmethanide), t-butylindenyl anion (i.e., 1-t-butylinden-1-ide and/or 3-t-butylinden-1-ide), and allylbenzene anion (i.e., 3-phenylpropen-3-ide and/or 1-phenylpropen-3-ide).

Embodiment 42 provides the compound of any one of Embodiments 22-41, wherein M is selected from the group consisting of Pd, Cu, Ag, Au, Ni, Pt, Co, Rh, Ir, Fe, Ru, and Os.

Embodiment 43 provides the compound of Embodiment 42, wherein M is selected from the group consisting of Pd, Cu Ag, and Rh.

Embodiment 44 provides the compound of any one of Embodiments 22-43, wherein L is selected from the group consisting of carbon monoxide (CO), optionally substituted C₂-C₁₂ alkene, and optionally substituted C₅-C₁₂ cycloalkene, optionally substituted benzylamine, optionally substituted C₂-C₈ heteroaryl, wherein each optional substituent in the C₂-C₁₂ alkene, C₅-C₁₂ cycloalkene, benzylamine, and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

Embodiment 45 provides the compound of Embodiment 44, wherein L is selected from the group consisting of cyclooctadiene (COD), carbon monoxide (CO), pyridine, and 3-chloropyridine.

Embodiment 46 provides the compound of any one of Embodiments 22-45, which is selected from the group consisting of:
cinnamyl [5-(2,6-diisopropylphenyl)-2-mesitylimidazo[1,5-α]pyridin-3-ylidene]chloropalladium;
cinnamyl [2-(2,6-dibenzhydryl-4-methylphenyl)-5-(2,4,6-triisopropylphenyl)imidazo[1,5-α]pyridin-3-ylidene]chloropalladium(II);
cinnamyl [5-(2,6-diisopropoxyphenyl)-2-(2,6-diisopropylphenyl)imidazo[1,5-α]pyridin-3-ylidene]chloropalladium(II);
2-dimethylaminomethylphenyl [2-(2,6-dibenzhydryl-4-methylphenyl)-5-(2,4,6-triisopropylphenyl)imidazo[1,5-α]pyridin-3-ylidene]chloropalladium(II);
2-acetamido-phenyl [2-(2,6-dibenzhydryl-4-methylphenyl)-5-(2,4,6-triisopropylphenyl)imidazo[1,5-α]pyridin-3-ylidene]chloropalladium(II);
cinnamyl [2,5-dimesitylimidazo[1,5-α]pyridin-3-ylidene]chloropalladium(II);
allyl [2,5-dimesitylimidazo[1,5-α]pyridin-3-ylidene]chloropalladium(II);
cinnamyl [2-(2,6-diisopropylphenyl)-5-mesitylimidazo[1,5-α]pyridin-3-ylidene]chloropalladium(II);
1-tert-butyl-indenyl [2-(2,6-diisopropylphenyl)-5-mesitylimidazo[1,5-α]pyridin-3-ylidene]chloropalladium(II);
cinnamyl [2-mesityl-5-(2,4,6-triisopropylphenyl)imidazo[1,5-α]pyridin-3-ylidene]chloropalladium(II);
pyridine [2-(2,6-diisopropylphenyl)-5-mesitylimidazo[1,5-α]pyridin-3-ylidene]dichloropalladium(II);
3-chloropyridine [2-(2,6-diisopropylphenyl)-5-mesitylimidazo[1,5-α]pyridin-3-ylidene]dichloropalladium(II);
3-chloropyridine [2-mesityl-5-(2,4,6-triisopropylphenyl)imidazo[1,5-α]pyridin-3-ylidene]dichloropalladium(II);
(2,5-dimesitylimidazo[1,5-α]pyridin-3(2H)-ylidene)gold(I) chloride; (5-(2,6-diisopropylphenyl)-2-mesitylimidazo[1,5-α]pyridin-3(2H)-ylidene)gold(I) chloride;
(2-(2,6-dibenzhydryl-4-methylphenyl)-5-mesityl-2,3-dihydroimidazo[1,5-α]pyridin-3-yl)gold(I) chloride;
(2-(2,6-dibenzhydryl-4-methoxyphenyl)-5-mesityl-2,3-dihydroimidazo[1,5-α]pyridin-3-yl)gold(I) chloride;
(2-(2,6-dibenzhydryl-4-methylphenyl)-5-(2,4,6-triisopropylphenyl)-2,3-dihydroimidazo[1,5-α]pyridin-3-yl)gold(I) chloride;
(5-(2,6-diisopropoxyphenyl)-2-(2,6-diisopropylphenyl)-2,3-dihydroimidazo[1,5-α]pyridin-3-yl)gold(I) chloride;
(2-(2,6-dibenzhydryl-4-methylphenyl)-5-mesityl-2,3-dihydroimidazo[1,5-α]pyridin-3-yl)copper(I) chloride;
(2-(2,6-dibenzhydryl-4-methoxyphenyl)-5-mesityl-2,3-dihydroimidazo[1,5-α]pyridin-3-yl)copper(I) chloride;
(2-(2,6-dibenzhydryl-4-methylphenyl)-5-(2,4,6-triisopropylphenyl)-2,3-dihydroimidazo[1,5-α]pyridin-3-yl)copper(I) chloride;
(2-(2,6-dibenzhydryl-4-methylphenyl)-5-mesityl-2,3-dihydroimidazo[1,5-α]pyridin-3-yl)silver(I) chloride;
(2-(2,6-dibenzhydryl-4-methoxyphenyl)-5-mesityl-2,3-dihydroimidazo[1,5-α]pyridin-3-yl)silver(I) chloride;
(2-(2,6-dibenzhydryl-4-methylphenyl)-5-(2,4,6-triisopropylphenyl)-2,3-dihydroimidazo[1,5-α]pyridin-3-yl)silver(I) chloride;
cyclooctadiene [5-(2,6-diisopropylphenyl)-2-mesitylimidazo[1,5-α]pyridin-3-ylidene]rhodium(I) chloride;
dicarbonyl [5-(2,6-diisopropylphenyl)-2-mesitylimidazo[1,5-α]pyridin-3-ylidene]rhodium(I) chloride;
cyclooctadiene [2,5-dimesitylimidazo[1,5-α]pyridin-2-ylidene]rhodium(I) chloride;
dicarbonyl [2,5-dimesitylimidazo[1,5-α]pyridin-2-ylidene]rhodium(I) chloride;
cyclooctadiene [2-(2,6-dibenzhydryl-4-methylphenyl)-5-(2,4,6-triisopropylphenyl)imidazo[1,5-α]pyridin-3-ylidene]rhodium(I) chloride;
dicarbonyl [2-(2,6-dibenzhydryl-4-methylphenyl)-5-(2,4,6-triisopropylphenyl)imidazo[1,5-α]pyridin-3-ylidene]rhodium(I) chloride;
(5-(2,4,6-triethylphenyl)-2-(2,6-diisopropylphenyl)-2,3-dihydroimidazo[1,5-α]pyridin-3-yl)gold(I) chloride;
cinnamyl (5-(2,4,6-triethylphenyl)-2-(2,6-diisopropylphenyl)-2,3-dihydroimidazo[1,5-α]pyridin-3-yl)chloropalladium (II);
(5-(2,4,6-trimethylphenyl)-2-(2,6-diisopropylphenyl)-2,3-dihydroimidazo[1,5-α]pyridin-3-yl)gold(I) chloride;
(5-(2,6-diisopropylphenyl)-2-(2,6-diisopropylphenyl)-2,3-dihydroimidazo[1,5-α]pyridin-3-yl)gold(I) chloride; and
(5-(2,6-diisopropylphenyl)-2-cyclohexyl-2,3-dihydroimidazo[1,5-α]pyridin-3-yl)gold(I) chloride.

Embodiment 47 provides a method of promoting a reaction between a boronic acid and a nitroarene, the method comprising contacting the boronic acid and the nitroarene in the presence of the compound of any one of Embodiments 22-45 and a base, and optionally in the presence of a phase transfer catalyst.

Embodiment 48 provides a method of promoting a reaction between an alkyl sulfoxide and an aniline, the method comprising contacting the alkyl sulfoxide and the aniline in the presence of the compound of any one of Embodiments 22-45 and a base.

Embodiment 49 provides a method of promoting a reaction between a thiophenol and an aniline, the method comprising contacting the thiophenol and the aniline in the presence of the compound of any one of Embodiments 22-45 and a base.

Embodiment 50 provides a method of promoting a reaction between an aryl halide and hydrazine, the method comprising contacting the aryl halide and the hydrazine in the presence of the compound of any one of Embodiments 22-45 and a Lewis acid to provide an aryl hydrazine.

Embodiment 51 provides the method of Embodiment 50, further comprising contacting the aryl hydrazine and a 1,3-dione to provide a 1-phenylpyrazole.

Embodiment 52 provides a method of promoting a reaction of an aryl mesylate or aryl tosylate with a boronic acid, the method comprising contacting the aryl mesylate or aryl tosylate and the boronic acid in the presence of the compound of any one of Embodiments 22-45 and a base.

Embodiment 53 provides a method of promoting a reaction of an aryl mesylate or tosylate with an aniline, the method comprising contacting the aryl mesylate or aryl tosylate and the aniline in the presence of the compound of any one of Embodiments 22-45 and a base.

Embodiment 54 provides a method of promoting a reaction between an aryl sulfamate and a boronic acid, the method comprising contacting the aryl sulfamate and the boronic acid in the presence of the compound of any one of Embodiments 22-45 and a base.

Embodiment 55 provides a method of promoting a reaction between an aryl sulfamate and an aniline, the method comprising contacting the aryl sulfamate and the aniline in the presence of the compound of any one of Embodiments 22-45 and a base.

Embodiment 56 provides a method of promoting a reaction between an aryl halide and a hydroxide salt, the method comprising contacting the aryl halide and the hydroxide salt in the presence of the compound of any one of Embodiments 22-45.

Embodiment 57 provides a method of promoting a reaction between an aryl methyl thioether and an aniline, the method comprising contacting the aryl methyl thioether and the aniline in the presence of the compound of any one of Embodiments 22-45 and a base.

Embodiment 58 provides a method of promoting a reaction between an aryl sulfoxide and an aniline, the method comprising contacting the aryl sulfoxide and the aniline in the presence of the compound of any one of Embodiments 22-45 and a base.

Embodiment 59 provides a method of promoting a reaction between an amine and an aryl chloride, the method comprising contacting the amine and the aryl chloride in the presence of the compound of any one of Embodiments 22-45 and a base.

Embodiment 60 provides a method of promoting a reaction between an aryl chloride and a boronic acid, the method comprising contacting the aryl chloride and the boronic acid in the presence of the compound of any one of Embodiments 22-45 and a base.

Embodiment 61 provides a method of promoting a reaction between an aryl fluoride and an aniline, the method comprising contacting the aryl fluoride and the aniline in the presence of the compound of any one of Embodiments 22-45 and a base.

Embodiment 62 provides a method of promoting a reaction between an alkyne and a nitroarene, the method comprising contacting the alkyne and the nitroarene in the presence of a compound of any one of Embodiments 22-45 and a reducing agent, wherein the contacting optionally further occurs in the presence of a Lewis acid.

Embodiment 63 provides the method of any one of Embodiments 46-62, wherein M is Pd in the compound of any one of Embodiments 22-45.

Embodiment 64 provides the method of any one of Embodiments 46-63, wherein at least one of the following applies:
(a) the boronic acid is a C₆-C₁₀ aryl or C₂-C₈ heteroaryl, wherein the C₆-C₁₀ aryl or C₂-C₈ heteroaryl is substituted with at least one boronate or boronic acid moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(b) the nitroarene is a C₆-C₁₀ aryl or C₂-C₈ heteroaryl, wherein the C₆-C₁₀ aryl or C₂-C₈ heteroaryl is substituted with at least one NO₂ moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(c) the alkyl sulfoxide is a C₆-C₁₀ aryl or C₂-C₈ heteroaryl, wherein the C₆-C₁₀ aryl or C₂-C₈ heteroaryl is substituted with at least one S(═O)(optionally substituted C₁-C₆ alkyl) moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(d) the aniline is a C₆-C₁₀ aryl or C₂-C₈ heteroaryl, wherein the C₆-C₁₀ aryl or C₂-C₈ heteroaryl is substituted with at least one NH₂ moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(e) the thiophenol is a C₆-C₁₀ aryl or C₂-C₈ heteroaryl, wherein the C₆-C₁₀ aryl or C₂-C₈ heteroaryl is substituted with at least one thiol moiety and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(f) the aryl halide is a C₆-C₁₀ aryl or C₂-C₈ heteroaryl, wherein the C₆-C₁₀ aryl or C₂-C₈ heteroaryl is substituted with at least one halogen selected from the group consisting of C₁, Br, and I, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(g) the aryl tosylate is a C₆-C₁₀ aryl or C₂-C₈ heteroaryl, wherein the C₆-C₁₀ aryl or C₂-C₈ heteroaryl is substituted with at least one toluenesulfonoxy moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(h) the aryl mesylate is a C₆-C₁₀ aryl or C₂-C₈ heteroaryl, wherein the C₆-C₁₀ aryl or C₂-C₈ heteroaryl is substituted with at least one methanesulfonoxy moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(i) the aryl sulfamate is a C₆-C₁₀ aryl or C₂-C₈ heteroaryl, wherein the C₆-C₁₀ aryl or C₂-C₈ heteroaryl is substituted with at least one sulfamate moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(j) the aryl chloride is a C₆-C₁₀ aryl or C₂-C₈ heteroaryl, wherein the C₆-C₁₀ aryl or C₂-C₈ heteroaryl is substituted with at least one C₁, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(k) the aryl methyl thioether is a C₆-C₁₀ aryl or C₂-C₈ heteroaryl, wherein the C₆-C₁₀ aryl or C₂-C₈ heteroaryl is substituted with at least one methylthio moiety (i.e., —SCH₃), and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(l) the aryl sulfoxide is C₆-C₁₀ aryl or C₂-C₈ heteroaryl, wherein the C₆-C₁₀ aryl or C₂-C₈ heteroaryl is substituted with at least one substituent selected from the group consisting of S(═O)(optionally substituted C₁-C₆ alkyl), S(═O)(optionally substituted C₆-C₁₀ aryl), and S(═O)(optionally substituted C₂-C₈ heteroaryl), and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(m) the amine is selected from the group consisting of a N (optionally substituted C₁-C₆ alkyl)₂, N (optionally substituted C₁-C₆ alkyl)(optionally substituted C₆-C₁₀ aryl), N (optionally substituted C₆-C₁₀ aryl)₂, and optionally substituted C₂-C₈ heterocycloalkyl, wherein the heterocycloalkyl comprises at least one secondary amine;
(n) the aryl bromide is a C₆-C₁₀ aryl or C₂-C₈ heteroaryl, wherein the C₆-C₁₀ aryl or C₂-C₈ heteroaryl is substituted with at least one Br, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂; and
(o) the aryl fluoride is a C₆-C₁₀ aryl or C₂-C₈ heteroaryl, wherein the C₆-C₁₀ aryl or C₂-C₈ heteroaryl is substituted with at least one F, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 65 provides a method of preparing a compound of formula (IV):

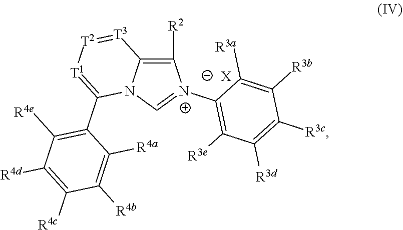

wherein:
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
   wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
   wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; and
X is a counter ion;
the method comprising contacting (Z):

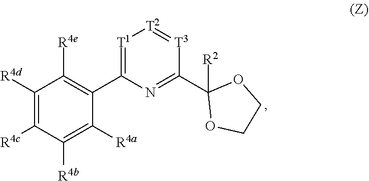

and (Y):

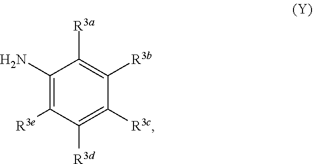

in the presence of paraformaldehyde and an acid.

Embodiment 66 provides the method of Embodiment 63, wherein the (Z) is prepared by reacting (X):

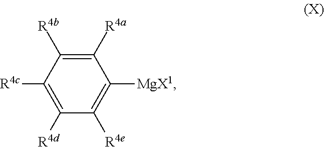

### and (W):

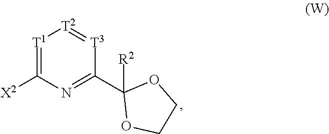

in the presence of a transition metal catalyst;
wherein X¹ and X² are each independently selected from the group consisting of Cl, Br, and I.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of formula (I): 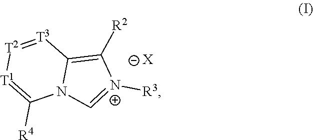
wherein:
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R³ is selected from the group consisting of optionally substituted C₆-C₁₀ aryl, optionally substituted C₂-C₈ heteroaryl, and optionally substituted C₃-C₈ cycloalkyl,
wherein each optional substituent in R³ is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R⁴ is selected from the group consisting of optionally substituted C₃-C₁₂ cycloalkyl and phenyl substituted with at least one substituent selected from the group consisting of C₁-C₆ alkyl, C₃-C₁₂ cycloalkyl, C₁-C₆ alkoxy, phenyl, and N(R^{A})(R^{B}),
wherein each optional substituent in the C₃-C₁₂ cycloalkyl is at least one selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
X is a counter anion;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen;
wherein if T¹ is CH, T² is CH, T³, is CH, and R² is H, then each of the following apply:
(a) if R⁴ is 2,4,6-triisopropylphenyl, then R³ is not 2,6-diisopropylphenyl, 2,4,6-trimethylphenyl, 2-methylphenyl, or 2,6-diethyl-4-methylphenyl;
(b) if R⁴ is 2,4,6-trimethylphenyl, then R³ is not 2,4,6-trimethylphenyl, 2,6-diisopropylphenyl, adamantly, 4-(2-(2-methoxyethoxy)ethoxy)-2,6-dimethylphenyl, or (2-(2-methoxyethoxy)ethoxy)phenyl;
(c) if R⁴ is 2-methylphenyl, then R³ is not 2,6-diisopropylphenyl;
(d) if R⁴ is 4-tert-butyl-2-pyridyl, then R³ is not 2,6-diisopropylphenyl; and
(e) if R⁴ is 5-tetrahydroisoquinolinyl, then R³ is not 2,6-diisopropylphenyl.

2. The compound of claim 1, wherein at least one of the following applies:
(a) T¹ is CR^{1a};
(b) T² is CR^{1b};
(c) T³ is CR^{1c};
(d) at least one of R^{1a}, R^{1b}, and R^{1c} is H;
(e) at least two of R^{1a}, R^{1b}, and R^{1c} are H;
(f) each of R^{1a}, R^{1b}, and R^{1c} are H; and
(g) wherein R² is H.

3-4. (canceled)

5. The compound of claim 1, wherein R³ is: 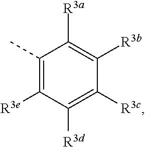
wherein:
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

6. (canceled)

7. The compound of claim 5, wherein one of the following applies:
(a) R^{3b} and R^{3d} are each independently H;
(b) R^{3b}, R^{3c}, and R^{3d} are each independently H,
(c) R^{3a} and R^{3e} are identical;
(d) R^{3a}, R^{3c}, and R^{3e} are identical;
(e) R^{3a} and R^{3e} are each independently optionally substituted C₁-C₆ alkyl, and R^{3b}, R^{3c}, and R^{3d} are H;
(f) R^{3a}, R^{3c}, and R^{3e} are each independently optionally substituted C₁-C₆ alkyl, and R^{3b} and R^{3d} are H;
(g) R^{3a} and R^{3e} are each independently optionally substituted C₁-C₆ alkyl, R^{3c} is selected from the group consisting of halogen and C₁-C₆ alkoxy, and R^{3b} and R^{3d} are H;
or
(h) R^{3a}, R^{3b}, R^{3c}, R^{3d}, and R^{3e} are each independently selected from the group consisting of H, methyl, i-propyl, diphenylmethyl, methoxy, and fluoro.

8-9. (canceled)

10. The compound of claim 1, wherein at least one of the following applies:
(a) R³ is selected from the group consisting of cyclohexyl, 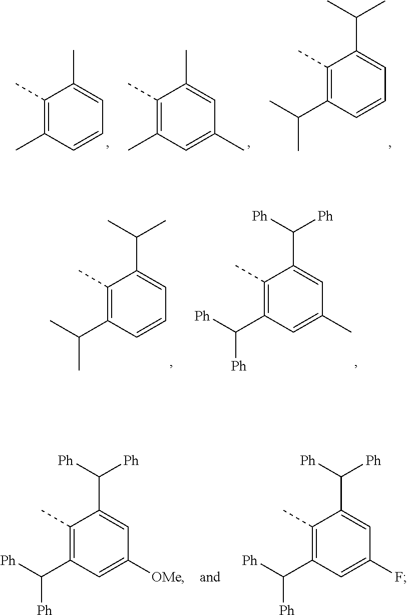
and
(b) R⁴ is selected from the group consisting of cyclohexyl, adamantyl,

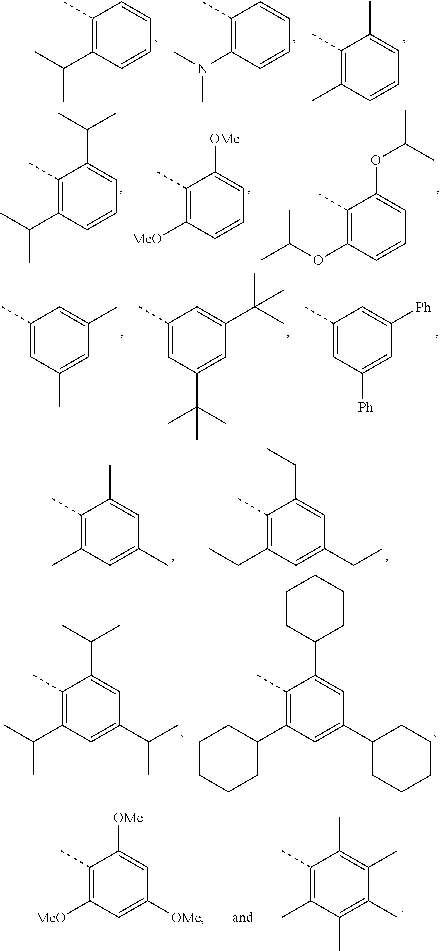

11. (canceled)

12. The compound of claim 1, wherein R⁴ is: 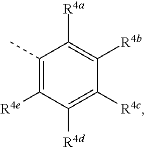
wherein:
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

13. (canceled)

14. The compound of claim 12, wherein one of the following applies:
(a) R^{4b}, R^{4c}, R^{4d}, and R^{4e} are each independently H;
(b) R^{4b}, R^{4c}, and R^{4d} are each independently H;
(c) R^{4a}, R^{4c}, and R^{4d} are each independently H;
(d) none of R^{4a}, R^{4b}, R^{4c}, R^{4d}, and R^{4e} are H,
(e) R^{4b} and R^{4d} are identical;
(f) R^{4a}, R^{4c}, and R^{4e} are identical;
(g) R^{4a}, R^{4b}, R^{4c}, R^{4d}, and R^{4e} are identical;
(h) R^{4a} is selected from the group consisting of C₁-C₆ alkyl and N(C₁-C₆ alkyl)₂, and R^{4b}, R^{4c}, R^{4d}, and R^{4e} are each independently H;
(i) R^{4a} and R^{4e} are each independently C₁-C₆ alkyl, and R^{4b}, R^{4c}, and R^{4d} are each independently H;
(j) R^{4a} and R^{4e} are each independently C₁-C₆ alkoxy, and R^{4b}, R^{4c}, and R^{4d} are each independently H;
(k) R^{4b} and R^{4d} are each independently C₁-C₆ alkyl or phenyl, and R^{4a}, R^{4c}, and R^{4e} are each independently H;
(l) R^{4a}, R^{4c}, and R^{4e} are each independently C₁-C₆ alkyl or C₃-C₈ cycloalkyl, and R^{4b} and R^{4d} are each independently H;
(m) R^{4a}, R^{4c}, and R^{4e} are each independently C₁-C₆ alkoxy, and R^{4b} and R^{4d} are each independently H;
(n) R^{4a}, R^{4b}, R^{4c}, R^{4d}, and R^{4e} are each independently C₁-C₆ alkyl; or
(o) R^{4a}, R^{4b}, R^{4c}, R^{4d}, and R^{4e} are each independently selected from the group consisting of H, methyl, ethyl, i-propyl, cyclohexyl, t-butyl, methoxy, i-propoxy, phenyl, and dimethylamino.

15-18. (canceled)

19. The compound of claim 1, wherein X is selected from the group consisting of halogen, OS(═O)₂R^{A}, OC(═O)R^{A}, N(C(═O)R^{A})₂, tetracoordinate boronate, and hexacoordinate phosphorus, optionally wherein X is Cl.

20. (canceled)

21. The compound of claim 1, which is selected from the group consisting of:
5-(2,6-dimethylphenyl)-2-mesitylimidazo[1,5-α]pyridin-2-ium chloride;
2-mesityl-5-(2,4,6-triethylphenyl)imidazo[1,5-α]pyridin-2-ium chloride;
2-(2,6-diisopropylphenyl)-5-(2,4,6-triethylphenyl)imidazo[1,5-α]pyridin-2-ium chloride;
2-(2,6-diisopropylphenyl)-5-(2,3,4,5,6-pentamethylphenyl)imidazo[1,5-α]pyridin-2-ium chloride;
2-mesityl-5-(2,4,6-tricyclohexylphenyl)imidazo[1,5-α]pyridin-2-ium chloride;
2-(2,6-diisopropylphenyl)-5-(2,4,6-tricyclohexylphenyl)imidazo[1,5-α]pyridin-2-ium chloride;
5-(2,6-diisopropylphenyl)-2-(2,6-dimethylphenyl)imidazo[1,5-α]pyridin-2-ium chloride;
5-(2,6-diisopropylphenyl)-2-mesitylimidazo[1,5-α]pyridin-2-ium chloride;
2,5-bis(2,6-diisopropylphenyl)imidazo[1,5-α]pyridin-2-ium chloride;
2-cyclohexyl-5-(2,6-diisopropylphenyl)imidazo[1,5-α]pyridin-2-ium chloride;
2-(2,6-diisopropylphenyl)-5-(2,6-dimethoxyphenyl)imidazo[1,5-α]pyridin-2-ium chloride;
2-(2,6-diisopropylphenyl)-5-(2,4,6-trimethoxyphenyl)imidazo[1,5-α]pyridin-2-ium chloride;
5-(2,6-diisopropoxyphenyl)-2-(2,6-diisopropylphenyl)imidazo[1,5-α]pyridin-2-ium chloride;
5-(2-(dimethylamino)phenyl)-2-mesitylimidazo[1,5-α]pyridin-2-ium chloride;
2-(2,6-diisopropylphenyl)-5-(2-(dimethylamino)phenyl)imidazo[1,5-α]pyridin-2-ium chloride;
2-(2,6-dibenzhydryl-4-methylphenyl)-5-mesitylimidazo[1,5-α]pyridin-2-ium chloride;
2-(2,6-dibenzhydryl-4-methoxyphenyl)-5-mesitylimidazo[1,5-α]pyridin-2-ium chloride;
2-(2,6-dibenzhydryl-4-methylphenyl)-5-(2,4,6-triisopropylphenyl)imidazo[1,5-α]pyridin-2-ium chloride;
2-(2,6-dibenzhydryl-4-fluorophenyl)-5-(2,4,6-triisopropylphenyl)imidazo[1,5-α]pyridin-2-ium chloride;
5-cyclohexyl-2-mesitylimidazo[1,5-α]pyridin-2-ium chloride;
5-cyclohexyl-2-(2,6-diisopropylphenyl)imidazo[1,5-α]pyridin-2-ium chloride;
5-(adamantan-1-yl)-2-mesitylimidazo[1,5-α]pyridin-2-ium chloride;
5-(adamantan-1-yl)-2-(2,6-diisopropylphenyl)imidazo[1,5-α]pyridin-2-ium chloride;
5-(3,5-dimethylphenyl)-2-mesitylimidazo[1,5-α]pyridin-2-ium chloride;
2-(2,6-Diisopropylphenyl)-5-(3,5-dimethylphenyl)imidazo[1,5-α]pyridin-2-ium chloride;
5-(3,5-di-tert-butylphenyl)-2-mesitylimidazo[1,5-α]pyridin-2-ium chloride;
5-(3,5-di-tert-butylphenyl)-2-(2,6-diisopropylphenyl)imidazo[1,5-α]pyridin-2-ium chloride;
5-([1,1′:3′,1″-terphenyl]-5′-yl)-2-mesitylimidazo[1,5-α]pyridin-2-ium chloride;
5-([1,1′:3′,1″-terphenyl]-5′-yl)-2-(2,6-diisopropylphenyl)imidazo[1,5-α]pyridin-2-ium chloride;
5-(2-isopropylphenyl)-2-mesitylimidazo[1,5-α]pyridin-2-ium chloride; and
2-(2,6-diisopropylphenyl)-5-(2-isopropylphenyl)imidazo[1,5-α]pyridin-2-ium chloride.

22. A compound of formula (II): 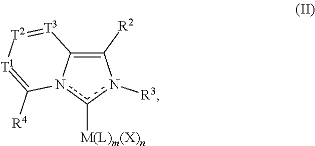
wherein:
M is a transition metal;
L is a ligand of M, wherein each occurrence of L can be the same or different;
each occurrence of [symbol] is a single or double bond,
wherein no more than one [symbol] bonding a C atom and a N atom is a double bond;
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R³ is selected from the group consisting of optionally substituted C₆-C₁₀ aryl, optionally substituted C₂-C₈ heteroaryl, and optionally substituted C₃-C₈ cycloalkyl,
wherein each optional substituent in R³ is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R⁴ is selected from the group consisting of optionally substituted C₃-C₁₂ cycloalkyl and phenyl substituted with at least one substituent selected from the group consisting of C₁-C₆ alkyl, C₃-C₁₂ cycloalkyl, C₁-C₆ alkoxy, phenyl, and N(R^{A})(R^{B}),
wherein each optional substituent in the C₃-C₁₂ cycloalkyl is at least one selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
X is a counter anion;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen;
m is an integer which is selected from the group consisting of 0, 1, 2, and 3;
n is an integer which is selected from the group consisting of 0, 1, and 2;
wherein if T¹ is CH, T² is CH, T³, is CH, R² is H, M is selected from the group consisting of Cu, Ag, and Au, and R⁴ is 2,4,6-triisopropylphenyl, then R³ is not 2,6-diisopropylphenyl or 2,4,6-trimethylphenyl;
wherein if T¹ is CH, T² is CH, T³, is CH, R² is H, M is Cu, and R⁴ is 2,4,6-trimethylphenyl, then R³ is not 2,4,6-trimethylphenyl, 2,6-diisopropylphenyl, adamantly, 4-(2-(2-methoxyethoxy)ethoxy)-2,6-dimethylphenyl, or (2-(2-methoxyethoxy)ethoxy)phenyl;
wherein if T¹ is CH, T² is CH, T³ is CH, R² is H, M is Pd, R⁴ is 2,4,6-trimethylphenyl, and L is 3-chloropyridyl or pyridyl, then R³ is not 2,4,6-trimethylphenyl or 4-(2-(2-methoxyethoxy)ethoxy)-2,6-dimethylphenyl;
wherein if T¹ is CH, T² is CH, T³ is CH, R² is H, M is Pd, R⁴ is 2,4,6-triisopropylphenyl, and X is allyl anion (i.e., vinylmethanide), then R³ is not 2,4,6-trimethylphenyl; and
wherein if T¹ is CH, T² is CH, T³, is CH, R² is H, M is Au, and R⁴ is 5-tetrahydroisoquinolinyl, then R³ is not 2,6-diisopropylphenyl.

23. The compound of claim 22, wherein at least one of the following applies:
(a) T¹ is CR^{1a};
(b) T² is CR^{1b};
(c) T³ is CR^{1c};
(d) at least one of R^{1a}, R^{1b}, and R^{1c} is H;
(e) at least two of R^{1a}, R^{1b}, and R^{1c} are H;
(f) each of R^{1a}, R^{1b}, and R^{1c} are H; or
(g) R₂ is H.

24-25. (canceled)

26. The compound of claim 22, wherein R³ is: 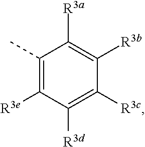
wherein:
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

27. (canceled)

28. The compound of claim 26, wherein one of the following applies:
(a) R^{3b} and R^{3d} are each independently H;
(b) R^{3b}, R^{3c}, and R^{3d} are each independently H,
(c) R^{3a} and R^{3e} are identical;
(d) R^{3a}, R^{3c}, and R^{3e} are identical;
(e) R^{3a} and R^{3e} are each independently optionally substituted C₁-C₆ alkyl, and R^{3b}, R^{3c}, and R^{3d} are H;
(f) R^{3a}, R^{3c}, and R^{3e} are each independently optionally substituted C₁-C₆ alkyl, and R^{3b} and R^{3d} are H;
(g) R^{3a} and R^{3e} are each independently optionally substituted C₁-C₆ alkyl, R^{3c} is selected from the group consisting of halogen and C₁-C₆ alkoxy, and R^{3b} and R^{3d} are H; or
(h) R^{3a}, R^{3b}, R^{3c}, R^{3d}, and R^{3e} are each independently selected from the group consisting of H, methyl, i-propyl, diphenylmethyl, methoxy, and fluoro.

29-30. (canceled)

31. The compound of claim 22, wherein at least one of the following applies:
(a) R³ is selected from the group consisting of cyclohexyl, 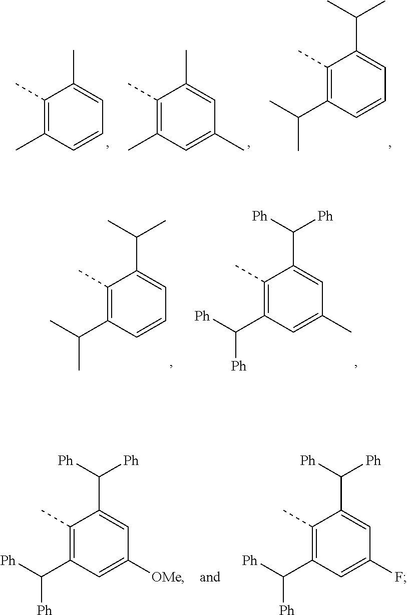
and
(b) R⁴ is selected from the group consisting of cyclohexyl, adamantyl,

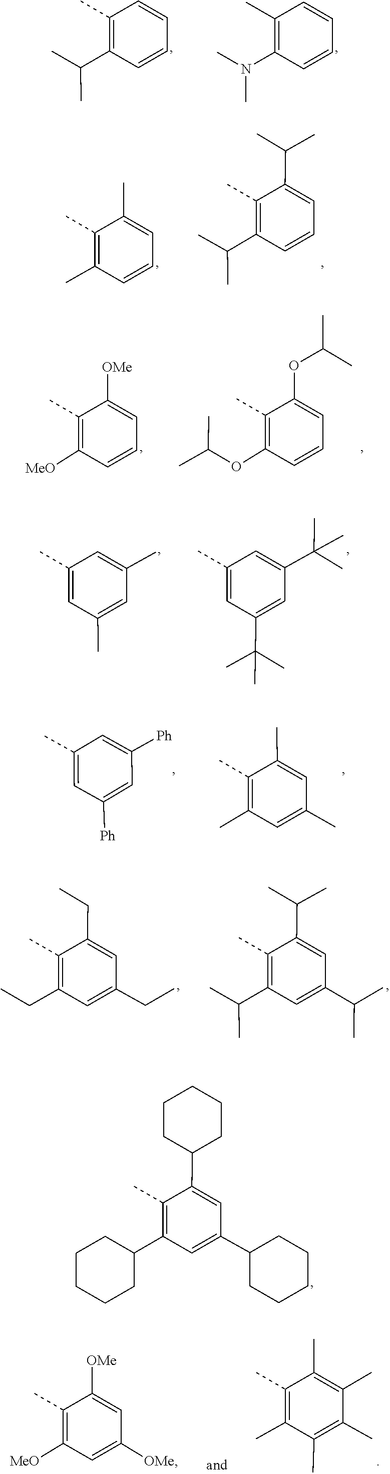

32. (canceled)

33. The compound of claim 22, wherein R⁴ is: 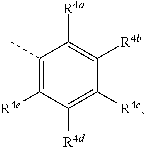
wherein:
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

34. (canceled)

35. The compound of claim 33, wherein one of the following applies:
(a) R^{4b}, R^{4c}, R^{4d}, and R^{4e} are each independently H;
(b) R^{4b}, R^{4c}, and R^{4d} are each independently H;
(c) R^{4a}, R^{4c}, and R^{4e} are each independently H;
(d) none of R^{4a}, R^{4b}, R^{4c}, R^{4d}, and R^{4e} are H;
(e) R^{4a} and R^{4e} are identical;
(f) R^{4b} and R^{4d} are identical;
(g) R^{4a}, R^{4c}, and R^{4e} are identical;
(h) R^{4a}, R^{4b}, R^{4c}, R^{4d}, and R^{4e} are identical;
(i) R^{4a} is selected from the group consisting of C₁-C₆ alkyl and N(C₁-C₆ alkyl)₂, and R^{4b}, R^{4c}, R^{4d}, and R^{4e} are each independently H;
(i) R^{4a} and R^{4e} are each independently C₁-C₆ alkyl, and R^{4b}, R^{4c}, and R^{4d} are each independently H;
(k) R^{4a} and R^{4e} are each independently C₁-C₆ alkoxy, and R^{4b}, R^{4c}, and R^{4d} are each independently H;
(l) R^{4b} and R^{4d} are each independently C₁-C₆ alkyl or phenyl, and R^{4a}, R^{4c}, and R^{4e} are each independently H;
(m) R^{4a}, R^{4c}, and R^{4e} are each independently C₁-C₆ alkyl or C₃-C₈ cycloalkyl, and R^{4b} and R^{4d} are each independently H;
(n) R^{4a}, R^{4c}, and R^{4e} are each independently C₁-C₆ alkoxy, and R^{4b} and R^{4d} are each independently H;
(o) R^{4a}, R^{4b}, R^{4c}, R^{4d}, and R^{4e} are each independently C₁-C₆ alkyl; or
(p) R^{4a}, R^{4b}, R^{4c}, R^{4d}, and R^{4e} are each independently selected from the group consisting of H, methyl, ethyl, i-propyl, cyclohexyl, t-butyl, methoxy, i-propoxy, phenyl, and dimethylamino.

36-39. (canceled)

40. The compound of claim 22, wherein at least one of the following applies:
(a) X is selected from the group consisting of halogen, OS(═O)₂R^{A}, OC(═O)R^{A}, N(C(═O)R^{A})₂, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl, optionally wherein X is selected from the group consisting of C₁, allyl anion (i.e., vinylmethanide), t-butylindenyl anion (i.e., 1-t-butylinden-1-ide and/or 3-t-butylinden-1-ide), and allylbenzene anion (i.e., 3-phenylpropen-3-ide and/or 1-phenylpropen-3-ide);
(b) M is selected from the group consisting of Pd, Cu, Ag, Au, Ni, Pt, Co, Rh, Ir, Fe, Ru, and Os; and
(c) L is selected from the group consisting of carbon monoxide (CO), optionally substituted C₂-C₁₂ alkene, and optionally substituted C₅-C₁₂ cycloalkene, optionally substituted benzylamine, optionally substituted C₂-C₈ heteroaryl, wherein each optional substituent in the C₂-C₁₂ alkene, C₅-C₁₂ cycloalkene, benzylamine, and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl, optionally wherein L is selected from the group consisting of cyclooctadiene (COD), carbon monoxide (CO), pyridine, and 3-chloropyridine.

41-45. (canceled)

46. The compound of claim 22, which is selected from the group consisting of:
cinnamyl [5-(2,6-diisopropylphenyl)-2-mesitylimidazo[1,5-α]pyridin-3-ylidene]chloropalladium;
cinnamyl [2-(2,6-dibenzhydryl-4-methylphenyl)-5-(2,4,6-triisopropylphenyl)imidazo[1,5-α]pyridin-3-ylidene]chloropalladium(II);
cinnamyl [5-(2,6-diisopropoxyphenyl)-2-(2,6-diisopropylphenyl)imidazo[1,5-α]pyridin-3-ylidene]chloropalladium(II);
2-dimethylaminomethylphenyl [2-(2,6-dibenzhydryl-4-methylphenyl)-5-(2,4,6-triisopropylphenyl)imidazo[1,5-α]pyridin-3-ylidene]chloropalladium(II);
2-acetamido-phenyl [2-(2,6-dibenzhydryl-4-methylphenyl)-5-(2,4,6-triisopropylphenyl)imidazo[1,5-α]pyridin-3-ylidene]chloropalladium(II);
cinnamyl [2,5-dimesitylimidazo[1,5-α]pyridin-3-ylidene]chloropalladium(II);
allyl [2,5-dimesitylimidazo[1,5-α]pyridin-3-ylidene]chloropalladium(II);
Cinnamyl [2-(2,6-diisopropylphenyl)-5-mesitylimidazo[1,5-α]pyridin-3-ylidene]chloropalladium(II);
1-tert-butyl-indenyl [2-(2,6-diisopropylphenyl)-5-mesitylimidazo[1,5-α]pyridin-3-ylidene]chloropalladium(II);
cinnamyl [2-mesityl-5-(2,4,6-triisopropylphenyl)imidazo[1,5-α]pyridin-3-ylidene]chloropalladium(II);
pyridine [2-(2,6-diisopropylphenyl)-5-mesitylimidazo[1,5-α]pyridin-3-ylidene]dichloropalladium(II);
3-chloropyridine [2-(2,6-diisopropylphenyl)-5-mesitylimidazo[1,5-α]pyridin-3-ylidene]dichloropalladium(II);
3-chloropyridine [2-mesityl-5-(2,4,6-triisopropylphenyl)imidazo[1,5-α]pyridin-3-ylidene]dichloropalladium(II);
(2,5-dimesitylimidazo[1,5-α]pyridin-3(2H)-ylidene)gold(I) chloride;
(5-(2,6-diisopropylphenyl)-2-mesitylimidazo[1,5-α]pyridin-3(2H)-ylidene)gold(I) chloride;
(2-(2,6-dibenzhydryl-4-methylphenyl)-5-mesityl-2,3-dihydroimidazo[1,5-α]pyridin-3-yl)gold(I) chloride;
(2-(2,6-dibenzhydryl-4-methoxyphenyl)-5-mesityl-2,3-dihydroimidazo[1,5-α]pyridin-3-yl)gold(I) chloride;
(2-(2,6-dibenzhydryl-4-methylphenyl)-5-(2,4,6-triisopropylphenyl)-2,3-dihydroimidazo[1,5-α]pyridin-3-yl)gold(I) chloride;
(5-(2,6-diisopropoxyphenyl)-2-(2,6-diisopropylphenyl)-2,3-dihydroimidazo[1,5-α]pyridin-3-yl)gold(I) chloride;
(2-(2,6-dibenzhydryl-4-methylphenyl)-5-mesityl-2,3-dihydroimidazo[1,5-α]pyridin-3-yl)copper(I) chloride;
(2-(2,6-dibenzhydryl-4-methoxyphenyl)-5-mesityl-2,3-dihydroimidazo[1,5-α]pyridin-3-yl)copper(I) chloride;
(2-(2,6-dibenzhydryl-4-methylphenyl)-5-(2,4,6-triisopropylphenyl)-2,3-dihydroimidazo[1,5-α]pyridin-3-yl)copper(I) chloride;
(2-(2,6-dibenzhydryl-4-methylphenyl)-5-mesityl-2,3-dihydroimidazo[1,5-α]pyridin-3-yl)silver(I) chloride;
(2-(2,6-dibenzhydryl-4-methoxyphenyl)-5-mesityl-2,3-dihydroimidazo[1,5-α]pyridin-3-yl)silver(I) chloride;
(2-(2,6-dibenzhydryl-4-methylphenyl)-5-(2,4,6-triisopropylphenyl)-2,3-dihydroimidazo[1,5-α]pyridin-3-yl)silver(I) chloride;
cyclooctadiene [5-(2,6-diisopropylphenyl)-2-mesitylimidazo[1,5-α]pyridin-3-ylidene]rhodium(I) chloride;
dicarbonyl [5-(2,6-diisopropylphenyl)-2-mesitylimidazo[1,5-α]pyridin-3-ylidene]rhodium(I) chloride;
cyclooctadiene [2,5-dimesitylimidazo[1,5-α]pyridin-2-ylidene]rhodium(I) chloride;
dicarbonyl [2,5-dimesitylimidazo[1,5-α]pyridin-2-ylidene]rhodium(I) chloride;
cyclooctadiene [2-(2,6-dibenzhydryl-4-methylphenyl)-5-(2,4,6-triisopropylphenyl)imidazo[1,5-α]pyridin-3-ylidene]rhodium(I) chloride;
dicarbonyl [2-(2,6-dibenzhydryl-4-methylphenyl)-5-(2,4,6-triisopropylphenyl)imidazo[1,5-α]pyridin-3-ylidene]rhodium(I) chloride;
(5-(2,4,6-triethylphenyl)-2-(2,6-diisopropylphenyl)-2,3-dihydroimidazo[1,5-α]pyridin-3-yl)gold(I) chloride;
cinnamyl (5-(2,4,6-triethylphenyl)-2-(2,6-diisopropylphenyl)-2,3-dihydroimidazo[1,5-α]pyridin-3-yl)chloropalladium (II);
(5-(2,4,6-trimethylphenyl)-2-(2,6-diisopropylphenyl)-2,3-dihydroimidazo[1,5-α]pyridin-3-yl)gold(I) chloride;
(5-(2,6-diisopropylphenyl)-2-(2,6-diisopropylphenyl)-2,3-dihydroimidazo[1,5-α]pyridin-3-yl)gold(I) chloride; and
(5-(2,6-diisopropylphenyl)-2-cyclohexyl-2,3-dihydroimidazo[1,5-α]pyridin-3-yl)gold(I) chloride.

47. A method of promoting a reaction which occurs in the presence of the compound of claim 22, wherein the reaction is selected from the group consisting of:
(a) a reaction between a boronic acid and a nitroarene, wherein the method comprises contacting the boronic acid and the nitroarene in the presence of the compound of claim 22and a base, and optionally in the presence of a phase transfer catalyst,
(b) a reaction between an alkyl sulfoxide and an aniline, wherein the method comprises contacting the alkyl sulfoxide and the aniline in the presence of the compound of claim 22 and a base;
(c) a reaction between a thiophenol and an aniline, wherein the method comprises contacting the thiophenol and the aniline in the presence of the compound of claim 22 and a base;
(d) a reaction between an aryl halide and hydrazine, wherein the method comprises contacting the aryl halide and the hydrazine in the presence of the compound of claim 22 and a Lewis acid to provide an aryl hydrazine;
(e) a reaction of an aryl mesylate or aryl tosylate with a boronic acid, wherein the method comprises contacting the aryl mesylate or aryl tosylate and the boronic acid in the presence of the compound of claim 22 and a base;
(f) a reaction of an aryl mesylate or tosylate with an aniline, wherein the method comprises contacting the aryl mesylate or aryl tosylate and the aniline in the presence of the compound of claim 22 and a base;
(g) a reaction between an aryl sulfamate and a boronic acid, wherein the method comprises contacting the aryl sulfamate and the boronic acid in the presence of the compound of claim 22 and a base;
(h) a reaction between an aryl sulfamate and an aniline, wherein the method comprises contacting the aryl sulfamate and the aniline in the presence of the compound of claim 22 and a base;
(i) a reaction between an aryl halide and a hydroxide salt, wherein the method comprises contacting the aryl halide and the hydroxide salt in the presence of the compound of claim 22;
(i) a reaction between an aryl methyl thioether and an aniline, wherein the method comprises contacting the aryl methyl thioether and the aniline in the presence of the compound of claim 22 and a base;
(k) a reaction between an aryl sulfoxide and an aniline, wherein the method comprises contacting the aryl sulfoxide and the aniline in the presence of the compound of claim 22 and a base;
(l) a reaction between an amine and an aryl chloride, wherein the method comprises contacting the amine and the aryl chloride in the presence of the compound of claim 22 and a base;
(m) a reaction between an aryl chloride and a boronic acid, wherein the method comprises contacting the aryl chloride and the boronic acid in the presence of the compound of claim 22 and a base;
(n) a reaction between an aryl fluoride and an aniline, wherein the method comprises contacting the aryl fluoride and the aniline in the presence of the compound of claim 22 and a base; and
(o) a reaction between an alkyne and a nitroarene, wherein the method comprises contacting the alkyne and the nitroarene in the presence of the compound of claim 22 and a reducing agent, wherein the contacting optionally further occurs in the presence of a Lewis acid;
optionally wherein at least one of the following applies:
(i) the boronic acid is a C₆-C₁₀ aryl or C₂-C₈ heteroaryl, wherein the C₆-C₁₀ aryl or C₂-C₈ heteroaryl is substituted with at least one boronate or boronic acid moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(ii) the nitroarene is a C₆-C₁₀ aryl or C₂-C₈ heteroaryl, wherein the C₆-C₁₀ aryl or C₂-C₈ heteroaryl is substituted with at least one NO₂ moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(iii) the alkyl sulfoxide is a C₆-C₁₀ aryl or C₂-C₈ heteroaryl, wherein the C₆-C₁₀ aryl or C₂-C₈ heteroaryl is substituted with at least one S(═O)(optionally substituted C₁-C₆ alkyl) moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(iv) the aniline is a C₆-C₁₀ aryl or C₂-C₈ heteroaryl, wherein the C₆-C₁₀ aryl or C₂-C₈ heteroaryl is substituted with at least one NH₂ moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(v) the thiophenol is a C₆-C₁₀ aryl or C₂-C₈ heteroaryl, wherein the C₆-C₁₀ aryl or C₂-C₈ heteroaryl is substituted with at least one thiol moiety and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(vi) the aryl halide is a C₆-C₁₀ aryl or C₂-C₈ heteroaryl, wherein the C₆-C₁₀ aryl or C₂-C₈ heteroaryl is substituted with at least one halogen selected from the group consisting of C₁, Br, and I, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(vii) the aryl tosylate is a C₆-C₁₀ aryl or C₂-C₈ heteroaryl, wherein the C₆-C₁₀ aryl or C₂-C₈ heteroaryl is substituted with at least one toluenesulfonoxy moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(viii) the aryl mesylate is a C₆-C₁₀ aryl or C₂-C₈ heteroaryl, wherein the C₆-C₁₀ aryl or C₂-C₈ heteroaryl is substituted with at least one methanesulfonoxy moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(ix) the aryl sulfamate is a C₆-C₁₀ aryl or C₂-C₈ heteroaryl, wherein the C₆-C₁₀ aryl or C₂-C₈ heteroaryl is substituted with at least one sulfamate moiety, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(x) the aryl chloride is a C₆-C₁₀ aryl or C₂-C₈ heteroaryl, wherein the C₆-C₁₀ aryl or C₂-C₈ heteroaryl is substituted with at least one C₁, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(xi) the aryl methyl thioether is a C₆-C₁₀ aryl or C₂-C₈ heteroaryl, wherein the C₆-C₁₀ aryl or C₂-C₈ heteroaryl is substituted with at least one methylthio moiety (i.e., —SCH₃), and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(xii) the aryl sulfoxide is C₆-C₁₀ aryl or C₂-C₈ heteroaryl, wherein the C₆-C₁₀ aryl or C₂-C₈ heteroaryl is substituted with at least one substituent selected from the group consisting of S(═O)(optionally substituted C₁-C₆ alkyl), S(═O)(optionally substituted C₆-C₁₀ aryl), and S(═O)(optionally substituted C₂-C₈ heteroaryl), and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂;
(xiii) the amine is selected from the group consisting of a N (optionally substituted C₁-C₆ alkyl)₂, N (optionally substituted C₁-C₆ alkyl)(optionally substituted C₆-C₁₀ aryl), N (optionally substituted C₆-C₁₀ aryl)₂, and optionally substituted C₂-C₈ heterocycloalkyl, wherein the heterocycloalkyl comprises at least one secondary amine;
(xiv) the aryl bromide is a C₆-C₁₀ aryl or C₂-C₈ heteroaryl, wherein the C₆-C₁₀ aryl or C₂-C₈ heteroaryl is substituted with at least one Br, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂; and
(xv) the aryl fluoride is a C₆-C₁₀ aryl or C₂-C₈ heteroaryl, wherein the C₆-C₁₀ aryl or C₂-C₈ heteroaryl is substituted with at least one F, and further optionally substituted with at least one substituent selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

48-64. (canceled)

65. A method of preparing a compound of formula (IV): 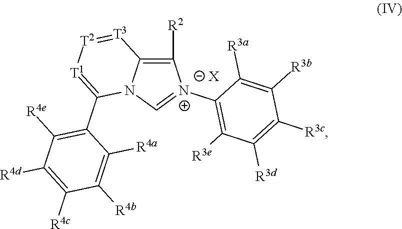
wherein:
T¹ is N or CR^{1a};
T² is N or CR^{1b};
T³ is N or CR^{1c};
R^{1a}, R^{1b}, and R^{1c} are each independently selected from the group consisting of H, halogen, C₁-C₃ haloalkyl, C₁-C₃ alkoxy, C₁-C₃ haloalkoxy, CN, NO₂, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
wherein each optional substituent in each of R^{1a}, R^{1b}, and R^{1c} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
wherein two vicinal substituents selected from the group consisting of R^{1a}, R^{1b}, and R^{1c} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R² is selected from the group consisting of H, C₁-C₆ alkyl, C₆-C₁₀ aryl, and C₂-C₁₂ heterocyclyl;
R^{3a}, R^{3b}, R^{3c}, R^{3d}, and R^{3e} are each independently selected from the group consisting of H, halogen, optionally substituted C₁-C₆ alkyl and optionally substituted C₁-C₆ alkoxy,
wherein each optional substituent in each of R^{3a}, R^{3b}, R^{3c}, R^{3d}, and R^{3e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{4a}, R^{4b}, R^{4c}, R^{4d}, and R^{4e} are each independently selected from the group consisting of H, halogen, N (optionally substituted C₁-C₆ alkyl)₂, optionally substituted C₁-C₆ alkyl and optionally substituted C₁-C₆ alkoxy,
wherein each optional substituent in each of R^{4a}, R^{4b}, R^{4c}, R^{4d}, and R^{4e} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; and
X is a counter ion;
the method comprising contacting (Z): 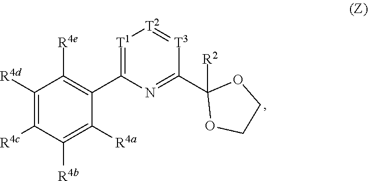
and (Y): 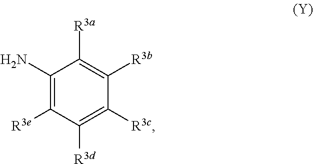
in the presence of paraformaldehyde and an acid,
optionally wherein the (Z) is prepared by reacting (X): 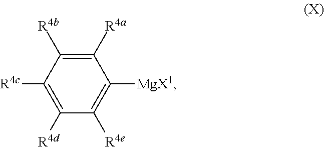
and (W) 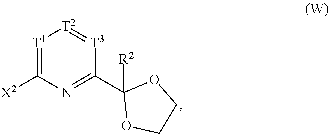
in the presence of a transition metal catalyst;
wherein X¹ and X² are each independently selected from the group consisting of Cl, Br, and I.

66. (canceled)
